(19) United States
(12) Patent Application Publication
(10) Pub. No.: US 2026/0124303 A1
(43) Pub. Date: May 7, 2026

(54) FIBROBLAST ACTIVATION PROTEIN (FAP) CAR-INVARIANT NATURAL KILLER T CELLS AND USES THEREOF

(71) Applicants: MiNK Therapeutics, Inc., New York, NY (US); Agenus Inc., Lexington, MA (US)

(72) Inventors: Xavier Michelet, Natick, MA (US); Eleni Chantzoura, Longstation (GB); Olivier Le Tonqueze, Belmont, MA (US); Efrat Altman-Sharoni, Cambridge (GB); Martyna Popis, Cambridge (GB); Magdalena Niedzielska, Cambridge (GB); Marc van Dijk, Bosch en Duin (NL); Dhan Chand, Lexington, MA (US); Olga Ignatovich, Lexington, MA (US); Vignesh Venkatraman, Lexington, MA (US); Paul lbbett, Cambridge (GB)

(73) Assignees: MiNK Therapeutics, Inc., New York, NY (US); Agenus Inc., Lexington, MA (US)

(21) Appl. No.: 19/118,248

(22) PCT Filed: Oct. 4, 2023
(86) PCT No.: PCT/US2023/076011

§ 371 Date: Apr. 3, 2025

Related U.S. Application Data
(60) Provisional application No. 63/413,236, filed on Oct. 4, 2022.

(51) Int. Cl. A61K 40/15; A61K 38/20; A61K 40/11; A61K 40/31; A61K 40/42; A61K 40/50; A61P 35/00; C07K 14/725; C07K 16/40

(52) U.S. Cl. CPC A61K 40/15; A61K 38/2086; A61K 40/11; A61K 40/31; A61K 40/4249; A61K 40/50; A61P 35/00; C07K 14/7051; C07K 16/40

(57) ABSTRACT

The present disclosure, at least in part, is based on the discovery of novel anti-Fibroblast activation protein (FAP) antibodies or antigen binding fragments thereof, and genetically modified cells (e.g., iNKT cells) expressing chimeric antigen receptors comprising the anti-FAP antibody or antigen binding fragment thereof demonstrate improved properties, including increased binding to FAP, killing of FAP-expressing cancer cells in vitro and in vivo; and enhanced persistent in a subject receiving the therapy.

### RELATED APPLICATIONS

This application claims the benefit under 35 U.S.C. 119(e) of the filing date of U.S. provisional Application Ser. No. 63/413,236, filed Oct. 4, 2022, the entire contents of which are incorporated by reference herein.

### REFERENCE TO AN ELECTRONIC SEQUENCE LISTING

The contents of the electronic sequence listing (A132770004WO00-SEQ-LGE.xml; Size: 179,363 bytes; and Date of Creation: Oct. 4, 2023) is herein incorporated by reference in its entirety.

### BACKGROUND

Fibroblast activation protein (FAP) is a cell surface protein high expressed on stroma cells (e.g., cancer-associated fibroblasts (CAFs) in the tumor microenvironment (TME). FAP can modulate the TME by remodeling the extracellular matrix (ECM), and its overexpression on CAFs is associated with poor prognosis in various cancers.

### SUMMARY

The present disclosure, at least in part, is based on the discovery that genetically modified cells (e.g., iNKT cells) expressing chimeric antigen receptors targeting fibroblast activation protein (FAP) are capable of killing FAP expressing cells (e.g., cancer-associated fibroblasts (CAFs)) in the tumor microenvironment (TME). In some embodiments, the anti-FAP CAR iNKT cell is engineered to express an armoring molecule (e.g., soluble IL-15). In some embodiments, the anti-FAP CAR iNKT cells kill FAP expressing cells (e.g., FAP expressing tumor cells and/or FAP expressing cancer-associated fibroblasts (CAFs)). In some embodiments, the anti-FAP CAR iNKT cells reduce the immunosuppression in the TME, thereby increasing the efficacy of other cancer therapy (e.g., CAR T cell targeting tumor antigen) In some embodiments, anti-FAP CAR iNKT cells described by the disclosure demonstrate improved properties relative to existing FAP-CAR cell therapy in the art, including but not limited to killing of FAP-expressing cells in vitro and in vivo; and enhanced persistence in a subject receiving the therapy.

In some aspects, the present disclosure provides an invariant natural killer T (iNKT) cell comprising a chimeric antigen receptor (CAR) that specifically binds fibroblast activation protein (FAP). In some embodiments, the chimeric antigen receptor of the iNKT cells comprises a CDRH1 having the amino acid sequence of SEQ ID NO: 1, a CDRH2 having the amino acid sequence of SEQ ID NO: 2, a CDRH3 having the amino acid sequence of SEQ ID NO: 3, a CDRL1 having the amino acid sequence of SEQ ID NO: 4, a CDRL2 having the amino acid sequence of SEQ ID NO: 5, and a CDRL3 having the amino acid sequence of SEQ ID NO: 6. In some embodiments, the chimeric antigen receptor of the iNKT cells comprises a CDRH1 having the amino acid sequence of SEQ ID NO: 10, a CDRH2 having the amino acid sequence of SEQ ID NO: 11, a CDRH3 having the amino acid sequence of SEQ ID NO: 12, a CDRL1 having the amino acid sequence of SEQ ID NO: 4, a CDRL2 having the amino acid sequence of SEQ ID NO: 5, and a CDRL3 having the amino acid sequence of SEQ ID NO: 13. In some embodiments, the chimeric antigen receptor of the iNKT cells comprises a CDRH1 having the amino acid sequence of SEQ ID NO: 40, a CDRH2 having the amino acid sequence of SEQ ID NO: 41, a CDRH3 having the amino acid sequence of SEQ ID NO: 42, a CDRL1 having the amino acid sequence of SEQ ID NO: 4, a CDRL2 having the amino acid sequence of SEQ ID NO: 5, and a CDRL3 having the amino acid sequence of SEQ ID NO: 43. In some embodiments, the chimeric antigen receptor of the iNKT cells comprises a CDRH1 having the amino acid sequence of SEQ ID NO: 10, a CDRH2 having the amino acid sequence of SEQ ID NO: 11, a CDRH3 having the amino acid sequence of SEQ ID NO: 42, a CDRL1 having the amino acid sequence of SEQ ID NO: 4, a CDRL2 having the amino acid sequence of SEQ ID NO: 5, and a CDRL3 having the amino acid sequence of SEQ ID NO: 43. In some embodiments, the chimeric antigen receptor of the iNKT cells comprises a CDRH1 having the amino acid sequence of SEQ ID NO: 10, a CDRH2 having the amino acid sequence of SEQ ID NO: 11, a CDRH3 having the amino acid sequence of SEQ ID NO: 54, a CDRL1 having the amino acid sequence of SEQ ID NO: 4, a CDRL2 having the amino acid sequence of SEQ ID NO: 5, and a CDRL3 having the amino acid sequence of SEQ ID NO: 55.

In some embodiments, the chimeric antigen receptor of the iNKT cells comprises a heavy chain variable region (VH) comprising the amino acid sequence of SEQ ID NO: 7 and a light chain variable region (VL) comprising the amino acid sequence of SEQ ID NO: 8. In some embodiments, the chimeric antigen receptor of the iNKT cells comprises a VH comprising the amino acid sequence of SEQ ID NO: 14 and a VL comprising the amino acid sequence of SEQ ID NO: 15. In some embodiments, the chimeric antigen receptor of the iNKT cells comprises a VH comprising the amino acid sequence of SEQ ID NO: 44 and a VL comprising the amino acid sequence of SEQ ID NO: 45. In some embodiments, the chimeric antigen receptor of the iNKT cells comprises a VH comprising the amino acid sequence of SEQ ID NO: 47 and a VL comprising the amino acid sequence of SEQ ID NO: 48. In some embodiments, the chimeric antigen receptor of the iNKT cells comprises a VH comprising the amino acid sequence of SEQ ID NO: 56 and a VL comprising the amino acid sequence of SEQ ID NO: 57.

In some embodiments, the chimeric antigen receptor of the iNKT cells comprises a scFv comprising the amino acid sequence of SEQ ID NO: 9. In some embodiments, the chimeric antigen receptor of the iNKT cells comprises a scFv comprising the amino acid sequence of SEQ ID NO: 16. In some embodiments, the chimeric antigen receptor of the iNKT cells comprises a scFv comprising the amino acid sequence of SEQ ID NO: 46. In some embodiments, the chimeric antigen receptor of the iNKT cells comprises a scFv comprising the amino acid sequence of SEQ ID NO: 49. In some embodiments, the chimeric antigen receptor of the iNKT cells comprises a scFv comprising the amino acid sequence of SEQ ID NO: 58.

In some embodiments, an iNKT cell comprises a CAR that further comprises a hinge region. In some embodiments, the hinge region comprises an amino acid sequence at least 80% identical to SEQ ID NO: 30. In some embodiments, an iNKT cell comprises a CAR that further comprises a transmembrane domain. In some embodiments, the transmembrane domain comprises an amino acid sequence at least 80% identical to SEQ ID NO: 31. In some embodiments, an iNKT cell comprises a CAR that further comprises a hinge/transmembrane domain comprising the amino acid sequence of any one of SEQ ID NOs: 83, 85, or 87. In some embodiments, an iNKT cell comprises a CAR that further comprises one or more cytoplasmic domain. In some embodiments, the one or more cytoplasmic domain comprises the amino acid sequence of any one of SEQ ID NO: 32, 33, 39, 90, 92, 94, 96, 98, 100, 102, or 105. In some embodiments, the cytoplasmic domain comprises an amino acid sequence at least 80% identical to SEQ ID NO: 32. In some embodiments, an iNKT cell comprises a CAR that further comprises an intracellular co-stimulatory domain. In some embodiments, the co-stimulatory domain comprises an amino acid sequence at least 80% identical to SEQ ID NO: 33. In some embodiments, the CAR comprises the amino acid sequence of any one of SEQ ID NOs: 17, 18, 59, 60, 61, or 62.

In some embodiments, an anti-FAP CAR iNKT cell is engineered to express one or more immunoregulatory gene products (i.e., armoring molecule). In some embodiments, the one or more immunoregulatory gene products comprises IL-15, IL-12, CD40L, or 4-1BB, IL-18, or IL-21. In some embodiments, the immunoregulatory gene product is a soluble IL-15 (sIL-15).

In some embodiments, the CAR binds to FAP with a KD in the range of 1E-10 M and 10E-7 M. In some embodiments, the CAR binds to FAP with a KD in the range of 1E-8 M and 3E-8 M.

In some aspects, the present disclosure provides an invariant natural killer T (iNKT) cell comprising a chimeric antigen receptor (CAR) that specifically binds fibroblast activation protein (FAP), wherein the iNKT cell expresses IL-15 (e.g., soluble IL-15). In some embodiments, an anti-FAP CAR iNKT cell comprises a CAR comprising an antigen binding moiety that specifically binds to FAP derived from the antigen binding moiety of any one of the anti-FAP antibodies set forth in Tables 2 and 3.

In some aspects, the present disclosure provides a composition comprising an FAP CAR iNKT cell described herein, and a pharmaceutically acceptable carrier.

In some aspects, the present disclosure provides a method for killing a cell, the method comprising contacting the cell with an anti-FAP iNKT cell described herein or the composition thereof. In some embodiments, the cell is a cancer cell. In some embodiments, the cancer cell is a lung cancer cell, a breast cancer cell, a colorectal cancer cell, a prostate cancer cell, a stomach cancer cell, a pancreatic cancer cell, a prostate cancer cell, a thyroid cancer cell, a cervical cancer cell, a urothelial cancer cell, or a sarcoma cell. In some embodiments, the lung cancer is non-small cell lung cancer. In some embodiments, the cell in the tumor microenvironment express FAP.

In some aspects, the present disclosure provides a method for treating tumor in a subject, the method comprising administering to a subject having or suspected of having cancer an anti-FAP iNKT described herein or a composition thereof.

In some aspects, the present disclosure provides a method for reducing tumor growth, the method comprising contacting the tumor in a subject an anti-FAP iNKT described herein or a composition thereof. In some embodiments, the administration is via injection. In some embodiments, the injection is intraperitoneal injection, intravenous injection, or intratumoral injection. In some embodiments, an anti-FAP CAR iNKT cell is administered in combination with a therapeutic agent. In some embodiments, the therapeutic agent is a CAR T cell specific for a tumor antigen. In some embodiments, the therapeutic agent is an immune checkpoint inhibitor or agonist.

In some aspects, the present disclosure provides a method for treating a tumor in a subject, the method comprising administering the subject a composition comprising an invariant natural killer T (iNKT) cell comprising a chimeric antigen receptor (CAR) having an anti-FAP binding moiety.

In some aspects, the present disclosure provides a method for killing tumor cells, the method comprising contacting the tumor cells with a composition comprising an invariant natural killer T (iNKT) cell comprising a chimeric antigen receptor (CAR) having an anti-FAP binding moiety.

In some aspects, the present disclosure provides method for reducing immunosuppression in the tumor microenvironment (TME) of a subject relative to immunosuppression in the subject prior to the administration, the method comprising administering the subject a composition comprising an invariant natural killer T (iNKT) cell comprising a chimeric antigen receptor (CAR) having an anti-FAP binding moiety.

In some embodiments, the anti-FAP CAR iNKT cells express an armoring molecule. In some embodiments, armoring molecule is soluble IL-15 (sIL-15).

In some embodiments, the tumor is a solid tumor. In some embodiments, the tumor is of epithelial cell origin. In some embodiments, the solid tumor is lung cancer tumor, breast cancer tumor, colorectal cancer tumor, prostate cancer tumor, stomach cancer tumor, pancreatic cancer tumor, prostate cancer tumor, thyroid cancer tumor, cervical cancer tumor, urothelial cancer tumor, or sarcoma tumor.

In some embodiments, the anti-FAP CAR iNKT cells kill FAP expressing cells in the TME. In some embodiments, the FAP expressing cells in the TME comprise tumor cells expressing FAP, and/or cancer-associated fibroblasts. In some embodiments, the anti-FAP CAR iNKT cells kill FAP expressing cells directly. In some embodiments, the anti-FAP CAR iNKT cells kill FAP expressing cells indirectly. In some embodiments, the killing of cancer-associated fibroblasts results in reduced immunosuppression in the TME relative to immunosuppression in the subject prior to the administration. In some embodiments, the killing of cancer-associated fibroblasts results in increased immune cell infiltration in the TME relative to immune cell infiltration in the subject prior to the administration.

In some embodiments, the method further comprises administering the subject a CAR T cell targeting a tumor antigen comprising NY-ESO-1, or BCMA.

In some embodiments, the administration of the anti-FAP CAR iNKT cells leads to reduced tumor burden relative to tumor burden in the subject prior to the administration.

In some embodiments, the administration of the anti-FAP CAR iNKT cells leads to resistance to T cell exhaustion, enhancement of tissue homing of anti-FAP iNKT cells, selective cytotoxicity towards M2 macrophages, and/or stimulation of dendritic cell maturation. In some embodiments, the anti-FAP CAR iNKT cells retain response to CD1d and/or NK receptor ligand.

In some embodiments, the anti-FAP CAR iNKT cells comprise a CAR comprising any one of the FAP binding moieties set forth in table 2 and Table 3.

In some embodiments, the anti-FAP CAR iNKT cells comprise a CAR comprising any one of the FAP antibodies set forth in Table 2.

In some embodiments, the subject is a human. In some embodiments, the subject is diagnosed as having a solid tumor comprising lung cancer, breast cancer, colorectal cancer, prostate cancer, stomach cancer, pancreatic cancer, prostate cancer, thyroid cancer, cervical cancer, urothelial cancer, or sarcoma.

In some aspects, the present disclosure provides an antibody or antigen binding fragment that specifically binds to an amino acid sequence having at least 85% identity to SEQ ID NOs: 35-38. In some embodiments, the antibody specifically binds an amino acid sequence set forth as: SEQ ID NOs: 35-38.

In some aspects, the present disclosure provides an antibody or antigen binding fragment that comprises a heavy chain variable region having the sequence set forth as in SEQ ID NOs: 7 or 14.

In some embodiments, the antibody comprises a heavy chain variable region having the sequence set forth as: SEQ ID NOs: 7 or 14 and a light chain variable region having the sequence set forth as: SEQ ID NO: 8 or 15.

In some embodiments, wherein the antibody comprises: (i) heavy chain variable region having the sequence set forth in SEQ ID NO: 7 and/or the light chain variable region having the sequence set forth in SEQ ID NO: 8; or (ii) heavy chain variable region having the sequence set forth in SEQ ID NO: 14 and/or the light chain variable region having the sequence set forth in SEQ ID NO: 15.

In some aspects, the present disclosure provides an antibody or antigen binding fragment that comprises a variable heavy chain region comprising a complementarity determining region 3 (CDRH3) having the sequence set forth in SEQ ID NOs: 3 or 12.

In some embodiments, the antibody or antigen binding fragment further comprising a variable light chain region comprising a complementarity determining region 3 (CDRL3) having the sequence set forth in SEQ ID NOs: 6 or 13.

In some aspects, the present disclosure provides ab antibody or antigen binding fragment that comprises six complementarity determining regions (CDRs): CDRH1, CDRH2, CDRH3, CDRL1, CDRL2, and CDRL3, wherein (i) CDRH1 comprises the sequence as set forth in SEQ ID NO: 1. CDRH2 comprises the sequence as set forth in SEQ ID NO: 2, CDRH3 comprises the sequence as set forth in SEQ ID NO: 3, CDRL1 comprises the sequence as set forth in SEQ ID NO: 4, CDRL2 comprises the sequence as set forth in SEQ ID NO: 5, and CDRL3 comprises the sequence as set forth in SEQ ID NO: 6; or (ii) CDRH1 comprises the sequence as set forth in SEQ ID NO: 10, CDRH2 comprises the sequence as set forth in SEQ ID NO: 11, CDRH3 comprises the sequence as set forth in SEQ ID NO: 12, CDRL1 comprises the sequence as set forth in SEQ ID NO: 4, CDRL2 comprises the sequence as set forth in SEQ ID NO: 5, and CDRL3 comprises the sequence as set forth in SEQ ID NO: 13.

In some embodiments, the antibody or antigen binding fragment is chimeric. In some embodiments, wherein the antibody or antigen binding fragment is humanized.

In some embodiments, the antibody or antigen binding fragment is a single-chain variable fragment (scFv). In some embodiments, the scFv comprises the amino acid sequence set forth in SEQ ID NOs: 9 or 16.

In some aspects, the present disclosure provides an isolated nucleic acid encoding the antibody or antigen binding fragment described herein.

In some embodiments, the nucleic acid sequence encoding an anti-FAP scFv is set forth in SEQ ID NOs: 21 or 26. In some embodiments, the nucleic acid sequence encoding an anti-FAP CAR is set forth in SEQ ID NOs: 22 or 27.

In some aspects, the present disclosure provides a vector comprising the isolated nucleic acid described herein. In some embodiments, the vector is a viral vector. In some embodiments, the viral vector is a lentiviral vector. In some embodiments, the lentiviral vector comprises the sequence set forth in SEQ ID NOs: 23 or 28.

In some aspects, the present disclosure provides a host cell comprising the antibody or antigen binding fragment, the isolated nucleic acid, or the vector described herein. In some embodiments, the cell is a mammalian cell, bacterial cell, yeast cell, or insect cell. In some embodiments, the cell is a hybridoma cell.

In some aspects, the present disclosure provides a chimeric antigen receptor (CAR) comprising the antigen binding fragment described herein. In some embodiments, the CAR further comprises a hinge region. In some embodiments, the hinge region comprises an amino acid sequence at least 80% identical to SEQ ID NO: 30.

In some embodiments, the CAR further comprises a transmembrane domain. In some embodiments, the transmembrane domain comprises an amino acid sequence at least 80% identical to SEQ ID NO: 31.

In some embodiments, the CAR further comprises a cytoplasmic domain. In some embodiments, the cytoplasmic domain comprises an amino acid sequence at least 80% identical to SEQ ID NO: 32.

In some embodiments, the CAR further comprises a co-stimulatory domain. In some embodiments, the co-stimulatory domain comprises an amino acid sequence at least 80% identical to SEQ ID NO: 33.

In some embodiments, the CAR comprises the amino acid sequence of SEQ ID NOs: 17 or 18.

In some aspects, the present disclosure comprises an immune cell comprising the CAR of described herein.

In some embodiments, the immune cell is a natural killer (NK) cell or a T cell. In some embodiments, the immune cell is an invariant natural killer T (iNKT) cell.

In some embodiments, the immune cell is engineered to express one or more immunoregulatory gene products. In some embodiments, the one or more immunoregulatory gene products comprises IL-15, IL-12, CD40L, or 4-1BB, IL-18, or IL-21.

In some aspects, the present disclosure provides a pharmaceutical composition comprising the antibody or antigen binding fragment or the immune cell described herein, and a pharmaceutically acceptable excipient.

In some aspects, the present disclosure provides a method for killing a cell, the method comprising contacting the cell with the antibody or antigen binding fragment, the immune cell, or the pharmaceutical composition described herein.

In some embodiments, the cell is a mammalian cell. In some embodiments, the cell is a human cell. In some embodiments, the cell is a cancer cell. In some embodiments, the cancer is a solid tumor. In some embodiments, the solid tumor is lung cancer, breast cancer, colorectal cancer, prostate cancer, stomach cancer, pancreatic cancer, prostate cancer, thyroid cancer, cervical cancer, urothelial cancer, or sarcoma. In some embodiments, the lung cancer is non-small cell lung cancer.

In some aspects, the present disclosure provides a method for treating tumor in a subject, the method comprising administering to a subject having or suspected of having cancer, the immune cell, or the pharmaceutical composition described herein.

In some aspects, the present disclosure provides a method for reducing tumor growth, the method comprising contacting the tumor in a subject with the antibody or antigen binding fragment, the immune cell, or the pharmaceutical composition described herein.

In certain embodiments, the subject does not undergo lymphodepletion prior to, or concurrent with, treatment with an anti-FAP CAR iNKT cell or pharmaceutical composition as disclosed herein. Lymphodepletion is frequently performed prior to immunotherapies, such as CAR T therapies, for instance, to debulk a tumor, alter tumor phenotype, modify the tumor microenvironment, remove cytokine sinks (e.g., make IL-2, IL-7, and IL-15 more available) and suppress the host immune system. However, lymphodepletion has multiple negative effects including, neutropenia, anemia, thrombocytopenia, and immunosuppression, and toxicities associated with lymphodepletion agents such as fludarabine and cyclophosphamide. In certain embodiments of the invention, the subject does not receive treatment with, fludarabine, or cyclophosphamide prior to, or concurrent with, administration of the anti-FAP CAR iNKT cells or a pharmaceutical composition as disclosed herein.

In some embodiments, the subject is a human. In some embodiments, the tumor is a solid tumor. In some embodiments, the solid tumor is lung cancer tumor, breast cancer tumor, colorectal cancer tumor r, prostate cancer tumor, stomach cancer tumor, pancreatic cancer tumor, prostate cancer tumor, thyroid cancer tumor, cervical cancer tumor, urothelial cancer tumor, or sarcoma. In some embodiments, the lung cancer is non-small cell lung cancer.

In some embodiments, the administration is via injection. In some embodiments, the injection is intraperitoneal, intravenous, intratumoral.

In some embodiments, the immune cell is administered in combination with a therapeutic agent. In some embodiments, the therapeutic agent is a CAR T cell specific for a tumor antigen. In some embodiments, the therapeutic agent is an immune checkpoint inhibitor or agonist.

### BRIEF DESCRIPTION OF THE DRAWINGS

The accompanying drawings, which are incorporated in and constitute a part of this specification, illustrate certain embodiments, and together with the written description, serve to provide non-limiting examples of certain aspects of the compositions and methods disclosed herein.

FIG. 1 shows an exemplary format for an anti-FAP CAR iNKT cell.

FIG. 2 shows a process for assessing a therapeutic window for targeting FAP positive cells (e.g., cancer-associated fibroblasts (CAFs)) in the tumor microenvironment. FAP expression in different types of cancer associated fibroblast. Quantitation of FAP molecule expression on the surface of different cell types was done using the Quantibrite™ approach. CAFs from 8 dissociated tumor samples (DTCs) of colorectal cancer (CRC) patients were assessed. FAP expression in CAF-S1 subpopulation was >10 fold higher than in the CAF-S3 subpopulation. Similarly, expression of FAP in CAF-S1 samples was 10-fold higher than in normal lung fibroblasts (NLF) post thawing. Passaging of NLF (P0, P3) led to an increase in the FAP expression to similar levels to CAFs. Different FAP expressing tumor cell lines from multiple passages exhibited a range of FAP expression between levels observed for Primary healthy fibroblasts to CAF S3. In the case of U138 cells, there was variability based on the passage. For discovery purposes we transfected T2 cells with low or high amount of FAP (T2low and T2high respectively). Finally, for the in vivo studies, A375 cells expressing FAP at the same levels as T2high were generated.

FIGS. 3A-3N are graphs showing the generation of anti-FAP CARs capable of binding to human and mouse FAP, and killing FAP expressing cells. FIG. 3A shows a process of identifying a panel of anti-FAP candidate scFvs. FIG. 3B shows an exemplary workflow of the transient anti-FAP iNKT assays. FIG. 3C shows expression level of FAP in target T2 cells electroporated with either 0.3 μg or 10 μg of either human or mouse FAP. FIG. 3D shows binding of human or mouse FAP of a pool of exemplary anti-FAP scFvs. FIG. 3E shows target cell killing of a pool of exemplary anti-FAP CAR iNKT cells. FIGS. 3F & G shows binding of a different pool of candidate anti-FAP CARs candidates expressed in 2 different donors respectively. FIG. 3F shows iNKT cells derived from donor 1 in binding human FAP. FIG. 3G shows a different pool of candidate anti-FAP CARs iNKT cells derived from donor 2 in binding human FAP. FIGS. 3H-3I show target cell killing at E:T ratio 1:1 (FIG. 3H), or 5:1 (FIG. 3I). All anti-FAP candidate CAR iNKT candidates were able to kill more U87 and HS683 cells compared to mock control. FIGS. 3J-3K show target cell induced iNKT cell activation by measuring the cytokines CD25 and CD69 markers at E:T ratio of 1:1 (FIG. 3J) or 5:1 (FIG. 3K). FIGS. 3L-3M show target cell induced iNKT cell activation by measuring the 4-1bb marker at ET ratio of 1:1 (FIG. 3K) or 5:1 (FIG. 3M). FIG. 3N shows that the candidate selection process identified two distinct groups of anti-FAP-CARs. Left plot. Cytotoxicity of T2 cells expressing high levels of mouse FAP vs high levels of human FAP. Top candidates selected through CARDIS were transiently transfected into iNKT cells. In parallel, T2 cells were transfected with high human or mouse FAP. The following day CAR-iNKT cells were co-cultured with mouse or human T2high in a 5:1 E:T ratio and killing of target cells was assessed 24 hours afterwards. All candidates kill robustly cells expressing high levels of mouse or human FAP. Right plot. Cytotoxicity of T2 cells expressing low levels of mouse vs low levels of human FAP. Transiently transfected CAR-iNKT cells were co-cultured with mouse or human T2low cells. Assessment of the killing of target cells 24 hours afterwards revealed two groups of cells: group 1 retained their ability to robustly kill T2low cells, whereas the second group spared those cells carrying similar levels of FAP to CAF-S3 or normal fibroblasts. Importantly, both groups recognize mouse and human FAP at equivalent levels.

FIGS. 4A-4D show in vitro assays evaluating candidate anti-FAP CAR iNKT cells. FIGS. 4A-4B show that FAP-CAR iNKT candidates respond to FAP+ tumors in vitro (FIG. 4A (donor 1) and FIG. 4B (donor 2)). FIGS. 4C and 4D show that anti-FAP CAR iNKT cells were polyfunctional in response to A-375-FAP tumor cells in vitro (FIG. 4C (donor 1) and FIG. 4D (donor 2)).

FIGS. 5A-5E show FAP-CAR iNKT demonstrating in vivo tumor control toward tumor expressing FAP in the melanoma A-375-FAP+ cancer model. FIG. 5A shows a schematic of in vivo A-375-FAP tumor challenge with FAP-CAR iNKT cells. FIG. 5B shows a curve graph illustrating tumor growth. FIG. 5C shows a curve graph representing tumor growth of individual mice for each group. FIG. 5D shows survival analysis of A-375-FAP tumor bearing mice untreated, treated with MINK FAP-CAR iNKT cells, sibrotuzumab-CAR NKT cells or control BCMA-CAR iNKT cells. FIG. 5E shows weight of tumor outgrowth in each treatment group.

FIGS. 6A-6B show FAP-CAR-IL-15 iNKT cells have the capacity to enhance cytotoxicity of T cells which have undergone repeated antigen stimulation. FIG. 6A shows cytotoxicity of NYESO-1-specific T cells which have undergone multiple rounds of antigen exposure (up to 8 rounds [R #]) against tumor cells which express HLA-A*02:01-SLLMWITQC. FIG. 6B shows cytotoxicity of NYESO-1-specific T cells against tumor cells expressing NYESO-1 which have been exposed with antigen twice (AE2), three times (AE3), or six times (AE6) and co-culture in either media alone or with supernatant from activated FAP-CAR-IL-15 iNKT cells.

FIGS. 7A-7B show E07 FAP-CAR-IL-15 iNKT cells have the capacity to target mouse FAP. FIG. 7A shows cytotoxicity of FAP-CAR-IL-15 iNKT cells to elicit cytotoxicity against MC38 tumor cells engineered to express mouse FAP at different effector-to-target (E:T) ratios. FIG. 7B shows cytotoxicity of FAP-CAR-IL-15 iNKT cells to elicit cytotoxicity against 4T1 tumor cells engineered to express mouse FAP at different effector-to-target (E:T) ratios.

FIGS. 8A-8H show schematic of in vivo NSCLC xenograft model developed to illustrate FAP-CAR-iNKT drug mode of action targeting FAP-expressing cancer associated fibroblasts (CAFs) while tumor cells (A549) do not express FAP. FIG. 8A shows experimental design of NSCLC xenograft tumor model treated with FAP-CAR iNKT cells.

FIG. 8B shows schematic illustrating of the FAP-CAR-IL-15 iNKT cells drug mode of action. FIG. 8C shows the establishment of tumor within the lungs of mice by bioluminescence imaging of NOG mice injected with 2 million A-549 tumor cells expressing HLA-A*02:01-SLLMWITQC and nano luciferase intravenously. Images shown are from treatment-naïve animals, demonstrating robust tumor take in the lungs of mice seven days after tumor inoculation. FIG. 8D shows H&E staining of lungs of mice administered with A-549 tumor cells 14 days post tumor implantation. FIG. 6E left panel shows tumor burden kinetics by bioluminescence imaging of tumor-bearing animals over 21 days. Right panel shows quantification of tumor area by histology over the same 21 days in the lungs of tumor-bearing mice. Both tumor burden quantification by bioluminescent imaging and histological characterization illustrates progressive tumor outgrowth in treatment-naïve conditions. FIG. 8F shows FAP expression as evaluated by immunohistochemistry. Three representative mice are shown for whole-lung (left panels) and representative section (right panel) for FAP expression for weeks 1-3 post-tumor establishment. Normal lungs and controls (no primary and isotype) are shown in the last three right panels. Graphical quantitation of FAP expression is shown below. FIG. 8G shows α-smooth muscle actin (α-SMA), as illustrated by immunohistochemistry at week 1 and week 3 post-tumor establishment. Graphical quantitation of α-SMA expression is shown to the right in which there is a modest, but small, increase in the expression of α-SMA in tumors from weeks 1-3 post-tumor establishment. FIG. 8H shows lungs analyzed by qPCR for murine fap, col11A1, Il6 and Tgfb expression.

FIGS. 9A-9B show FAP-CAR-IL-15 iNKT promote NSCLC tumor control. FIG. 9A shows individual tumor burdens of NOG mice challenged with A-549 tumor cells expressing HLA-A*02:01-SLLMWITQC which have been left untreated or treated with non-specific CAR-IL-15 iNKT cells alone, FAP-CAR-IL-15 iNKT cells alone, T cells expressing NYESO-1 TCR, or FAP-CAR-IL-15 iNKT and T cells expressing NYESO TCR. FIG. 9B shows survival analysis of mice treated under conditions described in FIG. 9A.

FIGS. 10A-10E show mice treated with MINK FAP-CAR-IL-15 demonstrating reduced tumor burdens in the lung tissue following T cells or FAP-CAR-IL-15 plus T cells. FIG. 10A shows bioluminescence imaging of mice treated with FAP-CAR-IL-15 iNKT+T cells (left panel) and T cells only (right panel) at day 18 and day 25. FIG. 10B shows percent change of tumor burden from day 3 to day 24 post tumor injection in mice treated with either T cells only or FAP-CAR-IL-15 iNKT cells+T cells. FIG. 10C shows H&E staining of lungs of mice treated with FAP-CAR-IL-15 iNKT+T cells or T cells harvested at day 25 (left panel). Right panel shows quantification of the tumoral surface area of the lungs from mice treated with FAP-CAR-IL-15 iNKT cells+T cells or T cells only. FIG. 10D shows immunohistochemistry staining of FAP in lungs of mice treated with T cells or FAP-CAR-IL-15+T cells harvested at day 25 (left panel). Right panel shows quantification of the FAP expression of the lungs from mice treated with FAP-CAR-IL-15 iNKT+T cells or T cells only. FIG. 10E shows immunohistochemistry staining of α-SMA in lungs of mice treated with T cells or FAP-CAR-IL-15+T cells harvested at day 25 (left panel). Right panel shows quantification of the α-SMA expression of the lungs from mice treated with FAP-CAR-IL-15 iNKT+T cells or T cells only.

FIGS. 11A-11E show that animals treated with FAP-CAR-IL-15 iNKT showed higher levels of immune cells activation and tumor tissue infiltration. FIG. 11A shows measurement of IFN-g in the serum of mice treated with FAP-CAR-IL-15 iNKT+T cells or T cells only. FIG. 11B shows quantification of the number of iNKT cells detected by flow cytometry in lung tissues from groups FAP-CAR-IL-15 iNKT+T cells and T cells only. FIG. 11C shows quantification of the number of T cells detected by flow cytometry in lung tissues from groups FAP-CAR-IL-15 iNKT+T cells and T cells only. FIG. 11D shows measurement of proliferation of iNKT cells in blood, lung, spleen and bone marrow tissues of animal treated with FAP-CAR-IL-15 iNKT+T cells. FIG. 11E shows measurement of activation and proliferation of T cells in blood, lung, spleen and bone marrow tissues of animal treated with FAP-CAR-IL-15 iNKT+T cells compare to T cells only.

FIGS. 12A-12F show configurations of 2^{nd} generation CAR and cell killing assay testing the same. FIG. 12A shows A modular FAP-CAR iNKT library comprised of 4 different modules was generated according to the structure of a 2nd generation CAR (scFv, TM/Hinge region, 1st ICD region and 2nd ICD region). Each region had the following number of possible domains: scFv (3), TM/hinge (3), 1st ICD region (24), 2nd ICD region (24) to give a library with a total possible diversity of 5,184 combinations. This library was packaged as lentivirus and transduced into iNKT cells and FAP-CAR+ iNKTs were enriched by sorting on day 17 post transduction. FIG. 12B shows that, on day 21 post transduction, iNKT cells were co-cultured with FAP expressing antigen presenting cells to specifically activate FAP-CAR iNKTs and enrich for ICDs having good activation/proliferation. On day 35 post transduction, a further round of co-culture with FAP expressing antigen presenting cells was performed for a further 14 days, giving over 15000 enrichment over the 4 weeks of co-culture. FIG. 12C shows results of nanopore sequencing of DNA recovered from the initial plasmid library and DNA extracted from aliquots taken on day 21 post iNKT transduction and day 14 post 1st and 2nd enrichment round. FIGS. 12D-12F are results showing cell killing by iNKT cells of different configuration. iNKT cells were electroporated with mRNA encoding FAP-CAR constructs (or water for mock) with the different domains of the tested CARs listed on the x-axis. Target cells (T2) were electroporated with mRNA encoding FAP at high levels (2 μg mRNA) or low levels (100 ng mRNA) or mock (water). At 24 hrs post electroporation, target cells were labelled with CFSE and were then mixed with iNKT cells at a 5:1 effector:target ratio and co-cultured for 24 hrs. Killing was then assessed by flow cytometry by analyzing the % of CFSE+ cells staining positive with live/dead dye.

FIGS. 13A-13G show Biacore biding kinetics and epitope mapping of the anti-FAP antibodies. FIG. 13A show SPR binding of E07, D01, B08, A07 and D05 to human and mouse FAP. FIG. 13B shows results of epitope binning using ‘Tandem using dual’ method. White-no competition. Yellow-competition. Experiment was run on the Biacore 8K. FIG. 13C shows crystal structure of FAP and its related homolog DPP4. FIG. 13D shows human FAP (hFAP) and DPP4 (hDPP4) chimeric constructs used for epitope mapping. FIGS. 13E-13G show D01, E07 and B08 do not bind to the human DPP4 but bind to human FAP domain from amino acid 141 to 290.

FIG. 14 shows expansion of FAP-CAR-IL15-iNKT cells.

FIG. 15 shows killing of FAP expressing cancer cell by iNKT cells expressing CAR comprising D01 or B08. iNKT cells were transiently transfected with either the D01 or the B08 CAR and co-cultured at 5:1 effector:target ratio with T2 cells transfected to express low or high levels of FAP. Killing of target cells was assessed 24 hours later via flow cytometry.

### DETAILED DESCRIPTION

The present disclosure, at least in part, is based on the discovery that genetically modified immune cells (e.g., iNKT cells) expressing chimeric antigen receptors targeting fibroblast activation protein (FAP) are capable of killing FAP expressing cells (e.g., cancer-associated fibroblasts (CAFs)) in the tumor microenvironment (TME). In some embodiments, the anti-FAP CAR iNKT cell is engineered to express an armoring molecule (e.g., soluble IL-15). In some embodiments, the anti-FAP CAR iNKT cells reduce the immunosuppression in the TME, thereby increasing the efficacy other cancer therapies (e.g., T cell targeting a tumor antigen). In some embodiments, anti-FAP CAR iNKT cells described by the disclosure demonstrate improved properties relative to existing FAP-CAR cell therapy in the art, including killing of FAP-expressing cancer cells in vitro and in vivo; and enhanced persistence in a subject receiving the therapy.

The foregoing and other aspects, implementations, acts, functionalities, features and embodiments of the present teachings can be more fully understood from the following description in conjunction with the accompanying drawings.

### I. Definitions

Additional terms of the present disclosure are defined throughout the specification

Administering: As used herein, the terms “administering” or “administration” means to provide a complex to a subject in a manner that is physiologically and/or pharmacologically useful (e.g., to treat a condition in the subject).

Affinity Matured Antibody: “Affinity Matured Antibody” is used herein to refer to an antibody with one or more alterations in one or more CDRs, which result in an improvement in the affinity (i.e., KD, kd or ka) of the antibody for a target antigen compared to a parent antibody, which does not possess the alteration(s). In some embodiments, affinity matured antibodies will have nanomolar or even picomolar affinities for the target antigen. A variety of procedures for producing affinity matured antibodies are known in the art, including the screening of a combinatory antibody library that has been prepared using bio-display. For example, Marks et al., BioTechnology, 10:779-783 (1992) describes affinity maturation by VH and VL domain shuffling. Random mutagenesis of CDR and/or framework residues is described by Barbas et al., Proc. Nat. Acad. Sci. USA, 91:3809-3813 (1994); Schier et al., Gene, 169:147-155 (1995); Yelton et al., J. Immunol., 155:1994-2004 (1995); Jackson et al., J. Immunol., 154(7): 3310-3319 (1995); and Hawkins et al, J. Mol. Biol., 226:889-896 (1992). Selective mutation at selective mutagenesis positions and at contact or hypermutation positions with an activity-enhancing amino acid residue is described in U.S. Pat. No. 6,914,128 B1.

Antibody: As used herein, the term “antibody” or “antibodies” refers to a polypeptide that includes at least one immunoglobulin variable domain or at least one antigenic determinant, e.g., paratope that specifically binds to an antigen. Examples of antibodies include monoclonal antibodies, recombinantly produced antibodies, monospecific antibodies, multi-specific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, antibody-drug conjugates, single domain antibodies, monovalent antibodies, single chain antibodies or single-chain Fvs (scFv), camelized antibodies, affybodies, Fab fragments, F(ab′)₂ fragments, disulfide-linked Fvs (sdFv), anti-idiotypic (anti-Id) antibodies (including, e.g., anti-anti-Id antibodies), and antigen-binding fragments of any of the above. In some embodiments, an antibody is a full-length antibody. In some embodiments, an antibody is a chimeric antibody. In some embodiments, an antibody is a humanized antibody. However, in some embodiments, an antibody is a Fab fragment, a F(ab′)₂ fragment, a Fv fragment or a scFv fragment. In some embodiments, an antibody is a nanobody derived from a camelid antibody or a nanobody derived from shark antibody. In some embodiments, an antibody is a diabody. In some embodiments, an antibody comprises a framework having germline sequence from a species (e.g., a human germline sequence). In some embodiments, an antibody comprises a heavy (H) chain variable region (abbreviated herein as VH), and/or a light (L) chain variable region (abbreviated herein as VL). In some embodiments, the VH comprises an amino acid sequence that is at least 70%, 75%, 80%, 85%, 90%, 95%, 98%, or at least 99% identical to any of the heavy chain variable domain provided herein. In some embodiments, the VL comprises an amino acid sequence that is at least 70%, 75%, 80%, 85%, 90%, 95%, 98%, or at least 99% identical to any of the light chain variable domain provided herein. In some embodiments, an antibody comprises a constant domain, e.g., an Fc region. An immunoglobulin constant domain refers to a heavy or light chain constant domain. Human IgG heavy chain and light chain constant domain amino acid sequences and their functional variations are known. In another embodiment, an antibody comprises a heavy chain constant domain selected from the group consisting of IgG, IgG1, IgG2, IgG2A, IgG2B, IgG2C, IgG3, IgG4, IgA1, IgA2, IgD, IgM, and IgE constant domains. With respect to the heavy chain, in some embodiments, the heavy chain of an antibody described herein can be an alpha (α), delta (Δ), epsilon (ε), gamma (γ) or mu (μ) heavy chain. In some embodiments, the heavy chain of an antibody described herein can comprise a human alpha (α), delta (Δ), epsilon (ε), gamma (γ) or mu (μ) heavy chain. In a particular embodiment, an antibody described herein comprises a human gamma 1 CH1, CH2, and/or CH3 domain. In some embodiments, the amino acid sequence of the V_{H} domain comprises the amino acid sequence of a human gamma (γ) heavy chain constant region, such as any known in the art. Non-limiting examples of human constant region sequences have been described in the art, e.g., see U.S. Pat. No. 5,693,780 and Kabat E A et al., (1991) supra. In some embodiments, an antibody is modified, e.g., modified via glycosylation, phosphorylation, sumoylation, and/or methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or phosphoglycosylation. In some embodiments, the one or more sugar or carbohydrate molecule are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, or a phospholipid unit.

Approximately: As used herein, the term “approximately” or “about,” as applied to one or more values of interest, refers to a value that is similar to a stated reference value. In certain embodiments, the term “approximately” or “about” refers to a range of values that fall within 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Binding affinity: As used herein, the term “binding affinity” generally refers to the strength of the sum total of non-covalent interactions between a single binding site of a molecule (e.g., an antibody) and its binding partner (e.g., an epitope). Unless indicated otherwise, as used herein, “binding affinity” refers to intrinsic binding affinity which reflects a 1:1 interaction between members of a binding pair. The affinity of a molecule X for its partner Y can generally be represented by the dissociation constant (K_{D}). Affinity can be measured and/or expressed in a number of ways known in the art, including, but not limited to, equilibrium dissociation constant (K_{D}) and equilibrium association constant (K_{A}). The K_{D} is calculated from the quotient of k_{off}/kₒₙ, whereas K_{A} is calculated from the quotient of kₒₙ/k_{off}. kₒₙ refers to the association rate constant of, e.g., an antibody to an epitope, and k_{off} refers to the dissociation rate constant of, e.g., an antibody to an epitope.

Binding affinity (or binding specificity) can be determined by a variety of methods including equilibrium dialysis, equilibrium binding, gel filtration, ELISA, surface plasmon resonance (SPR), florescent activated cell sorting (FACS) or spectroscopy (e.g., using a fluorescence assay). Exemplary conditions for evaluating binding affinity are in HBS-P buffer (10 mM HEPES pH7.4, 150 mM NaCl, 0.005% (v/v) surfactant P20) and PBS buffer (10 mM PO4-3, 137 mM NaCl, and 2.7 mM KCl). These techniques can be used to measure the concentration of bound proteins as a function of target protein concentration. The concentration of bound protein ([Bound]) is generally related to the concentration of free target protein ([Free]) by the following equation:

$$[ Bound ] = [ Free ] / ( Kd + [ Free ] )$$

It is not always necessary to make an exact determination of K_{A}, though, since sometimes it is sufficient to obtain a quantitative measurement of affinity, e.g., determined using a method such as ELISA or FACS analysis, is proportional to K_{A}, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay.

Binding Moiety: As used herein, the term “binding moiety” refers a molecule or part of a molecule that specifically bind or interact with a target molecule via covalent and/or non-coven lent interactions. Binding moieties of the present disclosure include but are not limited to antibodies or antigen-binding fragments thereof (e.g., antibodies, scFv, Fab, Fab′, single chain binding fragment); binding peptides, ligands, receptors, oligonucleotides, small molecules, or aptamers.

CDR: As used herein, the term “CDR” refers to the complementarity determining region within antibody variable sequences. A typical antibody molecule comprises a heavy chain variable region (VH) and a light chain variable region (VL), which are usually involved in antigen binding. The VH and VL regions can be further subdivided into regions of hypervariability, also known as “complementarity determining regions” (“CDR”), interspersed with regions that are more conserved, which are known as “framework regions” (“FR”). Each VH and VL is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the IMGT definition, the Chothia definition, the AbM definition, and/or the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242; IMGT®, the international ImMunoGeneTics information System® imgt.org, Lefranc, M.-P. et al., Nucleic Acids Res., 27:209-212 (1999); Ruiz, M. et al., Nucleic Acids Res., 28:219-221 (2000); Lefranc, M.-P., Nucleic Acids Res., 29:207-209 (2001); Lefranc, M.-P., Nucleic Acids Res., 31:307-310 (2003); Lefranc, M.-P. et al., In Silico Biol., 5, 0006 (2004) [Epub], 5:45-60 (2005); Lefranc, M.-P. et al., Nucleic Acids Res., 33: D593-597 (2005); Lefranc, M.-P. et al., Nucleic Acids Res., 37: D1006-1012 (2009); Lefranc, M.-P. et al., Nucleic Acids Res., 43: D413-422 (2015); Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). See also bioinf.org.uk/abs. As used herein, a CDR may refer to the CDR defined by any method known in the art. Two antibodies having the same CDR means that the two antibodies have the same amino acid sequence of that CDR as determined by the same method, for example, the Kabat definition.

Generally, there are three CDRs in each of the variable regions of the heavy chain and the light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Sub-portions of CDRs may be designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262 (5): 732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems.

The CDRs of an antibody may have different amino acid sequences when different definition systems are used (e.g., the IMGT definition, the Kabat definition, or the Chothia definition). A definition system annotates each amino acid in a given antibody sequence (e.g., VH or VL sequence as listed in Table 2) with a number, and numbers corresponding to the heavy chain and light chain CDRs are provided in Table 1. The CDRs of the anti-FAP antibodies provided herein (e.g., CDRs listed in Table 2) are defined in accordance with the Kabat definition. One skilled in the art is able to derive the CDR sequences using the different numbering systems for the anti-FAP antibodies provided in Table 2.

TABLE 1
CDR Definitions

| | IMGT¹ | Kabat² | Chothia³ |
|---|---|---|---|
| CDRH1 | 27-38 | 31-35 | 26-32 |
| CDRH2 | 56-65 | 50-65 | 53-55 |
| CDRH3 | 105-116/117 | 95-102 | 96-101 |
| CDRL1 | 27-38 | 24-34 | 26-32 |
| CDRL2 | 56-65 | 50-56 | 50-52 |
| CDRL3 | 105-116/117 | 89-97 | 91-96 |

¹IMGT ®, the international ImMunoGeneTics information system ®, imgt.org, Lefranc, M.-P. et al., Nucleic Acids Res., 27: 209-212 (1999)
²Kabat et al. (1991) Sequences of Proteins of Immunological Interest, Fifth Edition, U.S. Department of Health and Human Services, NIH Publication No. 91-3242
³Chothia et al., J. Mol. Biol. 196: 901-917 (1987))

Chimeric antigen receptor: As used herein, a “chimeric antigen receptor” or “CAR” refers to an engineered receptor that binds specificity for an antigen (e.g., FAP) or other ligand or molecule onto an immune effector cell (e.g., a T cell, NK cell, a NKT cell, an iNKT cell). A chimeric antigen receptor typically comprises at least an extracellular ligand-binding domain or moiety capable of specifically binding an antigen and an intracellular domain that comprises one or more signaling domains and/or co-stimulatory domains.

In some embodiments, extracellular ligand-binding domain of a CAR is in the form of a binding protein, small molecule, a peptide, a targeting agent, an agonist, or an antagonist. In some embodiments, the binding protein is an antibody, an antigen-binding fragment of an antibody (e.g., a scFv), a ligand, a cytokine, or a receptor. In some embodiments, the antigen binding fragment is a scFv (e.g., a scFv targeting FAP).

In a particular embodiment, the extracellular ligand-binding domain is in the form of a single-chain variable fragment (scFv) derived from an antibody (e.g., a monoclonal antibody), which provides specificity for a particular epitope or antigen (e.g., an epitope or antigen preferentially present on the surface of a cell, such as a cancer cell or other disease-causing cell or particle).

In some embodiments, the CAR comprises an intracellular signaling domain. Intracellular signaling domains are cytoplasmic domains that transmit an activation signal to the cell following binding of the extracellular domain. An intracellular signaling domain can be any intracellular signaling domain of interest that is known in the art. Such cytoplasmic signaling domains can include, without limitation, CD3 ζ. In some embodiments, the intracellular domain also includes one or more intracellular co-stimulatory domains, such as those described herein, which transmit a co-stimulatory signal which promotes cell proliferation, cell survival, and/or cytokine secretion after binding of the extracellular domain. Such intracellular co-stimulatory domains can include, without limitation, any co-stimulatory domain disclosed herein or those domains known in the art, such as, CD27, CD28, CD8, 4-1BB (CD137), OX40, CD30, CD40, CD127, PD-1, ICOS, lymphocyte function-associated antigen-1 (LFA-1), CD2, CD7, LIGHT, NKG2C, B7-H3, and a ligand that specifically binds with CD83, N1, N6, or any combination thereof. Additional suitable co-stimulatory domains are described in PCT International Application No. PCT/US2017/055133, which is incorporated by reference herein in its entirety. In some embodiments, the co-stimulatory domain is 4-1BB (CD137). In some embodiments, a chimeric antigen receptor further includes additional structural elements, including a transmembrane domain which is attached to the extracellular ligand-binding domain via a hinge or junction sequence. The transmembrane domain can be derived from any membrane-bound or transmembrane protein. For example, the transmembrane polypeptide can be a subunit of the T-cell receptor (i.e., an α, β, γ or ζ, polypeptide constituting CD3 complex), IL2 receptor p55 (α chain), p75 (β chain) or γ chain, subunit chain of Fc receptors (e.g., Fcy receptor III) or CD proteins such as the CD8 alpha chain. Alternatively, the transmembrane domain can be synthetic and can comprise predominantly hydrophobic residues such as leucine and valine. In some embodiments, the CAR comprises a CD8 transmembrane domain. The hinge region refers to any oligo- or polypeptide that functions to link the transmembrane domain to the extracellular ligand-binding domain. For example, a hinge region may comprise up to 300 amino acids, 10 to 100 amino acids and 25 to 50 amino acids. Hinge regions may be derived from all or part of naturally occurring molecules, such as from all or part of the extracellular region of CD8, CD4 or CD28, or from all or part of an antibody constant region. Alternatively, the hinge region may be a synthetic sequence that corresponds to a naturally occurring hinge sequence, or may be an entirely synthetic hinge sequence. In some embodiments, a hinge domain can comprise a part of a human CD8 alpha chain, FcyRllla receptor or IgGl. In some embodiments, the CAR comprises a CD8 hinge region.

CDR-grafted antibody: The term “CDR-grafted antibody” refers to antibodies which comprise heavy and light chain variable region sequences from one species but in which the sequences of one or more of the CDR regions of VH and/or VL are replaced with CDR sequences of another species, such as antibodies having murine heavy and light chain variable regions in which one or more of the murine CDRs (e.g., CDR3) has been replaced with human CDR sequences.

Chimeric antibody: The term “chimeric antibody” refers to antibodies which comprise heavy and light chain variable region sequences from one species and constant region sequences from another species, such as antibodies having murine heavy and light chain variable regions linked to human constant regions.

Complementary: As used herein, the term “complementary” refers to the capacity for precise pairing between two nucleotides or two sets of nucleotides. In particular, complementary is a term that characterizes an extent of hydrogen bond pairing that brings about binding between two nucleotides or two sets of nucleotides. For example, if a base at one position of an oligonucleotide is capable of hydrogen bonding with a base at the corresponding position of a target nucleic acid (e.g., an mRNA), then the bases are considered to be complementary to each other at that position. Base pairings may include both canonical Watson-Crick base pairing and non-Watson-Crick base pairing (e.g., Wobble base pairing and Hoogsteen base pairing). For example, in some embodiments, for complementary base pairings, adenosine-type bases (A) are complementary to thymidine-type bases (T) or uracil-type bases (U), that cytosine-type bases (C) are complementary to guanosine-type bases (G), and that universal bases such as 3-nitropyrrole or 5-nitroindole can hybridize to and are considered complementary to any A, C, U, or T. Inosine (I) has also been considered in the art to be a universal base and is considered complementary to any A, C, U or T.

Conservative amino acid substitution: As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Fourth Edition, Cold Spring Harbor Laboratory Press, Cold Spring Harbor, New York, 2012, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (c) S, T; (f) Q, N; and (g) E, D.

Co-stimulatory domain: As used herein, a “co-stimulatory domain” refers to a polypeptide domain which transmits an intracellular proliferative and/or cell-survival signal upon activation. Activation of a co-stimulatory domain may occur following homodimerization of two co-stimulatory domain polypeptides. Activation may also occur, for example, following activation of a construct comprising the co-stimulatory domain (e.g., a chimeric antigen receptor or an inducible regulatory construct). Generally, a co-stimulatory domain can be derived from a transmembrane co-stimulatory receptor, particularly from an intracellular portion of a co-stimulatory receptor. Non-limiting examples of co-stimulatory polypeptides include, but are not limited to, 4-1BB, CD28, ICOS, OX-40, and CD27 and any other co-stimulatory domain described further herein. In some embodiments, the CAR described herein comprises a 4-1BB co-stimulatory domain.

Co-stimulatory signal: As used herein, a “co-stimulatory signal” refers to an intracellular signal induced by a co-stimulatory domain that promotes cell proliferation, expansion of a cell population in vitro and/or in vivo, promotes cell survival, modulates (e.g., upregulates or downregulates) the secretion of cytokines, and/or modulates the production and/or secretion of other immunomodulatory molecules. In some embodiments, a co-stimulatory signal is induced following homodimerization of two co-stimulatory domain polypeptides. In some embodiments, a co-stimulatory signal is induced following activation of a construct comprising the co-stimulatory domain (e.g. a chimeric antigen receptor or an inducible regulatory construct).

Cross-reactive: As used herein and in the context of a targeting agent (e.g., antibody), the term “cross-reactive,” refers to a property of the agent being capable of specifically binding to more than one antigen of a similar type or class (e.g., antigens of multiple homologs, paralogs, or orthologs) with similar affinity or avidity. For example, in some embodiments, an antibody that is cross-reactive against human and non-human primate antigens of a similar type or class (e.g., a human FAP and non-human primate FAP) is capable of binding to the human antigen and non-human primate antigens with a similar affinity or avidity. In some embodiments, an antibody is cross-reactive against a human antigen and a rodent antigen of a similar type or class. In some embodiments, an antibody is cross-reactive against a rodent antigen and a non-human primate antigen of a similar type or class. In some embodiments, an antibody is cross-reactive against a human antigen, a non-human primate antigen, and a rodent antigen of a similar type or class.

Effective Amount: As used herein, “an effective amount” refers to the amount of each active agent (e.g., iNKT cells with FAP CAR binding moiety) required to confer therapeutic effect on the subject, either alone or in combination with one or more other active agents. In some embodiments, the therapeutic effect includes but is not limited to reducing tumor size, eradicating tumor, or alleviating symptoms associated with tumor.

Epitope: As used herein, an “epitope” is a term in the art and refers to a localized region of an antigen (e.g., a peptide or a peptide-MHC complex) to which an antibody or a chimeric antigen receptor can bind. In certain embodiments, the epitope to which an antibody or a chimeric antigen receptor can be determined by, e.g., NMR spectroscopy, X-ray diffraction crystallography studies, ELISA assays, hydrogen/deuterium exchange coupled with mass spectrometry (e.g., liquid chromatography electrospray mass spectrometry), flow cytometry analysis, mutagenesis mapping (e.g., site-directed mutagenesis mapping), and/or structural modeling. For X-ray crystallography, crystallization may be accomplished using any of the known methods in the art (e.g., Giegé R et al., (1994) Acta Crystallogr D Biol Crystallogr 50 (Pt 4): 339-350; McPherson A (1990) Eur J Biochem 189:1-23; Chayen NE (1997) Structure 5:1269-1274; McPherson A (1976) J Biol Chem 251:6300-6303, each of which is herein incorporated by reference in its entirety). Antibody:antigen crystals may be studied using well-known X-ray diffraction techniques and may be refined using computer software such as X-PLOR (Yale University, 1992, distributed by Molecular Simulations, Inc.; see, e.g., Meth Enzymol (1985) volumes 114 & 115, eds Wyckoff H W et al.,; US20040014194), and BUSTER (Bricogne G (1993) Acta Crystallogr D Biol Crystallogr 49(Pt 1): 37-60; Bricogne G (1997) Meth Enzymol 276A: 361-423, ed Carter C W; Roversi P et al., (2000) Acta Crystallogr D Biol Crystallogr 56 (Pt 10): 1316-1323), each of which is herein incorporated by reference in its entirety. Mutagenesis mapping studies may be accomplished using any method known to one of skill in the art. See, e.g., Champe M et al., (1995) J Biol Chem 270:1388-1394 and Cunningham BC & Wells JA (1989) Science 244:1081-1085, each of which is herein incorporated by reference in its entirety, for a description of mutagenesis techniques, including alanine scanning mutagenesis techniques. In some embodiments, the epitope of an antigen is determined using alanine scanning mutagenesis studies. In some embodiments, the epitope of an antigen is determined using hydrogen/deuterium exchange coupled with mass spectrometry.

Framework: As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, CDR-L2, and CDR-L3 of light chain and CDR-H1, CDR-H2, and CDR-H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FRs within the variable region of a single, naturally occurring immunoglobulin chain. As used herein, a FR represents one of the four sub-regions, and FRs represents two or more of the four sub-regions constituting a framework region. Human heavy chain and light chain acceptor sequences are known in the art. In one embodiment, the acceptor sequences known in the art may be used in the antibodies disclosed herein.

Fibroblast Activation Protein (FAP): The term “fibroblast activation protein” or “FAP”, as used herein, refers to a 97-kDa type II transmembrane serine protease. FAP is a member of the prolyl peptidase family, which also contains dipeptidyl peptidase IV (DPPIV, CD26), DPP7 (DPP II, quiescent cell proline dipeptidase), DPP8, DPP9, and prolyl carboxypeptidase (PCP, angiotensinase C). FAP contains dipeptidyl peptidase enzymatic activity and endopeptidase activity. Exemplary human FAP amino acid sequences are set forth in Accession Number AAB49652.1 (SEQ ID NO: 35). Accession Numbers AAB44837.1, AAE30605.1. AAX04090.1, ADL88098.1, AQN54508.1, ATK13500.1. ATK18094.1. AWT87270.1, AYI13559.1, QBE27403.1, QFN60450.1, QNB70086.1, and QYQ07220.1 also describe the same FAP amino acid sequence of SEQ ID NO: 35. Exemplary mouse FAP amino acid sequences are set forth in Accession Number CAA71116.1 (SEQ ID NO: 36), AND76664.1 (SEQ ID NO: 37), and AAH19190.1 (SEQ ID NO: 38).

Fibroblast activation protein-α (FAP) is a cell surface antigen expressed on various cells (e.g., reactive stromal fibroblasts in tumor microenvironment, soft tissue sarcomas, granulation tissue of wound healing and certain fetal mesenchymal fibroblasts). The expression level of FAP on certain cells is higher (e.g., fibroblasts in the tumor microenvironment) than FAP expression level on normal tissues. FAP can also be shed from the plasma membrane forming a soluble FAP (Lee et al., Antiplasmin-cleaving enzyme is a soluble form of fibroblast activation protein. Blood (2006) 107:1397-404. 10.118). Hence, it is not restricted to the cell surface.

Human antibody: The term “human antibody”, as used herein, is intended to include antibodies having variable and constant regions derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

Humanized antibody: The term “humanized antibody” refers to antibodies which comprise heavy and light chain variable region sequences from a non-human species (e.g., a mouse) but in which at least a portion of the V_{H} and/or V_{L} sequence has been altered to be more “human-like”, i.e., more similar to human germline variable sequences. One type of humanized antibody is a CDR-grafted antibody, in which human CDR sequences are introduced into non-human VH and VL sequences to replace the corresponding nonhuman CDR sequences. In one embodiment, humanized anti-FAP antibodies and antigen binding fragments thereof are provided. Such antibodies may be generated by obtaining murine anti-FAP monoclonal antibodies using traditional hybridoma technology followed by humanization using in vitro genetic engineering, such as those disclosed in Kasaian et al PCT Publication No. WO 2005/123126 A2. In some embodiments, a humanized antibody comprises human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some embodiments, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in WO 99/58572. Other forms of humanized antibodies have one or more CDRs (one, two, three, four, five, six) which are altered with respect to the original antibody, which are also termed one or more CDRs derived from one or more CDRs from the original antibody. Humanized antibodies may also involve affinity maturation.

Invariant natural killer T cells (iNKT): The term “invariant Natural Killer T cells”, or “invariant NKT cells”, “iNKT cells”, or “Type I NKT cell), as used herein, refer to a population of T lymphocytes expressing a conserved semi-invariant TCR specific for lipid antigens (Ags) restricted for the monomorphic MHC class I-related molecule CD1d. Natural killer T cells (NKT cells) were originally characterized in mice as T cells that express both a TCR and NK1.1 (NKR-Pla-c or CD161), a C-type lectin NK receptor. Invariant NKT (iNKT) cells express a semi-invariant αβ TCR (e.g., formed by an invariant TRAV11-TRAJ18 (4) rearrangement in mice, or the homologous invariant TRAV10-TRAJ18 chain in humans), paired with a limited set of diverse VB chains, predominantly TRBV1, TRBV29, or TRBV13 in mice (6) and TRBV25 in humans (sec., e.g., Dellabona et al., An invariant V alpha 24-J alpha Q/V beta 11 T cell receptor is expressed in all individuals by clonally expanded CD4-8-T cells. J Exp Med. (1994) 180:1171-6. 10.1084). The semi-invariant TCR recognizes exogenous and endogenous lipid Ags presented by the monomorphic MHC class I-related molecule CD1d (sec., e.g., Brennan et al., Invariant natural killer T cells: an innate activation scheme linked to diverse effector functions. Nat Rev Immunol. (2013) 13:101-17. 10.1038). Exogenous lipid Ags include the prototypical α-Galactosylceramide (α-GalCer) (Kawano et al., CD1d-restricted and TCR-mediated activation of valpha14 NKT cells by glycosylceramides. Science. (1997) 278:1626-9. 10.1126) and a number of bacterial-derived Ags can activate iNKT cells.

iNKT cells undergo a distinct developmental pathway compared to T cells, leading to the acquisition of innate effector functions already in the thymus. Thymic iNKT cells indeed express markers usually upregulated by peripheral effector/memory T cells, such as CD44 and CD69, together with distinctive NK differentiation markers, such as NK1.1 (in some mouse genetic backgrounds, CD161 in humans), CD122 (the IL-2R/IL-15R β-chain), CD94/NKG2 and Ly49(A-J), and a broad spectrum of TH1/2/17 effector cytokines. Once migrated in the periphery, iNKT cells form a tissue resident population that survey the cellular integrity and rapidly respond to local damage and inflammation, jump starting the reaction by cells of the innate and adaptive immune response.

Because iNKT cells can rapidly produce IFNγ, IL-4, or both, they have been found to play a role in various diseases by establishing a context-dependent Th1- or Th2-based immune response. In bacterial and viral infections, iNKT cells typically help in early control of the pathogen by establishing a productive Th1 response. In both mouse and human studies, roles for iNKT cells have been described in diseases associated with excessive Th1 responses like type 1 diabetes and chronic obstructive pulmonary disease. Roles have also been described for iNKT cells helping to suppress Th1 responses and drive tolerogenic responses to grafts. As an example, following hematopoietic stem cell transfer, the presence of iNKT cells is predictive for survival with a reduction in graft versus host disease (GvHD) in patients and preclinical models.

Isolated antibody: An “isolated antibody”, as used herein, is intended to refer to an antibody that is substantially free of other antibodies having different antigenic specificities (e.g., an isolated antibody that specifically binds FAP is substantially free of antibodies that specifically bind antigens other than FAP). Moreover, an isolated antibody may be substantially free of other cellular material and/or chemicals.

Percent identity: The determination of “percent identity” or “percent identical” between two sequences (e.g., amino acid sequences or nucleic acid sequences) can be accomplished using a mathematical algorithm. A specific, non-limiting example of a mathematical algorithm utilized for the comparison of two sequences is the algorithm of Karlin S & Altschul S F (1990) PNAS 87:2264-2268, modified as in Karlin S & Altschul S F (1993) PNAS 90:5873-5877, each of which is herein incorporated by reference in its entirety. Such an algorithm is incorporated into the NBLAST and XBLAST programs of Altschul S F et al., (1990) J Mol Biol 215:403, which is herein incorporated by reference in its entirety. BLAST nucleotide searches can be performed with the NBLAST nucleotide program parameters set, e.g., at score=100, word length=12 to obtain nucleotide sequences homologous to a nucleic acid molecule described herein. BLAST protein searches can be performed with the XBLAST program parameters set, e.g., at score=50, word length=3 to obtain amino acid sequences homologous to a protein molecule described herein. To obtain gapped alignments for comparison purposes, Gapped BLAST can be utilized as described in Altschul S F et al., (1997) Nuc Acids Res 25:3389-3402, which is herein incorporated by reference in its entirety. Alternatively, PSI BLAST can be used to perform an iterated search which detects distant relationships between molecules. Id. When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another specific, non-limiting example of a mathematical algorithm utilized for the comparison of sequences is the algorithm of Myers and Miller, 1988, CABIOS 4:11-17, which is herein incorporated by reference in its entirety. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

Recombinant antibody: The term “recombinant human antibody”, as used herein, is intended to include all human antibodies that are prepared, expressed, created or isolated by recombinant means, such as antibodies expressed using a recombinant expression vector transfected into a host cell (described in more details in this disclosure), antibodies isolated from a recombinant, combinatorial human antibody library (Hoogenboom H. R., (1997) TIB Tech. 15:62-70; Azzazy H., and Highsmith W. E., (2002) Clin. Biochem. 35:425-445; Gavilondo J. V., and Larrick J. W. (2002) BioTechniques 29:128-145; Hoogenboom H., and Chames P. (2000) Immunology Today 21:371-378), antibodies isolated from an animal (e.g., a mouse) that is transgenic for human immunoglobulin genes (see e.g., Taylor, L. D., et al. (1992) Nucl. Acids Res. 20:6287-6295; Kellermann S-A., and Green L. L. (2002) Current Opinion in Biotechnology 13:593-597; Little M. et al (2000) Immunology Today 21:364-370) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo. One embodiment of the disclosure provides fully human antibodies capable of binding human FAP which can be generated using techniques well known in the art, such as, but not limited to, using human Ig phage libraries such as those disclosed in Jermutus et al., PCT publication No. WO 2005/007699 A2.

Single-chain variable fragment (scFv): As used herein, the term “Single-chain variable fragment (scFv)”, refers to a fusion protein of the variable regions of the heavy (VH) and light chains (VL) of immunoglobulins, connected with a short linker peptide. A linker refers to a peptide or a short oligopeptide sequence used to join two subunits into a single polypeptide. A linker may have a sequence that is found in natural proteins, or may be an artificial sequence that is not found in any natural protein. A linker may be flexible and lacking in secondary structure or may have a propensity to form a specific three-dimensional structure under physiological conditions. In some embodiments, the linker is a glycine rich linker. In some embodiments, the linker is rich in serine or threonine. In some embodiments, a scFv is a fusion protein in which the N-terminus of the VH is linked with the C-terminus of the VL. In some embodiments, a scFv is a fusion protein in which the N-terminus of the VL is linked with the C-terminus of the VH. A scFv retains the specificity of the original immunoglobulin, despite removal of the constant regions and the introduction of the linker. In some embodiments, a scFv can be created to facilitate phage display. In some embodiments, a scFv can be created directly from subcloned heavy and light chains derived from a hybridoma. ScFvs have many uses, e.g., flow cytometry, immunohistochemistry, and as antigen-binding domains of a chimeric antigen receptor.

Specifically binds: As used herein, the term “specifically binds” refers to the ability of a molecule to bind to a binding partner with a degree of affinity or avidity that enables the molecule to be used to distinguish the binding partner from an appropriate control in a binding assay or other binding context. With respect to an antibody, the term, “specifically binds”, refers to the ability of the antibody to bind to a specific antigen with a degree of affinity or avidity, compared with an appropriate reference antigen or antigens, that enables the antibody to be used to distinguish the specific antigen from others, e.g., to an extent that permits preferential targeting to certain cells, e.g., muscle cells, through binding to the antigen, as described herein. In some embodiments, an antibody specifically binds to a target if the antibody has a K_{D} for binding the target of at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, or less. In some embodiments, an antibody specifically binds to FAP.

Subject: As used herein, the term “subject” refers to a mammal. In some embodiments, a subject is non-human primate, or rodent. In some embodiments, a subject is a human. In some embodiments, a subject is a patient, e.g., a human patient that has or is suspected of having a disease. In some embodiments, the subject is a human patient who has or is suspected of having cancer (e.g., FAP-expressing cancer) or other disorders associated with FAP.

Treatment: As used herein, the term “treating” or “treatment” refers to the application or administration of a composition including one or more active agents (e.g., anti-FAP antibodies) to a subject, who has a target disease or disorder (e.g., cancer), a symptom of the disease/disorder (e.g., tumor growth, tumor metastasis, fatigue, weight changes including unintended loss or gain, pain, fever, sores that don't heal, persistent cough or hoarseness, unusual bleeding, or anemia), or a predisposition toward the disease/disorder (e.g., cancer), with the purpose to cure, heal, alleviate, relieve, alter, remedy, ameliorate, improve, or affect the disorder (e.g., cancer), the symptom of the disease (e.g., tumor growth, tumor metastasis, fatigue, weight changes including unintended loss or gain, pain, fever, sores that don't heal, persistent cough or hoarseness, unusual bleeding, or anemia), or the predisposition toward the disease or disorder. Alleviating a target disease/disorder includes delaying or preventing the development or progression of the disease, or reducing disease severity.

Tumor microenvironment (TME): As used herein, the term “tumor microenvironment” or “TME” refers to the fluid, molecules, cells, and/or tissues around and/or at the tumor site. The TME can include normal cells, tumor cells, tumor stromal cells (e.g., stromal fibroblasts), blood vessels, blood, immune cells, and the non-cellular components (e.g., extracellular matrix such as collagen, fibronectin, hyaluronan, laminin, and secreted molecules such as cytokines, etc).

II. Method of Treating Cancer Using Anti-FAP CAR iNKT Cells

Aspects of the present disclosure provides a plurality of genetically modified immune cells (e.g., iNKT cells) expressing anti-fibroblast activation protein (FAP) chimeric antigen receptor for the treatment of cancer. In some embodiments, the iNKT cell can be engineered to express a chimeric antigen receptor that comprises any of the FAP binding moieties described herein (e.g., the anti-FAP antibody or antigen binding fragment as described in Table 2, and the existing FAP binding moieties as provided in Table 3) as the extracellular ligand-binding domain. In some embodiments, the genetically modified iNKT cells expressing anti-FAP CAR of the present disclosure can be engineered to further express soluble IL-15. In some embodiments, the present disclosure relates to methods of treating a disease (e.g., cancer) using the genetically modified immune cells (e.g., anti-FAP iNKT cells) described herein. As used herein, “engineered to express” means that the cell expresses or is capable of expressing the molecule (e.g., immunoregulatory gene product or armor) that it is engineered to express.

In some embodiments, FAP is associated with cancer. In some embodiments, FAP has been previously described to promote tumor growth via multiple mechanisms, including but limited to: promoting proliferation, invasion, angiogenesis, epithelial-to-mesenchymal transition, stem cell promotion, immunosuppression and/or drug resistance (e.g., Fitzgerald et al., The role of fibroblast activation protein in health and malignancy, Cancer Metastasis Rev. 2020 September; 39 (3): 783-803). In some embodiments, FAP is highly expressed in stroma cells (e.g., cancer associated fibroblasts (CAFs) in the tumor microenvironment (TME). In some embodiments, FAP plays an immunosuppressive role in the TME. Cancers expressing FAP include but are not limited to: breast cancer, colorectal cancer, pancreatic cancer, gastric cancer, brain cancer, ovarian cancer, myeloma, Melanoma, or sarcoma. In some embodiments, FAP is associated with non-cancer diseases, such as multiple human pathologies including fibrosis, arthritis, atherosclerosis, autoimmune diseases, metabolic diseases and cancer. In some embodiments, FAP is associated with progression and heightened severity of the disease (e.g., progression and heightened severity of cancer and/or non-cancer diseases).

Therapeutics targeting FAP have previously been described (e.g., Schuberth et al., Treatment of malignant pleural mesothelioma by fibroblast activation protein-specific re-directed T cells. J Transl Med (2013) 11:1-11), for example, FAP inhibitors (e.g., Talabostat), anti-FAP antibodies (e.g., sibrotuzumab), FAP vaccines, FAP CAR T Cells (e.g., Bughda et al., Fibroblast Activation Protein (FAP)-Targeted CAR-T Cells: Launching an Attack on Tumor Stroma, ImmunoTargets and Therapy 2021:10 313-323; Wang et al., Targeting Fibroblast Activation Protein in Tumor Stroma with Chimeric Antigen Receptor T Cells Can Inhibit Tumor Growth and Augment Host Immunity without Severe Toxicity, Cancer Immunol Res; 2 (2) February 2014). However, the clinical use of FAP CAR T cells were not successful. In some cases, FAP CAR T cells failed to regulate tumor growth, and induced lethal bone toxicity and cachexia, (e.g., through the lysis of multipotent bone marrow stromal cells) (Tran et al., Immune targeting of fibroblast activation protein triggers recognition of multipotent bone marrow stromal cells and cachexia. J Exp Med (2013) 210:1125-35. 10.1084).

In some embodiments, the genetically modified immune cells (e.g., iNKT cell) described herein (e.g., iNKT cell) express a CAR that combines an antigen recognition domains (e.g., an anti-FAP antibody or antigen binding fragment described herein), a transmembrane domain (e.g., a CD8 transmembrane domain), a cytoplasmic domain (e.g., an intracellular domain of CD3-zeta, CD28, OX40, 4-1BB, or any combinations thereof), and a co-stimulatory domain (e.g., a 4-1BB costimulatory domain). Therefore, in some embodiments, the transduced immune cell (e.g., iNKT cell) can elicit a direct immune response (e.g., CAR-mediated immune response)), and/or indirect immune response (e.g., NK cell response) in a subject. In some embodiments, the present disclosure provides the use of a CAR to redirect the specificity of a primary iNKT cell to a tumor antigen (e.g., FAP). Thus, in some embodiments, the present disclosure also provides a method for stimulating an iNKT cell-mediated immune response to a target cell population or tissue in a subject (e.g., a mammal) comprising the step of administering to the subject a plurality of iNKT cell that expresses a CAR (e.g., an anti-FAP CAR), wherein the CAR comprises a binding moiety that specifically interacts with a predetermined target (e.g., FAP), a transmembrane domain (e.g., CD8 transmembrane domain), a zeta chain portion comprising for example the intracellular domain of human CD3zeta, and a costimulatory signaling region. In some embodiments, the present disclosure includes a type of cellular therapy where iNKT cells are genetically modified (i.e., engineered) to express a CAR (e.g, anti-FAP CAR), and IL-15 (e.g., soluble IL-15 (sIL-15)). “engineered to express” means that the cell expresses or is capable of expressing the molecule (e.g., immunoregulatory gene product or armor) that it is engineered to expressIn some embodiments, the present disclosure includes a type of cellular therapy where anti-FAP CAR iNKT cells of the present disclosure are administered (e.g., injected, infused, etc.) to a subject in need thereof. The infused cells are able to kill tumor cells in the recipient. Unlike antibody therapies, CAR iNKT cells are able to replicate in vivo resulting in long-term persistence that can lead to sustained tumor control. In some embodiments, the secretion of sIL-15 by the iNKT cell expressing an anti-FAP CAR enhanced iNKT cell persistence in a subject. Further, previous studies have shown that, iNKT cells numbers are reduced in cancer tissue (e.g., solid tumor such as lung cancer, head and neck cancer, colorectal cancer, renal cancer, etc) compared to normal tissue. In some embodiments, in certain cancers, iNKT cells functions are impaired. Accordingly, administration of the anti-FAP iNKT cells described herein may restore and enhance iNKT function in the subject, thereby leading to better prognosis.

In some embodiments, the antigen binding domain in the CAR of the present disclosure targets a tumor antigen (e.g., FAP) for the purposes of treating a cancer. In some embodiments, the antigen binding moiety portion of the CAR of the present disclosure is designed to treat a particular cancer (e.g., cancer expressing FAP).

In some embodiments, anti-FAP CAR iNKT cells of the present disclosure kill FAP expressing stroma cells in the TME (e.g., CAFs). In some embodiments, anti-FAP CAR iNKT cells of the present disclosure do not kill normal cells expressing lower level of FAP compared to stroma cells (e.g., CAFs) in cancer. In some embodiments, FAP expression is high in cancers cells, including but not limited to breast cancer, lung cancer, colorectal cancer, prostate cancer, stomach cancer, pancreatic cancer, prostate cancer, thyroid cancer, cervical cancer, urothelial cancers, or sarcoma compared to FAP expression in normal tissues. FAP expression level can be measured by any suitable methods known in the art, e.g., by flow cytometry, immunoblot, RT-PCR, etc.

Expression of FAP is an important characteristic of CAFs (Garin-Chesa et al., Cell surface glycoprotein of reactive stromal fibroblasts as a potential antibody target in human epithelial cancers. Proc Natl Acad Sci USA. 1990; 87 (18): 7235-7239). FAP activity impacts the secreted CAF proteome, leading to reduced levels of anti-angiogenic factors (e.g., PEDF, angiopoietin-1, VEGFC), and regulates matrix processing enzymes (e.g., Koczorowska et al., Fibroblast activation protein-alpha, a stromal cell surface protease, shapes key features of cancer associated fibroblasts through proteome and degradome alterations. Mol Oncol. 2016; 10 (1): 40-58). Further, FAP remodels the ECM through the endopeptidase activity by cleaving the collagen and modifying bioactive signaling peptides in cancer (Kelly et al., Fibroblast activation protein-alpha and dipeptidyl peptidase IV (CD26): cell-surface proteases that activate cell signaling and are potential targets for cancer therapy. Drug Resist Updat. 2005; 8 (1-2): 51-58). FAP expression in cancer is thought to be associated with poor prognosis. In some embodiments, high level expression of FAP in the TME plays an immunosuppressive role (Kraman et al., Suppression of antitumor immunity by stromal cells expressing fibroblast activation protein-alpha. Science. 2010; 330 (6005): 827-830). Not wishing to be bound to any particular theory, FAP drives the CAFs towards mediating tumor immunosuppression by recruiting myeloid-derived suppressor cells (MDSCs) in the TME (e.g., Yang et al., FAP promotes immunosuppression by cancer-associated fibroblasts in the tumor microenvironment via STAT3-CCL2 signaling. Cancer Res. 2016; 76 (14): 4124-4135).

FAP-targeting CAR-T cells have been engineered to target CAFs in various solid cancers, such as mesothelioma, lung and pancreatic cancers (e.g., Bughda et al., Fibroblast Activation Protein (FAP)-Targeted CAR-T Cells: Launching an Attack on Tumor Stroma, ImmunoTargets and Therapy 2021:10 313-323; Wang et al., Targeting Fibroblast Activation Protein in Tumor Stroma with Chimeric Antigen Receptor T Cells Can Inhibit Tumor Growth and Augment Host Immunity without Severe Toxicity, Cancer Immunol Res; 2 (2) February 2014; Rodriguez-Garcia A, Palazon A, Noguera-Ortega E, Powell D J, Guedan S. CAR-T cells hit the tumor microenvironment: strategies to overcome tumor escape. Front Immunol. 2020; 11). However, on-target off-tumor toxicity was observed in these studies because while FAP is highly expressed in these cancers, it is also found to express at low level in normal cells (e.g., skeletal muscle, adipose tissue, and pancreas, etc.).

In some embodiments, the present disclosure provides anti-FAP CAR iNKT cells engineered to kill high FAP expressing stroma cells (e.g., CAFs in the TME). In some embodiments, anti-FAP CAR iNKT cells described herein show reduced or lack on-target off-tumor toxicity. In some embodiments, anti-FAP CAR iNKT cells kill FAP-expressing tumor cells and/or FAP expressing CAFs. In some embodiments, depletion of FAP expressing stroma cells (e.g., CAFs in the TME) by anti-FAP CAR iNKT cells described herein reduces immunosuppression in the TME. In some embodiments, depletion of FAP expressing stroma cells (e.g., CAFs in the TME) by anti-FAP CAR iNKT cells described herein lead to more immune cell infiltration into the tumor (e.g., solid tumor). In some embodiments, anti-FAP CAR iNKT cells described herein are armored (e.g., secreting IL-15) to prolong persistence of CAR iNKT cells in the TME. In some embodiments, anti-FAP CAR iNKT cells described herein are armored (e.g., secreting IL-15) to enhance certain properties of the anti-FAP CAR iNKT cells (e.g., enhanced killing efficiency, prolonged persistence in the subject, and/or potentiated tumor killing by an additional cancer therapy (e.g., CAR T cells targeting a tumor antigen) relative to the iNKT cells without the armoring molecule).

The CAR-modified iNKT cells of the present disclosure may also serve as a type of vaccine for ex vivo immunization and/or in vivo therapy in a mammal. Preferably, the mammal is a human.

In some embodiments, the iNKT cells as described herein may be utilized in the treatment and prevention of diseases (e.g., cancer) that arise in individuals who are immunocompromised, such as individuals having cancer. In particular, the iNKT cells expressing the anti-FAP CAR of the present disclosure are used in the treatment of cancer, such as FAP positive cancer (e.g., breast cancer, lung cancer, head and neck cancer, colorectal cancer, prostate cancer, stomach cancer, pancreatic cancer, prostate cancer, thyroid cancer, cervical cancer, renal cancer, urothelial cancers, or sarcoma). In certain embodiments, the iNKT cells expressing the anti-FAP CAR of the present disclosure are used in the treatment of patients at risk for developing cancer such as FAP positive cancer (e.g., breast cancer, lung cancer, head and neck cancer, colorectal cancer, prostate cancer, stomach cancer, pancreatic cancer, prostate cancer, thyroid cancer, cervical cancer, renal cancer, urothelial cancers, or sarcoma). In some embodiments, the cancer is a primary cancer. In some embodiments, the cancer is a metastatic cancer.

In certain embodiments, the subject does not undergo lymphodepletion prior to, or concurrent with, treatment with an anti-FAP CAR iNKT cell or pharmaceutical composition as disclosed herein. Lymphodepletion is frequently performed prior to immunotherapies, such as adoptive cell therapy such as CAR T therapies. In some instances, subject receiving adoptive cell therapy receive a course of chemotherapy to deplete the T cells from the subject, for instance, to debulk a tumor, alter tumor phenotype, modify the tumor microenvironment, remove cytokine sinks (e.g., make IL-2, IL-7, and IL-15 more available) and suppress the host immune system. In some instances, lymphodepletion can effectively prolong the persistence of infused cells and increase the effectiveness of treatment. However, lymphodepletion has multiple negative effects including, neutropenia, anemia, thrombocytopenia, and immunosuppression, and toxicities associated with lymphodepletion agents such as fludarabine and cyclophosphamide. In some embodiments, subject receiving iNKT cell therapy (e.g., anti-FAP CAR iNKT cell therapy) does not need lymphodepletion therapy. In certain embodiments, the subject does not receive treatment with, fludarabine, or cyclophosphamide prior to, or concurrent with, administration of the anti-FAP CAR iNKT cells or a pharmaceutical composition as disclosed herein. In some embodiments, iNKT cell therapy (e.g., anti-FAP CAR iNKT cell therapy or a composition thereof) without lymphodepletion are effective in reducing tumor burden and/or improve survival. In some embodiments, iNKT cell therapy (e.g., anti-FAP CAR iNKT cell therapy or a composition thereof) without lymphodepletion exhibit long term efficacy in killing cancer cells. In some embodiments, iNKT cell therapy (e.g., anti-FAP CAR iNKT cell therapy or a composition thereof) without lymphodepletion does not induce graft-versus-host disease (GVHD).

In some embodiments, the iNKT cells expressing the anti-FAP CAR of the present disclosure, or a composition comprising such cells, may be used, or may be administered to a subject in need thereof, to provide anti-tumor immunity; to treat or prevent cancer; to reduce immunosuppression in the TME; and/or to increase therapeutic efficacy of other therapeutic agents (e.g., CAR-T cells or immune check point inhibitors). In some embodiments, the cancer is a FAP-expressing cancer. In some embodiments, the cancer is breast cancer, lung cancer, head and neck cancer, colorectal cancer, prostate cancer, stomach cancer, pancreatic cancer, prostate cancer, thyroid cancer, cervical cancer, renal cancer, urothelial cancers, or sarcoma.

In some embodiments, the iNKT cells expressing the anti-FAP CAR of the present disclosure may be administered either alone, or as a composition (e.g., a pharmaceutical composition) in combination with diluents and/or with other components such as IL-2 or other cytokines or cell populations. Briefly, pharmaceutical compositions of the present disclosure may comprise a target cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

Pharmaceutical compositions of the present disclosure may be administered in a manner appropriate to the disease to be treated (or prevented). The quantity and frequency of administration will be determined by such factors as the condition of the patient, and the type and severity of the patient's disease, although appropriate dosages may be determined by clinical trials. In some embodiments, compositions of the present disclosure are formulated for intravenous administration.

When “an immunologically effective amount”, “an anti-tumor effective amount”, “a tumor-inhibiting effective amount”, or “therapeutic amount” is indicated, the precise amount of the compositions of the present disclosure to be administered can be determined by a physician with consideration of individual differences in age, weight, tumor size, extent of infection or metastasis, and condition of the patient (subject). It can generally be stated that a pharmaceutical composition comprising the CAR-modified immune cells (e.g., iNKT cells expressing anti-FAP CAR) described herein may be administered at a dosage of 10⁴ to 10⁹ cells/kg body weight, 10⁵ to 10⁶ cells/kg body weight, including all integer values within those ranges. iNKT cell compositions may also be administered multiple times at these dosages. The cells can be administered by using infusion techniques that are commonly known in immunotherapy (see, e.g., Rosenberg et al., New Eng. J. of Med. 319:1676, 1988).

The administration of the subject compositions may be carried out in any convenient manner, including infusion, injection, ingestion, transfusion, implantation or transplantation. The compositions described herein may be administered to a patient subcutaneously, intradermally, intratumorally, intranodally, intramedullary, intramuscularly, by intravenous (i.v.) injection, or intraperitoneally. In some embodiments, the immune cell (e.g., iNKT cell) compositions of the present disclosure are administered to a patient by intradermal or subcutaneous injection. In some embodiments, the immune cell (e.g., iNKT cell) compositions of the present disclosure are preferably administered by i.v. injection. The compositions of immune cells (e.g., iNKT cells) may also be injected directly into a tumor, lymph node, or site of disease.

In some embodiments, iNKT cells (e.g., anti-FAP CAR iNKT cell) activated and expanded using the methods described herein, or other methods known in the art, are expanded to therapeutic levels, are administered to a patient in conjunction with (e.g., before, simultaneously or following) any number of relevant treatment modalities, including but not limited to treatment with agents such as antiviral therapy, cidofovir and interleukin-2, Cytarabine (also known as ARA-C) or natalizumab treatment for MS patients or efalizumab treatment for psoriasis patients or other treatments for PML patients. In some embodiments, the iNKT cells of the present disclosure may be used in combination with chemotherapy, radiation, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxin, fludaribine, cyclosporin, FK506, rapamycin, mycophenolic acid, steroids, FR901228, cytokines, and irradiation. In some embodiments, the cell compositions of the present disclosure are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In some embodiments, the cell compositions of the present disclosure are administered following B-cell ablative therapy such as agents that react with CD20, e.g., Rituxan. For example, in some embodiments, subjects may undergo standard treatment with high dose chemotherapy followed by peripheral blood stem cell transplantation. In some embodiments, following the transplant, subjects receive an infusion of the expanded immune cells (e.g., iNKT cell) of the present disclosure. In an additional embodiment, iNKT cells are administered before or following surgery.

In some embodiments, the present disclosure also contemplates treating cancer using the anti-FAP CAR iNKT cells in combination with one or more additional therapeutic agents. In some embodiments, the additional therapeutic agent is a T cell targeting a tumor antigen (e.g., T cell or a CAR T cell), including but not limited to: ErbB2 (HER2/neu), carcinoembryonic antigen (CEA), epithelial cell adhesion molecule (EpCAM), epidermal growth factor receptor (EGFR), EGFR variant III (EGFRvIII), vascular endothelial growth factor receptor 2 (VEGFR2), IL13R, GD3, c-type lectin-like molecule 1 (CLL1), cholecytoskinin B receptor (CCKBR), gonadotropin releasing hormone receptor (GnRHR), somatostatin receptor 2 (SSRT2), gastrin-releasing peptide receptor (GRPR), neurokinin 1 receptor (NK1R), CD19, CD20, CD30, CD40, disialoganglioside GD2, ductal-epithelial mucine, folate receptor, gp36, TAG-72, glycosphingolipids, glioma-associated antigen, B-human chorionic gonadotropin, alphafetoprotein (AFP), neurotensin receptor 1 (NTSR1), lectin-reactive AFP, thyroglobulin, RAGE-1, MN-CA IX, human telomerase reverse transcriptase, RU1, RU2 (AS), intestinal carboxyl esterase, mut hsp70-2, M-CSF. prostase, prostate specific antigen (PSA), PAP, NY-ESO-1, LAGA-1a, p53, prostein, PSMA, surviving and telomerase, prostate-carcinoma tumor antigen-1 (PCTA-1), MAGE, ELF2M, neutrophil elastase, ephrin B2, CD22, insulin growth factor (IGF1)-1, IGF-II, IGFI receptor, mesothelin, a major histocompatibility complex (MHC) molecule presenting a tumor-specific peptide epitope, melanocortin 1 receptor (MC1R), 5T4, ROR1, Nkp30, NKG2D, tumor stromal antigens, the extra domain A (EDA) and extra domain B (EDB) of fibronectin and the Al domain of tenascin-C (TnC Al), lineage-specific or tissue specific antigen such as CD3, CD4, CD8, CD24, CD25, CD33, CD34, CD133, CD138, CTLA-4, B7-1 (CD80), B7-2 (CD86), endoglin, a major histocompatibility complex (MHC) molecule, FAP (CD269, TNFRSF 17), CS 1, or a virus-specific surface antigen such as an HIV-specific antigen (such as HIV gpl20); an EBV-specific antigen, a CMV-specific antigen, a HPV-specific antigen such as the E6 or E7 oncoproteins, a Lasse Virus-specific antigen, an Influenza Virus-specific antigen, as well as any derivate or variant of these surface markers. In some embodiments, treatment using the anti-FAP CAR iNKT cells described herein reduces the immunosuppression in the TME, increases CAR T cell infiltration into the tumor, thereby increasing the CAR T cell efficacy. In some embodiments, the anti-FAP CAR iNKT cells secrets IL-15, which potentiates the cytotoxic activity of CAR T cells.

In some embodiments, the additional therapeutic agent is an immune checkpoint inhibitor, such as PD1/PD-L1 inhibitor or CTLA-4 inhibitor. Any suitable known immune check point inhibitors can be combined with the anti-FAP CAR iNKT cells described herein.

The scaling of dosages for human administration can be performed according to art-accepted practices. The dose for CAMPATH, for example, will generally be in the range 1 to about 100 mg for an adult patient, usually administered daily for a period between 1 and 30 days. The preferred daily dose is 1 to 10 mg per day although in some instances larger doses of up to 40 mg per day may be used (described in U.S. Pat. No. 6,120,766). Strategies for CAR T cell dosing and scheduling have been discussed (Ertl et al., 2011, Cancer Res, 71:3175-81; Junghans, 2010, Journal of Translational Medicine, 8:55).

### III. FAP Binding Moieties as Chimeric Antigen Receptor

The present disclosure provides FAP binding moieties (e.g., antibodies or antigen binding fragments specific for fibroblast activation protein (FAP), FAP-binding peptides) that can be engineered to be an extracellular ligand-binding domain of a chimeric antigen receptor (CAR) expressed by a genetically modified cell (e.g., iNKT cell).

In some embodiments, the present disclosure provides anti-FAP antibodies or antigen binding fragments thereof as FAP binding moieties. In some embodiments, anti-FAP antibodies or antigen binding fragments thereof provided herein are antibodies that bind to FAP with high specificity and affinity. In some embodiments, the anti-FAP antibody antigen binding fragments thereof described herein specifically binds to an extracellular epitope of FAP or an epitope that becomes exposed to an antibody. In some embodiments, anti-FAP antibodies antigen binding fragments thereof provided herein bind specifically to FAP from human, non-human primates, mouse, rat, etc. In some embodiments, anti-FAP antibodies or antigen binding fragments thereof provided herein bind specifically to human FAP and mouse FAP. In some embodiments, an anti-FAP antibody or antigen binding fragment thereof described herein specifically binds to an epitope on human FAP and/or mouse FAP. In some embodiments, an anti-FAP antibody or antigen binding fragment thereof described herein may bind to a fragment of a human FAP and/or a mouse FAP. In some embodiments, an anti-FAP antibody or antigen binding fragment thereof described herein may bind to a fragment of FAP (e.g., human FAP and/or mouse FAP, e.g., SEQ ID NOs: 35-38) between about 5 and about 200 amino acids, between about 10 and about 200 amino acids, between about 20 and about 200 amino acids, between about 30 and about 150 amino acids, between about 30 and about 120 amino acids, between about 30 and about 100 amino acids, between about 30 and about 90 amino acids, between about 30 and about 80 amino acids, between about 30 and about 60 amino acids, between about 30 and about 50 amino acids, between about 40 and about 80 amino acids, or between about 40 and about 60 amino acids in length. In some embodiments, an anti-FAP antibody or antigen binding fragment thereof may bind to a FAP fragment (e.g., human FAP and/or mouse FAP, e.g., SEQ ID NOs: 35-38) comprising a contiguous number of amino acids from human FAP protein and/or mouse FAP protein. In some embodiments, an anti-FAP antibody or antigen binding fragment thereof described herein may bind to a fragment comprising at least 5, at least 10, at least 20, at least 30, at least 40, at least 50, at least 60, at least 70, at least 80, at least 90, at least 100, at least 110, at least 120, at least 130, at least 140, at least 150, at least 160, at least 170, or at least 180 contiguous amino acids of human FAP protein and/or mouse FAP protein. In some embodiments, an anti-FAP antibody described herein binds to one or more amino acids in amino acid fragment 141-290 of a human FAP (e.g., human FAP as set forth in SEQ ID NO: 35). In some embodiments, an anti-FAP antibody described herein binds one or more contiguous amino acids within amino acid fragment 141-290 of a human FAP (e.g., human FAP as set forth in SEQ ID NO: 35). In some embodiments, an anti-FAP antibody described herein binds one or more non-contiguous amino acids within amino acid fragment 141-290 of a human FAP (e.g., human FAP as set forth in SEQ ID NO: 35). In some embodiments, an anti-FAP antibody described herein binds one or more contiguous and non-contiguous amino acids within amino acid fragment 141-290 of a human FAP (e.g., human FAP as set forth in SEQ ID NO: 35). In some embodiments, an anti-FAP antibody or antigen binding fragment thereof described herein may bind to a fragment having an amino acid sequence having up to five amino acids, up to four amino acids, up to three amino acids, up to two amino acids, or up to one amino acid different from a fragment of human FAP protein and/or mouse FAP protein. Exemplary human FAP amino acid sequences are set forth in Accession Number AAB49652.1 (SEQ ID NO: 35). Accession Numbers AAB44837.1, AAE30605.1, AAX04090.1, ADL88098.1, AQN54508.1, ATK13500.1, ATK18094.1, AWT87270.1, AYI13559.1, QBE27403.1. QFN60450.1, QNB70086.1, and QYQ07220.1 also describe the same FAP amino acid sequence of SEQ ID NO: 35.

An exemplary human FAP amino acid sequence is set forth in SEQ ID NO: 35 (amino acid fragment 141-290 bolded and underlined):

MKTWVKIVFGVATSAVLALLVMCIVLRPSRVHNSEENTMRALTLKDILNGTFSYKTFFPNWI
SGQEYLHQSADNNIVLYNIETGQSYTILSNRTMKSVNASNYGLSPDRQFVYLESDYSKLWRY
SYTATYYIYDLSNGEFVRGNELPRPIQYLCWSPVGSKLAYVYQNNIYLKQRPGDPPFQITFN
GRENKIFNGIPDWVYEEEMLPTKYALWWSPNGKFLAYAEFNDKDIPVIAYSYYGDEQYPRTI
NIPYPKAGAKNPVVRIFIIDTTYPAYVGPQEVPVPAMIASSDYYFSWLTWVTDERVCLQWLK
RVQNVSVLSICDFREDWQTWDCPKTQEHIEESRTGWAGGFFVSRPVFSYDAISYYKIFSDKD
GYKHIHYIKDTVENAIQITSGKWEAINIFRVTQDSLFYSSNEFEEYPGRRNIYRISIGSYPP
SKKCVTCHLRKERCQYYTASFSDYAKYYALVCYGPGIPISTLHDGRIDQEIKILEENKELEN
ALKNIQLPKEEIKKLEVDEITLWYKMILPPQFDRSKKYPLLIQVYGGPCSQSVRSVFAVNWI
SYLASKEGMVIALVDGRGTAFQGDKLLYAVYRKLGVYEVEDQITAVRKFIEMGFIDEKRIAI
WGWSYGGYVSSLALASGTGLFKCGIAVAPVSSWEYYASVYTERFMGLPTKDDNLEHYKNSTV
MARAEYFRNVDYLLIHGTADDNVHFQNSAQIAKALVNAQVDFQAMWYSDQNHGLSGLSTNHL
YTHMTHFLKQCFSLSD

Exemplary mouse FAP amino acid sequences are set forth in Accession Number CAA71116.1 (SEQ ID NO: 36), AND76664.1 (SEQ ID NO: 37), and AAH19190.1 (SEQ ID NO: 38).

An exemplary mouse FAP amino acid sequence is set forth in SEQ ID NO: 36:

MKTWLKTVFGVTTLAALALVVICIVLRPSRVYKPEGNTKRALTLKDILNGTFSYKTYFPNWI
SEQEYLHQSEDDNIVFYNIETRESYIILSNSTMKSVNATDYGLSPDRQFVYLESDYSKLWRY
SYTATYYIYDLQNGEFVRGYELPRPIQYLCWSPVGSKLAYVYQNNIYLKQRPGDPPFQITYT
GRENRIFNGIPDWVYEEEMLATKYALWWSPDGKFLAYVEFNDSDIPIIAYSYYGDGQYPRTI
NIPYPKAGAKNPVVRVFIVDTTYPHHVGPMEVPVPEMIASSDYYFSWLTWVSSERVCLQWLK
RVQNVSVLSICDFREDWHAWECPKNQEHVEESRTGWAGGFFVSTPAFSQDATSYYKIFSDKD
GYKHIHYIKDTVENAIQITSGKWEAIYIFRVTQDSLFYSSNEFEGYPGRRNIYRISIGNSPP
SKKCVTCHLRKERCQYYTASFSYKAKYYALVCYGPGLPISTLHDGRTDQEIQVLEENKELEN
SLRNIQLPKVEIKKLKDGGLTFWYKMILPPQFDRSKKYPLLIQVYGGPCSQSVKSVFAVNWI
TYLASKEGIVIALVDGRGTAFQGDKFLHAVYRKLGVYEVEDQLTAVRKFIEMGFIDEERIAI
WGWSYGGYVSSLALASGTGLFKCGIAVAPVSSWEYYASIYSERFMGLPTKDDNLEHYKNSTV
MARAEYFRNVDYLLIHGTADDNVHFQNSAQIAKALVNAQVDFQAMWYSDQNHGISSGRSQNH
LYTHMTHFLKQCFSLSD

An exemplary mouse FAP amino acid sequence is set forth in SEQ ID NO: 37:

LRPSRVYKPEGNTKRALTLKDILNGTFSYKTYFPNWISEQEYLHQSEDDNIVFYNIETRESY
IILSNSTMKSVNATDYGLSPDRQFVYLESDYSKLWRYSYTATYYIYDLQNGEFVRGYELPRP
IQYLCWSPVGSKLAYVYQNNIYLKQRPGDPPFQITYTGRENRIFNGIPDWVYEEEMLATKYA
LWWSPDGKFLAYVEFNDSDIPIIAYSYYGDGQYPRTINIPYPKAGAKNPVVRVFIVDTTYPH
HVGPMEVPVPEMIASSDYYFSWLTWVSSERVCLQWLKRVQNVSVLSICDFREDWHAWECPKN
QEHVEESRTGWAGGFFVSTPAFSQDATSYYKIFSDKDGYKHIHYIKDTVENAIQITSGKWEA
IYIFRVTQDSLFYSSNEFEGYPGRRNIYRISIGNSPPSKKCVTCHLRKERCQYYTASFSYKA
KYYALVCYGPGLPISTLHDGRTDQEIQVLEENKELENSLRNIQLPKVEIKKLKDGGLTFWYK
MILPPQFDRSKKYPLLIQVYGGPCSQSVKSVFAVNWITYLASKEGIVIALVDGRGTAFQGDK
FLHAVYRKLGVYEVEDQLTAVRKFIEMGFIDEERIAIWGWSYGGYVSSLALASGTGLFKCGI
AVAPVSSWEYYASIYSERFMGLPTKDDNLEHYKNSTVMARAEYFRNVDYLLIHGTADDNVHF
QNSAQIAKALVNAQVDFQAMWYSDQNHGISSGRSQNHLYTHMTHFLKQCFSLSD

An exemplary mouse FAP amino acid sequence is set forth in SEQ ID NO: 38:

MKTWLKTVFGVTTLAALALVVICIVLRPSRVYKPEGNTKRALTLKDILNGTFSYKTYFPNWI
SEQEYLHQSEDDNIVFYNIETRESYIILSNSTMKSVNATDYGLSPDRQFVYLESDYSKLWRY
SYTATYYIYDLQNGEFVRGYELPRPIQYLCWSPVGSKLAYVYQNNIYLKQRPGDPPFQITYT
GRENRIFNGIPDWVYEEEMLATKYALWWSPDGKFLAYVEFNDSDIPIIAYSYYGDGQYPRTI
NIPYPKAGAKNPVVRVFIVDTTYPHHVGPMEVPVPEMIASSDYYFSWLTWVSSERVCLQWLK
RVQNVSVLSICDFREDWHAWECPKNQEHVEESRTGWAGGFFVSTPAFSQDATSYYKIFSDKD
GYKHIHYIKDTVENAIQITSGKWEAIYIFRVTQDSLFYSSNEFEGYPGRRNIYRISIGNSPP
SKKCVTCHLRKERCQYYTASFSYKAKYYALVCYGPGLPISTLHDGRTDQEIQVLEENKELEN
SLRNIQLPKVEIKKLKDGGLTFWYKMILPPQFDRSKKYPLLIQVYGGPCSQSVKSVFAVNWI
TYLASKEGIVIALVDGRGTAFQGDKFLHAVYRKLGVYEVEDQLTAVRKFIEMGFIDEERIAI
WGWSYGGYVSSLALASGTGLFKCGIAVAPVSSWEYYASIYSERFMGLPTKDDNLEHYKNSTV
MARAEYFRNVDYLLIHGTADDNVHFQNSAQIAKALVNAQVDFQAMWYSDQNHGILSGRSQNH

In some embodiments, an anti FAP antibody or antigen binding fragment thereof specifically binds FAP (e.g., any one of human and/or mouse FAP as set forth to any one of SEQ ID NOs: 35-38) with binding affinity (e.g., as indicated by K_{D}) of at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, or less. For example, an anti FAP antibody or antigen binding fragment thereof of the present disclosure can bind to a FAP protein (e.g., human and/or mouse FAP as set forth to any one of SEQ ID NOs: 35-38) with an affinity between 5 PM and 500 nM, between 10 pM and 450 nM, between 20 pM and 400 nM, between 30 pM and 350 nM, between 40 pM and 300 nM, between 50 pM and 250 nM, between 60 pM and 200 nM, between 70 pM and 150 nM, between 80 pM and 100 nM, between 80 pM and 90 nM, between 90 pM and 80 nM, between 100 pM and 70 nM, between 200 pM and 60 nM, between 300 PM and 50 nM, between 400 pM and 40 nM, between 500 pM and 30 nM, between 600 pM and 20 nM, between 700 pM and 10 nM, between 800 pM and 5 nM, or between 900 pM and 2 nM, between 30 nM and 700 nM, between 50 nM and 500 nM, between 100 nM and 500 nM, between 100 nM and 200 nM, or between 150 nM and 200 nM.

In some embodiments, the present disclosure made the discovery that iNKT cells expressing a FAP CAR comprising an anti-FAP antibody having a binding affinity to FAP (e.g., human FAP or mouse FAP) in the range of 0.1 nM to 100 nM (e.g., 0.1 nM to 80 nM, 0.1 nM to 50 nM, 0.1 nM to 25 nM, 0.1 nM to 10 nM, 0.1 nM to 5 nM, 0.1 nM to 1 nM, 0.1 nM to 0.5 nM, 0.2 nM to 0.4 nM, 0.3 nM to 0.4 nM, 10 nM to 80 nM, 10 nM to 70 nM, 10 nM to 60 nM, 10 nM to 50 nM, 10 nM to 40 nM, 10 nM to 30 nM, 10 nM to 20 nM, 20 nM to 80 nM, 20 nM to 70 nM, 20 nM to 60 nM, 20 nM to 50 nM, 20 nM to 40 nM, 20 nM to 30 nM, 10 nM to 15 nM, 30 nM to 80 nM, 30 nM to 70 nM, 30 nM to 60 nM, 30 nM to 50 nM, 30 nM to 40 nM, 40 nM to 80 nM, 40 nM to 70 nM, 40 nM to 60 nM, 40 nM to 50 nM, 50 nM to 80 nM, 50 nM to 70 nM, 50 nM to 60 nM, 60 nM to 80 nM, 60 nM to 70 nM, or 70 nM to 80 nM) perform better in killing tumor cells expressing FAP (e.g., human or mouse FAP) as compared to iNKT cells expressing a FAP CAR comprising an anti-FAP antibody having higher binding affinities.

The disclosure also includes antibodies that compete with any of the antibodies described herein for binding to a FAP protein (e.g., human and/or mouse FAP protein as set forth to any one of SEQ ID NOs: 35-38) and that have an affinity of 100 nM or lower (e.g., 80 nM or lower, 50 nM or lower, 20 nM or lower, 10 nM or lower, 500 pM or lower, 50 pM or lower, or 5 pM or lower). The affinity and binding kinetics of the anti-FAP antibodies or antigen binding fragment thereof can be tested using any suitable method including but not limited to biosensor technology (e.g., OCTET or BIACORE). In some embodiments, the anti-FAP antibodies or antigen binding fragments thereof bind to FAP with a K_{D} of sub-nanomolar range.

Non-limiting examples of anti-FAP antibodies are provided in Table 2.

TABLE 2
Exemplary anti-FAP antibodies (CDRs according to the Kabat definition)

| | | | SEQ ID |
|---|---|---|---|
| Anti-FAP antibodies | | Sequences | NO |
| A07 | CDRH1 | SYAIS | 1 |
| | CDRH2 | GIIPIFGTANYAQKFQG | 2 |
| | CDRH3 | PRDCSSTSCYAGIDAFDI | 3 |
| | CDRL1 | RASQSVSSSYLA | 4 |
| | CDRL2 | GASSRAT | 5 |
| | CDRL3 | QQYGNSPTT | 6 |
| | VH | QVQLVQSGAEVKKPGSSVKVSCKASGGTFSSYAISWVRQA | 7 |
| | | PGQGLEWMGGIIPIFGTANYAQKFQGRVTITADESTSTAY | |
| | | MELSSLRSEDTAVYYCASPRDCSSTSCYAGIDAFDIWGQG | |
| | | TMVTVSS | |
| | VL | EIVLTQSPGTLSLSPGERATLSCRASQSVSSSYLAWYQQK | 8 |
| | | PGQAPRLLIYGASSRATGIPDRFSGSGSGTDFTLTISRLE | |
| | | PEDFAVYYCQQYGNSPTTFGGGTKLEIKRT | |
| | scFv | QVQLVQSGAEVKKPGSSVKVSCKASGGTFSSYAISWVRQA | 9 |
| | | PGQGLEWMGGIIPIFGTANYAQKFQGRVTITADESTSTAY | |
| | | MELSSLRSEDTAVYYCASPRDCSSTSCYAGIDAFDIWGQG | |
| | | TMVTVSSGGGGSGGGGSGGGASEIVLTQSPGTLSLSPGER | |
| | | ATLSCRASQSVSSSYLAWYQQKPGQAPRLLIYGASSRATG | |
| | | IPDRFSGSGSGTDFTLTISRLEPEDFAVYYCQQYGNSPTT | |
| | | FGGGTKLEIKRT | |
| D05 | CDRH1 | SYAMS | 10 |
| | CDRH2 | AISGSGGSTYYADSVKG | 11 |
| | CDRH3 | THSTSWYYFDY | 12 |
| | CDRL1 | RASQSVSSSYLA | 4 |
| | CDRL2 | GASSRAT | 5 |
| | CDRL3 | QQYGSSPRYT | 13 |
| | VH | EVQLLESGGGLVQPGGSLRLSCAASGFTFSSYAMSWVRQA | 14 |
| | | PGKGLEWVSAISGSGGSTYYADSVKGRFTISRDNSKNTLY | |
| | | LQMNSLRAEDTAVYYCAKTHSTSWYYFDYWGRGTLVTVSS | |
| | VL | EIVLTQSPGTLSLSPGERATLSCRASQSVSSSYLAWYQQK | 15 |
| | | PGQAPRLLIYGASSRATGIPDRFSGSGSGTDFTLTISRLE | |
| | | PEDFAVYYCQQYGSSPRYTFGQGTKVDIKRT | |
| | scFv | EVQLLESGGGLVQPGGSLRLSCAASGFTFSSYAMSWVRQA | 16 |
| | | PGKGLEWVSAISGSGGSTYYADSVKGRFTISRDNSKNTLY | |
| | | LQMNSLRAEDTAVYYCAKTHSTSWYYFDYWGRGTLVTVSS | |
| | | GGGGSGGGGSGGGASEIVLTQSPGTLSLSPGERATLSCRA | |
| | | SQSVSSSYLAWYQQKPGQAPRLLIYGASSRATGIPDRFSG | |
| | | SGSGTDFTLTISRLEPEDFAVYYCQQYGSSPRYTFGQGTK | |
| | | VDIKRT | |
| E07 | CDRH1 | SYVMS | 40 |
| | CDRH2 | AIVPSGGDTYYADSVKG | 41 |
| | CDRH3 | DYYGGWFDP | 42 |
| | CDRL1 | RASQSVSSSYLA | 4 |
| | CDRL2 | GASSRAT | 5 |
| | CDRL3 | QQYGSSPRT | 43 |
| | VH | EVQLLESGGGLVQPGGSLRLSCAASGFTFKSYVMSWVRQA | 44 |
| | | PGKGLEWVSAIVPSGGDTYYADSVKGRFTISRDNSKNTLY | |
| | | LQMNSLRAEDTAVYYCAKDYYGGWFDPWGQGTLVTVSS | |
| | VL | EIVLTQSPGTLSLSPGERATLSCRASQSVSSSYLAWYQQK | 45 |
| | | PGQAPRLLIYGASSRATGIPDRFSGSGSGTDFTLTISRLE | |
| | | PEDFAVYYCQQYGSSPRTFGQGTKVEIK | |
| | scFv | EVQLLESGGGLVQPGGSLRLSCAASGFTFKSYVMSWVRQA | 46 |
| | | PGKGLEWVSAIVPSGGDTYYADSVKGRFTISRDNSKNTLY | |
| | | LQMNSLRAEDTAVYYCAKDYYGGWFDPWGQGTLVTVSSGG | |
| | | GGSGGGGSGGGASEIVLTQSPGTLSLSPGERATLSCRASQ | |
| | | SVSSSYLAWYQQKPGQAPRLLIYGASSRATGIPDRFSGSG | |
| | | SGTDFTLTISRLEPEDFAVYYCQQYGSSPRTFGQGTKVEI | |
| | | K | |
| D01 | CDRH1 | SYAMS | 10 |
| | CDRH2 | AISGSGGSTYYADSVKG | 11 |
| | CDRH3 | DYYGGWFDP | 42 |
| | CDRL1 | RASQSVSSSYLA | 4 |
| | CDRL2 | GASSRAT | 5 |
| | CDRL3 | QQYGSSPRT | 43 |
| | VH | EVQLLESGGGLVQPGGSLRLSCAASGFTFSSYAMSWVRQA | 47 |
| | | PGKGLEWVSAISGSGGSTYYADSVKGRFTISRDNSKNTLY | |
| | | LQMNSLRAEDTAVYYCAKDYYGGWFDPWGQGTLVTVSS | |
| | VL | EIVLTQSPGTLSLSPGERATLSCRASQSVSSSYLAWYQQK | 48 |
| | | PGQAPRLLIYGASSRATGIPDRFSGSGSGTDFTLTISRLE | |
| | | PEDFAVYYCQQYGSSPRTFGHGTKLEIK | |
| | scFv | EVQLLESGGGLVQPGGSLRLSCAASGFTFSSYAMSWVRQA | 49 |
| | | PGKGLEWVSAISGSGGSTYYADSVKGRFTISRDNSKNTLY | |
| | | LQMNSLRAEDTAVYYCAKDYYGGWFDPWGQGTLVTVSSGG | |
| | | GGSGGGGSGGGASEIVLTQSPGTLSLSPGERATLSCRASQ | |
| | | SVSSSYLAWYQQKPGQAPRLLIYGASSRATGIPDRFSGSG | |
| | | SGTDFTLTISRLEPEDFAVYYCQQYGSSPRTFGHGTKLEI | |
| | | K | |
| B08 | CDRH1 | SYAMS | 10 |
| | CDRH2 | GISEGGGSTYYADSVKG | 50 |
| | CDRH3 | DYYGGWFDP | 42 |
| | CDRL1 | RASQSVSSSYLA | 4 |
| | CDRL2 | GASSRAT | 5 |
| | CDRL3 | QQYGSSPRT | 43 |
| | VH | EVQLLESGGGLVQPGGSLRLSCAASGFTFSSYAMSWVRQA | 51 |
| | | PGKGLEWVSGISEGGGSTYYADSVKGRFTISRDNSKNTLY | |
| | | LQMNSLRAEDTAVYYCAKDYYGGWFDPWGRGTLVTVSS | |
| | VL | EIVLTQSPGTLSLSPGERATLSCRASQSVSSSYLAWYQQK | 52 |
| | | PGQAPRLLIYGASSRATGIPDRFSGSGSGTDFTLTISRLE | |
| | | PEDFAVYYCQQYGSSPRTFGQGTRLEIK | |
| | scFv | EVQLLESGGGLVQPGGSLRLSCAASGFTFSSYAMSWVRQA | 53 |
| | | PGKGLEWVSGISEGGGSTYYADSVKGRFTISRDNSKNTLY | |
| | | LQMNSLRAEDTAVYYCAKDYYGGWFDPWGRGTLVTVSSGG | |
| | | GGSGGGGSGGGASEIVLTQSPGTLSLSPGERATLSCRASQ | |
| | | SVSSSYLAWYQQKPGQAPRLLIYGASSRATGIPDRFSGSG | |
| | | SGTDFTLTISRLEPEDFAVYYCQQYGSSPRTFGQGTRLEI | |
| | | K | |
| H02 | CDRH1 | SYAMS | 10 |
| | CDRH2 | AISGSGGSTYYADSVKG | 11 |
| | CDRH3 | DLSWYGANFDY | 54 |
| | CDRL1 | RASQSVSSSYLA | 4 |
| | CDRL2 | GASSRAT | 5 |
| | CDRL3 | QQYGSSPVMYT | 55 |
| | VH | EVQLLESGGGLVQPGGSLRLSCAASGFTFSSYAMSWVRQA | 56 |
| | | PGKGLEWVSAISGSGGSTYYADSVKGRFTISRDNSKNTLY | |
| | | LQMNSLRAEDTAVYYCAKDLSWYGANFDYWGQGTLVTVSS | |
| | VL | EIVLTQSPGTLSLSPGERATLSCRASQSVSSSYLAWYQQK | 57 |
| | | PGQAPRLLIYGASSRATGIPDRFSGSGSGTDFTLTISRLE | |
| | | PEDFAVYYCQQYGSSPVMYTFGQGTKVEIK | |
| | scFv | EVQLLESGGGLVQPGGSLRLSCAASGFTFSSYAMSWVRQA | 58 |
| | | PGKGLEWVSAISGSGGSTYYADSVKGRFTISRDNSKNTLY | |
| | | LQMNSLRAEDTAVYYCAKDLSWYGANFDYWGQGTLVTVSS | |
| | | GGGGSGGGGSGGGASEIVLTQSPGTLSLSPGERATLSCRA | |
| | | SQSVSSSYLAWYQQKPGQAPRLLIYGASSRATGIPDRFSG | |
| | | SGSGTDFTLTISRLEPEDFAVYYCQQYGSSPVMYTFGQGT | |
| | | KVEIK | |

In some embodiments, an anti-FAP antibody or antigen binding fragment thereof of the present disclosure comprises one or more of the heavy chain CDRs (e.g., CDRH1, CDRH2, or CDRH3) amino acid sequences from any one of the anti-FAP antibodies selected from Table 2. In some embodiments, an anti-FAP antibody or antigen binding fragment thereof of the present disclosure comprise the CDRH1, CDRH2, and CDRH3 as provided for any one of the antibodies elected from Table 2. In some embodiments, an anti-FAP antibody or antigen binding fragment thereof of the present disclosure comprises one or more of the light chain CDRs (e.g., CDRL1, CDRL2, or CDRL3) amino acid sequences from any one of the anti-FAP antibodies selected from Table 2. In some embodiments, an anti-FAP antibody or antigen binding fragment thereof of the present disclosure comprise the CDRL1, CDRL2, and CDRL3 as provided for any one of the anti-FAP antibodies selected from Table 2.

In some embodiments, an anti-FAP antibody or antigen binding fragment thereof comprises the CDRH1, CDRH2, CDRH3, CDRL1, CDRL2, and CDRL3 as provided for any one of the anti-FAP antibodies selected from Table 2. In some embodiments, antibody heavy and light chain CDR3 domains may play a particularly important role in the binding specificity/affinity of an antibody for an antigen. Accordingly, an anti-FAP antibody or antigen binding fragment thereof may include at least the heavy and/or light chain CDR3s of any one of the anti-FAP antibodies selected from Table 2.

Also within the scope of the present disclosure are functional variants of any of the exemplary anti-FAP antibody or antigen binding fragment thereof as disclosed herein. A functional variant may contain one or more amino acid residue variations in the V_{H} and/or V_{L}, or in one or more of the heavy chain CDRs and/or one or more of the light chain CDRs as relative to the reference antibody, while retaining substantially similar binding and biological activities (e.g., substantially similar binding affinity, binding specificity, inhibitory activity, anti-inflammatory activity, or a combination thereof) as the reference antibody.

In some embodiments, any of the anti-FAP antibody or antigen binding fragment thereof of the disclosure have one or more CDRs (e.g., heavy chain CDR or light chain CDR) sequences substantially similar to any of the CDRH1, CDRH2, CDRH3, CDRL1. CDRL2, and/or CDRL3 sequences from one of the anti-FAP antibodies selected from Table 2. In some embodiments, the position of one or more CDRs along the VH (e.g., CDRH1, CDRH2, or CDRH3) and/or VL (e.g., CDRL1, CDRL2, or CDRL3) region of an antibody described herein can vary by one, two, three, four, five, or six amino acid positions so long as immunospecific binding to FAP (e.g., human FAP) is maintained (e.g., substantially maintained, for example, at least 50%, at least 60%, at least 70%, at least 80%, at least 90%, at least 95% of the binding of the original antibody from which it is derived). For example, in some embodiments, the position defining a CDR of any antibody described herein can vary by shifting the N-terminal and/or C-terminal boundary of the CDR by one, two, three, four, five, or six amino acids, relative to the CDR position of any one of the antibodies described herein, so long as immunospecific binding to FAP (e.g., human FAP) is maintained (e.g., substantially maintained, for example, at least 50%, at least 60%, at least 70%, at least 80%, at least 90%, at least 95% of the binding of the original antibody from which it is derived). In another embodiment, the length of one or more CDRs along the VH (e.g., CDRH1, CDRH2, or CDRH3) and/or VL (e.g., CDRL1, CDRL2, or CDRL3) region of an antibody described herein can vary (e.g., be shorter or longer) by one, two, three, four, five, or more amino acids, so long as immunospecific binding to FAP (e.g., human FAP) is maintained (e.g., substantially maintained, for example, at least 50%, at least 60%, at least 70%, at least 80%, at least 90%, at least 95% of the binding of the original antibody from which it is derived).

Accordingly, in some embodiments, a CDRH1, CDRH2, CDRH3, CDRL1, CDRL2, and/or CDRL3 of an anti-FAP antibody or antigen binding fragment thereof may be one, two, three, four, five or more amino acids shorter than one or more of the CDRs described herein (e.g., CDRs from any of the anti-FAP antibodies selected from Table 2) so long as immunospecific binding to FAP (e.g., human FAP) is maintained (e.g., substantially maintained, for example, at least 50%, at least 60%, at least 70%, at least 80%, at least 90%, at least 95% relative to the binding of the original antibody from which it is derived). In some embodiments, a CDRH1, CDRH2, CDRH3, CDRL1, CDRL2, and/or CDRL3 of an anti-FAP antibody or antigen binding fragment thereof may be one, two, three, four, five or more amino acids longer than one or more of the CDRs described herein (e.g., CDRs from any of the anti-FAP antibodies selected from Table 2) so long as immunospecific binding to FAP (e.g., human FAP) is maintained (e.g., substantially maintained, for example, at least 50%, at least 60%, at least 70%, at least 80%, at least 90%, at least 95% relative to the binding of the original antibody from which it is derived). In some embodiments, the amino portion of a CDRH1, CDRH2, CDRH3, CDRL1, CDRL2, and/or CDRL3 of an anti-FAP antibody or antigen binding fragment thereof can be extended by one, two, three, four, five or more amino acids compared to one or more of the CDRs described herein (e.g., CDRs from any of the anti-FAP antibodies selected from Table 2) so long as immunospecific binding to FAP (e.g., human FAP) is maintained (e.g., substantially maintained, for example, at least 50%, at least 60%, at least 70%, at least 80%, at least 90%, at least 95% relative to the binding of the original antibody from which it is derived). In some embodiments, the carboxy portion of a CDRH1, CDRH2, CDRH3, CDRL1, CDRL2, and/or CDRL3 of an anti-FAP antibody or antigen binding fragment thereof can be extended by one, two, three, four, five or more amino acids compared to one or more of the CDRs described herein (e.g., CDRs from any of the anti-FAP antibodies selected from Table 2) so long as immunospecific binding to FAP (e.g., human FAP) is maintained (e.g., substantially maintained, for example, at least 50%, at least 60%, at least 70%, at least 80%, at least 90%, at least 95% relative to the binding of the original antibody from which it is derived). In some embodiments, the amino portion of a CDRH1, CDRH2, CDRH3, CDRL1, CDRL2, and/or CDRL3 of an anti-FAP antibody or antigen binding fragment thereof can be shortened by one, two, three, four, five or more amino acids compared to one or more of the CDRs described herein (e.g., CDRs from any of the anti-FAP antibodies selected from Table 2) so long as immunospecific binding to FAP (e.g., human FAP) is maintained (e.g., substantially maintained, for example, at least 50%, at least 60%, at least 70%, at least 80%, at least 90%, at least 95% relative to the binding of the original antibody from which it is derived). In some embodiments, the carboxy portion of a CDRH1, CDRH2, CDRH3, CDRL1, CDRL2, and/or CDRL3 of an anti-FAP antibody or antigen binding fragment thereof can be shortened by one, two, three, four, five or more amino acids compared to one or more of the CDRs described herein (e.g., CDRs from any of the anti-FAP antibodies selected from Table 2) so long as immunospecific binding to FAP (e.g., human FAP) is maintained (e.g., substantially maintained, for example, at least 50%, at least 60%, at least 70%, at least 80%, at least 90%, at least 95% relative to the binding of the original antibody from which it is derived). Any method can be used to ascertain whether immunospecific binding to FAP (e.g., human FAP) is maintained, for example, using binding assays and conditions described in the art.

In some examples, an anti-FAP antibody or antigen binding fragment thereof comprises one or more CDR (e.g., heavy chain CDRs or light chain CDR) sequences substantially similar to any one of the anti-FAP antibodies selected from Table 2. For example, the antibodies may include one or more CDR sequence(s) from any of the anti-FAP antibodies selected from Table 2 containing up to 5, 4, 3, 2, or 1 amino acid residue variations as compared to the corresponding CDR region in any one of the CDRs provided herein (e.g., CDRs from any of the anti-FAP antibodies selected from Table 2) so long as immunospecific binding to FAP (e.g., human FAP) is maintained (e.g., substantially maintained, for example, at least 50%, at least 60%, at least 70%, at least 80%, at least 90%, at least 95% relative to the binding of the original antibody from which it is derived). In some embodiments, any of the amino acid variations in any of the CDRs provided herein may be conservative variations. Conservative variations can be introduced into the CDRs at positions where the residues are not likely to be involved in interacting with a FAP protein (e.g., human FAP), for example, as determined based on a crystal structure.

Some aspects of the disclosure provide anti-FAP antibodies that comprise one or more of the heavy chain variable (VH) and/or light chain variable (VL) domains provided herein. In some embodiments, any of the VH domains provided herein include one or more of the heavy chain CDR sequences (e.g., CDRH1, CDRH2, and CDRH3) provided herein, for example, any of the heavy chain CDR sequences provided in any one of the anti-FAP antibodies selected from Table 2. In some embodiments, any of the VL domains provided herein include one or more of the CDR-L sequences (e.g., CDRL1, CDRL2, and CDRL3) provided herein, for example, any of the light chain CDR sequences provided in any one of the anti-FAP antibodies selected from Table 2.

In some embodiments, an anti-FAP antibody or antigen binding fragment thereof includes any antibody that comprises a heavy chain variable domain and/or a light chain variable domain of any one of the anti-FAP antibodies selected from Table 2, and variants thereof. In some embodiments, an anti-FAP antibody or antigen binding fragment thereof includes any antibody that comprises the heavy chain variable and light chain variable pairs of any anti-FAP antibodies selected from Table 2.

Aspects of the disclosure provide anti-FAP antibodies or antigen binding fragments thereof comprising a heavy chain variable (VH) and/or a light chain variable (VL) domain amino acid sequence homologous to any of those described herein. In some embodiments, an anti-FAP antibody or antigen binding fragment thereof comprises a VH or a VL that is at least 75% (e.g., at least 80%, at least 85%, at least 90%, at least 91%, at least 92%, at least 93%, at least 94%, at least 95%, at least 96%, at least 97%, at least 98%, at least 99%, or 100%) identical to the VH and/or any VL of any one of the anti-FAP antibodies selected from Table 2. In some embodiments, the homologous VH and/or a VL amino acid sequences do not vary within any of the CDR sequences provided herein. For example, in some embodiments, the degree of sequence variation (e.g., at least 80%, at least 85%, at least 90%, at least 91%, at least 92%, at least 93%, at least 94%, at least 95%, at least 96%, at least 97%, at least 98%, at least 99%, or 100%) may occur within a VH and/or a VL sequence excluding any of the CDR sequences provided herein. In some embodiments, any of the anti-FAP antibodies or antigen binding fragments thereof provided herein comprise a VH sequence and a VL sequence that comprises a framework sequence that is at least 75%, 80%, 85%, 90%, 95%, 98%, or 99% identical to the framework sequence of any anti-FAP antibodies selected from Table 2. In some embodiments, an anti-FAP antibody or antigen binding fragment thereof comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH of any of the anti-FAP antibodies listed in Table 2. Alternatively or in addition, an anti-FAP antibody or antigen binding fragment thereof comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL of any one of the anti-FAP antibodies listed in Table 2.

In some embodiments, an anti-FAP antibody or antigen binding fragment thereof is a humanized antibody (e.g., a humanized variant containing one or more CDRs of Table 2). In some embodiments, an anti-FAP antibody or antigen binding fragment thereof comprises a CDRH1, a CDRH2, a CDRH3, a CDRL1, a CDRL2, and a CDRL3 that are the same as the CDRH1, CDRH2, CDRH3, CDRL1, CDRL2, and CDRL3 shown in Table 2, and comprises a humanized VH and/or a humanized VL. In some embodiments, an anti-FAP antibody or antigen binding fragment thereof is a humanized variant comprising one or more amino acid substitutions (e.g., in the VH framework region) as compared with any one of the VHs listed in Table 2, and/or one or more amino acid substitutions (e.g., in the VL framework region) as compared with any one of the VLs listed in Table 2.

In some embodiments, an anti-FAP antibody or antigen binding fragment thereof comprises a CDRH1, a CDRH2 and a CDRH3 of a heavy chain variable domain (VH) having the amino acid sequence of SEQ ID NO: 7. Alternatively or in addition, an anti-FAP antibody or antigen binding fragment thereof comprises a CDRL1, a CDRL2 and a CDRL3 of a light chain variable domain (VL) having the amino acid sequence of SEQ ID NO: 8.

In some embodiments, according to the Kabat definition system, an anti-FAP antibody or antigen binding fragment thereof comprises a CDRH3 having the amino acid sequence of SEQ ID NO: 3. In some embodiments, according to the Kabat definition system, an anti-FAP antibody or antigen binding fragment thereof comprises a CDRH1 having the amino acid sequence of SEQ ID NO: 1, a CDRH2 having the amino acid sequence of SEQ ID NO: 2, and a CDRH3 having the amino acid sequence of SEQ ID NO: 3. In some embodiments, according to the Kabat definition system, an anti-FAP antibody or antigen binding fragment thereof comprises a CDRH1 having the amino acid sequence of SEQ ID NO: 1, a CDRH2 having the amino acid sequence of SEQ ID NO: 2, a CDRH3 having the amino acid sequence of SEQ ID NO: 3, a CDRL1 having the amino acid sequence of SEQ ID NO: 4, a CDRL2 having the amino acid sequence of SEQ ID NO: 5, and a CDRL3 having the amino acid sequence of SEQ ID NO: 6.

In some embodiments, an anti-FAP antibody or antigen binding fragment thereof comprises a CDRH1, a CDRH2, and a CDRH3, which collectively contains no more than 5 amino acid variations (e.g., no more than 5, 4, 3, 2, or 1 amino acid variation) as compared with the CDRH1 having the amino acid sequence of SEQ ID NO: 1, CDRH2 having the amino acid sequence of SEQ ID NO: 2, and CDRH3 having the amino acid sequence of SEQ ID NO: 3. “Collectively,” as used anywhere in the present disclosure, means that the total number of amino acid variations in all of the three heavy chain CDRs is within the defined range. Alternatively or in addition, an anti-FAP antibody or antigen binding fragment thereof comprises a CDRL1, a CDRL2, and a CDRL3, which collectively contains no more than 5 amino acid variations (e.g., no more than 5, 4, 3, 2 or 1 amino acid variation) as compared with the CDRL1 having the amino acid sequence of SEQ ID NO: 4. CDRL2 having the amino acid sequence of SEQ ID NO: 5, and CDRL3 having the amino acid sequence of SEQ ID NO: 6.

In some embodiments, an anti-FAP antibody or antigen binding fragment thereof comprises a CDRH1, a CDRH2, and a CDRH3 that collectively are at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the CDRH1 having the amino acid sequence of SEQ ID NO: 1, CDRH2 having the amino acid sequence of SEQ ID NO: 2, and CDRH3 having the amino acid sequence of SEQ ID NO: 3. Alternatively or in addition, an anti-FAP antibody or antigen binding fragment thereof comprises a CDRL1, a CDRL2, and a CDRL3 that collectively are at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the to the CDRL1 having the amino acid sequence of SEQ ID NO: 4, CDRL2 having the amino acid sequence of SEQ ID NO: 5, and CDRL3 having the amino acid sequence of SEQ ID NO: 6.

In some embodiments, an anti-FAP antibody or antigen binding fragment thereof comprises: a CDRH1 having no more than 3 amino acid variations (e.g., no more than 3, 2, or 1 amino acid variation) as compared with the CDRH1 having the amino acid sequence of SEQ ID NO: 1; a CDRH2 having no more than 3 amino acid variations (e.g., no more than 3, 2, or 1 amino acid variation) as compared with the CDRH2 having the amino acid sequence of SEQ ID NO: 2; and/or a CDRH3 having no more than 3 amino acid variations (e.g., no more than 3, 2, or 1 amino acid variation) as compared with the CDRH3 having the amino acid sequence of SEQ ID NO: 3. Alternatively or in addition, an anti-FAP antibody or antigen binding fragment thereof comprises: a CDRL1 having no more than 3 amino acid variations (e.g., no more than 3, 2, or 1 amino acid variation) as compared with the CDRL1 having the amino acid sequence of SEQ ID NO: 4; a CDRL2 having no more than 3 amino acid variations (e.g., no more than 3, 2, or 1 amino acid variation) as compared with the CDRL2 having the amino acid sequence of SEQ ID NO: 5; and/or a CDRL3 having no more than 3 amino acid variations (e.g., no more than 3, 2, or 1 amino acid variation) as compared with the CDRL3 having the amino acid sequence of SEQ ID NO: 6.

In some embodiments, an anti-FAP antibody or antigen binding fragment thereof comprises a VH comprising the amino acid sequence of SEQ ID NO: 7. Alternatively or in addition, an anti-FAP antibody or antigen binding fragment thereof comprises a VL comprising the amino acid sequence of SEQ ID NO: 8. In some embodiments, an anti-FAP antibody or antigen binding fragment thereof comprises a VH comprising the amino acid sequence of SEQ ID NO: 7 and a VL comprising the amino acid sequence of SEQ ID NO: 8.

In some embodiments, an anti-FAP antibody or antigen binding fragment thereof comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 7. Alternatively or in addition, an anti-FAP antibody or antigen binding fragment thereof comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 8.

In some embodiments, an anti-FAP antibody or antigen binding fragment thereof comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VH as set forth in SEQ ID NO: 7. Alternatively or in addition, an anti-FAP antibody or antigen binding fragment thereof comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VL as set forth in SEQ ID NO: 8.

In some embodiments, an anti-FAP antibody or antigen binding fragment thereof comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 7. Alternatively or in addition, an anti-FAP antibody or antigen binding fragment thereof comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 8. In some embodiments, the number of amino acid variations (e.g., no more than 20, 19, 18, 17, 16, 15, 14, 13, 12, 11, 10, 9, 8, 7, 6, 5, 4, 3, 2, or 1 amino acid variation) may occur within a VH of SEQ ID NO: 7 and/or a VL of SEQ ID NO: 8 excluding any of the CDR sequences therein. In some embodiments, an anti-FAP antibody or antigen binding fragment thereof comprises a heavy chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VH of SEQ ID NO: 7, and/or a light chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VL of SEQ ID NO: 8.

In some embodiments, an anti-FAP antibody or antigen binding fragment thereof comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VH as set forth in SEQ ID NO: 7. Alternatively or in addition, an anti-FAP antibody or antigen binding fragment thereof comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VL as set forth in SEQ ID NO: 8. In some embodiments, the degree of sequence variation (e.g., at least 80%, at least 85%, at least 90%, at least 91%, at least 92%, at least 93%, at least 94%, at least 95%, at least 96%, at least 97%, at least 98%, at least 99%, or 100%) may occur within a VH of SEQ ID NO: 7, and/or a VL of SEQ ID NO: 8 excluding any of the CDR sequences therein. In some embodiments, an anti-FAP antibody or antigen binding fragment thereof comprises a heavy chain variable sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the framework sequence of a VH of SEQ ID NO: 7, and/or a light chain variable sequence that comprises a framework sequence that at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the framework sequence of a VL of SEQ ID NO: 8.

In some embodiments, an anti-FAP antibody or antigen binding fragment thereof comprises a CDRH1, a CDRH2 and a CDRH3 of a heavy chain variable domain (VH) having the amino acid sequence of SEQ ID NO: 14. Alternatively or in addition, an anti-FAP antibody or antigen binding fragment thereof comprises a CDRL1, a CDRL2 and a CDRL3 of a light chain variable domain (VL) having the amino acid sequence of SEQ ID NO: 15.

In some embodiments, according to the Kabat definition system, an anti-FAP antibody or antigen binding fragment thereof comprises a CDRH3 having the amino acid sequence of SEQ ID NO: 12. In some embodiments, according to the Kabat definition system, an anti-FAP antibody or antigen binding fragment thereof comprises a CDRH1 having the amino acid sequence of SEQ ID NO: 10, a CDRH2 having the amino acid sequence of SEQ ID NO: 11, and a CDRH3 having the amino acid sequence of SEQ ID NO: 12. In some embodiments, according to the Kabat definition system, an anti-FAP antibody or antigen binding fragment thereof comprises a CDRH1 having the amino acid sequence of SEQ ID NO: 10, a CDRH2 having the amino acid sequence of SEQ ID NO: 11, a CDRH3 having the amino acid sequence of SEQ ID NO: 12, a CDRL1 having the amino acid sequence of SEQ ID NO: 4, a CDRL2 having the amino acid sequence of SEQ ID NO: 5, and a CDRL3 having the amino acid sequence of SEQ ID NO: 13.

In some embodiments, an anti-FAP antibody or antigen binding fragment thereof comprises a CDRH1, a CDRH2, and a CDRH3, which collectively contains no more than 5 amino acid variations (e.g., no more than 5, 4, 3, 2, or 1 amino acid variation) as compared with the CDRH1 having the amino acid sequence of SEQ ID NO: 10, CDRH2 having the amino acid sequence of SEQ ID NO: 11, and CDRH3 having the amino acid sequence of SEQ ID NO: 12. Alternatively or in addition, an anti-FAP antibody or antigen binding fragment thereof comprises a CDRL1, a CDRL2, and a CDRL3, which collectively contains no more than 5 amino acid variations (e.g., no more than 5, 4, 3, 2 or 1 amino acid variation) as compared with the CDRL1 having the amino acid sequence of SEQ ID NO: 4. CDRL2 having the amino acid sequence of SEQ ID NO: 5, and CDRL3 having the amino acid sequence of SEQ ID NO: 13.

In some embodiments, an anti-FAP antibody or antigen binding fragment thereof comprises a CDRH1, a CDRH2, and a CDRH3 that collectively are at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the CDRH1 having the amino acid sequence of SEQ ID NO: 10, CDRH2 having the amino acid sequence of SEQ ID NO: 11, and CDRH3 having the amino acid sequence of SEQ ID NO: 12. Alternatively or in addition, an anti-FAP antibody or antigen binding fragment thereof comprises a CDRL1, a CDRL2, and a CDRL3 that collectively are at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the to the CDRL1 having the amino acid sequence of SEQ ID NO: 4, CDRL2 having the amino acid sequence of SEQ ID NO: 5, and CDRL3 having the amino acid sequence of SEQ ID NO: 13.

In some embodiments, an anti-FAP antibody or antigen binding fragment thereof comprises: a CDRH1 having no more than 3 amino acid variations (e.g., no more than 3, 2, or 1 amino acid variation) as compared with the CDRH1 having the amino acid sequence of SEQ ID NO: 10; a CDRH2 having no more than 3 amino acid variations (e.g., no more than 3, 2, or 1 amino acid variation) as compared with the CDRH2 having the amino acid sequence of SEQ ID NO: 11; and/or a CDRH3 having no more than 3 amino acid variations (e.g., no more than 3, 2, or 1 amino acid variation) as compared with the CDRH3 having the amino acid sequence of SEQ ID NO: 12. Alternatively or in addition, an anti-FAP antibody or antigen binding fragment thereof comprises: a CDRL1 having no more than 3 amino acid variations (e.g., no more than 3, 2, or 1 amino acid variation) as compared with the CDRL1 having the amino acid sequence of SEQ ID NO: 4; a CDRL2 having no more than 3 amino acid variations (e.g., no more than 3, 2, or 1 amino acid variation) as compared with the CDRL2 having the amino acid sequence of SEQ ID NO: 5; and/or a CDRL3 having no more than 3 amino acid variations (e.g., no more than 3, 2, or 1 amino acid variation) as compared with the CDRL3 having the amino acid sequence of SEQ ID NO: 13.

In some embodiments, an anti-FAP antibody or antigen binding fragment thereof comprises a VH comprising the amino acid sequence of SEQ ID NO: 14. Alternatively or in addition, an anti-FAP antibody or antigen binding fragment thereof comprises a VL comprising the amino acid sequence of SEQ ID NO: 15. In some embodiments, an anti-FAP antibody or antigen binding fragment thereof comprises a VH comprising the amino acid sequence of SEQ ID NO: 14 and a VL comprising the amino acid sequence of SEQ ID NO: 15.

In some embodiments, an anti-FAP antibody or antigen binding fragment thereof comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 14. Alternatively or in addition, an anti-FAP antibody or antigen binding fragment thereof comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 15.

In some embodiments, an anti-FAP antibody or antigen binding fragment thereof comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VH as set forth in SEQ ID NO: 14. Alternatively or in addition, an anti-FAP antibody or antigen binding fragment thereof comprises a VL comprising an amino acid sequence that is at least 80 (e.g., at least 80%, at least 85%, at least 90%, at least 91%, at least 92%, at least 93%, at least 94%, at least 95%, at least 96%, at least 97%, at least 98%, at least 99%, or 100%) identical to the VL as set forth in SEQ ID NO: 15.

In some embodiments, an anti-FAP antibody or antigen binding fragment thereof comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 14. Alternatively or in addition, an anti-FAP antibody or antigen binding fragment thereof comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 15. In some embodiments, the number of amino acid variations (e.g., no more than 20, 19, 18, 17, 16, 15, 14, 13, 12, 11, 10, 9, 8, 7, 6, 5, 4, 3, 2, or 1 amino acid variation) may occur within a VH of SEQ ID NO: 14 and/or a VL of SEQ ID NO: 15 excluding any of the CDR sequences therein. In some embodiments, an anti-FAP antibody or antigen binding fragment thereof comprises a heavy chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VH of SEQ ID NO: 14, and/or a light chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VL of SEQ ID NO: 15.

In some embodiments, an anti-FAP antibody or antigen binding fragment thereof comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VH as set forth in SEQ ID NO: 14. Alternatively or in addition, an anti-FAP antibody or antigen binding fragment thereof comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VL as set forth in SEQ ID NO: 15. In some embodiments, the degree of sequence variation (e.g., at least 80%, at least 85%, at least 90%, at least 91%, at least 92%, at least 93%, at least 94%, at least 95%, at least 96%, at least 97%, at least 98%, at least 99%, or 100%) may occur within a VH of SEQ ID NO: 14, and/or a VL of SEQ ID NO: 15 excluding any of the CDR sequences therein. In some embodiments, an anti-FAP antibody or antigen binding fragment thereof comprises a heavy chain variable sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the framework sequence of a VH of SEQ ID NO: 14, and/or a light chain variable sequence that comprises a framework sequence that at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the framework sequence of a VL of SEQ ID NO: 15.

In some embodiments, an anti-FAP antibody or antigen binding fragment thereof comprises a CDRH1, a CDRH2 and a CDRH3 of a heavy chain variable domain (VH) having the amino acid sequence of SEQ ID NO: 44. Alternatively or in addition, an anti-FAP antibody or antigen binding fragment thereof comprises a CDRL1, a CDRL2 and a CDRL3 of a light chain variable domain (VL) having the amino acid sequence of SEQ ID NO: 45.

In some embodiments, according to the Kabat definition system, an anti-FAP antibody or antigen binding fragment thereof comprises a CDRH3 having the amino acid sequence of SEQ ID NO: 42. In some embodiments, according to the Kabat definition system, an anti-FAP antibody or antigen binding fragment thereof comprises a CDRH1 having the amino acid sequence of SEQ ID NO: 40, a CDRH2 having the amino acid sequence of SEQ ID NO: 41, and a CDRH3 having the amino acid sequence of SEQ ID NO: 42. In some embodiments, according to the Kabat definition system, an anti-FAP antibody or antigen binding fragment thereof comprises a CDRH1 having the amino acid sequence of SEQ ID NO: 40, a CDRH2 having the amino acid sequence of SEQ ID NO: 41, a CDRH3 having the amino acid sequence of SEQ ID NO: 42, a CDRL1 having the amino acid sequence of SEQ ID NO: 4, a CDRL2 having the amino acid sequence of SEQ ID NO: 5, and a CDRL3 having the amino acid sequence of SEQ ID NO: 43.

In some embodiments, an anti-FAP antibody or antigen binding fragment thereof comprises a CDRH1, a CDRH2, and a CDRH3, which collectively contains no more than 5 amino acid variations (e.g., no more than 5, 4, 3, 2, or 1 amino acid variation) as compared with the CDRH1 having the amino acid sequence of SEQ ID NO: 40, CDRH2 having the amino acid sequence of SEQ ID NO: 41, and CDRH3 having the amino acid sequence of SEQ ID NO: 42. Alternatively or in addition, an anti-FAP antibody or antigen binding fragment thereof comprises a CDRL1, a CDRL2, and a CDRL3, which collectively contains no more than 5 amino acid variations (e.g., no more than 5, 4, 3, 2 or 1 amino acid variation) as compared with the CDRL1 having the amino acid sequence of SEQ ID NO: 4, CDRL2 having the amino acid sequence of SEQ ID NO: 5, and CDRL3 having the amino acid sequence of SEQ ID NO: 43.

In some embodiments, an anti-FAP antibody or antigen binding fragment thereof comprises a CDRH1, a CDRH2, and a CDRH3 that collectively are at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the CDRH1 having the amino acid sequence of SEQ ID NO: 40, CDRH2 having the amino acid sequence of SEQ ID NO: 41, and CDRH3 having the amino acid sequence of SEQ ID NO: 42. Alternatively or in addition, an anti-FAP antibody or antigen binding fragment thereof comprises a CDRL1, a CDRL2, and a CDRL3 that collectively are at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the to the CDRL1 having the amino acid sequence of SEQ ID NO: 4, CDRL2 having the amino acid sequence of SEQ ID NO: 5, and CDRL3 having the amino acid sequence of SEQ ID NO: 43.

In some embodiments, an anti-FAP antibody or antigen binding fragment thereof comprises: a CDRH1 having no more than 3 amino acid variations (e.g., no more than 3, 2, or 1 amino acid variation) as compared with the CDRH1 having the amino acid sequence of SEQ ID NO: 40; a CDRH2 having no more than 3 amino acid variations (e.g., no more than 3, 2, or 1 amino acid variation) as compared with the CDRH2 having the amino acid sequence of SEQ ID NO: 41; and/or a CDRH3 having no more than 3 amino acid variations (e.g., no more than 3, 2, or 1 amino acid variation) as compared with the CDRH3 having the amino acid sequence of SEQ ID NO: 42. Alternatively or in addition, an anti-FAP antibody or antigen binding fragment thereof comprises: a CDRL1 having no more than 3 amino acid variations (e.g., no more than 3, 2, or 1 amino acid variation) as compared with the CDRL1 having the amino acid sequence of SEQ ID NO: 4; a CDRL2 having no more than 3 amino acid variations (e.g., no more than 3, 2, or 1 amino acid variation) as compared with the CDRL2 having the amino acid sequence of SEQ ID NO: 5; and/or a CDRL3 having no more than 3 amino acid variations (e.g., no more than 3, 2, or 1 amino acid variation) as compared with the CDRL3 having the amino acid sequence of SEQ ID NO: 43.

In some embodiments, an anti-FAP antibody or antigen binding fragment thereof comprises a VH comprising the amino acid sequence of SEQ ID NO: 44. Alternatively or in addition, an anti-FAP antibody or antigen binding fragment thereof comprises a VL comprising the amino acid sequence of SEQ ID NO: 45. In some embodiments, an anti-FAP antibody or antigen binding fragment thereof comprises a VH comprising the amino acid sequence of SEQ ID NO: 44 and a VL comprising the amino acid sequence of SEQ ID NO: 45.

In some embodiments, an anti-FAP antibody or antigen binding fragment thereof comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 44. Alternatively or in addition, an anti-FAP antibody or antigen binding fragment thereof comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 45.

In some embodiments, an anti-FAP antibody or antigen binding fragment thereof comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VH as set forth in SEQ ID NO: 44. Alternatively or in addition, an anti-FAP antibody or antigen binding fragment thereof comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VL as set forth in SEQ ID NO: 45.

In some embodiments, an anti-FAP antibody or antigen binding fragment thereof comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 44. Alternatively or in addition, an anti-FAP antibody or antigen binding fragment thereof comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 45. In some embodiments, the number of amino acid variations (e.g., no more than 20, 19, 18, 17, 16, 15, 14, 13, 12, 11, 10, 9, 8, 7, 6, 5, 4, 3, 2, or 1 amino acid variation) may occur within a VH of SEQ ID NO: 44 and/or a VL of SEQ ID NO: 45 excluding any of the CDR sequences therein. In some embodiments, an anti-FAP antibody or antigen binding fragment thereof comprises a heavy chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VH of SEQ ID NO: 44, and/or a light chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VL of SEQ ID NO: 45.

In some embodiments, an anti-FAP antibody or antigen binding fragment thereof comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VH as set forth in SEQ ID NO: 44. Alternatively or in addition, an anti-FAP antibody or antigen binding fragment thereof comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VL as set forth in SEQ ID NO: 45. In some embodiments, the degree of sequence variation (e.g., at least 80%, at least 85%, at least 90%, at least 91%, at least 92%, at least 93%, at least 94%, at least 95%, at least 96%, at least 97%, at least 98%, at least 99%, or 100%) may occur within a VH of SEQ ID NO: 44, and/or a VL of SEQ ID NO: 45 excluding any of the CDR sequences therein. In some embodiments, an anti-FAP antibody or antigen binding fragment thereof comprises a heavy chain variable sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the framework sequence of a VH of SEQ ID NO: 44, and/or a light chain variable sequence that comprises a framework sequence that at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the framework sequence of a VL of SEQ ID NO: 45.

In some embodiments, an anti-FAP antibody or antigen binding fragment thereof comprises a CDRH1, a CDRH2 and a CDRH3 of a heavy chain variable domain (VH) having the amino acid sequence of SEQ ID NO: 47. Alternatively or in addition, an anti-FAP antibody or antigen binding fragment thereof comprises a CDRL1, a CDRL2 and a CDRL3 of a light chain variable domain (VL) having the amino acid sequence of SEQ ID NO: 48.

In some embodiments, according to the Kabat definition system, an anti-FAP antibody or antigen binding fragment thereof comprises a CDRH3 having the amino acid sequence of SEQ ID NO: 42. In some embodiments, according to the Kabat definition system, an anti-FAP antibody or antigen binding fragment thereof comprises a CDRH1 having the amino acid sequence of SEQ ID NO: 10, a CDRH2 having the amino acid sequence of SEQ ID NO: 11, and a CDRH3 having the amino acid sequence of SEQ ID NO: 42. In some embodiments, according to the Kabat definition system, an anti-FAP antibody or antigen binding fragment thereof comprises a CDRH1 having the amino acid sequence of SEQ ID NO: 10, a CDRH2 having the amino acid sequence of SEQ ID NO: 11, a CDRH3 having the amino acid sequence of SEQ ID NO: 42, a CDRL1 having the amino acid sequence of SEQ ID NO: 4, a CDRL2 having the amino acid sequence of SEQ ID NO: 5, and a CDRL3 having the amino acid sequence of SEQ ID NO: 43.

In some embodiments, an anti-FAP antibody or antigen binding fragment thereof comprises a CDRH1, a CDRH2, and a CDRH3, which collectively contains no more than 5 amino acid variations (e.g., no more than 5, 4, 3, 2, or 1 amino acid variation) as compared with the CDRH1 having the amino acid sequence of SEQ ID NO: 10, CDRH2 having the amino acid sequence of SEQ ID NO:11, and CDRH3 having the amino acid sequence of SEQ ID NO: 42. Alternatively or in addition, an anti-FAP antibody or antigen binding fragment thereof comprises a CDRL1, a CDRL2, and a CDRL3, which collectively contains no more than 5 amino acid variations (e.g., no more than 5, 4, 3, 2 or 1 amino acid variation) as compared with the CDRL1 having the amino acid sequence of SEQ ID NO: 4, CDRL2 having the amino acid sequence of SEQ ID NO: 5, and CDRL3 having the amino acid sequence of SEQ ID NO: 43.

In some embodiments, an anti-FAP antibody or antigen binding fragment thereof comprises a CDRH1, a CDRH2, and a CDRH3 that collectively are at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the CDRH1 having the amino acid sequence of SEQ ID NO: 10, CDRH2 having the amino acid sequence of SEQ ID NO: 11, and CDRH3 having the amino acid sequence of SEQ ID NO: 42. Alternatively or in addition, an anti-FAP antibody or antigen binding fragment thereof comprises a CDRL1, a CDRL2, and a CDRL3 that collectively are at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the to the CDRL1 having the amino acid sequence of SEQ ID NO: 4, CDRL2 having the amino acid sequence of SEQ ID NO: 5, and CDRL3 having the amino acid sequence of SEQ ID NO: 43.

In some embodiments, an anti-FAP antibody or antigen binding fragment thereof comprises: a CDRH1 having no more than 3 amino acid variations (e.g., no more than 3, 2, or 1 amino acid variation) as compared with the CDRH1 having the amino acid sequence of SEQ ID NO: 10; a CDRH2 having no more than 3 amino acid variations (e.g., no more than 3, 2, or 1 amino acid variation) as compared with the CDRH2 having the amino acid sequence of SEQ ID NO: 11; and/or a CDRH3 having no more than 3 amino acid variations (e.g., no more than 3, 2, or 1 amino acid variation) as compared with the CDRH3 having the amino acid sequence of SEQ ID NO: 42. Alternatively or in addition, an anti-FAP antibody or antigen binding fragment thereof comprises: a CDRL1 having no more than 3 amino acid variations (e.g., no more than 3, 2, or 1 amino acid variation) as compared with the CDRL1 having the amino acid sequence of SEQ ID NO: 4; a CDRL2 having no more than 3 amino acid variations (e.g., no more than 3, 2, or 1 amino acid variation) as compared with the CDRL2 having the amino acid sequence of SEQ ID NO: 5; and/or a CDRL3 having no more than 3 amino acid variations (e.g., no more than 3, 2, or 1 amino acid variation) as compared with the CDRL3 having the amino acid sequence of SEQ ID NO: 43.

In some embodiments, an anti-FAP antibody or antigen binding fragment thereof comprises a VH comprising the amino acid sequence of SEQ ID NO: 47. Alternatively or in addition, an anti-FAP antibody or antigen binding fragment thereof comprises a VL comprising the amino acid sequence of SEQ ID NO: 48. In some embodiments, an anti-FAP antibody or antigen binding fragment thereof comprises a VH comprising the amino acid sequence of SEQ ID NO: 47 and a VL comprising the amino acid sequence of SEQ ID NO: 48.

In some embodiments, an anti-FAP antibody or antigen binding fragment thereof comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 47. Alternatively or in addition, an anti-FAP antibody or antigen binding fragment thereof comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 48.

In some embodiments, an anti-FAP antibody or antigen binding fragment thereof comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VH as set forth in SEQ ID NO: 47. Alternatively or in addition, an anti-FAP antibody or antigen binding fragment thereof comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VL as set forth in SEQ ID NO: 48.

In some embodiments, an anti-FAP antibody or antigen binding fragment thereof comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 47. Alternatively or in addition, an anti-FAP antibody or antigen binding fragment thereof comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 48. In some embodiments, the number of amino acid variations (e.g., no more than 20, 19, 18, 17, 16, 15, 14, 13, 12, 11, 10, 9, 8, 7, 6, 5, 4, 3, 2, or 1 amino acid variation) may occur within a VH of SEQ ID NO: 47 and/or a VL of SEQ ID NO: 48 excluding any of the CDR sequences therein. In some embodiments, an anti-FAP antibody or antigen binding fragment thereof comprises a heavy chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VH of SEQ ID NO: 47, and/or a light chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VL of SEQ ID NO: 48.

In some embodiments, an anti-FAP antibody or antigen binding fragment thereof comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VH as set forth in SEQ ID NO: 47. Alternatively or in addition, an anti-FAP antibody or antigen binding fragment thereof comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VL as set forth in SEQ ID NO: 48. In some embodiments, the degree of sequence variation (e.g., at least 80%, at least 85%, at least 90%, at least 91%, at least 92%, at least 93%, at least 94%, at least 95%, at least 96%, at least 97%, at least 98%, at least 99%, or 100%) may occur within a VH of SEQ ID NO: 47, and/or a VL of SEQ ID NO: 48 excluding any of the CDR sequences therein. In some embodiments, an anti-FAP antibody or antigen binding fragment thereof comprises a heavy chain variable sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the framework sequence of a VH of SEQ ID NO: 47, and/or a light chain variable sequence that comprises a framework sequence that at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the framework sequence of a VL of SEQ ID NO: 48.

In some embodiments, an anti-FAP antibody or antigen binding fragment thereof comprises a CDRH1, a CDRH2 and a CDRH3 of a heavy chain variable domain (VH) having the amino acid sequence of SEQ ID NO: 51. Alternatively or in addition, an anti-FAP antibody or antigen binding fragment thereof comprises a CDRL1, a CDRL2 and a CDRL3 of a light chain variable domain (VL) having the amino acid sequence of SEQ ID NO: 52.

In some embodiments, according to the Kabat definition system, an anti-FAP antibody or antigen binding fragment thereof comprises a CDRH3 having the amino acid sequence of SEQ ID NO: 42. In some embodiments, according to the Kabat definition system, an anti-FAP antibody or antigen binding fragment thereof comprises a CDRH1 having the amino acid sequence of SEQ ID NO: 10, a CDRH2 having the amino acid sequence of SEQ ID NO: 50, and a CDRH3 having the amino acid sequence of SEQ ID NO: 42. In some embodiments, according to the Kabat definition system, an anti-FAP antibody or antigen binding fragment thereof comprises a CDRH1 having the amino acid sequence of SEQ ID NO: 10, a CDRH2 having the amino acid sequence of SEQ ID NO: 50, a CDRH3 having the amino acid sequence of SEQ ID NO: 42, a CDRL1 having the amino acid sequence of SEQ ID NO: 4, a CDRL2 having the amino acid sequence of SEQ ID NO: 5, and a CDRL3 having the amino acid sequence of SEQ ID NO: 43.

In some embodiments, an anti-FAP antibody or antigen binding fragment thereof comprises a CDRH1, a CDRH2, and a CDRH3, which collectively contains no more than 5 amino acid variations (e.g., no more than 5, 4, 3, 2, or 1 amino acid variation) as compared with the CDRH1 having the amino acid sequence of SEQ ID NO: 10, CDRH2 having the amino acid sequence of SEQ ID NO:5, and CDRH3 having the amino acid sequence of SEQ ID NO: 42. Alternatively or in addition, an anti-FAP antibody or antigen binding fragment thereof comprises a CDRL1, a CDRL2, and a CDRL3, which collectively contains no more than 5 amino acid variations (e.g., no more than 5, 4, 3, 2 or 1 amino acid variation) as compared with the CDRL1 having the amino acid sequence of SEQ ID NO: 4. CDRL2 having the amino acid sequence of SEQ ID NO: 5, and CDRL3 having the amino acid sequence of SEQ ID NO: 43.

In some embodiments, an anti-FAP antibody or antigen binding fragment thereof comprises a CDRH1, a CDRH2, and a CDRH3 that collectively are at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the CDRH1 having the amino acid sequence of SEQ ID NO: 10, CDRH2 having the amino acid sequence of SEQ ID NO: 50, and CDRH3 having the amino acid sequence of SEQ ID NO: 42. Alternatively or in addition, an anti-FAP antibody or antigen binding fragment thereof comprises a CDRL1, a CDRL2, and a CDRL3 that collectively are at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the to the CDRL1 having the amino acid sequence of SEQ ID NO: 4, CDRL2 having the amino acid sequence of SEQ ID NO: 5, and CDRL3 having the amino acid sequence of SEQ ID NO: 43.

In some embodiments, an anti-FAP antibody or antigen binding fragment thereof comprises: a CDRH1 having no more than 3 amino acid variations (e.g., no more than 3, 2, or 1 amino acid variation) as compared with the CDRH1 having the amino acid sequence of SEQ ID NO: 10; a CDRH2 having no more than 3 amino acid variations (e.g., no more than 3, 2, or 1 amino acid variation) as compared with the CDRH2 having the amino acid sequence of SEQ ID NO: 50; and/or a CDRH3 having no more than 3 amino acid variations (e.g., no more than 3, 2, or 1 amino acid variation) as compared with the CDRH3 having the amino acid sequence of SEQ ID NO: 42. Alternatively or in addition, an anti-FAP antibody or antigen binding fragment thereof comprises: a CDRL1 having no more than 3 amino acid variations (e.g., no more than 3, 2, or 1 amino acid variation) as compared with the CDRL1 having the amino acid sequence of SEQ ID NO: 4; a CDRL2 having no more than 3 amino acid variations (e.g., no more than 3, 2, or 1 amino acid variation) as compared with the CDRL2 having the amino acid sequence of SEQ ID NO: 5; and/or a CDRL3 having no more than 3 amino acid variations (e.g., no more than 3, 2, or 1 amino acid variation) as compared with the CDRL3 having the amino acid sequence of SEQ ID NO: 43.

In some embodiments, an anti-FAP antibody or antigen binding fragment thereof comprises a VH comprising the amino acid sequence of SEQ ID NO: 51. Alternatively or in addition, an anti-FAP antibody or antigen binding fragment thereof comprises a VL comprising the amino acid sequence of SEQ ID NO: 52. In some embodiments, an anti-FAP antibody or antigen binding fragment thereof comprises a VH comprising the amino acid sequence of SEQ ID NO: 51 and a VL comprising the amino acid sequence of SEQ ID NO: 52.

In some embodiments, an anti-FAP antibody or antigen binding fragment thereof comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 51. Alternatively or in addition, an anti-FAP antibody or antigen binding fragment thereof comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 52.

In some embodiments, an anti-FAP antibody or antigen binding fragment thereof comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VH as set forth in SEQ ID NO: 51. Alternatively or in addition, an anti-FAP antibody or antigen binding fragment thereof comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VL as set forth in SEQ ID NO: 52.

In some embodiments, an anti-FAP antibody or antigen binding fragment thereof comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 51. Alternatively or in addition, an anti-FAP antibody or antigen binding fragment thereof comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 52. In some embodiments, the number of amino acid variations (e.g., no more than 20, 19, 18, 17, 16, 15, 14, 13, 12, 11, 10, 9, 8, 7, 6, 5, 4, 3, 2, or 1 amino acid variation) may occur within a VH of SEQ ID NO: 51 and/or a VL of SEQ ID NO: 52 excluding any of the CDR sequences therein. In some embodiments, an anti-FAP antibody or antigen binding fragment thereof comprises a heavy chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VH of SEQ ID NO: 51, and/or a light chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VL of SEQ ID NO: 52.

In some embodiments, an anti-FAP antibody or antigen binding fragment thereof comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VH as set forth in SEQ ID NO: 51. Alternatively or in addition, an anti-FAP antibody or antigen binding fragment thereof comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VL as set forth in SEQ ID NO: 52. In some embodiments, the degree of sequence variation (e.g., at least 80%, at least 85%, at least 90%, at least 91%, at least 92%, at least 93%, at least 94%, at least 95%, at least 96%, at least 97%, at least 98%, at least 99%, or 100%) may occur within a VH of SEQ ID NO: 51, and/or a VL of SEQ ID NO: 52 excluding any of the CDR sequences therein. In some embodiments, an anti-FAP antibody or antigen binding fragment thereof comprises a heavy chain variable sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the framework sequence of a VH of SEQ ID NO: 51, and/or a light chain variable sequence that comprises a framework sequence that at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the framework sequence of a VL of SEQ ID NO: 52.

In some embodiments, an anti-FAP antibody or antigen binding fragment thereof comprises a CDRH1, a CDRH2 and a CDRH3 of a heavy chain variable domain (VH) having the amino acid sequence of SEQ ID NO: 56. Alternatively or in addition, an anti-FAP antibody or antigen binding fragment thereof comprises a CDRL1, a CDRL2 and a CDRL3 of a light chain variable domain (VL) having the amino acid sequence of SEQ ID NO: 57.

In some embodiments, according to the Kabat definition system, an anti-FAP antibody or antigen binding fragment thereof comprises a CDRH3 having the amino acid sequence of SEQ ID NO: 54. In some embodiments, according to the Kabat definition system, an anti-FAP antibody or antigen binding fragment thereof comprises a CDRH1 having the amino acid sequence of SEQ ID NO: 10, a CDRH2 having the amino acid sequence of SEQ ID NO: 11, and a CDRH3 having the amino acid sequence of SEQ ID NO: 54. In some embodiments, according to the Kabat definition system, an anti-FAP antibody or antigen binding fragment thereof comprises a CDRH1 having the amino acid sequence of SEQ ID NO: 10, a CDRH2 having the amino acid sequence of SEQ ID NO: 11, a CDRH3 having the amino acid sequence of SEQ ID NO: 54, a CDRL1 having the amino acid sequence of SEQ ID NO: 4, a CDRL2 having the amino acid sequence of SEQ ID NO: 5, and a CDRL3 having the amino acid sequence of SEQ ID NO: 55.

In some embodiments, an anti-FAP antibody or antigen binding fragment thereof comprises a CDRH1, a CDRH2, and a CDRH3, which collectively contains no more than 5 amino acid variations (e.g., no more than 5, 4, 3, 2, or 1 amino acid variation) as compared with the CDRH1 having the amino acid sequence of SEQ ID NO: 10, CDRH2 having the amino acid sequence of SEQ ID NO:11, and CDRH3 having the amino acid sequence of SEQ ID NO: 54. Alternatively or in addition, an anti-FAP antibody or antigen binding fragment thereof comprises a CDRL1, a CDRL2, and a CDRL3, which collectively contains no more than 5 amino acid variations (e.g., no more than 5, 4, 3, 2 or 1 amino acid variation) as compared with the CDRL1 having the amino acid sequence of SEQ ID NO: 4, CDRL2 having the amino acid sequence of SEQ ID NO: 5, and CDRL3 having the amino acid sequence of SEQ ID NO: 55.

In some embodiments, an anti-FAP antibody or antigen binding fragment thereof comprises a CDRH1, a CDRH2, and a CDRH3 that collectively are at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the CDRH1 having the amino acid sequence of SEQ ID NO: 10, CDRH2 having the amino acid sequence of SEQ ID NO: 11, and CDRH3 having the amino acid sequence of SEQ ID NO: 54. Alternatively or in addition, an anti-FAP antibody or antigen binding fragment thereof comprises a CDRL1, a CDRL2, and a CDRL3 that collectively are at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the to the CDRL1 having the amino acid sequence of SEQ ID NO: 4, CDRL2 having the amino acid sequence of SEQ ID NO: 5, and CDRL3 having the amino acid sequence of SEQ ID NO: 55.

In some embodiments, an anti-FAP antibody or antigen binding fragment thereof comprises: a CDRH1 having no more than 3 amino acid variations (e.g., no more than 3, 2, or 1 amino acid variation) as compared with the CDRH1 having the amino acid sequence of SEQ ID NO: 10; a CDRH2 having no more than 3 amino acid variations (e.g., no more than 3, 2, or 1 amino acid variation) as compared with the CDRH2 having the amino acid sequence of SEQ ID NO: 11; and/or a CDRH3 having no more than 3 amino acid variations (e.g., no more than 3, 2, or 1 amino acid variation) as compared with the CDRH3 having the amino acid sequence of SEQ ID NO: 54. Alternatively or in addition, an anti-FAP antibody or antigen binding fragment thereof comprises: a CDRL1 having no more than 3 amino acid variations (e.g., no more than 3, 2, or 1 amino acid variation) as compared with the CDRL1 having the amino acid sequence of SEQ ID NO: 4; a CDRL2 having no more than 3 amino acid variations (e.g., no more than 3, 2, or 1 amino acid variation) as compared with the CDRL2 having the amino acid sequence of SEQ ID NO: 5; and/or a CDRL3 having no more than 3 amino acid variations (e.g., no more than 3, 2, or 1 amino acid variation) as compared with the CDRL3 having the amino acid sequence of SEQ ID NO: 55.

In some embodiments, an anti-FAP antibody or antigen binding fragment thereof comprises a VH comprising the amino acid sequence of SEQ ID NO: 51. Alternatively or in addition, an anti-FAP antibody or antigen binding fragment thereof comprises a VL comprising the amino acid sequence of SEQ ID NO: 52. In some embodiments, an anti-FAP antibody or antigen binding fragment thereof comprises a VH comprising the amino acid sequence of SEQ ID NO: 51 and a VL comprising the amino acid sequence of SEQ ID NO: 52.

In some embodiments, an anti-FAP antibody or antigen binding fragment thereof comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 51. Alternatively or in addition, an anti-FAP antibody or antigen binding fragment thereof comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 52.

In some embodiments, an anti-FAP antibody or antigen binding fragment thereof comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VH as set forth in SEQ ID NO: 51. Alternatively or in addition, an anti-FAP antibody or antigen binding fragment thereof comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VL as set forth in SEQ ID NO: 52.

In some embodiments, an anti-FAP antibody or antigen binding fragment thereof comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 51. Alternatively or in addition, an anti-FAP antibody or antigen binding fragment thereof comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 52. In some embodiments, the number of amino acid variations (e.g., no more than 20, 19, 18, 17, 16, 15, 14, 13, 12, 11, 10, 9, 8, 7, 6, 5, 4, 3, 2, or 1 amino acid variation) may occur within a VH of SEQ ID NO: 51 and/or a VL of SEQ ID NO: 52 excluding any of the CDR sequences therein. In some embodiments, an anti-FAP antibody or antigen binding fragment thereof comprises a heavy chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VH of SEQ ID NO: 51, and/or a light chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VL of SEQ ID NO: 52.

In some embodiments, an anti-FAP antibody or antigen binding fragment thereof comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VH as set forth in SEQ ID NO: 51. Alternatively or in addition, an anti-FAP antibody or antigen binding fragment thereof comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VL as set forth in SEQ ID NO: 52. In some embodiments, the degree of sequence variation (e.g., at least 80%, at least 85%, at least 90%, at least 91%, at least 92%, at least 93%, at least 94%, at least 95%, at least 96%, at least 97%, at least 98%, at least 99%, or 100%) may occur within a VH of SEQ ID NO: 51, and/or a VL of SEQ ID NO: 51 excluding any of the CDR sequences therein. In some embodiments, an anti-FAP antibody or antigen binding fragment thereof comprises a heavy chain variable sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the framework sequence of a VH of SEQ ID NO: 51, and/or a light chain variable sequence that comprises a framework sequence that at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the framework sequence of a VL of SEQ ID NO: 52.

An anti-FAP antibody or antigen binding fragment thereof described herein can be in any antibody form, including, but not limited to, intact (i.e., full-length) antibodies, antigen-binding fragments thereof (such as Fab, F(ab′), F(ab′)2, Fv), single chain antibodies (e.g., scFv), bi-specific antibodies, or nanobodies. In some embodiments, an anti-FAP antibody or antigen binding fragment thereof described herein is a single-chain variable fragment (scFv). In some embodiments, an anti-FAP antibody or antigen binding fragment thereof described herein is a scFv-Fab (e.g., scFv fused to a portion of a constant region).

In some embodiments, an anti-FAP antibody or antigen binding fragment thereof comprises a VL domain and/or VH domain of any one of the anti-FAP antibodies selected from Table 2, and comprises a constant region comprising the amino acid sequences of the constant regions of an IgG, IgE, IgM, IgD, IgA or IgY immunoglobulin molecule, any class (e.g., IgG1, IgG2, IgG3, IgG4, IgA1 and IgA2), or any subclass (e.g., IgG2a and IgG2b) of immunoglobulin molecule. Non-limiting examples of human constant regions are described in the art, e.g., see Kabat E A et al., (1991) supra. Other antibody heavy and light chain constant regions are well known in the art, e.g., those provided in the IMGT database (imgt.org) or at vbase2.org/vbstat.php., both of which are incorporated by reference herein.

In some embodiments, an anti-FAP antibody or antigen binding fragment thereof is a single-chain variable fragment (scFv). In some embodiments, an anti-FAP scFv comprises a VH and a VL of any one of the anti-FAP antibodies selected from Table 2. In some embodiments, the VH and the VL of an anti-FAP scFv are joined together by a linker. In some embodiments, a linker may have a length of about 2 to 10 amino acids, 5 to 20 amino acids, 10 to 30 amino acids, 20-50 amino acids, 40 to 60 amino acids, 60 to 80 amino acids, or more than 80 amino acids. In some embodiment, a linker may include a sequence that substantially comprises glycine and serine. An exemplary linker sequence is GGGGSGGGGSGGGAS (SEQ ID NO: 29). In some embodiments, a linker may include, without limitation, any of those encompassed by U.S. Pat. Nos. 8,445,251 and 9,434,931. In some embodiments, an anti-FAP comprises a linker between the VH and the VL, and the linker comprises an amino acid sequence at least 80%, at least 85%, at least 90%, at least 92%, at least 95%, at least 98%, at least 99%, or 100% identical to SEQ ID NO: 29.

In some embodiments, an anti-FAP scFv comprises a VH and a VL, and the C-terminus of the VH is joined with the N terminus of the VL via a linker (e.g., the linker as set forth in SEQ ID NO: 29). In some embodiments, an anti-FAP scFv comprises a VH and a VL, and the C-terminus of the VL is joined with the N terminus of the VH via a linker (e.g., the linker as set forth in SEQ ID NO: 29).

In some embodiments, an anti-FAP scFv comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH of any of the anti-FAP antibodies listed in Table 2. Alternatively or in addition, an anti-FAP scFv comprises a VL containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH of any of the anti-FAP antibodies listed in Table 2. In some embodiments, an anti-FAP scFv comprises a VH comprising an amino acid sequence that is at least 80% ((e.g., at least 80%, at least 85%, at least 90%, at least 91%, at least 92%, at least 93%, at least 94%, at least 95%, at least 96%, at least 97%, at least 98%, at least 99%, or 100%) identical to the VH of any of the anti-FAP antibodies listed in Table 2. Alternatively, or in addition, an anti-FAP scFv comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VH of any of the anti-FAP antibodies listed in Table 2.

In some embodiments, an anti-FAP scFv comprises an amino acid sequence containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the any of the anti-FAP scFv listed in Table 2. In some embodiments, an anti-FAP scFv comprises an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to any of the anti-FAP scFv listed in Table 2.

In some embodiments, an anti-FAP scFv comprises a VH comprising the amino acid sequence of SEQ ID NO: 7, and/or a VL comprising the amino acid sequence of SEQ ID NO: 8. In some embodiments, an anti-FAP scFv comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 7, and/or a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 8. In some embodiments, an anti-FAP scFv comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VH as set forth in SEQ ID NO: 7, and/or a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VL as set forth in SEQ ID NO: 8.

In some embodiments, an anti-FAP scFv comprises an amino acid sequence containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the scFv amino acid sequence as set forth in SEQ ID NO: 9. In some embodiments, an anti-FAP scFv comprises an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the scFv amino acid sequence as set forth in SEQ ID NO: 9. In some embodiments, an anti-FAP scFv comprises an amino acid sequence of SEQ ID NO: 9.

In some embodiments, an anti-FAP scFv comprises a VH comprising the amino acid sequence of SEQ ID NO: 14, and/or a VL comprising the amino acid sequence of SEQ ID NO: 15. In some embodiments, an anti-FAP scFv comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 14, and/or a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 15. In some embodiments, an anti-FAP scFv comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VH as set forth in SEQ ID NO: 14, and/or a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VL as set forth in SEQ ID NO: 15.

In some embodiments, an anti-FAP scFv comprises an amino acid sequence containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the scFv amino acid sequence as set forth in SEQ ID NO: 16. In some embodiments, an anti-FAP scFv comprises an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the scFv amino acid sequence as set forth in SEQ ID NO: 16. In some embodiments, an anti-FAP scFv comprises an amino acid sequence of SEQ ID NO: 16.

In some embodiments, an anti-FAP scFv comprises a VH comprising the amino acid sequence of SEQ ID NO: 44, and/or a VL comprising the amino acid sequence of SEQ ID NO: 45. In some embodiments, an anti-FAP scFv comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 44, and/or a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 45. In some embodiments, an anti-FAP scFv comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VH as set forth in SEQ ID NO: 44, and/or a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VL as set forth in SEQ ID NO: 55.

In some embodiments, an anti-FAP scFv comprises an amino acid sequence containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the scFv amino acid sequence as set forth in SEQ ID NO: 46. In some embodiments, an anti-FAP scFv comprises an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the scFv amino acid sequence as set forth in SEQ ID NO: 46. In some embodiments, an anti-FAP scFv comprises an amino acid sequence of SEQ ID NO: 46.

In some embodiments, an anti-FAP scFv comprises a VH comprising the amino acid sequence of SEQ ID NO: 47, and/or a VL comprising the amino acid sequence of SEQ ID NO: 48. In some embodiments, an anti-FAP scFv comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 47, and/or a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 48. In some embodiments, an anti-FAP scFv comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VH as set forth in SEQ ID NO: 47, and/or a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VL as set forth in SEQ ID NO: 48.

In some embodiments, an anti-FAP scFv comprises an amino acid sequence containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the scFv amino acid sequence as set forth in SEQ ID NO: 49. In some embodiments, an anti-FAP scFv comprises an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the scFv amino acid sequence as set forth in SEQ ID NO: 49. In some embodiments, an anti-FAP scFv comprises an amino acid sequence of SEQ ID NO: 49.

In some embodiments, an anti-FAP scFv comprises a VH comprising the amino acid sequence of SEQ ID NO: 51, and/or a VL comprising the amino acid sequence of SEQ ID NO: 52. In some embodiments, an anti-FAP scFv comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 51, and/or a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 52. In some embodiments, an anti-FAP scFv comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VH as set forth in SEQ ID NO: 51, and/or a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VL as set forth in SEQ ID NO: 52.

In some embodiments, an anti-FAP scFv comprises an amino acid sequence containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the scFv amino acid sequence as set forth in SEQ ID NO: 53. In some embodiments, an anti-FAP scFv comprises an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the scFv amino acid sequence as set forth in SEQ ID NO: 53. In some embodiments, an anti-FAP scFv comprises an amino acid sequence of SEQ ID NO: 53.

In some embodiments, an anti-FAP scFv comprises a VH comprising the amino acid sequence of SEQ ID NO: 56, and/or a VL comprising the amino acid sequence of SEQ ID NO: 57. In some embodiments, an anti-FAP scFv comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 56, and/or a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 57. In some embodiments, an anti-FAP scFv comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VH as set forth in SEQ ID NO: 56, and/or a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VL as set forth in SEQ ID NO: 57.

In some embodiments, an anti-FAP scFv comprises an amino acid sequence containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the scFv amino acid sequence as set forth in SEQ ID NO: 58. In some embodiments, an anti-FAP scFv comprises an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the scFv amino acid sequence as set forth in SEQ ID NO: 16. In some embodiments, an anti-FAP scFv comprises an amino acid sequence of SEQ ID NO: 58.

In some embodiments, any of the anti-FAP antibody or antigen binding fragment described herein is modified, e.g., modified via glycosylation, phosphorylation, sumoylation, and/or methylation. In some embodiments, the anti-FAP antibody or antigen binding fragment is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or phosphoglycosylation. In some embodiments, the one or more sugar or carbohydrate molecules are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, an N-acetylgalactosamine unit, a galactose unit, a fucose unit, or a phospholipid unit. In some embodiments, there are about 1-10, about 1-5, about 5-10, about 1-4, about 1-3, or about 2 sugar molecules. In some embodiments, a glycosylated antibody is fully or partially glycosylated. In some embodiments, an antibody is glycosylated by chemical reactions or by enzymatic means. In some embodiments, an antibody is glycosylated in vitro or inside a cell, which may optionally be deficient in an enzyme in the N- or O-glycosylation pathway, e.g. a glycosyltransferase. In some embodiments, an antibody is functionalized with sugar or carbohydrate molecules as described in International Patent Application Publication WO2014065661, published on May 1, 2014, entitled, “Modified antibody, antibody-conjugate and process for the preparation thereof”.

In some embodiments, conservative mutations can be introduced into antibody sequences (e.g., CDRs or framework sequences) at positions where the residues are not likely to be involved in interacting with a target antigen (e.g., FAP), for example, as determined based on a crystal structure.

In some embodiments, any one of the anti-FAP antibody or antigen binding fragment described herein may comprise a signal peptide in the heavy and/or light chain sequence (e.g., a N-terminal signal peptide). In some embodiments, the anti-FAP antibody or antigen binding fragment described herein comprises any one of the VH and VL sequences, any one of the IgG heavy chain and light chain sequences, or any one of the scFv sequences described herein, and further comprises a signal peptide (e.g., a N-terminal signal peptide).

In some embodiments, the present disclosure also contemplates engineering any of the FAP binding moieties known in the art to be an extracellular ligand-binding domain of a CAR expressed by the genetically modified immune cells (e.g., iNKT cells) described herein. Non-limiting examples of FAP binding moieties are set forth in Table 3.

TABLE 3
Listing of Known FAP Binding Moieties

| FAP Binding Moiety Name/ | |
|---|---|
| clone | Reference(s) |
| Murine anti-FAP antibody | WO1999057151 |
| clone F19 | |
| Humanized F19 clone BIBH 1 | WO2007077173 |
| (sibrotuzumab) | |
| Clone MO36, MO33, Hu33, and | WO2015118030 |
| Hu36 | |
| Mab 73.3 | WO2014055442 |
| Clones 2D9, 4B8, 7A1, 13B2, | WO2012020006 |
| 13C2, 13E8, 14C10, and 17A11, | |
| 3F2, 3D9, 4G8, 4B3, 2C6, 19G1, | |
| 4B9, 5B8, 5F1, 14B3, 16F1, | |
| 15F8, 03C9, 22A3, 29B 11, | |
| 02D7, 28H1, and 23C10, | |
| Clones NI-206.82C2, NI- | WO2016110598 |
| 206.59B4, NI-206.22F7, | |
| NI-206.27E8, NI-206.12G4, and | |
| NI-206.17A6 | |
| Clones D8, D28 and D43 | WO2009074275 |
| NI-206.18H2, | US20180022822 |
| NI-206.20A8, and NI-206.6D3 | |
| Clone 4G5 | WO2021061708 |
| Clones F5, F8, and KSF | WO2016116399 |
| Clones ESC 11 and ESC 14 | WO2011040972 |
| Clones FAP-FR2-A01, FAP- | WO2022133094 |
| FR2-B01, FAP-FR2-C01, FAP- | |
| FR2-E01, FAP-FR2-F01, FAP- | |
| FR2-G01. FAP-FR2-H01, FAP- | |
| FR2-B02, FAP-FR2-C02, FAP- | |
| FR2-D02, FAP-FR2-E02, FAP- | |
| FR2-H02, FAP-FR2-B03, FAP- | |
| FR2-C03, FAP-FR2-D03, FAP- | |
| FR2-F03, FAP-FR2-H03, FAP- | |
| FR2-B04, FAP-FR2-D04, FAP- | |
| FR2-E04, FAP-FR2-G04, FAP- | |
| FR2-A05, FAP-FR2-C05, FAP- | |
| FR2-D05, FAP-FR2-E05, FAP- | |
| FR2-F05, FAP-FR2-G05, FAP- | |
| FR2-H05, FAP-FR2-A06, FAP- | |
| FR2-B06, FAP-FR2-C06, FAP- | |
| FR2-D06, FAP-FR2-E06, FAP- | |
| FR2-F06, FAP-FR2-A07, FAP- | |
| FR2-B07, FAP-FR2-C07, FAP- | |
| FR2-D07, FAP-FR2-E07, FAP- | |
| FR2-F07, FAP-FR2-G07, FAP- | |
| FR2-H07, FAP-FR2-A08, FAP- | |
| FR2-C08, FAP-FR2-D08, FAP- | |
| FR2-E08, FAP-FR2-G08, FAP- | |
| FR2-A09, FAP-FR2-B09, FAP- | |
| FR2-C09, FAP-FR2-D09, FAP- | |
| FR2-E09, FAP-FR2-F09, FAP- | |
| FR2-H09, FAP-FR2-B10, FAP- | |
| FR2-C10, FAP-FR2-D10, FAP- | |
| FR2-E10, FAP-FR2-F10, FAP- | |
| FR2-G10, FAP-FR2-H10, FAP- | |
| FR2-A11, FAP-FR2-C11, FAP- | |
| FR2-D11, FAP-FR2-F11, FAP- | |
| FR2-G11, FAP-FR2-H11, FAP- | |
| FR2-B12, FAP-FR2-C12, FAP- | |
| FR2-D12, FAP-FR2-F12, FAP- | |
| FR2-G12, FAP-FR2-H12, FAP- | |
| FR3-A01, FAP-FR3-B01, FAP- | |
| FR3-E01, FAP-FR3-F01, FAP- | |
| FR3-H01, FAP-FR3-A02, FAP- | |
| FR3-C02, FAP-FR3-D02, FAP- | |
| FR3-F02, FAP-FR3-G02, FAP- | |
| FR3-A03, FAP-FR3-B03, FAP- | |
| FR3-C03, FAP-FR3-D03, FAP- | |
| FR3-A04, FAP-FR3-B04, FAP- | |
| FR3-E04, FAP-FR3-G04, FAP- | |
| FR3-H04, FAP-FR3-D05, FAP- | |
| FR3-E05, FAP-FR3-G05, FAP- | |
| FR3-H05, FAP-FR3-C06, FAP- | |
| FR3-E06, FAP-FR3-F06, FAP- | |
| FR3-G06, FAP-FR3-H06, FAP- | |
| FR3-A07, FAP-FR3-B07, FAP- | |
| FR3-E07, FAP-FR3-F07, and | |
| FAP-FR3-G07 | |
| Recombinant protein having an | WO2020245173 |
| ankyrin repeat domain | |
| FAP5 | Ostermann et al., Effective |
| | immunoconjugate therapy in cancer |
| | models targeting a serine protease |
| | of tumor fibroblasts, Clin Cancer |
| | Res. 2008 Jul. 15; 14(14): 4584-92. |
| | Tran et al., Immune targeting of |
| | fibroblast activation protein triggers |
| | recognition of multipotent bone |
| | marrow stromal cells and cachexia, |
| | J Exp Med. 2013 Jun. 3; 210(6): |
| | 1125-1135. |
| F11-24 | ThermoFisher Catalog # BMS168 |
| 427819 | Novus Biologicals MAB3715-SP |
| FAP-2286 | Zboralski et al., Preclinical evaluation |
| | of FAP-2286, a peptide-targeted |
| | radionuclide therapy (PTRT) to |
| | fibroblast activation protein alpha |
| | (FAP), Annals of Oncology, |
| | Volume 31, Supplement 4, S488, |
| | Sep. 1, 2020 |

Aspects of the present disclosure also provides chimeric antigen receptors (CARs) comprising an extracellular ligand-binding domain. In some embodiments, the choice of ligand-binding domain depends upon the type and number of ligands that define the surface of a target cell. For example, the ligand-binding domain may be chosen to recognize one or more ligands that act as a cell surface marker on target cells associated with a particular disease state. Thus, examples of cell surface markers that may act as ligands for the ligand-binding domain in the CAR of the present disclosure can include those associated with viral, bacterial and parasitic infections, autoimmune disease, and, more preferably, cancer cells. In some embodiments, a CAR of the present disclosure is engineered to target one or more tumor antigens of interest by way of engineering a desired ligand-binding moiety that specifically binds to one or more antigens on a tumor cell. In the context of the present disclosure, “tumor antigen” refers to an antigen that is common to or characterizes specific hyperproliferative disorders such as cancer. Generally, a CAR (e.g., anti-FAP CAR) of the present disclosure will comprise at least an extracellular domain and an intracellular domain. In some embodiments, the extracellular domain comprises a target-specific binding element (e.g., a scFv that specifically binds to FAP (e.g., FAP protein set forth in SEQ ID NOs: 35-38)) otherwise referred to herein as a ligand-binding domain (also referred to herein as an antigen-binding domain). In some embodiments, the extracellular domain is an antigen-binding domain or a portion thereof. In some embodiments, the extracellular ligand-binding domain is a Fab. In some embodiments, the extracellular ligand-binding domain is a scFv. In some embodiments, an extracellular ligand-binding domain of a CAR described herein comprises an antigen binding fragment that specifically binds to FAP (e.g., human FAP). In some embodiments, an extracellular ligand-binding domain of a CAR described herein comprises any one of the FAP antibodies or antigen binding fragments thereof (e.g., anti-FAP scFv).

In some embodiments, an anti-FAP CAR of the present disclosure comprises an extracellular ligand-binding domain specifically binds FAP with binding affinity (e.g., as indicated by K_{D}) of at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, or less. For example, an anti-FAP CAR of the present disclosure can bind to a FAP protein (e.g., human FAP protein and/or mouse FAP protein as set forth in SEQ ID NOs: 35-38) with an affinity between 5 pM and 500 nM, between 10 pM and 450 nM, between 20 pM and 400 nM, between 30 pM and 350 nM, between 40 pM and 300 nM, between 50 pM and 250 nM, between 60 pM and 200 nM, between 70 pM and 150 nM, between 80 pM and 100 nM, between 80 pM and 90 nM, between 90 pM and 80 nM. between 100 pM and 70 nM, between 200 pM and 60 nM, between 300 PM and 50 nM, between 400 pM and 40 nM, between 500 pM and 30 nM, between 600 pM and 20 nM, between 700 pM and 10 nM, between 800 pM and 5 nM, or between 900 pM and 2 nM.

The disclosure also includes CARs that compete with any of the CARs described herein for binding to a FAP protein (e.g., human FAP protein and/or mouse FAP protein as set forth in SEQ ID NOs: 35-38) and that have an affinity of 100 nM or lower (e.g., 80 nM or lower, 50 nM or lower, 20 nM or lower, 10 nM or lower, 500 pM or lower, 50 pM or lower, or 5 pM or lower). The affinity and binding kinetics of the anti-FAP CARs can be tested using any suitable method including but not limited to biosensor technology (e.g., OCTET or BIACORE). In some embodiments, the anti-FAP CARs described herein binds to FAP with a K_{D} of sub-nanomolar range.

In some embodiments, an anti-FAP CAR of the present disclosure comprises one or more of the heavy chain CDRs (e.g., CDRH1, CDRH2, or CDRH3) amino acid sequences from any one of the anti-FAP antibodies selected from Table 2. In some embodiments, an anti-FAP CAR of the present disclosure comprise the CDRH1, CDRH2, and CDRH3 as provided for any one of the antibodies elected from Table 2. In some embodiments, an anti-FAP CAR of the present disclosure comprises one or more of the light chain CDRs (e.g., CDRL1, CDRL2, or CDRL3) amino acid sequences from any one of the anti-FAP antibodies selected from Table 2. In some embodiments, an anti-FAP CAR of the present disclosure comprise the CDRL1, CDRL2, and CDRL3 as provided for any one of the anti-FAP antibodies selected from Table 2.

In some embodiments, an anti-FAP CAR comprises the CDRH1, CDRH2, CDRH3, CDRL1, CDRL2, and CDRL3 as provided for any one of the anti-FAP antibodies selected from Table 2. In some embodiments, antibody heavy and light chain CDR3 domains may play a particularly important role in the binding specificity/affinity of an antibody for an antigen. Accordingly, an anti-FAP CAR may include at least the heavy and/or light chain CDR3s of any one of the anti-FAP antibodies selected from Table 2.

In some embodiments, any of the anti-FAP CAR of the disclosure have one or more CDRs (e.g., heavy chain CDR or light chain CDR) sequences substantially similar to any of the CDRH1, CDRH2, CDRH3, CDRL1, CDRL2, and/or CDRL3 sequences from one of the anti-FAP antibodies selected from Table 2. In some embodiments, the position of one or more CDRs along the VH (e.g., CDRH1, CDRH2, or CDRH3) and/or VL (e.g., CDRL1, CDRL2, or CDRL3) region of a chimeric antigen receptor described herein can vary by one, two, three, four, five, or six amino acid positions so long as immunospecific binding to FAP (e.g., human FAP) is maintained (e.g., substantially maintained, for example, at least 50%, at least 60%, at least 70%, at least 80%, at least 90%, at least 95% of the binding of the original antibody from which it is derived).

In some examples, an anti-FAP CAR comprises one or more CDR (e.g., heavy chain CDRs or light chain CDR) sequences substantially similar to any one of the anti-FAP antibodies selected from Table 2. For example, the anti-FAP CAR may include one or more CDR sequence(s) from any of the anti-FAP antibodies selected from Table 2 containing up to 5, 4, 3, 2, or 1 amino acid residue variations as compared to the corresponding CDR region in any one of the CDRs provided herein (e.g., CDRs from any of the anti-FAP antibodies selected from Table 2) so long as immunospecific binding to FAP (e.g., human FAP) is maintained (e.g., substantially maintained, for example, at least 50%, at least 60%, at least 70%, at least 80%, at least 90%, at least 95% relative to the binding of the original antibody from which it is derived).

Some aspects of the disclosure provide anti-FAP CARs that comprise one or more of the heavy chain variable (VH) and/or light chain variable (VL) domains provided herein. In some embodiments, any of the VH domains provided herein include one or more of the heavy chain CDR sequences (e.g., CDRH1, CDRH2, and CDRH3) provided herein, for example, any of the heavy chain CDR sequences provided in any one of the anti-FAP antibodies selected from Table 2. In some embodiments, any of the VL domains provided herein include one or more of the CDR-L sequences (e.g., CDRL1, CDRL2, and CDRL3) provided herein, for example, any of the light chain CDR sequences provided in any one of the anti-FAP antibodies selected from Table 2.

In some embodiments, an anti-FAP CAR comprises a heavy chain variable domain and/or a light chain variable domain of any one of the anti-FAP antibodies selected from Table 2, and variants thereof. In some embodiments, an anti-FAP CAR comprises the heavy chain variable and light chain variable pairs of any anti-FAP antibodies selected from Table 2.

Aspects of the disclosure provide anti-FAP CAR comprising a heavy chain variable (VH) and/or a light chain variable (VL) domain amino acid sequence homologous to any of those described herein. In some embodiments, an anti-FAP CAR comprises a VH or a VL that is at least 75% (e.g., 80%, 85%, 90%, 95%, 98%, or 99%) identical to the VH and/or any VL of any one of the anti-FAP antibodies selected from Table 2. In some embodiments, the homologous VH and/or a VL amino acid sequences of the anti-FAP CAR do not vary within any of the CDR sequences provided herein. For example, in some embodiments, the degree of sequence variation (e.g., 75%, 80%, 85%, 90%, 95%, 98%, or 99%) may occur within a VH and/or a VL sequence of an anti-FAP CAR excluding any of the CDR sequences provided herein. In some embodiments, any of the anti-FAP CAR provided herein comprise a VH sequence and a VL sequence that comprises a framework sequence that is at least 75%, 80%, 85%, 90%, 95%, 98%, or 99% identical to the framework sequence of any anti-FAP antibodies selected from Table 2. In some embodiments, an anti-FAP CAR comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH of any of the anti-FAP antibodies listed in Table 2. Alternatively or in addition, an anti-FAP CAR comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL of any one of the anti-FAP antibodies listed in Table 2.

In some embodiments, an anti-FAP CAR comprises a CDRH1, a CDRH2, a CDRH3, a CDRL1, a CDRL2, and a CDRL3 that are the same as the CDRH1, CDRH2, CDRH3, CDRL1, CDRL2, and CDRL3 shown in Table 2, and comprises a humanized VH and/or a humanized VL. In some embodiments, an anti-FAP CAR is a humanized variant comprising one or more amino acid substitutions (e.g., in the VH framework region) as compared with any one of the VHs listed in Table 2, and/or one or more amino acid substitutions (e.g., in the VL framework region) as compared with any one of the VLs listed in Table 2.

In some embodiments, an anti-FAP CAR comprises a CDRH1, a CDRH2 and a CDRH3 of a heavy chain variable domain (VH) having the amino acid sequence of SEQ ID NO: 7. Alternatively or in addition, an anti-FAP CAR comprises a CDRL1, a CDRL2 and a CDRL3 of a light chain variable domain (VL) having the amino acid sequence of SEQ ID NO: 8.

In some embodiments, an anti-FAP CAR comprises a CDRH3 having the amino acid sequence of SEQ ID NO: 3. In some embodiments, an anti-FAP CAR comprises a CDRH1 having the amino acid sequence of SEQ ID NO: 1, a CDRH2 having the amino acid sequence of SEQ ID NO: 2, and a CDRH3 having the amino acid sequence of SEQ ID NO: 3. In some embodiments, an anti-FAP CAR comprises a CDRH1 having the amino acid sequence of SEQ ID NO: 1, a CDRH2 having the amino acid sequence of SEQ ID NO: 2, a CDRH3 having the amino acid sequence of SEQ ID NO: 3, a CDRL1 having the amino acid sequence of SEQ ID NO: 4, a CDRL2 having the amino acid sequence of SEQ ID NO: 5, and a CDRL3 having the amino acid sequence of SEQ ID NO: 6.

In some embodiments, an anti-FAP CAR comprises a CDRH1, a CDRH2, and a CDRH3, which collectively contains no more than 5 amino acid variations (e.g., no more than 5, 4, 3, 2, or 1 amino acid variation) as compared with the CDRH1 having the amino acid sequence of SEQ ID NO: 1, CDRH2 having the amino acid sequence of SEQ ID NO: 2, and CDRH3 having the amino acid sequence of SEQ ID NO: 3. Alternatively or in addition, an anti-FAP CAR comprises a CDRL1, a CDRL2, and a CDRL3, which collectively contains no more than 5 amino acid variations (e.g., no more than 5, 4, 3, 2 or 1 amino acid variation) as compared with the CDRL1 having the amino acid sequence of SEQ ID NO: 4, CDRL2 having the amino acid sequence of SEQ ID NO: 5, and CDRL3 having the amino acid sequence of SEQ ID NO: 6.

In some embodiments, an anti-FAP CAR comprises a CDRH1, a CDRH2, and a CDRH3 that collectively are at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the CDRH1 having the amino acid sequence of SEQ ID NO: 1, CDRH2 having the amino acid sequence of SEQ ID NO: 2, and CDRH3 having the amino acid sequence of SEQ ID NO: 3. Alternatively or in addition, an anti-FAP CAR comprises a CDRL1, a CDRL2, and a CDRL3 that collectively are at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the to the CDRL1 having the amino acid sequence of SEQ ID NO: 4, CDRL2 having the amino acid sequence of SEQ ID NO: 5, and CDRL3 having the amino acid sequence of SEQ ID NO: 6.

In some embodiments, an anti-FAP CAR comprises: a CDRH1 having no more than 3 amino acid variations (e.g., no more than 3, 2, or 1 amino acid variation) as compared with the CDRH1 having the amino acid sequence of SEQ ID NO: 1; a CDRH2 having no more than 3 amino acid variations (e.g., no more than 3, 2, or 1 amino acid variation) as compared with the CDRH2 having the amino acid sequence of SEQ ID NO: 2; and/or a CDRH3 having no more than 3 amino acid variations (e.g., no more than 3, 2, or 1 amino acid variation) as compared with the CDRH3 having the amino acid sequence of SEQ ID NO: 3. Alternatively or in addition, an anti-FAP CAR comprises: a CDRL1 having no more than 3 amino acid variations (e.g., no more than 3, 2, or 1 amino acid variation) as compared with the CDRL1 having the amino acid sequence of SEQ ID NO: 4; a CDRL2 having no more than 3 amino acid variations (e.g., no more than 3, 2, or 1 amino acid variation) as compared with the CDRL2 having the amino acid sequence of SEQ ID NO: 5; and/or a CDRL3 having no more than 3 amino acid variations (e.g., no more than 3, 2, or 1 amino acid variation) as compared with the CDRL3 having the amino acid sequence of SEQ ID NO: 6.

In some embodiments, an anti-FAP CAR comprises a VH comprising the amino acid sequence of SEQ ID NO: 7. Alternatively or in addition, an anti-FAP CAR comprises a VL comprising the amino acid sequence of SEQ ID NO: 8. In some embodiments, an anti-FAP CAR comprises a VH comprising the amino acid sequence of SEQ ID NO: 7 and a VL comprising the amino acid sequence of SEQ ID NO: 8.

In some embodiments, an anti-FAP CAR comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 7. Alternatively or in addition, an anti-FAP CAR comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 8.

In some embodiments, an anti-FAP CAR comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VH as set forth in SEQ ID NO: 7. Alternatively or in addition, an anti-FAP CAR comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VL as set forth in SEQ ID NO: 8.

In some embodiments, an anti-FAP CAR comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 7. Alternatively or in addition, an anti-FAP CAR comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 8. In some embodiments, the number of amino acid variations (e.g., no more than 20, 19, 18, 17, 16, 15, 14, 13, 12, 11, 10, 9, 8, 7, 6, 5, 4, 3, 2, or 1 amino acid variation) may occur within a VH of SEQ ID NO: 7 and/or a VL of SEQ ID NO: 8 excluding any of the CDR sequences therein. In some embodiments, an anti-FAP CAR comprises a heavy chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VH of SEQ ID NO: 7, and/or a light chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VL of SEQ ID NO: 8.

In some embodiments, an anti-FAP CAR comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VH as set forth in SEQ ID NO: 7. Alternatively or in addition, an anti-FAP CAR comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VL as set forth in SEQ ID NO: 8. In some embodiments, the degree of sequence variation (e.g., at least 80%, at least 85%, at least 90%, at least 91%, at least 92%, at least 93%, at least 94%, at least 95%, at least 96%, at least 97%, at least 98%, at least 99%, or 100%) may occur within a VH of SEQ ID NO: 7, and/or a VL of SEQ ID NO: 8 excluding any of the CDR sequences therein. In some embodiments, an anti-FAP CAR comprises a heavy chain variable sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the framework sequence of a VH of SEQ ID NO: 7, and/or a light chain variable sequence that comprises a framework sequence that at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the framework sequence of a VL of SEQ ID NO: 8.

In some embodiments, an anti-FAP CAR comprises a CDRH1, a CDRH2 and a CDRH3 of a heavy chain variable domain (VH) having the amino acid sequence of SEQ ID NO: 14. Alternatively or in addition, an anti-FAP CAR comprises a CDRL1, a CDRL2 and a CDRL3 of a light chain variable domain (VL) having the amino acid sequence of SEQ ID NO: 15.

In some embodiments, an anti-FAP CAR comprises a CDRH3 having the amino acid sequence of SEQ ID NO: 12. In some embodiments, an anti-FAP CAR comprises a CDRH1 having the amino acid sequence of SEQ ID NO: 10, a CDRH2 having the amino acid sequence of SEQ ID NO: 11, and a CDRH3 having the amino acid sequence of SEQ ID NO: 12. In some embodiments, an anti-FAP CAR comprises a CDRH1 having the amino acid sequence of SEQ ID NO: 10, a CDRH2 having the amino acid sequence of SEQ ID NO: 11, a CDRH3 having the amino acid sequence of SEQ ID NO: 12, a CDRL1 having the amino acid sequence of SEQ ID NO: 4, a CDRL2 having the amino acid sequence of SEQ ID NO: 5, and a CDRL3 having the amino acid sequence of SEQ ID NO: 13.

In some embodiments, an anti-FAP CAR comprises a CDRH1, a CDRH2, and a CDRH3, which collectively contains no more than 5 amino acid variations (e.g., no more than 5, 4, 3, 2, or 1 amino acid variation) as compared with the CDRH1 having the amino acid sequence of SEQ ID NO: 10, CDRH2 having the amino acid sequence of SEQ ID NO: 11, and CDRH3 having the amino acid sequence of SEQ ID NO: 12. Alternatively or in addition, an anti-FAP CAR comprises a CDRL1, a CDRL2, and a CDRL3, which collectively contains no more than 5 amino acid variations (e.g., no more than 5, 4, 3, 2 or 1 amino acid variation) as compared with the CDRL1 having the amino acid sequence of SEQ ID NO: 4, CDRL2 having the amino acid sequence of SEQ ID NO: 5, and CDRL3 having the amino acid sequence of SEQ ID NO: 13.

In some embodiments, an anti-FAP CAR comprises a CDRH1, a CDRH2, and a CDRH3 that collectively are at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the CDRH1 having the amino acid sequence of SEQ ID NO: 10, CDRH2 having the amino acid sequence of SEQ ID NO: 11, and CDRH3 having the amino acid sequence of SEQ ID NO: 12. Alternatively or in addition, an anti-FAP CAR comprises a CDRL1, a CDRL2, and a CDRL3 that collectively are at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the to the CDRL1 having the amino acid sequence of SEQ ID NO: 4, CDRL2 having the amino acid sequence of SEQ ID NO: 5, and CDRL3 having the amino acid sequence of SEQ ID NO: 13.

In some embodiments, an anti-FAP CAR comprises: a CDRH1 having no more than 3 amino acid variations (e.g., no more than 3, 2, or 1 amino acid variation) as compared with the CDRH1 having the amino acid sequence of SEQ ID NO: 10; a CDRH2 having no more than 3 amino acid variations (e.g., no more than 3, 2, or 1 amino acid variation) as compared with the CDRH2 having the amino acid sequence of SEQ ID NO: 11; and/or a CDRH3 having no more than 3 amino acid variations (e.g., no more than 3, 2, or 1 amino acid variation) as compared with the CDRH3 having the amino acid sequence of SEQ ID NO: 12. Alternatively or in addition, an anti-FAP CAR comprises: a CDRL1 having no more than 3 amino acid variations (e.g., no more than 3, 2, or 1 amino acid variation) as compared with the CDRL1 having the amino acid sequence of SEQ ID NO: 4; a CDRL2 having no more than 3 amino acid variations (e.g., no more than 3, 2, or 1 amino acid variation) as compared with the CDRL2 having the amino acid sequence of SEQ ID NO: 5; and/or a CDRL3 having no more than 3 amino acid variations (e.g., no more than 3, 2, or 1 amino acid variation) as compared with the CDRL3 having the amino acid sequence of SEQ ID NO: 13.

In some embodiments, an anti-FAP CAR comprises a VH comprising the amino acid sequence of SEQ ID NO: 14. Alternatively or in addition, an anti-FAP CAR comprises a VL comprising the amino acid sequence of SEQ ID NO: 15. In some embodiments, an anti-FAP CAR comprises a VH comprising the amino acid sequence of SEQ ID NO: 14 and a VL comprising the amino acid sequence of SEQ ID NO: 15.

In some embodiments, an anti-FAP CAR comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 14. Alternatively or in addition, an anti-FAP CAR comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 15.

In some embodiments, an anti-FAP CAR comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VH as set forth in SEQ ID NO: 14. Alternatively or in addition, an anti-FAP CAR comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VL as set forth in SEQ ID NO: 15.

In some embodiments, an anti-FAP CAR comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 14. Alternatively or in addition, an anti-FAP CAR comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 15. In some embodiments, the number of amino acid variations (e.g., no more than 20, 19, 18, 17, 16, 15, 14, 13, 12, 11, 10, 9, 8, 7, 6, 5, 4, 3, 2, or 1 amino acid variation) may occur within a VH of SEQ ID NO: 14 and/or a VL of SEQ ID NO: 15 excluding any of the CDR sequences therein. In some embodiments, an anti-FAP CAR comprises a heavy chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VH of SEQ ID NO: 14, and/or a light chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VL of SEQ ID NO: 15.

In some embodiments, an anti-FAP CAR comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VH as set forth in SEQ ID NO: 14. Alternatively or in addition, an anti-FAP CAR comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VL as set forth in SEQ ID NO: 15. In some embodiments, the degree of sequence variation (e.g., at least 80%, at least 85%, at least 90%, at least 91%, at least 92%, at least 93%, at least 94%, at least 95%, at least 96%, at least 97%, at least 98%, at least 99%, or 100%) may occur within a VH of SEQ ID NO: 14, and/or a VL of SEQ ID NO: 15 excluding any of the CDR sequences therein. In some embodiments, an anti-FAP CAR comprises a heavy chain variable sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the framework sequence of a VH of SEQ ID NO: 14, and/or a light chain variable sequence that comprises a framework sequence that at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the framework sequence of a VL of SEQ ID NO: 15.

In some embodiments, an anti-FAP CAR comprises a CDRH1, a CDRH2 and a CDRH3 of a heavy chain variable domain (VH) having the amino acid sequence of SEQ ID NO: 44. Alternatively or in addition, an anti-FAP CAR comprises a CDRL1, a CDRL2 and a CDRL3 of a light chain variable domain (VL) having the amino acid sequence of SEQ ID NO: 45.

In some embodiments, an anti-FAP CAR comprises a CDRH3 having the amino acid sequence of SEQ ID NO: 42. In some embodiments, an anti-FAP CAR comprises a CDRH1 having the amino acid sequence of SEQ ID NO: 40, a CDRH2 having the amino acid sequence of SEQ ID NO: 41, and a CDRH3 having the amino acid sequence of SEQ ID NO: 42. In some embodiments, an anti-FAP CAR comprises a CDRH1 having the amino acid sequence of SEQ ID NO: 40, a CDRH2 having the amino acid sequence of SEQ ID NO: 41, a CDRH3 having the amino acid sequence of SEQ ID NO: 42, a CDRL1 having the amino acid sequence of SEQ ID NO: 4, a CDRL2 having the amino acid sequence of SEQ ID NO: 5, and a CDRL3 having the amino acid sequence of SEQ ID NO: 43.

In some embodiments, an anti-FAP CAR comprises a CDRH1, a CDRH2, and a CDRH3, which collectively contains no more than 5 amino acid variations (e.g., no more than 5, 4, 3, 2, or 1 amino acid variation) as compared with the CDRH1 having the amino acid sequence of SEQ ID NO: 40, CDRH2 having the amino acid sequence of SEQ ID NO: 41, and CDRH3 having the amino acid sequence of SEQ ID NO: 42. Alternatively or in addition, an anti-FAP CAR comprises a CDRL1, a CDRL2, and a CDRL3, which collectively contains no more than 5 amino acid variations (e.g., no more than 5, 4, 3, 2 or 1 amino acid variation) as compared with the CDRL1 having the amino acid sequence of SEQ ID NO: 4, CDRL2 having the amino acid sequence of SEQ ID NO: 5, and CDRL3 having the amino acid sequence of SEQ ID NO: 43.

In some embodiments, an anti-FAP CAR comprises a CDRH1, a CDRH2, and a CDRH3 that collectively are at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the CDRH1 having the amino acid sequence of SEQ ID NO: 40, CDRH2 having the amino acid sequence of SEQ ID NO: 41, and CDRH3 having the amino acid sequence of SEQ ID NO: 42. Alternatively or in addition, an anti-FAP CAR comprises a CDRL1, a CDRL2, and a CDRL3 that collectively are at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the to the CDRL1 having the amino acid sequence of SEQ ID NO: 4, CDRL2 having the amino acid sequence of SEQ ID NO: 5, and CDRL3 having the amino acid sequence of SEQ ID NO: 43.

In some embodiments, an anti-FAP CAR comprises: a CDRH1 having no more than 3 amino acid variations (e.g., no more than 3, 2, or 1 amino acid variation) as compared with the CDRH1 having the amino acid sequence of SEQ ID NO: 40; a CDRH2 having no more than 3 amino acid variations (e.g., no more than 3, 2, or 1 amino acid variation) as compared with the CDRH2 having the amino acid sequence of SEQ ID NO: 41; and/or a CDRH3 having no more than 3 amino acid variations (e.g., no more than 3, 2, or 1 amino acid variation) as compared with the CDRH3 having the amino acid sequence of SEQ ID NO: 42. Alternatively or in addition, an anti-FAP CAR comprises: a CDRL1 having no more than 3 amino acid variations (e.g., no more than 3, 2, or 1 amino acid variation) as compared with the CDRL1 having the amino acid sequence of SEQ ID NO: 4; a CDRL2 having no more than 3 amino acid variations (e.g., no more than 3, 2, or 1 amino acid variation) as compared with the CDRL2 having the amino acid sequence of SEQ ID NO: 5; and/or a CDRL3 having no more than 3 amino acid variations (e.g., no more than 3, 2, or 1 amino acid variation) as compared with the CDRL3 having the amino acid sequence of SEQ ID NO: 43.

In some embodiments, an anti-FAP CAR comprises a VH comprising the amino acid sequence of SEQ ID NO: 44. Alternatively or in addition, an anti-FAP CAR comprises a VL comprising the amino acid sequence of SEQ ID NO: 45. In some embodiments, an anti-FAP CAR comprises a VH comprising the amino acid sequence of SEQ ID NO: 44 and a VL comprising the amino acid sequence of SEQ ID NO: 45.

In some embodiments, an anti-FAP CAR comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 44. Alternatively or in addition, an anti-FAP CAR comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 45.

In some embodiments, an anti-FAP CAR comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VH as set forth in SEQ ID NO: 44. Alternatively or in addition, an anti-FAP CAR comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VL as set forth in SEQ ID NO: 5.

In some embodiments, an anti-FAP CAR comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 44. Alternatively or in addition, an anti-FAP CAR comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 45. In some embodiments, the number of amino acid variations (e.g., no more than 20, 19, 18, 17, 16, 15, 14, 13, 12, 11, 10, 9, 8, 7, 6, 5, 4, 3, 2, or 1 amino acid variation) may occur within a VH of SEQ ID NO: 44 and/or a VL of SEQ ID NO: 45 excluding any of the CDR sequences therein. In some embodiments, an anti-FAP CAR comprises a heavy chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VH of SEQ ID NO: 44, and/or a light chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VL of SEQ ID NO: 45.

In some embodiments, an anti-FAP CAR comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VH as set forth in SEQ ID NO: 44. Alternatively or in addition, an anti-FAP CAR comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VL as set forth in SEQ ID NO: 45. In some embodiments, the degree of sequence variation (e.g., at least 80%, at least 85%, at least 90%, at least 91%, at least 92%, at least 93%, at least 94%, at least 95%, at least 96%, at least 97%, at least 98%, at least 99%, or 100%) may occur within a VH of SEQ ID NO: 44, and/or a VL of SEQ ID NO: 45 excluding any of the CDR sequences therein. In some embodiments, an anti-FAP CAR comprises a heavy chain variable sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the framework sequence of a VH of SEQ ID NO: 44, and/or a light chain variable sequence that comprises a framework sequence that at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the framework sequence of a VL of SEQ ID NO: 45.

In some embodiments, an anti-FAP CAR comprises a CDRH1, a CDRH2 and a CDRH3 of a heavy chain variable domain (VH) having the amino acid sequence of SEQ ID NO: 47. Alternatively or in addition, an anti-FAP CAR comprises a CDRL1, a CDRL2 and a CDRL3 of a light chain variable domain (VL) having the amino acid sequence of SEQ ID NO: 48.

In some embodiments, an anti-FAP CAR comprises a CDRH3 having the amino acid sequence of SEQ ID NO: 42. In some embodiments, an anti-FAP CAR comprises a CDRH1 having the amino acid sequence of SEQ ID NO: 10, a CDRH2 having the amino acid sequence of SEQ ID NO: 11, and a CDRH3 having the amino acid sequence of SEQ ID NO: 42. In some embodiments, an anti-FAP CAR comprises a CDRH1 having the amino acid sequence of SEQ ID NO: 10, a CDRH2 having the amino acid sequence of SEQ ID NO: 11, a CDRH3 having the amino acid sequence of SEQ ID NO: 42, a CDRL1 having the amino acid sequence of SEQ ID NO: 4, a CDRL2 having the amino acid sequence of SEQ ID NO: 5, and a CDRL3 having the amino acid sequence of SEQ ID NO: 43.

In some embodiments, an anti-FAP CAR comprises a CDRH1, a CDRH2, and a CDRH3, which collectively contains no more than 5 amino acid variations (e.g., no more than 5, 4, 3, 2, or 1 amino acid variation) as compared with the CDRH1 having the amino acid sequence of SEQ ID NO: 10, CDRH2 having the amino acid sequence of SEQ ID NO: 11, and CDRH3 having the amino acid sequence of SEQ ID NO: 42. Alternatively or in addition, an anti-FAP CAR comprises a CDRL1, a CDRL2, and a CDRL3, which collectively contains no more than 5 amino acid variations (e.g., no more than 5, 4, 3, 2 or 1 amino acid variation) as compared with the CDRL1 having the amino acid sequence of SEQ ID NO: 4, CDRL2 having the amino acid sequence of SEQ ID NO: 5, and CDRL3 having the amino acid sequence of SEQ ID NO: 43.

In some embodiments, an anti-FAP CAR comprises a CDRH1, a CDRH2, and a CDRH3 that collectively are at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the CDRH1 having the amino acid sequence of SEQ ID NO: 10, CDRH2 having the amino acid sequence of SEQ ID NO: 11, and CDRH3 having the amino acid sequence of SEQ ID NO: 42. Alternatively or in addition, an anti-FAP CAR comprises a CDRL1, a CDRL2, and a CDRL3 that collectively are at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the to the CDRL1 having the amino acid sequence of SEQ ID NO: 4, CDRL2 having the amino acid sequence of SEQ ID NO: 5, and CDRL3 having the amino acid sequence of SEQ ID NO: 43.

In some embodiments, an anti-FAP CAR comprises: a CDRH1 having no more than 3 amino acid variations (e.g., no more than 3, 2, or 1 amino acid variation) as compared with the CDRH1 having the amino acid sequence of SEQ ID NO: 10; a CDRH2 having no more than 3 amino acid variations (e.g., no more than 3, 2, or 1 amino acid variation) as compared with the CDRH2 having the amino acid sequence of SEQ ID NO: 11; and/or a CDRH3 having no more than 3 amino acid variations (e.g., no more than 3, 2, or 1 amino acid variation) as compared with the CDRH3 having the amino acid sequence of SEQ ID NO: 42. Alternatively or in addition, an anti-FAP CAR comprises: a CDRL1 having no more than 3 amino acid variations (e.g., no more than 3, 2, or 1 amino acid variation) as compared with the CDRL1 having the amino acid sequence of SEQ ID NO: 4; a CDRL2 having no more than 3 amino acid variations (e.g., no more than 3, 2, or 1 amino acid variation) as compared with the CDRL2 having the amino acid sequence of SEQ ID NO: 5; and/or a CDRL3 having no more than 3 amino acid variations (e.g., no more than 3, 2, or 1 amino acid variation) as compared with the CDRL3 having the amino acid sequence of SEQ ID NO: 43.

In some embodiments, an anti-FAP CAR comprises a VH comprising the amino acid sequence of SEQ ID NO: 47. Alternatively or in addition, an anti-FAP CAR comprises a VL comprising the amino acid sequence of SEQ ID NO: 48. In some embodiments, an anti-FAP CAR comprises a VH comprising the amino acid sequence of SEQ ID NO: 47 and a VL comprising the amino acid sequence of SEQ ID NO: 48.

In some embodiments, an anti-FAP CAR comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 47. Alternatively or in addition, an anti-FAP CAR comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 48.

In some embodiments, an anti-FAP CAR comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VH as set forth in SEQ ID NO: 47. Alternatively or in addition, an anti-FAP CAR comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VL as set forth in SEQ ID NO: 48.

In some embodiments, an anti-FAP CAR comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 47. Alternatively or in addition, an anti-FAP CAR comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 48. In some embodiments, the number of amino acid variations (e.g., no more than 20, 19, 18, 17, 16, 15, 14, 13, 12, 11, 10, 9, 8, 7, 6, 5, 4, 3, 2, or 1 amino acid variation) may occur within a VH of SEQ ID NO: 47 and/or a VL of SEQ ID NO: 48 excluding any of the CDR sequences therein. In some embodiments, an anti-FAP CAR comprises a heavy chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VH of SEQ ID NO: 47, and/or a light chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VL of SEQ ID NO: 48.

In some embodiments, an anti-FAP CAR comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VH as set forth in SEQ ID NO: 47. Alternatively or in addition, an anti-FAP CAR comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VL as set forth in SEQ ID NO: 48. In some embodiments, the degree of sequence variation (e.g., at least 80%, at least 85%, at least 90%, at least 91%, at least 92%, at least 93%, at least 94%, at least 95%, at least 96%, at least 97%, at least 98%, at least 99%, or 100%) may occur within a VH of SEQ ID NO: 47, and/or a VL of SEQ ID NO: 48 excluding any of the CDR sequences therein. In some embodiments, an anti-FAP CAR comprises a heavy chain variable sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the framework sequence of a VH of SEQ ID NO: 47, and/or a light chain variable sequence that comprises a framework sequence that at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the framework sequence of a VL of SEQ ID NO: 48.

In some embodiments, an anti-FAP CAR comprises a CDRH1, a CDRH2 and a CDRH3 of a heavy chain variable domain (VH) having the amino acid sequence of SEQ ID NO: 51. Alternatively or in addition, an anti-FAP CAR comprises a CDRL1, a CDRL2 and a CDRL3 of a light chain variable domain (VL) having the amino acid sequence of SEQ ID NO: 52.

In some embodiments, an anti-FAP CAR comprises a CDRH3 having the amino acid sequence of SEQ ID NO: 42. In some embodiments, an anti-FAP CAR comprises a CDRH1 having the amino acid sequence of SEQ ID NO: 10, a CDRH2 having the amino acid sequence of SEQ ID NO: 50, and a CDRH3 having the amino acid sequence of SEQ ID NO: 42. In some embodiments, an anti-FAP CAR comprises a CDRH1 having the amino acid sequence of SEQ ID NO: 10, a CDRH2 having the amino acid sequence of SEQ ID NO: 50, a CDRH3 having the amino acid sequence of SEQ ID NO: 42, a CDRL1 having the amino acid sequence of SEQ ID NO: 4, a CDRL2 having the amino acid sequence of SEQ ID NO: 5, and a CDRL3 having the amino acid sequence of SEQ ID NO: 43.

In some embodiments, an anti-FAP CAR comprises a CDRH1, a CDRH2, and a CDRH3, which collectively contains no more than 5 amino acid variations (e.g., no more than 5, 4, 3, 2, or 1 amino acid variation) as compared with the CDRH1 having the amino acid sequence of SEQ ID NO: 10, CDRH2 having the amino acid sequence of SEQ ID NO: 50, and CDRH3 having the amino acid sequence of SEQ ID NO: 42. Alternatively or in addition, an anti-FAP CAR comprises a CDRL1, a CDRL2, and a CDRL3, which collectively contains no more than 5 amino acid variations (e.g., no more than 5, 4, 3, 2 or 1 amino acid variation) as compared with the CDRL1 having the amino acid sequence of SEQ ID NO: 4, CDRL2 having the amino acid sequence of SEQ ID NO: 5, and CDRL3 having the amino acid sequence of SEQ ID NO: 43.

In some embodiments, an anti-FAP CAR comprises a CDRH1, a CDRH2, and a CDRH3 that collectively are at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the CDRH1 having the amino acid sequence of SEQ ID NO: 10, CDRH2 having the amino acid sequence of SEQ ID NO: 50, and CDRH3 having the amino acid sequence of SEQ ID NO: 42. Alternatively or in addition, an anti-FAP CAR comprises a CDRL1, a CDRL2, and a CDRL3 that collectively are at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the to the CDRL1 having the amino acid sequence of SEQ ID NO: 4, CDRL2 having the amino acid sequence of SEQ ID NO: 5, and CDRL3 having the amino acid sequence of SEQ ID NO: 43.

In some embodiments, an anti-FAP CAR comprises: a CDRH1 having no more than 3 amino acid variations (e.g., no more than 3, 2, or 1 amino acid variation) as compared with the CDRH1 having the amino acid sequence of SEQ ID NO: 10; a CDRH2 having no more than 3 amino acid variations (e.g., no more than 3, 2, or 1 amino acid variation) as compared with the CDRH2 having the amino acid sequence of SEQ ID NO: 50; and/or a CDRH3 having no more than 3 amino acid variations (e.g., no more than 3, 2, or 1 amino acid variation) as compared with the CDRH3 having the amino acid sequence of SEQ ID NO: 42. Alternatively or in addition, an anti-FAP CAR comprises: a CDRL1 having no more than 3 amino acid variations (e.g., no more than 3, 2, or 1 amino acid variation) as compared with the CDRL1 having the amino acid sequence of SEQ ID NO: 4; a CDRL2 having no more than 3 amino acid variations (e.g., no more than 3, 2, or 1 amino acid variation) as compared with the CDRL2 having the amino acid sequence of SEQ ID NO: 5; and/or a CDRL3 having no more than 3 amino acid variations (e.g., no more than 3, 2, or 1 amino acid variation) as compared with the CDRL3 having the amino acid sequence of SEQ ID NO: 43.

In some embodiments, an anti-FAP CAR comprises a VH comprising the amino acid sequence of SEQ ID NO: 51. Alternatively or in addition, an anti-FAP CAR comprises a VL comprising the amino acid sequence of SEQ ID NO: 52. In some embodiments, an anti-FAP CAR comprises a VH comprising the amino acid sequence of SEQ ID NO: 51 and a VL comprising the amino acid sequence of SEQ ID NO: 52.

In some embodiments, an anti-FAP CAR comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 51. Alternatively or in addition, an anti-FAP CAR comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 52.

In some embodiments, an anti-FAP CAR comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VH as set forth in SEQ ID NO: 51. Alternatively or in addition, an anti-FAP CAR comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VL as set forth in SEQ ID NO: 52.

In some embodiments, an anti-FAP CAR comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 51. Alternatively or in addition, an anti-FAP CAR comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 52. In some embodiments, the number of amino acid variations (e.g., no more than 20, 19, 18, 17, 16, 15, 14, 13, 12, 11, 10, 9, 8, 7, 6, 5, 4, 3, 2, or 1 amino acid variation) may occur within a VH of SEQ ID NO: 51 and/or a VL of SEQ ID NO: 52 excluding any of the CDR sequences therein. In some embodiments, an anti-FAP CAR comprises a heavy chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VH of SEQ ID NO: 51, and/or a light chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VL of SEQ ID NO: 52.

In some embodiments, an anti-FAP CAR comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VH as set forth in SEQ ID NO: 51. Alternatively or in addition, an anti-FAP CAR comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VL as set forth in SEQ ID NO: 52. In some embodiments, the degree of sequence variation (e.g., at least 80%, at least 85%, at least 90%, at least 91%, at least 92%, at least 93%, at least 94%, at least 95%, at least 96%, at least 97%, at least 98%, at least 99%, or 100%) may occur within a VH of SEQ ID NO: 51, and/or a VL of SEQ ID NO: 52 excluding any of the CDR sequences therein. In some embodiments, an anti-FAP CAR comprises a heavy chain variable sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the framework sequence of a VH of SEQ ID NO: 51, and/or a light chain variable sequence that comprises a framework sequence that at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the framework sequence of a VL of SEQ ID NO: 52.

In some embodiments, an anti-FAP CAR comprises a CDRH1, a CDRH2 and a CDRH3 of a heavy chain variable domain (VH) having the amino acid sequence of SEQ ID NO: 56. Alternatively or in addition, an anti-FAP CAR comprises a CDRL1, a CDRL2 and a CDRL3 of a light chain variable domain (VL) having the amino acid sequence of SEQ ID NO: 57.

In some embodiments, an anti-FAP CAR comprises a CDRH3 having the amino acid sequence of SEQ ID NO: 54. In some embodiments, an anti-FAP CAR comprises a CDRH1 having the amino acid sequence of SEQ ID NO: 10, a CDRH2 having the amino acid sequence of SEQ ID NO: 11, and a CDRH3 having the amino acid sequence of SEQ ID NO: 54. In some embodiments, an anti-FAP CAR comprises a CDRH1 having the amino acid sequence of SEQ ID NO: 10, a CDRH2 having the amino acid sequence of SEQ ID NO: 11, a CDRH3 having the amino acid sequence of SEQ ID NO: 54, a CDRL1 having the amino acid sequence of SEQ ID NO: 4, a CDRL2 having the amino acid sequence of SEQ ID NO: 5, and a CDRL3 having the amino acid sequence of SEQ ID NO: 55.

In some embodiments, an anti-FAP CAR comprises a CDRH1, a CDRH2, and a CDRH3, which collectively contains no more than 5 amino acid variations (e.g., no more than 5, 4, 3, 2, or 1 amino acid variation) as compared with the CDRH1 having the amino acid sequence of SEQ ID NO: 10, CDRH2 having the amino acid sequence of SEQ ID NO: 11, and CDRH3 having the amino acid sequence of SEQ ID NO: 54. Alternatively or in addition, an anti-FAP CAR comprises a CDRL1, a CDRL2, and a CDRL3, which collectively contains no more than 5 amino acid variations (e.g., no more than 5, 4, 3, 2 or 1 amino acid variation) as compared with the CDRL1 having the amino acid sequence of SEQ ID NO: 4, CDRL2 having the amino acid sequence of SEQ ID NO: 5, and CDRL3 having the amino acid sequence of SEQ ID NO: 55.

In some embodiments, an anti-FAP CAR comprises a CDRH1, a CDRH2, and a CDRH3 that collectively are at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the CDRH1 having the amino acid sequence of SEQ ID NO: 10, CDRH2 having the amino acid sequence of SEQ ID NO: 11, and CDRH3 having the amino acid sequence of SEQ ID NO: 54. Alternatively or in addition, an anti-FAP CAR comprises a CDRL1, a CDRL2, and a CDRL3 that collectively are at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the to the CDRL1 having the amino acid sequence of SEQ ID NO: 4, CDRL2 having the amino acid sequence of SEQ ID NO: 5, and CDRL3 having the amino acid sequence of SEQ ID NO: 55.

In some embodiments, an anti-FAP CAR comprises: a CDRH1 having no more than 3 amino acid variations (e.g., no more than 3, 2, or 1 amino acid variation) as compared with the CDRH1 having the amino acid sequence of SEQ ID NO: 10; a CDRH2 having no more than 3 amino acid variations (e.g., no more than 3, 2, or 1 amino acid variation) as compared with the CDRH2 having the amino acid sequence of SEQ ID NO: 11; and/or a CDRH3 having no more than 3 amino acid variations (e.g., no more than 3, 2, or 1 amino acid variation) as compared with the CDRH3 having the amino acid sequence of SEQ ID NO: 54. Alternatively or in addition, an anti-FAP CAR comprises: a CDRL1 having no more than 3 amino acid variations (e.g., no more than 3, 2, or 1 amino acid variation) as compared with the CDRL1 having the amino acid sequence of SEQ ID NO: 4; a CDRL2 having no more than 3 amino acid variations (e.g., no more than 3, 2, or 1 amino acid variation) as compared with the CDRL2 having the amino acid sequence of SEQ ID NO: 5; and/or a CDRL3 having no more than 3 amino acid variations (e.g., no more than 3, 2, or 1 amino acid variation) as compared with the CDRL3 having the amino acid sequence of SEQ ID NO: 55.

In some embodiments, an anti-FAP CAR comprises a VH comprising the amino acid sequence of SEQ ID NO: 56. Alternatively or in addition, an anti-FAP CAR comprises a VL comprising the amino acid sequence of SEQ ID NO: 57. In some embodiments, an anti-FAP CAR comprises a VH comprising the amino acid sequence of SEQ ID NO: 56 and a VL comprising the amino acid sequence of SEQ ID NO: 57.

In some embodiments, an anti-FAP CAR comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 56. Alternatively or in addition, an anti-FAP CAR comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 57.

In some embodiments, an anti-FAP CAR comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VH as set forth in SEQ ID NO: 56. Alternatively or in addition, an anti-FAP CAR comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VL as set forth in SEQ ID NO: 57.

In some embodiments, an anti-FAP CAR comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 56. Alternatively or in addition, an anti-FAP CAR comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 57. In some embodiments, the number of amino acid variations (e.g., no more than 20, 19, 18, 17, 16, 15, 14, 13, 12, 11, 10, 9, 8, 7, 6, 5, 4, 3, 2, or 1 amino acid variation) may occur within a VH of SEQ ID NO: 56 and/or a VL of SEQ ID NO: 57 excluding any of the CDR sequences therein. In some embodiments, an anti-FAP CAR comprises a heavy chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VH of SEQ ID NO: 56, and/or a light chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VL of SEQ ID NO: 57.

In some embodiments, an anti-FAP CAR comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VH as set forth in SEQ ID NO: 56. Alternatively or in addition, an anti-FAP CAR comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VL as set forth in SEQ ID NO: 57. In some embodiments, the degree of sequence variation (e.g., at least 80%, at least 85%, at least 90%, at least 91%, at least 92%, at least 93%, at least 94%, at least 95%, at least 96%, at least 97%, at least 98%, at least 99%, or 100%) may occur within a VH of SEQ ID NO: 56, and/or a VL of SEQ ID NO: 57 excluding any of the CDR sequences therein. In some embodiments, an anti-FAP CAR comprises a heavy chain variable sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the framework sequence of a VH of SEQ ID NO: 56, and/or a light chain variable sequence that comprises a framework sequence that at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the framework sequence of a VL of SEQ ID NO: 57.

Some aspects of the disclosure provide anti-FAP CARs that comprise a FAP binder known in the art, for example, any of the FAP binders provided in Table 3. In some embodiments, an anti-FAP CAR of the present disclosure comprises a FAP binding peptide known in the art, for example, a FAP binding peptide as provided in Table 3. In some embodiments, an anti-FAP CAR of the present disclosure comprises one or more of the heavy chain variable (VH) and/or light chain variable (VL) domains of an anti-FAP antibody know in the art, for example, an anti-FAP antibody as provided in Table 3. In some embodiments, an anti-FAP CAP of the present disclosure comprises a VH domain that include one or more of the heavy chain CDR sequences (e.g., CDRH1, CDRH2, and CDRH3) of any known anti-FAP antibody in the art, for example, an anti-FAP antibody as provided in Table 3. In some embodiments, an anti-FAP CAP of the present disclosure comprises a VL domain that include one or more of the light chain CDR sequences (e.g., CDRL1, CDRL2, and CDRL3) of any known anti-FAP antibody in the art, for example, an anti-FAP antibody as provided in Table 3.

In some embodiments, an anti-FAP CAR of the present disclosure comprises a heavy chain variable domain and/or a light chain variable domain of any one of the anti-FAP antibodies known in the art, for example, an anti-FAP antibody selected from Table 3, and variants thereof. In some embodiments, an anti-FAP CAR comprises the heavy chain variable and light chain variable pairs of any anti-FAP antibodies known in the art, for example, an anti-FAP antibody selected from Table 3.

Aspects of the disclosure provide anti-FAP CAR comprising a heavy chain variable (VH) and/or a light chain variable (VL) domain amino acid sequence homologous to any of those known anti-FAP antibody in the art, for example, an anti-FAP antibody as provided in Table 3. In some embodiments, an anti-FAP CAR comprises a VH or a VL that is at least 75% (e.g., 80%, 85%, 90%, 95%, 98%, or 99%) identical to the VH and/or any VL of any of those known anti-FAP antibody in the art, for example, an anti-FAP antibody as provided in Table 3. In some embodiments, the homologous VH and/or a VL amino acid sequences of the anti-FAP CAR do not vary within any of the CDR sequences of any of the known anti-FAP antibody in the art, e.g., an anti-FAP antibody as provided in Table 3. For example, in some embodiments, the degree of sequence variation (e.g., 75%, 80%, 85%, 90%, 95%, 98%, or 99%) may occur within a VH and/or a VL sequence of an anti-FAP CAR excluding any of the CDR sequences of any of the known anti-FAP antibody in the art, e.g., an anti-FAP antibody as provided in Table 3. In some embodiments, any of the anti-FAP CAR provided herein comprise a VH sequence and a VL sequence that comprises a framework sequence that is at least 75%, 80%, 85%, 90%, 95%, 98%, or 99% identical to the framework sequence of any anti-FAP antibodies of any of the known anti-FAP antibody in the art, e.g., an anti-FAP antibody as provided in Table 3. In some embodiments, an anti-FAP CAR comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH of any of the of any of the known anti-FAP antibody in the art, e.g., an anti-FAP antibody as provided in Table 3. Alternatively or in addition, an anti-FAP CAR comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL of any of the known anti-FAP antibody in the art, e.g., an anti-FAP antibody as provided in Table 3.

In some embodiments, an anti-FAP CAR comprises a CDRH1, a CDRH2, a CDRH3, a CDRL1, a CDRL2, and a CDRL3 that are the same as the CDRH1, CDRH2, CDRH3, CDRL1, CDRL2, and CDRL3 of any the known anti-FAP antibody in the art, e.g., the anti-FAP antibody as provided in Table 3, and comprises a humanized VH and/or a humanized VL. In some embodiments, an anti-FAP CAR is a humanized variant comprising one or more amino acid substitutions (e.g., in the VH framework region) as compared with of any the known anti-FAP antibody in the art, e.g., the anti-FAP antibody as provided in Table 3, and/or one or more amino acid substitutions (e.g., in the VL framework region) as compared with of any the known anti-FAP antibody in the art, e.g., the anti-FAP antibody as provided in Table 3.

An anti-FAP antibody or antigen binding fragment thereof described herein can be grafted into a chimeric antigen receptor including, but not limited to, antigen-binding fragments thereof (such as Fab, F(ab′), F(ab′)2, Fv), single chain antibodies (e.g., scFv), bi-specific antibodies, or nanobodies. In some embodiments, an anti-FAP CAR described herein comprises a single-chain variable fragment (scFv) as the extracellular ligand-binding domain.

In some embodiments, an anti-FAP CAR comprises an extracellular ligand-binding domain that comprises a single-chain variable fragment (scFv). In some embodiments, an anti-FAP CAR comprises an extracellular ligand-binding domain that comprises a VH and a VL of any one of the anti-FAP scFv selected from Table 2. In some embodiments, an anti-FAP CAR comprises a VH and a VL of any one of the anti-FAP antibody known in the art, for example, an anti-FAP antibody as provided in Table 3. In some embodiments, the VH and the VL of an anti-FAP CAR are joined together by a linker. In some embodiments, a linker may have a length of about 2 to 10 amino acids, 5 to 20 amino acids, 10 to 30 amino acids, 20-50 amino acids, 40 to 60 amino acids, 60 to 80 amino acids, or more than 80 amino acids. In some embodiment, a linker may include a sequence that substantially comprises glycine and serine. An exemplary linker sequence is GGGGSGGGGSGGGAS (SEQ ID NO: 29). In some embodiments, a linker may include, without limitation, any of those encompassed by U.S. Pat. Nos. 8,445,251 and 9,434,931. In some embodiments, an anti-FAP CAR comprises a linker between the VH and the VL, and the linker comprises an amino acid sequence at least 80%, at least 85%, at least 90%, at least 92%, at least 95%, at least 98%, at least 99%, or 100% identical to SEQ ID NO: 29.

In some embodiments, an anti-FAP CAR comprises an extracellular ligand-binding domain that comprises a scFv comprising a VH and a VL, and the C-terminus of the VH is joined with the N terminus of the VL via a linker (e.g., the linker as set forth in SEQ ID NO: 29). In some embodiments, an anti-FAP CAR comprises an extracellular ligand-binding domain that comprises scFv comprising a VH and a VL, and the C-terminus of the VL is joined with the N terminus of the VH via a linker (e.g., the linker as set forth in SEQ ID NO: 29).

In some embodiments, an anti-FAP CAR comprises an extracellular ligand-binding domain that comprises a scFv comprising a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH of any of the anti-FAP antibodies listed in Table 2. Alternatively or in addition, an anti-FAP CAR comprises an extracellular ligand-binding domain that comprises a scFv comprising a VL containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH of any of the anti-FAP antibodies listed in Table 2. In some embodiments, an anti-FAP CAR comprises an extracellular ligand-binding domain that comprises a scFv comprising a VH comprising an amino acid sequence that is at least 80% (e.g., 80%, 85%, 90%, 95%, 98%, or 99%) identical to the VH of any of the anti-FAP antibodies listed in Table 2. Alternatively or in addition, an anti-FAP CAR comprises an extracellular ligand-binding domain that comprises a scFv comprising a VL comprising an amino acid sequence that is at least 80% (e.g., 80%, 85%, 90%, 95%, 98%, or 99%) identical to the VH of any of the anti-FAP antibodies listed in Table 2.

In some embodiments, an anti-FAP CAR comprises an extracellular ligand-binding domain that comprises a scFv comprising an amino acid sequence containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the any of the anti-FAP scFv listed in Table 2. In some embodiments, an anti-FAP CAR comprises an extracellular ligand-binding domain that comprises a scFv comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%)) identical to any of the anti-FAP scFv listed in Table 2.

In some embodiments, an anti-FAP CAR comprises an extracellular ligand-binding domain that comprises a scFv comprising an amino acid sequence containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the scFv amino acid sequence as set forth in SEQ ID NO: 9. In some embodiments, an anti-FAP CAR comprises an extracellular ligand-binding domain that comprises a scFv comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the scFv amino acid sequence as set forth in SEQ ID NO: 9. In some embodiments, an anti-FAP CAR comprises an extracellular ligand-binding domain that comprises a scFv comprising an amino acid sequence of SEQ ID NO: 9.

In some embodiments, an anti-FAP CAR comprises an extracellular ligand-binding domain that comprises a scFv comprising an amino acid sequence containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the scFv amino acid sequence as set forth in SEQ ID NO: 16. In some embodiments, an anti-FAP CAR comprises an extracellular ligand-binding domain that comprises a scFv comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the scFv amino acid sequence as set forth in SEQ ID NO: 16. In some embodiments, an anti-FAP CAR comprises an extracellular ligand-binding domain that comprises a scFv comprising an amino acid sequence of SEQ ID NO: 16.

In some embodiments, an anti-FAP CAR comprises an extracellular ligand-binding domain that comprises a scFv comprising an amino acid sequence containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the scFv amino acid sequence as set forth in SEQ ID NO: 46. In some embodiments, an anti-FAP CAR comprises an extracellular ligand-binding domain that comprises a scFv comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the scFv amino acid sequence as set forth in SEQ ID NO: 46. In some embodiments, an anti-FAP CAR comprises an extracellular ligand-binding domain that comprises a scFv comprising an amino acid sequence of SEQ ID NO: 46.

In some embodiments, an anti-FAP CAR comprises an extracellular ligand-binding domain that comprises a scFv comprising an amino acid sequence containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the scFv amino acid sequence as set forth in SEQ ID NO: 49. In some embodiments, an anti-FAP CAR comprises an extracellular ligand-binding domain that comprises a scFv comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the scFv amino acid sequence as set forth in SEQ ID NO: 49. In some embodiments, an anti-FAP CAR comprises an extracellular ligand-binding domain that comprises a scFv comprising an amino acid sequence of SEQ ID NO: 49

In some embodiments, an anti-FAP CAR comprises an extracellular ligand-binding domain that comprises a scFv comprising an amino acid sequence containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the scFv amino acid sequence as set forth in SEQ ID NO: 53. In some embodiments, an anti-FAP CAR comprises an extracellular ligand-binding domain that comprises a scFv comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the scFv amino acid sequence as set forth in SEQ ID NO: 53. In some embodiments, an anti-FAP CAR comprises an extracellular ligand-binding domain that comprises a scFv comprising an amino acid sequence of SEQ ID NO: 53.

In some embodiments, an anti-FAP CAR comprises an extracellular ligand-binding domain that comprises a scFv comprising an amino acid sequence containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the scFv amino acid sequence as set forth in SEQ ID NO: 58. In some embodiments, an anti-FAP CAR comprises an extracellular ligand-binding domain that comprises a scFv comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the scFv amino acid sequence as set forth in SEQ ID NO: 58. In some embodiments, an anti-FAP CAR comprises an extracellular ligand-binding domain that comprises a scFv comprising an amino acid sequence of SEQ ID NO: 58.

In some embodiments, an anti-FAP CAR comprises an extracellular ligand-binding domain that comprises a scFv comprising a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH of any of the known anti-FAP antibodies in the art, for example, an anti-FAP antibody as provided in Table 3. Alternatively or in addition, an anti-FAP CAR comprises an extracellular ligand-binding domain that comprises a scFv comprising a VL containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH any of the known anti-FAP antibodies in the art, for example, an anti-FAP antibody as provided in Table 3. In some embodiments, an anti-FAP CAR comprises an extracellular ligand-binding domain that comprises a scFv comprising a VH comprising an amino acid sequence that is at least 80% (e.g., 80%, 85%, 90%, 95%, 98%, or 99%) identical to the VH of any of the known anti-FAP antibodies in the art, for example, an anti-FAP antibody as provided in Table 3. Alternatively or in addition, an anti-FAP CAR comprises an extracellular ligand-binding domain that comprises a scFv comprising a VL comprising an amino acid sequence that is at least 80% (e.g., 80%, 85%, 90%, 95%, 98%, or 99%) identical to the VH of any of the known anti-FAP antibodies in the art, for example, an anti-FAP antibody as provided in Table 3.

In some embodiments, an anti-FAP CAR of the present disclosure further comprises a hinge region. In some embodiments, the hinge region is a CD8 hinge region. An exemplary amino acid sequence for a CD8 hinge region is set forth in SEQ ID NO: 30:

TTTPAPRPPTPAPTIASOPLSLRPEACRPAAGGAVHTRGLDFACD

In some embodiments, an anti-FAP CAR of the present disclosure comprises a CD8 hinge region having the amino acid sequence as set forth in SEQ ID NO: 30, or a variant thereof. In some embodiments, an anti-FAP CAR comprises a hinge region comprising an amino acid sequence at least 70%, at least 80%, at least 85%, at least 90%, at least 92%, at least 95%, at least 98%, at least 99%, or 100% identical to SEQ ID NO: 30. In some embodiments, the hinge region can be any suitable hinge regions known in the art, e.g., the hinge regions described by Guedan et al., Engineering and Design of Chimeric Antigen Receptors, Mol Ther Methods Clin Dev. 2019 Mar. 15; 12:145-156, such as hinge regions derived from IgG1, IgG2, IgG4, CD28, CD8, or a hybrid thereof.

In some embodiments, an anti-FAP CAR of the present disclosure further comprises a transmembrane domain, which links the extracellular ligand-binding domain with the intracellular signaling and co-stimulatory domains. With respect to the transmembrane domain, the CAR can be designed to comprise a transmembrane domain that is fused to the extracellular domain (e.g., the antigen binding domain) of the CAR. Any transmembrane domain is contemplated for use herein as long as the domain is capable of anchoring a CAR comprising the domain to a cell membrane. In some embodiments, the transmembrane domain that naturally is associated with one of the domains in the CAR is used. In some instances, the transmembrane domain can be selected or modified by amino acid substitution to avoid binding of such domains to the transmembrane domains of the same or different surface membrane proteins to minimize interactions with other members of the receptor complex. One skilled in the art would appreciate that the full transmembrane domain, or portion thereof, is implemented with the cytoplasmic domain, or a portion thereof. The transmembrane domain may be derived either from a natural or from a synthetic source. Where the source is natural, the domain may be derived from any membrane-bound or transmembrane protein. In some embodiments, the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

In some embodiments, the transmembrane domain is a CD8 transmembrane domain. An exemplary amino acid sequence for a CD8 transmembrane domain is set forth in SEQ ID NO: 31:

IYIWAPLAGTCGVLLLSLVITLYC

In some embodiments, an anti-FAP CAR of the present disclosure comprises a CD8 transmembrane domain (TM) having the amino acid sequence as set forth in SEQ ID NO: 31, or a variant thereof. In some embodiments, an anti-FAP CAR comprises a transmembrane domain comprising an amino acid sequence at least 70%, at least 80%, at least 85%, at least 90%, at least 92%, at least 95%, at least 98%, at least 99%, or 100% identical to SEQ ID NO: 31. In some embodiments, the transmembrane domain can be any suitable transmembrane domain known in the art, e.g., transmembrane domain derived from TCRα, TCRβ, TCRζ, CD2, CD3ζ, CD3ε, CD3γ, CD3δ, CD4, CD5, CD8, CD9, CD16, CD22, CD28, CD32, CD33, CD34, CD37, CD45, CD64, CD80, CD86, CD134, CD137, CD154, SLAMF4, or inducible T cell costimulator (ICOS). However, any transmembrane domain is contemplated for use herein as long as the domain is capable of anchoring a CAR comprising the extracellular domain to a cell membrane. Transmembrane domains can be identified using any method known in the art or described herein, e.g., by using the UniProt Database. In some embodiments, the transmembrane domain is connected to a hinge region. Exemplary pairs of hinge region and transmembrane domain include CD8 hinge/TM, IgG1 Hinge/CD2 TM, or IgG1 Hinge/SLAMF4 TM. Table 6 shows the amino acid sequence and nucleotide coding sequence for the exemplary hinge/TM domains.

TABLE 6
Exemplary Hinge/TM Domains

| Molecule | Amino Acid Sequence | Nucleotide Sequence |
|---|---|---|
| CD8 Hinge/TM | TTTPAPRPPTPAPTIASQPLSLRPE | ACCACAACCCCTGCTCCAAGGCCCC |
| | ACRPAAGGAVHTRGLDFACDIYIWA | CCACACCCGCTCCAACTATAGCCAG |
| | PLAGTCGVLLLSLVITLYC | CCAACCATTGAGCCTCAGACCTGAA |
| | (SEQ ID NO: 83) | GCTTGCAGGCCCGCAGCAGGAGGCG |
| | | CCGTCCATACGCGAGGCCTGGACTT |
| | | CGCGTGTGATATTTATATTTGGGCC |
| | | CCTTTGGCCGGAACATGTGGGGTGT |
| | | TGCTTCTCTCCCTTGTGATCACTCT |
| | | GTATTGT |
| | | (SEQ ID NO: 84) |
| IgG1 Hinge/CD2 TM | EPKSPDKTHTCPPCPIYLIIGICGG | GAGCCGAAAAGTCCGGACAAAACTC |
| | GSLLMVFVALLVFYIT | ATACATGTCCACCATGCCCAATTTA |
| | (SEQ ID NO: 85) | CCTCATAATCGGCATTTGTGGAGGA |
| | | GGCTCCCTTCTTATGGTGTTTGTTG |
| | | CTCTGCTGGTATTTTATATTACC |
| | | (SEQ ID NO: 86) |
| IgG1 Hinge/SLAMF4 | EPKSPDKTHTCPPCPFLVIIVILSA | GAGCCGAAAAGTCCGGACAAAACTC |
| TM | LFLGTLACFCV | ATACATGTCCACCATGCCCATTCCT |
| | (SEQ ID NO: 87) | TGTTATCATAGTAATTTTGTCCGCG |
| | | CTTTTTTTGGGGACCTTGGCATGTT |
| | | TCTGTGTT |
| | | (SEQ ID NO: 88) |

In some embodiments, an anti-FAP CAR further comprises one or more intracellular (or cytoplasmic) domain (ICD). In some embodiments, the one or more intracellular domain (e.g., ICD1 and ICD2) of the CAR is responsible for activation of at least one of the normal effector functions of the immune cell in which the CAR has been placed in. The term “effector function” refers to a specialized function of a cell (e.g., an iNKT cell). Effector function of a cell (e.g., an iNKT cell), for example, may be cytolytic activity or helper activity including the secretion of cytokines. Thus, the term “intracellular signaling domain” refers to the portion of a protein which transduces the effector function signal and directs the cell to perform a specialized function. While usually the entire intracellular signaling domain from a molecule can be employed, in many cases it is not necessary to use the entire domain. To the extent that a truncated portion of the intracellular signaling domain is used, such truncated portion may be used in place of the intact domain as long as it transduces the effector function signal. The term intracellular signaling domain is thus meant to include any truncated portion of the intracellular signaling domain sufficient to transduce the effector function signal. In some embodiments, intracellular domains derived from different molecules can be used to form the intracellular domain of the anti-FAP CAR descried herein. In certain embodiments, the intracellular (or cytoplasmic) domain of a chimeric antigen receptor as disclosed herein may include, but is not limited to, a 4-1BB intracellular domain, a OX40 intracellular domain, a CD30 intracellular domain, a CD40 intracellular domain, an ICOS intracellular domain, a LFA-1 intracellular domain, a CD2 intracellular domain, a CD3 ζ intracellular domain, a CD3 γ intracellular domain, a CD3 δ intracellular domain, a CD3 ε intracellular domain, and a CD7 intracellular domain, a CD 27 intracellular domain, a CD28/IL15RA intracellular domain, DAP10 intracellular domain, DAP12 intracellular domain, a DNAM1 intracellular domain, a SLAMF6 intracellular domain, and a CD22 intracellular domain. In some embodiments, an anti-FAP CAR comprises a CD3 ζ intracellular domain. An exemplary amino acid sequence of a CD3 ζ intracellular domain is set forth in SEQ ID NO: 32:

RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR

In some embodiments, an anti-FAP CAR of the present disclosure comprises a CD3 ζ intracellular domain (e.g., ICD2) having the amino acid sequence as set forth in SEQ ID NO: 32, or a variant thereof. In some embodiments, an anti-FAP CAR comprises an intracellular domain comprising an amino acid sequence at least 70%, at least 80%, at least 85%, at least 90%, at least 92%, at least 95%, at least 98%, at least 99%, or 100% identical to SEQ ID NO: 32.

In some embodiments, the intracellular domain further comprises one or more intracellular co-stimulatory domains (i.e., ICD1), such as those described herein, which transmit a co-stimulatory signal which promotes cell proliferation, cell survival, and/or cytokine secretion after binding of the extracellular domain. In some embodiments, such intracellular co-stimulatory domains include, without limitation, any co-stimulatory domain disclosed herein or those domains known in the art, including but not limited to CD28, ICOS, 4-1BB, OX40, or CD27. In some embodiments, an anti-FAP CAR comprises a 4-1BB co-stimulatory domain. An exemplary amino acid sequence of a 4-1BB co-stimulatory domain is set forth in SEQ ID NO: 33:

KRGRKKLLYIFKOPFMRPVQTTQEEDGCSCRFPEEEEGGCEL

In some embodiments, an anti-FAP CAR of the present disclosure comprises a 4-1BB costimulatory domain having the amino acid sequence as set forth in SEQ ID NO: 33, or a variant thereof. In some embodiments, an anti-FAP CAR comprises a 4-1BB co-stimulatory domain comprising an amino acid sequence at least 70%, at least 80%, at least 85%, at least 90%, at least 92%, at least 95%, at least 98%, at least 99%, or 100% identical to SEQ ID NO: 33.

Exemplary amino acid sequence and coding sequence for intracellular domains (e.g., ICD2 and ICD2) are set forth in Table 6

TABLE 7
Intracellular domains for anti-FAP CAR

| Molecule | Amino Acid Sequence | Coding Sequence |
|---|---|---|
| CD3z | RVKFSRSADAPAYQQGQNQLYNELN | AGGGTGAAATTTTCTAGAAGCGCCGATG |
| | LGRREEYDVLDKRRGRDPEMGGKPR | CTCCCGCATATCAGCAGGGTCAGAATCA |
| | RKNPQEGLYNELQKDKMAEAYSEIG | GCTCTACAATGAATTGAATCTCGGCAGG |
| | MKGERRRGKGHDGLYQGLSTATKDT | CGAGAAGAGTACGATGTTCTGGACAAGA |
| | YDALHMQALPPR | GACGGGGCAGGGATCCCGAGATGGGGGG |
| | (SEQ ID NO: 32) | AAAGCCCCGGAGAAAAAATCCTCAGGAG |
| | | GGGTTGTACAATGAGCTGCAGAAGGACA |
| | | AGATGGCTGAAGCCTATAGCGAGATCGG |
| | | AATGAAAGGCGAAAGACGCAGAGGCAAG |
| | | GGGCATGACGGTCTGTACCAGGGTCTCT |
| | | CTACAGCCACCAAGGACACTTATGATGC |
| | | GTTGCATATGCAAGCCTTGCCACCCCGC |
| | | (SEQ ID NO: 89) |
| CD3z(YXXQ) | RVKFSRSADAPAYQQGONQLYNELN | AGAGTTAAATTTTCTAGAAGCGCGGATG |
| | LGRREEYDVLDKRRGRDPEMGGKPR | CGCCCGCATATCAACAAGGACAAAATCA |
| | RKNPQEGLYNELQKDKMAEAYSEIG | GCTCTACAACGAACTGAACCTCGGACGC |
| | MKGERRRGKGHDGLYQGLSTATKDT | AGGGAAGAATATGACGTATTGGACAAAA |
| | YDAYRHQALPPR | GGAGGGGAAGAGATCCTGAGATGGGAGG |
| | (SEQ ID NO: 90) | CAAGCCTAGACGGAAAAACCCGCAAGAA |
| | | GGCCTCTATAACGAACTCCAGAAAGACA |
| | | AGATGGCGGAAGCATATTCAGAAATAGG |
| | | CATGAAAGGAGAGCGCAGAAGGGGAAAG |
| | | GGACATGACGGCCTGTATCAGGGCCTTT |
| | | CCACGGCAACAAAGGATACTTACGACGC |
| | | TTATAGGCATCAGGCTCTACCTCCGCGA |
| | | (SEQ ID NO: 91) |
| CD27 | ILVIFSGMFLVFTLAGALFLHQRRK | ATCCTCGTTATATTCTCAGGAATGTTTC |
| | YRSNKGESPVEPAEPCHYSCPREEE | TGGTGTTCACCCTCGCGGGGGCGCTTTT |
| | GSTIPIQEDYRKPEPACSP | TCTCCATCAGCGGAGGAAATATAGGTCC |
| | (SEQ ID NO: 92) | AACAAGGGGGAGTCCCCTGTGGAACCGG |
| | | CTGAGCCGTGCCACTACTCCTGTCCGCG |
| | | CGAAGAGGAAGGGTCTACGATTCCGATC |
| | | CAAGAGGATTATAGGAAGCCCGAGCCTG |
| | | CGTGCAGTCCT |
| | | (SEQ ID NO: 93) |
| CD28/IL15RA | RSKRSRLLHSDYMNMTPRRPGPTRK | AGGTCCAAAAGAAGTCGGCTGCTTCATA |
| | HYQPYAPPRDFAAYRSKSRQTPPLA | GTGACTACATGAATATGACTCCACGCCG |
| | SVEMEAMEALPVTWGTSSRDEDLEN | CCCTGGGCCGACCAGAAAGCACTACCAA |
| | CSHHL | CCTTACGCTCCCCCGCGAGACTTCGCCG |
| | (SEQ ID NO: 94) | CTTACCGCTCTAAGTCAAGGCAAACTCC |
| | | CCCGCTGGCCAGCGTTGAAATGGAAGCG |
| | | ATGGAGGCTCTGCCGGTGACTTGGGGGA |
| | | CCAGCAGCAGAGATGAGGACTTGGAAAA |
| | | CTGCTCTCACCACCTA |
| | | (SEQ ID NO: 95) |
| CD40 | KKVAKKPTNKAPHP KQEPQEINFPD | AAAAAGGTGGCCAAGAAGCCAACCAATA |
| | DLPGSNTAAPVQETLHGCQPVTQED | AGGCCCCCCACCCCAAGCAGGAACCCCA |
| | GKESRISVQERQ | GGAGATCAATTTTCCCGACGATCTTCCT |
| | (SEQ ID NO: 96) | GGCTCCAACACTGCTGCTCCAGTGCAGG |
| | | AGACTTTACATGGATGCCAACCGGTCAC |
| | | CCAGGAGGATGGCAAAGAGAGTCGCATC |
| | | TCAGTGCAGGAGAGACAG |
| | | (SEQ ID NO: 97) |
| DAP10 | LCARPRRSPAQEDGKVYINMPGRG | TTGTGTGCTCGCCCAAGGCGCAGTCCGG |
| | (SEQ ID NO: 98) | CCCAAGAAGATGGAAAAGTCTATATTAA |
| | | CATGCCTGGGAGAGGT |
| | | (SEQ ID NO: 99) |
| DAP12 | YFLGRLVPRGRGAAEAATRKQRITE | TACTTCTTGGGCCGACTGGTACCCAGGG |
| | TESPYQELQGQRSDVYSDLNTQRPY | GACGGGGGGCCGCAGAAGCGGCTACACG |
| | YK | AAAACAGAGAATCACGGAAACAGAGTCA |
| | (SEQ ID NO: 100) | CCTTATCAGGAATTGCAGGGCCAGAGGA |
| | | GCGATGTTTATAGTGACCTTAATACACA |
| | | ACGACCGTACTACAAA |
| | | (SEQ ID NO: 101) |
| DNAM1 | NRRRRRERRDLFTESWDTQKAPNNY | AATAGGAGAAGGCGACGCGAAAGGCGAG |
| | RSPISTSQPTNQSMDDTREDIYVNY | ATTTGTTCACGGAGTCATGGGATACTCA |
| | PTFSRRPKTRV | GAAAGCGCCGAACAATTACCGAAGCCCT |
| | (SEQ ID NO: 102) | ATTTCAACGAGTCAGCCAACAAATCAGT |
| | | CTATGGATGATACAAGGGAAGATATATA |
| | | TGTAAACTACCCAACATTCAGCCGGCGC |
| | | CCCAAAACGCGAGTT |
| | | (SEQ ID NO: 103) |
| 41BB | KRGRKKLLYIFKQPFMRPVQTTQEE | AAGCGCGGGAGAAAGAAGCTCCTGTACA |
| | DGCSCRFPEEEEGGCEL | TCTTCAAGCAGCCTTTTATGCGACCTGT |
| | (SEQ ID NO: 33) | GCAAACCACTCAGGAAGAAGATGGGTGT |
| | | TCATGCCGCTTCCCCGAGGAGGAAGAAG |
| | | GAGGGTGTGAACTG |
| | | (SEQ ID NO: 104) |
| OX40 | ALYLLRRDQRLPPDAHKPPGGGSFR | GCCCTGTACCTGCTCCGGAGGGACCAGA |
| | TPIQEEQADAHSTLAKI | GGCTGCCCCCCGATGCCCACAAGCCCCC |
| | (SEQ ID NO: 105) | TGGGGGAGGCAGTTTCCGGACCCCCATC |
| | | CAAGAGGAGCAGGCCGACGCCCACTCCA |
| | | CCCTGGCCAAGATC |
| | | (SEQ ID NO: 106) |
| SLAMF6 | LRKRRDSLSLSTQRTQGPAESARNL | TTGAGGAAAAGAAGAGATTCCCTATCTT |
| | EYVSVSPTNNTVYASVTHSNRETEI | TGTCTACTCAGCGAACACAGGGCCCCGC |
| | WTPRENDTITIYSTINHSKESKPTF | AGAGTCCGCAAGGAACCTAGAGTATGTT |
| | SRATALDNVV | TCAGTGTCTCCAACGAACAACACTGTGT |
| | (SEQ ID NO: 39) | ATGCTTCAGTCACTCATTCAAACAGGGA |
| | | AACAGAAATCTGGACACCTAGAGAAAAT |
| | | GATACTATCACAATTTACTCCACAATTA |
| | | ATCATTCCAAAGAGAGTAAACCCACTTT |
| | | TTCCAGGGCAACTGCCCTTGACAATGTC |
| | | GTG |
| | | (SEQ ID NO: 107) |

In some embodiments, an anti-FAP CAR of the present disclosure comprises a extracellular domain selected from any one of the anti-FAP scFv in Table 2, a transmembrane domain selected from any one of the hinge/TM domains listed in Table 6, a ICD1 selected from any one of the ICD in Table 7, and a ICD2 selected from any one of the ICD in Table 7. Exemplary configuration of an anti-FAP CAR is provided in Table 8.

TABLE 8
Exemplary Configuration of an anti-FAP CAR

| scFvs | Hinge TM | ICD1 | ICD2 |
|---|---|---|---|
| D05 | CD8 Hinge/TM | DAP10 | CD3z(YXXQ) |
| D05 | CD8 Hinge/TM | DAP10 | CD3z |
| D05 | CD8 Hinge/TM | DAP10 | DAP12 |
| D05 | CD8 Hinge/TM | 41BB | CD3z |
| D05 | CD8 Hinge/TM | DAP12 | DAP10 |
| D01 | CD8 Hinge/TM | 41BB | CD3z |
| D01 | IgG1 Hinge/SLAMF4 TM | CD27 | CD3z(YXXQ) |
| D01 | IgG1 Hinge/SLAMF4 TM | OX40 | CD3z |
| D01 | IgG1 Hinge/CD2 TM | CD28/IL15RA | CD3z(YXXQ) |
| D01 | IgG1 Hinge/SLAMF4 TM | CD40 | CD3z |
| B08 | CD8 Hinge/TM | 41BB | CD3z |
| B08 | IgG1 Hinge/CD2 TM | CD40 | DAP12 |
| B08 | CD8 Hinge/TM | SLAMF6 | DAP12 |
| D05 | CD8 Hinge/TM | CD28/IL15RA | CD3z |
| D05 | CD8 Hinge/TM | CD40 | CD3z |
| D05 | CD8 Hinge/TM | OX40 | CD3z |
| D05 | IgG1 Hinge/SLAMF4 TM | CD27 | CD3z |
| D05 | IgG1 Hinge/SLAMF4 TM | CD40 | CD3z |
| D05 | IgG1 Hinge/SLAMF4 TM | OX40 | CD3z |

The intracellular signaling domain of a chimeric antigen receptor of the present disclosure is responsible for activation of at least one of the normal effector functions of the cell in which the CAR has been placed and/or activation of proliferative and cell survival pathways.

It is to be understood that a chimeric antigen receptor as disclosed herein can include a domain (e.g., an extracellular domain, a transmembrane domain, an intracellular (cytoplasmic) domain, a co-stimulatory domain, a signaling domain, or any combination thereof) having a sequence as set forth herein, or a variant thereof, or a fragment thereof, of any one or more of the domains disclosed herein (e.g., a variant and/or fragment that retains the function required for the chimeric antigen receptor activity).

In some embodiments, exemplary anti-FAP CAR amino acid sequences are set forth in Table 4.

TABLE 4
anti-FAP CAR amino acid sequences

| Anti-FAP CAR | Sequences | SEQ ID NO |
|---|---|---|
| CAR comprising | MALPVTALLLPLALLLHAARPQVQLVQSGAEVKKPGSS | 17 |
| A07 scFv | VKVSCKASGGTFSSYAISWVRQAPGQGLEWMGGIIPIF | |
| | GTANYAQKFQGRVTITADESTSTAYMELSSLRSEDTAV | |
| | YYCASPRDCSSTSCYAGIDAFDIWGQGTMVTVSSGGGG | |
| | SGGGGSGGGASEIVLTQSPGTLSLSPGERATLSCRASQ | |
| | SVSSSYLAWYQQKPGQAPRLLIYGASSRATGIPDRFSG | |
| | SGSGTDFTLTISRLEPEDFAVYYCQQYGNSPTTFGGGT | |
| | KLEIKRTAAATTTPAPRPPTPAPTIASQPLSLRPEACR | |
| | PAAGGAVHTRGLDFACDIYIWAPLAGTCGVLLLSLVIT | |
| | LYCKRGRKKLLYIFKQPFMRPVQTTQEEDGCSCRFPEE | |
| | EEGGCELRVKFSRSADAPAYQQGQNQLYNELNLGRREE | |
| | YDVLDKRRGRDPEMGGKPRRKNPQEGLYNELQKDKMAE | |
| | AYSEIGMKGERRRGKGHDGLYQGLSTATKDTYDALHMQ | |
| | ALPPR | |
| CAR comprising | MALPVTALLLPLALLLHAARPEVQLLESGGGLVQPGGS | 18 |
| D05 scFv | LRLSCAASGFTFSSYAMSWVRQAPGKGLEWVSAISGSG | |
| | GSTYYADSVKGRFTISRDNSKNTLYLQMNSLRAEDTAV | |
| | YYCAKTHSTSWYYFDYWGRGTLVTVSSGGGGSGGGGSG | |
| | GGASEIVLTQSPGTLSLSPGERATLSCRASQSVSSSYL | |
| | AWYQQKPGQAPRLLIYGASSRATGIPDRFSGSGSGTDF | |
| | TLTISRLEPEDFAVYYCQQYGSSPRYTFGQGTKVDIKR | |
| | TAAATTTPAPRPPTPAPTIASQPLSLRPEACRPAAGGA | |
| | VHTRGLDFACDIYIWAPLAGTCGVLLLSLVITLYCKRG | |
| | RKKLLYIFKQPFMRPVQTTQEEDGCSCRFPEEEEGGCE | |
| | LRVKFSRSADAPAYQQGQNQLYNELNLGRREEYDVLDK | |
| | RRGRDPEMGGKPRRKNPQEGLYNELQKDKMAEAYSEIG | |
| | MKGERRRGKGHDGLYQGLSTATKDTYDALHMQALPPR | |
| CAR comprising | MALPVTALLLPLALLLHAARPEVQLLESGGGLVQPGGS | 59 |
| E07 scFv | LRLSCAASGFTFKSYVMSWVRQAPGKGLEWVSAIVPSG | |
| | GDTYYADSVKGRFTISRDNSKNTLYLQMNSLRAEDTAV | |
| | YYCAKDYYGGWFDPWGQGTLVTVSSGGGGSGGGGSGGG | |
| | ASEIVLTQSPGTLSLSPGERATLSCRASQSVSSSYLAW | |
| | YQQKPGQAPRLLIYGASSRATGIPDRFSGSGSGTDFTL | |
| | TISRLEPEDFAVYYCQQYGSSPRTFGQGTKVEIKAAAT | |
| | TTPAPRPPTPAPTIASQPLSLRPEACRPAAGGAVHTRG | |
| | LDFACDIYIWAPLAGTCGVLLLSLVITLYCKRGRKKLL | |
| | YIFKQPFMRPVQTTQEEDGCSCRFPEEEEGGCELRVKF | |
| | SRSADAPAYQQGQNQLYNELNLGRREEYDVLDKRRGRD | |
| | PEMGGKPRRKNPQEGLYNELQKDKMAEAYSEIGMKGER | |
| | RRGKGHDGLYQGLSTATKDTYDALHMQALPPR | |
| CAR comprising | MALPVTALLLPLALLLHAARPEVQLLESGGGLVQPGGS | 60 |
| D01 scFv | LRLSCAASGFTFSSYAMSWVRQAPGKGLEWVSAISGSG | |
| | GSTYYADSVKGRFTISRDNSKNTLYLQMNSLRAEDTAV | |
| | YYCAKDYYGGWFDPWGQGTLVTVSSGGGGSGGGGSGGG | |
| | ASEIVLTQSPGTLSLSPGERATLSCRASQSVSSSYLAW | |
| | YQQKPGQAPRLLIYGASSRATGIPDRFSGSGSGTDFTL | |
| | TISRLEPEDFAVYYCQQYGSSPRTFGHGTKLEIKAAAT | |
| | TTPAPRPPTPAPTIASQPLSLRPEACRPAAGGAVHTRG | |
| | LDFACDIYIWAPLAGTCGVLLLSLVITLYCKRGRKKLL | |
| | YIFKQPFMRPVQTTQEEDGCSCRFPEEEEGGCELRVKF | |
| | SRSADAPAYQQGQNQLYNELNLGRREEYDVLDKRRGRD | |
| | PEMGGKPRRKNPQEGLYNELQKDKMAEAYSEIGMKGER | |
| | RRGKGHDGLYQGLSTATKDTYDALHMQALPPR | |
| CAR comprising | MALPVTALLLPLALLLHAARPEVQLLESGGGLVQPGGS | 61 |
| B08 scFv | LRLSCAASGFTFSSYAMSWVRQAPGKGLEWVSGISEGG | |
| | GSTYYADSVKGRFTISRDNSKNTLYLQMNSLRAEDTAV | |
| | YYCAKDYYGGWFDPWGRGTLVTVSSGGGGSGGGGSGGG | |
| | ASEIVLTQSPGTLSLSPGERATLSCRASQSVSSSYLAW | |
| | YQQKPGQAPRLLIYGASSRATGIPDRFSGSGSGTDFTL | |
| | TISRLEPEDFAVYYCQQYGSSPRTFGQGTRLEIKAAAT | |
| | TTPAPRPPTPAPTIASQPLSLRPEACRPAAGGAVHTRG | |
| | LDFACDIYIWAPLAGTCGVLLLSLVITLYCKRGRKKLL | |
| | YIFKQPFMRPVQTTQEEDGCSCRFPEEEEGGCELRVKF | |
| | SRSADAPAYQQGQNQLYNELNLGRREEYDVLDKRRGRD | |
| | PEMGGKPRRKNPQEGLYNELQKDKMAEAYSEIGMKGER | |
| | RRGKGHDGLYQGLSTATKDTYDALHMQALPPR | |
| CAR comprising | MALPVTALLLPLALLLHAARPEVQLLESGGGLVQPGGS | 62 |
| H02 scFv | LRLSCAASGFTFSSYAMSWVRQAPGKGLEWVSAISGSG | |
| | GSTYYADSVKGRFTISRDNSKNTLYLQMNSLRAEDTAV | |
| | YYCAKDLSWYGANFDYWGQGTLVTVSSGGGGSGGGGSG | |
| | GGASEIVLTQSPGTLSLSPGERATLSCRASQSVSSSYL | |
| | AWYQQKPGQAPRLLIYGASSRATGIPDRFSGSGSGTDF | |
| | TLTISRLEPEDFAVYYCQQYGSSPVMYTFGQGTKVEIK | |
| | AAATTTPAPRPPTPAPTIASQPLSLRPEACRPAAGGAV | |
| | HTRGLDFACDIYIWAPLAGTCGVLLLSLVITLYCKRGR | |
| | KKLLYIFKQPFMRPVQTTQEEDGCSCRFPEEEEGGCEL | |
| | RVKFSRSADAPAYQQGQNQLYNELNLGRREEYDVLDKR | |
| | RGRDPEMGGKPRRKNPQEGLYNELQKDKMAEAYSEIGM | |
| | KGERRRGKGHDGLYQGLSTATKDTYDALHMQALPPR | |

In some embodiments, an anti-FAP CAR comprises an amino acid sequence containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the anti-FAP CAR amino acid sequence as set forth in SEQ ID NO: 17. In some embodiments, an anti-FAP CAR comprises an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the anti-FAP CAR amino acid sequence as set forth in SEQ ID NO: 17. In some embodiments, an anti-FAP CAR comprises an amino acid sequence of SEQ ID NO: 17.

In some embodiments, an anti-FAP CAR comprises an amino acid sequence containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the anti-FAP CAR amino acid sequence as set forth in SEQ ID NO: 18. In some embodiments, an anti-FAP CAR comprises an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the anti-FAP CAR amino acid sequence as set forth in SEQ ID NO: 18. In some embodiments, an anti-FAP CAR comprises an amino acid sequence of SEQ ID NO: 18.

In some embodiments, an anti-FAP CAR comprises an amino acid sequence containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the anti-FAP CAR amino acid sequence as set forth in SEQ ID NO: 59. In some embodiments, an anti-FAP CAR comprises an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the anti-FAP CAR amino acid sequence as set forth in SEQ ID NO: 59. In some embodiments, an anti-FAP CAR comprises an amino acid sequence of SEQ ID NO: 59.

In some embodiments, an anti-FAP CAR comprises an amino acid sequence containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the anti-FAP CAR amino acid sequence as set forth in SEQ ID NO: 60. In some embodiments, an anti-FAP CAR comprises an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the anti-FAP CAR amino acid sequence as set forth in SEQ ID NO: 60. In some embodiments, an anti-FAP CAR comprises an amino acid sequence of SEQ ID NO: 60.

In some embodiments, an anti-FAP CAR comprises an amino acid sequence containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the anti-FAP CAR amino acid sequence as set forth in SEQ ID NO: 61. In some embodiments, an anti-FAP CAR comprises an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the anti-FAP CAR amino acid sequence as set forth in SEQ ID NO: 61. In some embodiments, an anti-FAP CAR comprises an amino acid sequence of SEQ ID NO: 61.

In some embodiments, an anti-FAP CAR comprises an amino acid sequence containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the anti-FAP CAR amino acid sequence as set forth in SEQ ID NO: 62. In some embodiments, an anti-FAP CAR comprises an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the anti-FAP CAR amino acid sequence as set forth in SEQ ID NO: 62. In some embodiments, an anti-FAP CAR comprises an amino acid sequence of SEQ ID NO: 62.

IV. iNKT Cells Expressing the Anti-FAP Chimeric Antigen Receptor and Methods of Producing the Same

Also provided by the present disclosure are genetically modified cells that comprise at least one of the FAP binding moieties described herein (e.g., an anti-FAP antibodies or antigen binding fragments thereof, or at least one of the anti-FAP CARs as disclosed herein).

In some embodiments, a cell can be genetically modified to express any one of the chimeric antigen receptors described herein. In some embodiments, the genetically modified cells described herein are eukaryotic cells. In some embodiments, the genetically modified cells comprising the CARs (e.g., anti-FAP CAR) are human cells. In some embodiments, the genetically modified cells comprising the CARs (e.g., anti-FAP CAR) are immune cells (e.g., T cells such as cytotoxic T lymphocytes, regulatory T cells, NK cells, NKT cells such as invariant NKT cells, macrophages, monocytes, neutrophils, eosinophils, or any combination thereof).

In some embodiments, the genetically modified immune cells are iNKT cells modified to express one or more of the anti-FAP CARs as described herein. iNKT cells infiltrate tumors and play an important role in the immune surveillance against tumors (e.g., solid and/or hematological malignancies) (see, e.g., Wolf et al., Novel Approaches to Exploiting Invariant NKT Cells in Cancer Immunotherapy, Front Immunol. 2018 Mar. 2; 9:384). In some instances, iNKT cells can be attractive platforms for adoptive cells immunotherapy of cancer compared to conventional T cells. In some embodiments, iNKT cells can directly kill cancer cells (e.g., CD1d-expressing cancer cells). In some embodiments, iNKT cells can restrict immunosuppressive myelomonocytic populations in the tumor microenvironment (TME) (e.g., via CD1d-cognate recognition), promoting anti-tumor responses (e.g., irrespective of the CD1d expression by cancer cells). In some embodiments, iNKT cells can be adoptively transferred across MHC barriers without risk of alloreaction because CD1d molecules are identical in all individuals, in addition to their ability to suppress graft vs. host disease (GvHD) without impairing the anti-tumor responses. In other embodiments, iNKT cells can be engineered to acquire a second antigen-specificity by expressing recombinant TCRs and/or Chimeric Antigen Receptor (CAR) specific for tumor-associated antigens, enabling the direct targeting of antigen-expressing cancer cells, while maintaining their CD1d-dependent functions. In some embodiments, iNKT cells can be used for donor unrestricted, and off the shelf, adoptive cell therapies enabling the concurrent targeting of cancer cells and suppressive microenvironment. Compared to conventional αβ T cells, iNKT cell adoptive immunotherapy include but are not limited to following advantages: (i) Control the tumor microenvironment (TME); (ii) Can be redirected against cancer cells by engineering with a tumor-specific CAR and/or TCR while maintaining their intrinsic control of the TME; (iii) Are devoid of alloreactivity, being restricted for the monomorphic CD1d molecule, allowing their possible use off the shelf in a donor-unrestricted manner.

In some embodiments, the present disclosure provides a population of genetically modified immune cells (e.g., iNKT cells) comprising an anti-FAP CAR described herein. In some embodiments, the present disclosure provides a population of genetically modified immune cells (e.g., iNKT cells) comprising an anti-FAP CAR which comprises a CDRH1, a CDRH2 and a CDRH3 of a heavy chain variable domain (VH) set forth in Table 2, and a light chain variable domain (VL) set forth in Table 2. In some embodiments, the present disclosure provides a population of genetically modified immune cells (e.g., iNKT cells) comprising an anti-FAP CAR which comprises a CDRH1, a CDRH2 and a CDRH3 of a heavy chain variable domain (VH) having the amino acid sequence of any one of SEQ ID NOs: 7, and/or a CDRL1, a CDRL2 and a CDRL3 of a light chain variable domain (VL) having the amino acid sequence of any one of SEQ ID NOs: 8. In some embodiments, the present disclosure provides a population of genetically modified immune cells (e.g., iNKT cells) comprising an anti-FAP CAR which comprises a CDRH1, a CDRH2 and a CDRH3 of a heavy chain variable domain (VH) having the amino acid sequence of any one of SEQ ID NO: 14, and/or a CDRL1, a CDRL2 and a CDRL3 of a light chain variable domain (VL) having the amino acid sequence of any one of SEQ ID NO: 15. In some embodiments, the present disclosure provides a population of genetically modified immune cells (e.g., iNKT cells) comprising an anti-FAP CAR which comprises a CDRH1, a CDRH2 and a CDRH3 of a heavy chain variable domain (VH) having the amino acid sequence of any one of SEQ ID NO: 44, and/or a CDRL1, a CDRL2 and a CDRL3 of a light chain variable domain (VL) having the amino acid sequence of any one of SEQ ID NO: 45. In some embodiments, the present disclosure provides a population of genetically modified immune cells (e.g., iNKT cells) comprising an anti-FAP CAR which comprises a CDRH1, a CDRH2 and a CDRH3 of a heavy chain variable domain (VH) having the amino acid sequence of any one of SEQ ID NO: 47, and/or a CDRL1, a CDRL2 and a CDRL3 of a light chain variable domain (VL) having the amino acid sequence of any one of SEQ ID NO: 48. In some embodiments, the present disclosure provides a population of genetically modified immune cells (e.g., iNKT cells) comprising an anti-FAP CAR which comprises a CDRH1, a CDRH2 and a CDRH3 of a heavy chain variable domain (VH) having the amino acid sequence of any one of SEQ ID NO: 51, and/or a CDRL1, a CDRL2 and a CDRL3 of a light chain variable domain (VL) having the amino acid sequence of any one of SEQ ID NO: 52. In some embodiments, the present disclosure provides a population of genetically modified immune cells (e.g., iNKT cells) comprising an anti-FAP CAR which comprises a CDRH1, a CDRH2 and a CDRH3 of a heavy chain variable domain (VH) having the amino acid sequence of any one of SEQ ID NO: 56, and/or a CDRL1, a CDRL2 and a CDRL3 of a light chain variable domain (VL) having the amino acid sequence of any one of SEQ ID NO: 57.

In some embodiments, the present disclosure provides a population of genetically modified immune cells (e.g., iNKT cells) comprising an anti-FAP CAR comprises a CDRH1 having the amino acid sequence of SEQ ID NO: 1, a CDRH2 having the amino acid sequence of SEQ ID NO: 2, a CDRH3 having the amino acid sequence of SEQ ID NO: 3, a CDRL1 having the amino acid sequence of SEQ ID NO: 4, a CDRL2 having the amino acid sequence of SEQ ID NO: 5, and a CDRL3 having the amino acid sequence of SEQ ID NO: 6. In some embodiments, the present disclosure provides a population of genetically modified immune cells (e.g., iNKT cells) comprising an anti-FAP CAR comprises a CDRH1 having the amino acid sequence of SEQ ID NO: 10, a CDRH2 having the amino acid sequence of SEQ ID NO: 11, a CDRH3 having the amino acid sequence of SEQ ID NO: 12, a CDRL1 having the amino acid sequence of SEQ ID NO: 4, a CDRL2 having the amino acid sequence of SEQ ID NO: 5, and a CDRL3 having the amino acid sequence of SEQ ID NO: 13. In some embodiments, the present disclosure provides a population of genetically modified immune cells (e.g., iNKT cells) comprising an anti-FAP CAR comprises a CDRH1 having the amino acid sequence of SEQ ID NO: 40, a CDRH2 having the amino acid sequence of SEQ ID NO: 41, a CDRH3 having the amino acid sequence of SEQ ID NO: 42, a CDRL1 having the amino acid sequence of SEQ ID NO: 4, a CDRL2 having the amino acid sequence of SEQ ID NO: 5, and a CDRL3 having the amino acid sequence of SEQ ID NO: 43. In some embodiments, the present disclosure provides a population of genetically modified immune cells (e.g., iNKT cells) comprising an anti-FAP CAR comprises a CDRH1 having the amino acid sequence of SEQ ID NO: 10, a CDRH2 having the amino acid sequence of SEQ ID NO: 11, a CDRH3 having the amino acid sequence of SEQ ID NO: 42, a CDRL1 having the amino acid sequence of SEQ ID NO: 4, a CDRL2 having the amino acid sequence of SEQ ID NO: 5, and a CDRL3 having the amino acid sequence of SEQ ID NO: 43. In some embodiments, the present disclosure provides a population of genetically modified immune cells (e.g., iNKT cells) comprising an anti-FAP CAR comprises a CDRH1 having the amino acid sequence of SEQ ID NO: 10, a CDRH2 having the amino acid sequence of SEQ ID NO: 50, a CDRH3 having the amino acid sequence of SEQ ID NO: 42, a CDRL1 having the amino acid sequence of SEQ ID NO: 4, a CDRL2 having the amino acid sequence of SEQ ID NO: 5, and a CDRL3 having the amino acid sequence of SEQ ID NO: 43. In some embodiments, the present disclosure provides a population of genetically modified immune cells (e.g., iNKT cells) comprising an anti-FAP CAR comprises a CDRH1 having the amino acid sequence of SEQ ID NO: 10, a CDRH2 having the amino acid sequence of SEQ ID NO: 11, a CDRH3 having the amino acid sequence of SEQ ID NO: 54, a CDRL1 having the amino acid sequence of SEQ ID NO: 4, a CDRL2 having the amino acid sequence of SEQ ID NO: 5, and a CDRL3 having the amino acid sequence of SEQ ID NO: 55.

In some embodiments, the present disclosure provides a population of genetically modified immune cells (e.g., iNKT cells) comprising an anti-FAP CAR comprises a VH comprising the amino acid sequence of SEQ ID NO: 7, and/or a VL comprising the amino acid sequence of SEQ ID NO: 8. In some embodiments, the present disclosure provides a population of genetically modified immune cells (e.g., iNKT cells) comprising an anti-FAP CAR comprises a VH comprising the amino acid sequence of SEQ ID NO: 14, and/or a VL comprising the amino acid sequence of SEQ ID NO: 15. In some embodiments, the present disclosure provides a population of genetically modified immune cells (e.g., iNKT cells) comprising an anti-FAP CAR comprises a VH comprising the amino acid sequence of SEQ ID NO: 44, and/or a VL comprising the amino acid sequence of SEQ ID NO: 45. In some embodiments, the present disclosure provides a population of genetically modified immune cells (e.g., iNKT cells) comprising an anti-FAP CAR comprises a VH comprising the amino acid sequence of SEQ ID NO: 47, and/or a VL comprising the amino acid sequence of SEQ ID NO: 48. In some embodiments, the present disclosure provides a population of genetically modified immune cells (e.g., iNKT cells) comprising an anti-FAP CAR comprises a VH comprising the amino acid sequence of SEQ ID NO: 51, and/or a VL comprising the amino acid sequence of SEQ ID NO: 52. In some embodiments, the present disclosure provides a population of genetically modified immune cells (e.g., iNKT cells) comprising an anti-FAP CAR comprises a VH comprising the amino acid sequence of SEQ ID NO: 56, and/or a VL comprising the amino acid sequence of SEQ ID NO: 57.

In some embodiments, the present disclosure provides a population of genetically modified immune cells (e.g., iNKT cells) comprising an anti-FAP CAR, which comprises a scFv as the extracellular ligand-binding domain comprising an amino acid sequence of any one of SEQ ID NOs: 9, 16, 46, 49, 53, or 58.

In some embodiments, the present disclosure provides a population of genetically modified immune cells (e.g., iNKT cells) comprising an anti-FAP CAR comprising an amino acid sequence of any one of SEQ ID NOs: 17,18, 59, 60, 61, or 62.

In some embodiments, the present disclosure provides a population of genetically modified immune cells (e.g., iNKT cells) comprising an anti-FAP CAR comprises a CDRH1, a CDRH2, a CDRH3 of the VH any of the known anti-FAP antibody in the art, for example, an anti-FAP antibody as provided in Table 3, and/or a CDRL1, a CDRL2, and a CDRL3 of the VL of any of the known anti-FAP antibody in the art, for example, an anti-FAP antibody as provided in Table 3.

In some embodiments, the present disclosure provides a population of genetically modified immune cells (e.g., iNKT cells) comprising an anti-FAP CAR comprises a VH, and/or a VL of any of the known anti-FAP antibody in the art, for example, an anti-FAP antibody as provided in Table 2.

In some embodiments, the present disclosure provides a population of genetically modified immune cells (e.g., iNKT cells) comprising an anti-FAP CAR, which comprises a scFv which comprises a VH, and/or a VL of any of the known anti-FAP antibody in the art, for example, an anti-FAP antibody as provided in Table 3 as the extracellular ligand-binding domain. In some embodiments, the present disclosure provides a population of genetically modified immune cells (e.g., iNKT cells) comprising an anti-FAP CAR, which comprises a scFv which comprises a VH, and/or a VL of any of the known anti-FAP antibody in the art, for example, an anti-FAP antibody as provided in Table 3 as the extracellular ligand-binding domain, and any of the transmembrane domain (e.g., CD8 transmembrane domain), intracellular domain (e.g., CD3 ζ intracellular domain), and the co-stimulatory domain (e.g., 4-1BB co-stimulatory domain) as described herein.

In some embodiments, the genetically modified cells (e.g., iNKT cells) are armored CAR expressing cells engineered to express another molecule (e.g., a cytokine or ligand) capable of enhancing one or more properties (e.g., survival/persistence, immune interaction with other cells such as macrophages and/or dendritic cells, disrupt immunosuppressive tumor microenvironment) of the genetically modified cells. The genetically modified cells (e.g., iNKT cells) can be engineered to express any suitable armoring molecule known in the art, e.g., armoring molecule described by Yeku et al., Armored CAR T-cells: utilizing cytokines and pro-inflammatory ligands to enhance CAR T-cell anti-tumour efficacy, Biochem Soc Trans. 2016 Apr. 15; 44 (2): 412-418; Hawkins et al., Armored CAR T-Cells: The Next Chapter in T-Cell Cancer Immunotherapy, Biologics. 2021; 15:95-105). Non-limiting examples of armoring molecules include IL-15, IL-2, IL-12, CD40L, 4-1BBL, IL-18, IL-7, IL-33, constitutively active Akt (caAkt), hybrid IL-4/IL-7 receptor, checkpoint inhibitors such as anti-PD1 antibodies, nanobodies targeting CD47, or bispecific T-cell engagers (BiTEs).

In some embodiments, the present disclosure provides an iNKT cell comprising a CAR that specifically binds FAP (e.g., any suitable FAP binding moiety), and the iNKT cell is engineered to express an armor molecule (i.e., immunoregulatory molecule). In some embodiments, the genetically modified cells (e.g., iNKT cells) expressing the CAR described herein (e.g., an anti-FAP CAR) are also engineered to express IL-15. In some embodiments, the genetically modified cells expressing the CAR described herein (e.g., an anti-FAP CAR) are also engineered to express soluble IL-15 (sIL-15). The role of IL-15 in enhancing the expansion and function of T cells and natural killer T cells (e.g., iNKT cells) have been previously described (see, e.g., Lin et al., Interleukin-15 enhances the expansion and function of natural killer T cells from adult peripheral and umbilical cord blood, Cytokine. 2015 December; 76 (2): 348-355; Battram et al., IL-15 Enhances the Persistence and Function of FAP-Targeting CAR-T Cells Compared to IL-2 or IL-15/IL-7 by Limiting CAR-T Cell Dysfunction and Differentiation; Cancers (Basel). 2021 July; 13 (14): 3534). There is evidence that numerous cell types are responsible for the production of IL-15, including macrophages and DCs, and once released, IL-15 stimulates CD8+ T-cells and NK cells which increases their proliferation and cytotoxic capacity. Administration of IL-15 to mice has been shown to enhance anti-tumor activity of adoptively transferred CD8+ tumor-reactive T-cells, which suggests IL-15 could also enhance anti-tumor activity of CAR T-cell therapy. Further, IL-15 can increase antigen-independent T-cell proliferation, while enabling T-cell persistence after tumor clearance. Certain previous studies used a form of IL-15 tethered to the membrane and found that this promoted the CAR T-cells to develop a memory phenotype. These data suggest that IL-15 could provide long-term CAR T-cell-mediated immunity toward the cancer antigen along with enhancing CAR T-cell function within the tumor microenvironment.

In some embodiments, the present disclosure, at least in part, is based on the discovery of using soluble IL-15 as an armor molecule for the genetically modified immune cells (e.g., iNKT cells) expressing an anti-FAP CAR. In some embodiments, an iNKT cell described herein express an anti-FAP CAR and soluble IL-15. In some embodiments, soluble IL-15 enhances the persistence of the genetically modified immune cells (e.g., iNKT cells) in the tumor microenvironment. In some embodiments, soluble IL-15 potentiates the cytotoxicity ability of CAR T cells, which can be used as a combination therapy with the anti-FAP CAR iNKT cells of the present disclosure.

In some embodiments, the nucleic acid encoding the CAR (e.g., anti-FAP CAR) is a multicistronic vector also encoding a soluble IL-15. An exemplary IL-15 coding sequence is set forth in SEQ ID NO: 34:

ATGCGGATCTCCAAGCCCCACCTCCGAAGCATCAGTATCCAGTGTTAT
CTGTGCCTTCTTCTGAATTCCCATTTTTTGACAGAGGCAGGAATTCAT
GTATTCATATTGGGTTGTTTCTCCGCGGGGCTTCCTAAAACCGAAGCT
AACTGGGTTAATGTCATTTCAGATCTGAAGAAAATTGAAGATCTTATT
CAATCAATGCATATCGACGCGACCTTGTACACGGAGAGTGATGTCCAC
CCGAGCTGCAAGGTAACGGCCATGAAATGCTTCTTGCTGGAATTGCAG
GTAATTAGTCTTGAGAGTGGAGATGCGTCTATACACGACACCGTAGAG
AATCTCATTATTTTGGCGAACAATTCTCTCAGCAGTAACGGCAATGTT
ACTGAATCAGGATGTAAGGAGTGTGAAGAGCTGGAAGAAAAGAACATT
AAGGAATTTTTGCAGAGCTTCGTACATATCGTCCAAATGTTCATAAAT
ACAAGC

In some embodiments, the nucleic acid encoding the CAR (e.g., anti-FAP CAR) further comprises a nucleic acid sequence at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleic acid sequence of SEQ ID NO: 34.

In some embodiments, a multicistronic vector comprises two or more expression cassettes encoding one or more anti-FAP CAR, and a sIL-15.

In some embodiments, multicistronic expression constructs are comprise expression cassettes that are positioned in different ways. For example, in some embodiments, a multicistronic expression construct is provided in which a first expression cassette (e.g., an expression cassette encoding an anti-FAP CAR, or portion thereof) is positioned adjacent to a second expression cassette (e.g., an expression cassette encoding sIL-15). In some embodiments, the first expression cassette (e.g., an expression cassette encoding an anti-FAP CAR, or portion thereof) is positioned 5′ to the second expression cassette (e.g., an expression cassette encoding sIL-15). In some embodiments, the first expression cassette (e.g., an expression cassette encoding an anti-FAP CAR, or portion thereof) is positioned 3′ to the second expression cassette (e.g., an expression cassette encoding sIL-15). Multicistronic vectors have been previously described, for example, by Shaimardanova et al., Production and Application of Multicistronic Constructs for Various Human Disease Therapies, Pharmaceutics 2019, 11, 580. In some embodiments, the multicistronic vector comprises a nucleic acid sequence encoding a self-cleaving 2A peptide between the first expression cassette (e.g., an expression cassette encoding an anti-FAP CAR, or portion thereof) and the second expression cassette (e.g., an expression cassette encoding sIL-15). In some embodiments, the self-cleaving 2A peptide is a P2A peptide. In some embodiments, the self-cleaving 2A peptide is a T2A peptide. In some embodiments, the multicistronic vector comprises a nucleic acid sequence encoding an internal ribosome entry site (IRES) between the first expression cassette (e.g., an expression cassette encoding an anti-FAP CAR, or portion thereof) and the second expression cassette (e.g., an expression cassette encoding sIL-15).

In some embodiments, the nucleic acid sequence encoding sIL-15 (e.g., sIL-15 coding sequence as set forth in SEQ ID NO: 34) are delivered to the cell in a separate construct from the construct encoding the CAR. In some embodiments, the cell already express soluble IL-15 (e.g., engineered to express sIL-15) prior to receiving the nucleic acid construct encoding the CAR (e.g., anti-FAP CAR).

Also provided herein are cells (e.g., iNKT cells) comprising a nucleic acid molecule encoding a chimeric antigen receptor as disclosed herein. In some embodiments, a nucleic acid molecule encoding a CAR (e.g., anti-FAP CAR) as described herein is present (i.e., integrated) within the genome of the genetically modified cell or, alternatively, is not integrated into the genome of the cell. In some embodiments, where the nucleic acid molecule encoding a CAR (e.g., anti-FAP CAR) is not integrated into the genome, the nucleic acid molecule is present in the genetically modified cell in a recombinant DNA construct, in an mRNA, in a viral genome, or in another nucleic acid which is not integrated into the genome of the cell.

In one embodiment, genetically modified cells (e.g., iNKT cells) contain a nucleic acid molecule encoding a chimeric antigen receptor (e.g., an anti-FAP CAR) as disclosed herein, positioned within the genome of a cell. In other embodiments, genetically modified cells (e.g., an iNKT cell) contain a nucleic acid molecule encoding a chimeric antigen receptor (e.g., an anti-FAP CAR) as disclosed herein, positioned within the endogenous T cell receptor alpha gene of the cell.

In some embodiments, the present disclosure encompasses an isolated nucleic acid comprising sequences of a CAR (e.g., an anti-FAP CAR), wherein the sequence comprises the nucleic acid sequence of an extracellular ligand-binding domain operably linked to the nucleic acid sequence of transmembrane domain and a cytoplasmic domain. The nucleic acid sequences coding for the desired molecules can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned. The present disclosure also provides vectors in which a DNA of the present disclosure is inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. In some embodiments, the desired CAR can be expressed in the cells by way of transposons.

In brief summary, the expression of natural or synthetic nucleic acids encoding CARs is typically achieved by operably linking a nucleic acid encoding the CAR polypeptide (e.g., an anti-FAP CAR) or portions thereof to a promoter, and incorporating the construct into an expression vector (e.g., a lentiviral vector). The vectors can be suitable for replication and integration into eukaryotes. Typical cloning vectors contain transcription and translation terminators, initiation sequences, and promoters useful for regulation of the expression of the desired nucleic acid sequence. The expression constructs of the present disclosure may also be used for nucleic acid immunization and gene therapy, using standard gene delivery protocols. Methods for gene delivery are known in the art. Sec, e.g., U.S. Pat. Nos. 5,399,346, 5,580,859, 5,589,466, incorporated by reference herein in their entireties.

The nucleic acid can be cloned into a number of types of vectors. For example, the nucleic acid can be cloned into a vector including, but not limited to a plasmid, a viral vector a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, lentiviruses provide a convenient platform for gene delivery systems. A selected gene can be inserted into a vector and packaged in lentiviral particles using techniques known in the art. The recombinant virus can then be isolated and delivered to cells of the subject either in vivo or ex vivo. A number of lentiviral systems are known in the art.

In some embodiments, the vector described herein further comprises a promoter operably linked to the nucleic acid encoding the CAR described herein (e.g., an anti-FAP CAR). Non-limiting examples of suitable promoters include cytomegalovirus (CMV) promoter, Elongation Factor-1α (EF-1α) promoter, simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. In some embodiments, the promoter is an EF-1α promoter. Further, the present disclosure also contemplates the use of inducible promoters. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In order to assess the expression of a CAR polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as kanamycin resistant gene and the like, and fluorescent genes such as GFP, YFP, RFP and the like. In some embodiments, reporter genes or selectable marker genes are excluded from a CAR polypeptide used in a therapy as described herein.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means. Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York). A preferred method for the introduction of a polynucleotide into a host cell is calcium phosphate transfection. Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Regardless of the method used to introduce exogenous nucleic acids into a host cell or otherwise expose a cell to the inhibitor of the present disclosure, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the present disclosure.

In some embodiments, the nucleic acid sequences encoding the anti-FAP antibodies or antigen binding fragments thereof, the FAP CAR, and the lentiviral vector sequences are set forth in Table 5.

TABLE 5
Nucleic acid sequences encoding the anti-FAP antibodies, the anti-FAP CAR, and
the lentiviral vector sequences.

| | | | SEQ |
|---|---|---|---|
| | | Coding Sequences | ID NO |
| A07 | VH | CAGGTGCAGCTGGTGCAGTCTGGGGCTGAGGTGAAGAAGCCTGGGTCCTCGGTG | 19 |
| | | AAGGTCTCCTGCAAGGCTTCTGGAGGCACCTTCAGCAGCTATGCTATCAGCTGG | |
| | | GTGCGACAGGCCCCTGGACAAGGGCTTGAGTGGATGGGAGGGATCATCCCTATC | |
| | | TTTGGTACAGCAAACTACGCACAGAAGTTCCAGGGCAGAGTCACGATTACCGCG | |
| | | GACGAATCCACGAGCACAGCCTACATGGAGCTGAGCAGCCTGAGATCTGAGGAC | |
| | | ACGGCCGTGTATTACTGTGCGAGCCCCCGCGATTGTAGTAGTACCAGCTGCTAT | |
| | | GCGGGGATTGATGCTTTTGATATCTGGGGCCAAGGGACAATGGTCACCGTCTCG | |
| | | AGT | |
| | VL | GAAATTGTGTTGACGCAGTCTCCAGGCACCCTGTCTTTGTCTCCAGGGGAAAGA | 20 |
| | | GCCACCCTCTCCTGCAGGGCCAGTCAGAGTGTTAGCAGCAGCTACTTAGCCTGG | |
| | | TACCAGCAGAAACCTGGCCAGGCTCCCAGGCTCCTCATCTATGGTGCATCCAGC | |
| | | AGGGCCACTGGCATCCCAGACAGGTTCAGTGGCAGTGGGTCTGGGACAGACTTC | |
| | | ACTCTCACCATCAGCAGACTGGAGCCTGAAGATTTTGCAGTGTATTATTGTCAG | |
| | | CAGTATGGTAACTCACCTACCACTTTCGGCGGAGGGACCAAGCTGGAGATCAAA | |
| | | CGGACC | |
| | scFv | CAGGTGCAGCTGGTGCAGTCTGGGGCTGAGGTGAAGAAGCCTGGGTCCTCGGTG | 21 |
| | | AAGGTCTCCTGCAAGGCTTCTGGAGGCACCTTCAGCAGCTATGCTATCAGCTGG | |
| | | GTGCGACAGGCCCCTGGACAAGGGCTTGAGTGGATGGGAGGGATCATCCCTATC | |
| | | TTTGGTACAGCAAACTACGCACAGAAGTTCCAGGGCAGAGTCACGATTACCGCG | |
| | | GACGAATCCACGAGCACAGCCTACATGGAGCTGAGCAGCCTGAGATCTGAGGAC | |
| | | ACGGCCGTGTATTACTGTGCGAGCCCCCGCGATTGTAGTAGTACCAGCTGCTAT | |
| | | GCGGGGATTGATGCTTTTGATATCTGGGGCCAAGGGACAATGGTCACCGTCTCG | |
| | | AGTGGTGGAGGCGGTTCAGGCGGAGGTGGCTCTGGCGGTGGCGCTAGCGAAATT | |
| | | GTGTTGACGCAGTCTCCAGGCACCCTGTCTTTGTCTCCAGGGGAAAGAGCCACC | |
| | | CTCTCCTGCAGGGCCAGTCAGAGTGTTAGCAGCAGCTACTTAGCCTGGTACCAG | |
| | | CAGAAACCTGGCCAGGCTCCCAGGCTCCTCATCTATGGTGCATCCAGCAGGGCC | |
| | | ACTGGCATCCCAGACAGGTTCAGTGGCAGTGGGTCTGGGACAGACTTCACTCTC | |
| | | ACCATCAGCAGACTGGAGCCTGAAGATTTTGCAGTGTATTATTGTCAGCAGTAT | |
| | | GGTAACTCACCTACCACTTTCGGCGGAGGGACCAAGCTGGAGATCAAACGGACC | |
| | CAR | CAGGTGCAGCTGGTGCAGTCTGGGGCTGAGGTGAAGAAGCCTGGGTCCTCGGTG | 22 |
| | | AAGGTCTCCTGCAAGGCTTCTGGAGGCACCTTCAGCAGCTATGCTATCAGCTGG | |
| | | GTGCGACAGGCCCCTGGACAAGGGCTTGAGTGGATGGGAGGGATCATCCCTATC | |
| | | TTTGGTACAGCAAACTACGCACAGAAGTTCCAGGGCAGAGTCACGATTACCGCG | |
| | | GACGAATCCACGAGCACAGCCTACATGGAGCTGAGCAGCCTGAGATCTGAGGAC | |
| | | ACGGCCGTGTATTACTGTGCGAGCCCCCGCGATTGTAGTAGTACCAGCTGCTAT | |
| | | GCGGGGATTGATGCTTTTGATATCTGGGGCCAAGGGACAATGGTCACCGTCTCG | |
| | | AGTGGTGGAGGCGGTTCAGGCGGAGGTGGCTCTGGCGGTGGCGCTAGCGAAATT | |
| | | GTGTTGACGCAGTCTCCAGGCACCCTGTCTTTGTCTCCAGGGGAAAGAGCCACC | |
| | | CTCTCCTGCAGGGCCAGTCAGAGTGTTAGCAGCAGCTACTTAGCCTGGTACCAG | |
| | | CAGAAACCTGGCCAGGCTCCCAGGCTCCTCATCTATGGTGCATCCAGCAGGGCC | |
| | | ACTGGCATCCCAGACAGGTTCAGTGGCAGTGGGTCTGGGACAGACTTCACTCTC | |
| | | ACCATCAGCAGACTGGAGCCTGAAGATTTTGCAGTGTATTATTGTCAGCAGTAT | |
| | | GGTAACTCACCTACCACTTTCGGCGGAGGGACCAAGCTGGAGATCAAACGGACC | |
| | | GCGGCCGCAACCACAACCCCTGCTCCAAGGCCCCCCACACCCGCTCCAACTATA | |
| | | GCCAGCCAACCATTGAGCCTCAGACCTGAAGCTTGCAGGCCCGCAGCAGGAGGC | |
| | | GCCGTCCATACGCGAGGCCTGGACTTCGCGTGTGATATTTATATTTGGGCCCCT | |
| | | TTGGCCGGAACATGTGGGGTGTTGCTTCTCTCCCTTGTGATCACTCTGTATTGT | |
| | | AAGCGCGGGAGAAAGAAGCTCCTGTACATCTTCAAGCAGCCTTTTATGCGACCT | |
| | | GTGCAAACCACTCAGGAAGAAGATGGGTGTTCATGCCGCTTCCCCGAGGAGGAA | |
| | | GAAGGAGGGTGTGAACTGAGGGTGAAATTTTCTAGAAGCGCCGATGCTCCCGCA | |
| | | TATCAGCAGGGTCAGAATCAGCTCTACAATGAATTGAATCTCGGCAGGCGAGAA | |
| | | GAGTACGATGTTCTGGACAAGAGACGGGGCAGGGATCCCGAGATGGGGGGAAAG | |
| | | CCCCGGAGAAAAAATCCTCAGGAGGGGTTGTACAATGAGCTGCAGAAGGACAAG | |
| | | ATGGCTGAAGCCTATAGCGAGATCGGAATGAAAGGCGAAAGACGCAGAGGCAAG | |
| | | GGGCATGACGGTCTGTACCAGGGTCTCTCTACAGCCACCAAGGACACTTATGAT | |
| | | GCGTTGCATATGCAAGCCTTGCCACCCCGC | |
| | Lentiviral | CGTTCAGCCCGACCGCTGCGCCTTATCCGGTAACTATCGTCTTGAGTCCAACCC | 23 |
| | vector | GGTAAGACACGACTTATCGCCACTGGCAGCAGCCACTGGTAACAGGATTAGCAG | |
| | | AGCGAGGTATGTAGGCGGTGCTACAGAGTTCTTGAAGTGGTGGCCTAACTACGG | |
| | | CTACACTAGAAGAACAGTATTTGGTATCTGCGCTCTGCTGAAGCCAGTTACCTT | |
| | | CGGAAAAAGAGTTGGTAGCTCTTGATCCGGCAAACAAACCACCGCTGGTAGCGG | |
| | | TGGTTTTTTTGTTTGCAAGCAGCAGATTACGCGCAGAAAAAAAGGATCTCAAGA | |
| | | AGATCCTTTGATCTTTTCTACGGGGTCTGACGCTCAGTGGAACGAAAACTCACG | |
| | | TTAAGGGATTTTGGTCATGAGATTATCAAAAAGGATCTTCACCTAGATCCTTTT | |
| | | AAATTAAAAATGAAGTTTTAAATCAATCTAAAGTATATATGAGTAAACTTGGTC | |
| | | TGACAGTTAGAAAAACTCATCGAGCATCAAATGAAACTGCAATTTATTCATATC | |
| | | AGGATTATCAATACCATATTTTTGAAAAAGCCGTTTCTGTAATGAAGGAGAAAA | |
| | | CTCACCGAGGCAGTTCCATAGGATGGCAAGATCCTGGTATCGGTCTGCGATTCC | |
| | | GACTCGTCCAACATCAATACAACCTATTAATTTCCCCTCGTCAAAAATAAGGTT | |
| | | ATCAAGTGAGAAATCACCATGAGTGACGACTGAATCCGGTGAGAATGGCAAAAG | |
| | | TTTATGCATTTCTTTCCAGACTTGTTCAACAGGCCAGCCATTACGCTCGTCATC | |
| | | AAAATCACTCGCATCAACCAAACCGTTATTCATTCGTGATTGCGCCTGAGCGAG | |
| | | ACGAAATACGCGATCGCTGTTAAAAGGACAATTACAAACAGGAATCGAATGCAA | |
| | | CCGGCGCAGGAACACTGCCAGCGCATCAACAATATTTTCACCTGAATCAGGATA | |
| | | TTCTTCTAATACCTGGAATGCTGTTTTTCCGGGGATCGCAGTGGTGAGTAACCA | |
| | | TGCATCATCAGGAGTACGGATAAAATGCTTGATGGTCGGAAGAGGCATAAATTC | |
| | | CGTCAGCCAGTTTAGTCTGACCATCTCATCTGTAACATCATTGGCAACGCTACC | |
| | | TTTGCCATGTTTCAGAAACAACTCTGGCGCATCGGGCTTCCCATACAAGCGATA | |
| | | GATTGTCGCACCTGATTGCCCGACATTATCGCGAGCCCATTTATACCCATATAA | |
| | | ATCAGCATCCATGTTGGAATTTAATCGCGGCCTCGACGTTTCCCGTTGAATATG | |
| | | GCTCATACTCTTCCTTTTTCAATATTATTGAAGCATTTATCAGGGTTATTGTCT | |
| | | CATGAGCGGATACATATTTGAATGTATTTAGAAAAATAAACAAATAGGGGTTCC | |
| | | GCGCACATTTCCCCGAAAAGTGCCACCTGACGGATCGGGAGATCTCCCGATCCC | |
| | | CTATGGTGCACTCTCAGTACAATCTGCTCTGATGCCGCATAGTTAAGCCAGTAT | |
| | | CTGCTCCCTGCTTGTGTGTTGGAGGTCGCTGAGTAGTGCGCGAGCAAAATTTAA | |
| | | GCTACAACAAGGCAAGGCTTGACCGACAATTGCATGAAGAATCTGCTTAGGGTT | |
| | | AGGCGTTTTGCGCTGCTTCGCGATGTACGGGCCAGATATACCCGTTGACATTGA | |
| | | TTATTGACTAGTTATTAATAGTAATCAATTACGGGGTCATTAGTTCATAGCCCA | |
| | | TATATGGAGTTCCGCGTTACATAACTTACGGTAAATGGCCCGCCTGGCTGACCG | |
| | | CCCAACGACCCCCGCCCATTGACGTCAATAATGACGTATGTTCCCATAGTAACG | |
| | | CCAATAGGGACTTTCCATTGACGTCAATGGGTGGAGTATTTACGGTAAACTGCC | |
| | | CACTTGGCAGTACATCAAGTGTATCATATGCCAAGTACGCCCCCTATTGACGTC | |
| | | AATGACGGTAAATGGCCCGCCTGGCATTATGCCCAGTACATGACCTTATGGGAC | |
| | | TTTCCTACTTGGCAGTACATCTACGTATTAGTCATCGCTATTACCATGGTGATG | |
| | | CGGTTTTGGCAGTACATCAATGGGCGTGGATAGCGGTTTGACTCACGGGGATTT | |
| | | CCAAGTCTCCACCCCATTGACGTCAATGGGAGTTTGTTTTGGCACCAAAATCAA | |
| | | CGGGACTTTCCAAAATGTCGTAACAACTCCGCCCCATTGACGCAAATGGGCGGT | |
| | | AGGCGTGTACGGTGGGAGGTCTATATAAGCAGCGCGTTTTGCCTGTACTGGGTC | |
| | | TCTCTGGTTAGACCAGATCTGAGCCTGGGAGCTCTCTGGCTAACTAGGGAACCC | |
| | | ACTGCTTAAGCCTCAATAAAGCTTGCCTTGAGTGCTTCAAGTAGTGTGTGCCCG | |
| | | TCTGTTGTGTGACTCTGGTAACTAGAGATCCCTCAGACCCTTTTAGTCAGTGTG | |
| | | GAAAATCTCTAGCAGTGGCGCCCGAACAGGGACTTGAAAGCGAAAGGGAAACCA | |
| | | GAGGAGCTCTCTCGACGCAGGACTCGGCTTGCTGAAGCGCGCACGGCAAGAGGC | |
| | | GAGGGGCGGCGACTGGTGAGTACGCCAAAAATTTTGACTAGCGGAGGCTAGAAG | |
| | | GAGAGAGATGGGTGCGAGAGCGTCAGTATTAAGCGGGGGAGAATTAGATCGCGA | |
| | | TGGGAAAAAATTCGGTTAAGGCCAGGGGGAAAGAAAAAATATAAATTAAAACAT | |
| | | ATAGTATGGGCAAGCAGGGAGCTAGAACGATTCGCAGTTAATCCTGGCCTGTTA | |
| | | GAAACATCAGAAGGCTGTAGACAAATACTGGGACAGCTACAACCATCCCTTCAG | |
| | | ACAGGATCAGAAGAACTTAGATCATTATATAATACAGTAGCAACCCTCTATTGT | |
| | | GTGCATCAAAGGATAGAGATAAAAGACACCAAGGAAGCTTTAGACAAGATAGAG | |
| | | GAAGAGCAAAACAAAAGTAAGACCACCGCACAGCAAGCGGCCGCTGATCTTCAG | |
| | | ACCTGGAGGAGGAGATATGAGGGACAATTGGAGAAGTGAATTATATAAATATAA | |
| | | AGTAGTAAAAATTGAACCATTAGGAGTAGCACCCACCAAGGCAAAGAGAAGAGT | |
| | | GGTGCAGAGAGAAAAAAGAGCAGTGGGAATAGGAGCTTTGTTCCTTGGGTTCTT | |
| | | GGGAGCAGCAGGAAGCACTATGGGCGCAGCGTCAATGACGCTGACGGTACAGGC | |
| | | CAGACAATTATTGTCTGGTATAGTGCAGCAGCAGAACAATTTGCTGAGGGCTAT | |
| | | TGAGGCGCAACAGCATCTGTTGCAACTCACAGTCTGGGGCATCAAGCAGCTCCA | |
| | | GGCAAGAATCCTGGCTGTGGAAAGATACCTAAAGGATCAACAGCTCCTGGGGAT | |
| | | TTGGGGTTGCTCTGGAAAACTCATTTGCACCACTGCTGTGCCTTGGAATGCTAG | |
| | | TTGGAGTAATAAATCTCTGGAACAGATTTGGAATCACACGACCTGGATGGAGTG | |
| | | GGACAGAGAAATTAACAATTACACAAGCTTAATACACTCCTTAATTGAAGAATC | |
| | | GCAAAACCAGCAAGAAAAGAATGAACAAGAATTATTGGAATTAGATAAATGGGC | |
| | | AAGTTTGTGGAATTGGTTTAACATAACAAATTGGCTGTGGTATATAAAATTATT | |
| | | CATAATGATAGTAGGAGGCTTGGTAGGTTTAAGAATAGTTTTTGCTGTACTTTC | |
| | | TATAGTGAATAGAGTTAGGCAGGGATATTCACCATTATCGTTTCAGACCCACCT | |
| | | CCCAACCCCGAGGGGACCCGACAGGCCCGAAGGAATAGAAGAAGAAGGTGGAGA | |
| | | GAGAGACAGAGACAGATCCATTCGATTAGTGAACGGATCTCGACGGTATCGGTT | |
| | | AACTTTTAAAAGAAAAGGGGGGATTGGGGGGTACAGTGCAGGGGAAAGAATAGT | |
| | | AGACATAATAGCAACAGACATACAAACTAAAGAATTACAAAAACAAATTACAAA | |
| | | ATTCAAAATTTTATCGATACTAGTATTATGCCCAGTACATGACCTTATGGGACT | |
| | | TTCCTACTTGGCAGTACATCTACAAACCCCGGGTGCAAAGATGGATAAAGTTTT | |
| | | AAACAGAGAGGAATCTTTGCAGCTAATGGACCTTCTAGGTCTTGAAAGGAGTGG | |
| | | GAATTGGCTCCGGTGCCCGTCAGTGGGCAGAGCGCACATCGCCCACAGTCCCCG | |
| | | AGAAGTTGGGGGGAGGGGTCGGCAATTGATCCGGTGCCTAGAGAAGGTGGCGCG | |
| | | GGGTAAACTGGGAAAGTGATGTCGTGTACTGGCTCCGCCTTTTTCCCGAGGGTG | |
| | | GGGGAGAACCGTATATAAGTGCAGTAGTCGCCGTGAACGTTCTTTTTCGCAACG | |
| | | GGTTTGCCGCCAGAACACAGGTAAGTGCCGTGTGTGGTTCCCGCGGGCCTGGCC | |
| | | TCTTTACGGGTTATGGCCCTTGCGTGCCTTGAATTACTTCCACTGGCTGCAGTA | |
| | | CGTGATTCTTGATCCCGAGCTTCGGGTTGGAAGTGGGTGGGAGAGTTCGAGGCC | |
| | | TTGCGCTTAAGGAGCCCCTTCGCCTCGTGCTTGAGTTGAGGCCTGGCCTGGGCG | |
| | | CTGGGGCCGCCGCGTGCGAATCTGGTGGCACCTTCGCGCCTGTCTCGCTGCTTT | |
| | | CGATAAGTCTCTAGCCATTTAAAATTTTTGATGACCTGCTGCGACGCTTTTTTT | |
| | | CTGGCAAGATAGTCTTGTAAATGCGGGCCAAGATCTGCACACTGGTATTTCGGT | |
| | | TTTTGGGGCCGCGGGCGGCGACGGGGCCCGTGCGTCCCAGCGCACATGTTCGGC | |
| | | GAGGCGGGGCCTGCGAGCGCGGCCACCGAGAATCGGACGGGGGTAGTCTCAAGC | |
| | | TGGCCGGCCTGCTCTGGTGCCTGGCCTCGCGCCGCCGTGTATCGCCCCGCCCTG | |
| | | GGCGGCAAGGCTGGCCCGGTCGGCACCAGTTGCGTGAGCGGAAAGATGGCCGCT | |
| | | TCCCGGCCCTGCTGCAGGGAGCTCAAAATGGAGGACGCGGCGCTCGGGAGAGCG | |
| | | GGCGGGTGAGTCACCCACACAAAGGAAAAGGGCCTTTCCGTCCTCAGCCGTCGC | |
| | | TTCATGTGACTCCACGGAGTACCGGGCGCCGTCCAGGCACCTCGATTAGTTCTC | |
| | | GAGCTTTTGGAGTACGTCGTCTTTAGGTTGGGGGGAGGGGTTTTATGCGATGGA | |
| | | GTTTCCCCACACTGAGTGGGTGGAGACTGAAGTTAGGCCAGCTTGGCACTTGAT | |
| | | GTAATTCTCCTTGGAATTTGCCCTTTTTGAGTTTGGATCTTGGTTCATTCTCAA | |
| | | GCCTCAGACAGTGGTTCAAAGTTTTTTTCTTCCATTTCAGGTGTCGTGACGTAG | |
| | | CGGCCCTAATACGACTCACTATAGGGCCCGCGTACGTCTAGACCTCCAGGCATC | |
| | | GATTCCACCATGGCACTCCCCGTCACCGCCCTTCTCTTGCCCCTCGCCCTGCTG | |
| | | CTGCATGCTGCCAGGCCCCAGGTGCAGCTGGTGCAGTCTGGGGCTGAGGTGAAG | |
| | | AAGCCTGGGTCCTCGGTGAAGGTCTCCTGCAAGGCTTCTGGAGGCACCTTCAGC | |
| | | AGCTATGCTATCAGCTGGGTGCGACAGGCCCCTGGACAAGGGCTTGAGTGGATG | |
| | | GGAGGGATCATCCCTATCTTTGGTACAGCAAACTACGCACAGAAGTTCCAGGGC | |
| | | AGAGTCACGATTACCGCGGACGAATCCACGAGCACAGCCTACATGGAGCTGAGC | |
| | | AGCCTGAGATCTGAGGACACGGCCGTGTATTACTGTGCGAGCCCCCGCGATTGT | |
| | | AGTAGTACCAGCTGCTATGCGGGGATTGATGCTTTTGATATCTGGGGCCAAGGG | |
| | | ACAATGGTCACCGTCTCGAGTGGTGGAGGCGGTTCAGGCGGAGGTGGCTCTGGC | |
| | | GGTGGCGCTAGCGAAATTGTGTTGACGCAGTCTCCAGGCACCCTGTCTTTGTCT | |
| | | CCAGGGGAAAGAGCCACCCTCTCCTGCAGGGCCAGTCAGAGTGTTAGCAGCAGC | |
| | | TACTTAGCCTGGTACCAGCAGAAACCTGGCCAGGCTCCCAGGCTCCTCATCTAT | |
| | | GGTGCATCCAGCAGGGCCACTGGCATCCCAGACAGGTTCAGTGGCAGTGGGTCT | |
| | | GGGACAGACTTCACTCTCACCATCAGCAGACTGGAGCCTGAAGATTTTGCAGTG | |
| | | TATTATTGTCAGCAGTATGGTAACTCACCTACCACTTTCGGCGGAGGGACCAAG | |
| | | CTGGAGATCAAACGGACCGCGGCCGCAACCACAACCCCTGCTCCAAGGCCCCCC | |
| | | ACACCCGCTCCAACTATAGCCAGCCAACCATTGAGCCTCAGACCTGAAGCTTGC | |
| | | AGGCCCGCAGCAGGAGGCGCCGTCCATACGCGAGGCCTGGACTTCGCGTGTGAT | |
| | | ATTTATATTTGGGCCCCTTTGGCCGGAACATGTGGGGTGTTGCTTCTCTCCCTT | |
| | | GTGATCACTCTGTATTGTAAGCGCGGGAGAAAGAAGCTCCTGTACATCTTCAAG | |
| | | CAGCCTTTTATGCGACCTGTGCAAACCACTCAGGAAGAAGATGGGTGTTCATGC | |
| | | CGCTTCCCCGAGGAGGAAGAAGGAGGGTGTGAACTGAGGGTGAAATTTTCTAGA | |
| | | AGCGCCGATGCTCCCGCATATCAGCAGGGTCAGAATCAGCTCTACAATGAATTG | |
| | | AATCTCGGCAGGCGAGAAGAGTACGATGTTCTGGACAAGAGACGGGGCAGGGAT | |
| | | CCCGAGATGGGGGGAAAGCCCCGGAGAAAAAATCCTCAGGAGGGGTTGTACAAT | |
| | | GAGCTGCAGAAGGACAAGATGGCTGAAGCCTATAGCGAGATCGGAATGAAAGGC | |
| | | GAAAGACGCAGAGGCAAGGGGCATGACGGTCTGTACCAGGGTCTCTCTACAGCC | |
| | | ACCAAGGACACTTATGATGCGTTGCATATGCAAGCCTTGCCACCCCGCGGATCA | |
| | | GGAGCGACTAACTTCTCTTTGTTGAAGCAAGCGGGTGACGTTGAAGAGAATCCC | |
| | | GGTCCATCCGGGGGATCCATGCGGATCTCCAAGCCCCACCTCCGAAGCATCAGT | |
| | | ATCCAGTGTTATCTGTGCCTTCTTCTGAATTCCCATTTTTTGACAGAGGCAGGA | |
| | | ATTCATGTATTCATATTGGGTTGTTTCTCCGCGGGGCTTCCTAAAACCGAAGCT | |
| | | AACTGGGTTAATGTCATTTCAGATCTGAAGAAAATTGAAGATCTTATTCAATCA | |
| | | ATGCATATCGACGCGACCTTGTACACGGAGAGTGATGTCCACCCGAGCTGCAAG | |
| | | GTAACGGCCATGAAATGCTTCTTGCTGGAATTGCAGGTAATTAGTCTTGAGAGT | |
| | | GGAGATGCGTCTATACACGACACCGTAGAGAATCTCATTATTTTGGCGAACAAT | |
| | | TCTCTCAGCAGTAACGGCAATGTTACTGAATCAGGATGTAAGGAGTGTGAAGAG | |
| | | CTGGAAGAAAAGAACATTAAGGAATTTTTGCAGAGCTTCGTACATATCGTCCAA | |
| | | ATGTTCATAAATACAAGCTAAGTCGACGGCCGGCCGCGAGCATCTTACCGCCAT | |
| | | TTATACCCATATTTGTTCTGTTTTTCTTGATTTGGGTATACATTTAAATGTTAA | |
| | | TAAAACAAAATGGTGGGGCAATCATTTACATTTTATAGGATATGTAATTACTAG | |
| | | TTCAGGTGTATTGCCACAAGACAAACATCTTAAGAAACTTTCCCGTTATTTACG | |
| | | CTCTGTTCCTGTTAATCAACCTCTGGATTACAAAATTTGTGAAAGATTGACTGA | |
| | | TATTCTTAACTATGTTGCTCCTTTTACGCTGTGTGGATATGCTGCTTTAATACC | |
| | | TCTGTATCATACTATTGCTTCCTGTACGGCTTTCGTTTTCTCCTCCTTGTATAA | |
| | | ATCCTGGTTGCTGTCTCTTTATCAGGAGTTGTGGCCCGTTGTCCGTCAACGTGG | |
| | | CGTGGTGTGCTCTGTGTTTGCTGACGCAACCCCCACTGGCTGGGGCATTGCCAC | |
| | | CACCTGTCAACTCCTTTCTGGGACTTTCGCTTTCCCCCTCCCGATCGCCACGGC | |
| | | AGAACTCATCGCCGCCTGCCTTGCCCGCTGCTGGACAGGGGCTAGGTTGCTGGG | |
| | | CACTGATAATTCCGTGGTGTTGTCCTGGTACCTTTAAGACCAATGACTTACAAG | |
| | | GCAGCTGTAGATCTTAGCCACTTTTTAAAAGAAAAGGGGGGACTGGAAGGGCTA | |
| | | ATTCACTCCCAACGAAGATAAGATCTGCTTTTTGCTTGTACTGGGTCTCTCTGG | |
| | | TTAGACCAGATCTGAGCCTGGGAGCTCTCTGGCTAACTAGGGAACCCACTGCTT | |
| | | AAGCCTCAATAAAGCTTGCCTTGAGTGCTTCAAGTAGTGTGTGCCCGTCTGTTG | |
| | | TGTGACTCTGGTAACTAGAGATCCCTCAGACCCTTTTAGTCAGTGTGGAAAATC | |
| | | TCTAGCAGTAGTAGTTCATGTCATCTTATTATTCAGTATTTATAACTTGCAAAG | |
| | | AAATGAATATCAGAGAGTGAGAGGAACTTGTTTATTGCAGCTTATAATGGTTAC | |
| | | AAATAAAGCAATAGCATCACAAATTTCACAAATAAAGCATTTTTTTCACTGCAT | |
| | | TCTAGTTGTGGTTTGTCCAAACTCATCAATGTATCTTATCATGTCTGGCTCTAG | |
| | | CTATCCCGCCCCTAACTCCGCCCATCCCGCCCCTAACTCCGCCCAGTTCCGCCC | |
| | | ATTCTCCGCCCCATGGCTGACTAATTTTTTTTATTTATGCAGAGGCCGAGGCCG | |
| | | CCTCGGCCTCTGAGCTATTCCAGAAGTAGTGAGGAGGCTTTTTTGGAGGCCTAG | |
| | | ACTTTTGCAGAGACCAAATTCGTAATCATGTCATAGCTGTTTCCTGTGTGAAAT | |
| | | TGTTATCCGCTCACAATTCCACACAACATACGAGCCGGAAGCATAAAGTGTAAA | |
| | | GCCTGGGGTGCCTAATGAGTGAGCTAACTCACATTAATTGCGTTGCGCTCACTG | |
| | | CCCGCTTTCCAGTCGGGAAACCTGTCGTGCCAGCTGCATTAATGAATCGGCCAA | |
| | | CGCGCGGGGAGAGGCGGTTTGCGTATTGGGCGCTCTTCCGCTTCCTCGCTCACT | |
| | | GACTCGCTGCGCTCGGTCGTTCGGCTGCGGCGAGCGGTATCAGCTCACTCAAAG | |
| | | GCGGTAATACGGTTATCCACAGAATCAGGGGATAACGCAGGAAAGAACATGTGA | |
| | | GCAAAAGGCCAGCAAAAGGCCAGGAACCGTAAAAAGGCCGCGTTGCTGGCGTTT | |
| | | TTCCATAGGCTCCGCCCCCCTGACGAGCATCACAAAAATCGACGCTCAAGTCAG | |
| | | AGGTGGCGAAACCCGACAGGACTATAAAGATACCAGGCGTTTCCCCCTGGAAGC | |
| | | TCCCTCGTGCGCTCTCCTGTTCCGACCCTGCCGCTTACCGGATACCTGTCCGCC | |
| | | TTTCTCCCTTCGGGAAGCGTGGCGCTTTCTCATAGCTCACGCTGTAGGTATCTC | |
| | | AGTTCGGTGTAGGTCGTTCGCTCCAAGCTGGGCTGTGTGCACGAACCCCC | |
| D05 | VH | GAGGTGCAGCTGTTGGAGTCTGGGGGAGGCTTGGTACAGCCTGGGGGGTCCCTG | 24 |
| | | AGACTCTCCTGTGCAGCCTCTGGATTCACCTTTAGCAGCTATGCCATGAGCTGG | |
| | | GTCCGCCAGGCTCCAGGGAAGGGGCTGGAGTGGGTCTCAGCTATTAGTGGTAGT | |
| | | GGTGGTAGCACATACTACGCAGACTCCGTGAAGGGCCGGTTCACCATCTCCAGA | |
| | | GACAATTCCAAGAACACGCTGTATCTGCAAATGAACAGCCTGAGAGCCGAGGAC | |
| | | ACGGCCGTATATTACTGTGCGAAAACCCACAGCACCAGCTGGTACTACTTTGAC | |
| | | TACTGGGGCCGTGGCACCCTGGTCACTGTCTCGAGT | |
| | VL | GAAATTGTGTTGACGCAGTCTCCAGGCACCCTGTCTTTGTCTCCAGGGGAAAGA | 25 |
| | | GCCACCCTCTCCTGCAGGGCCAGTCAGAGTGTTAGCAGCAGCTACTTAGCCTGG | |
| | | TACCAGCAGAAACCTGGCCAGGCTCCCAGGCTCCTCATCTATGGTGCATCCAGC | |
| | | AGGGCCACTGGCATCCCAGACAGGTTCAGTGGCAGTGGGTCTGGGACAGACTTC | |
| | | ACTCTCACCATCAGCAGACTGGAGCCTGAAGATTTTGCAGTGTATTACTGTCAG | |
| | | CAGTATGGTAGCTCACCCCGGTACACTTTTGGCCAGGGGACCAAAGTGGATATC | |
| | | AAACGGACC | |
| | scFv | GAGGTGCAGCTGTTGGAGTCTGGGGGAGGCTTGGTACAGCCTGGGGGGTCCCTG | 26 |
| | | AGACTCTCCTGTGCAGCCTCTGGATTCACCTTTAGCAGCTATGCCATGAGCTGG | |
| | | GTCCGCCAGGCTCCAGGGAAGGGGCTGGAGTGGGTCTCAGCTATTAGTGGTAGT | |
| | | GGTGGTAGCACATACTACGCAGACTCCGTGAAGGGCCGGTTCACCATCTCCAGA | |
| | | GACAATTCCAAGAACACGCTGTATCTGCAAATGAACAGCCTGAGAGCCGAGGAC | |
| | | ACGGCCGTATATTACTGTGCGAAAACCCACAGCACCAGCTGGTACTACTTTGAC | |
| | | TACTGGGGCCGTGGCACCCTGGTCACTGTCTCGAGTGGTGGAGGCGGTTCAGGC | |
| | | GGAGGTGGCTCTGGCGGTGGCGCTAGCGAAATTGTGTTGACGCAGTCTCCAGGC | |
| | | ACCCTGTCTTTGTCTCCAGGGGAAAGAGCCACCCTCTCCTGCAGGGCCAGTCAG | |
| | | AGTGTTAGCAGCAGCTACTTAGCCTGGTACCAGCAGAAACCTGGCCAGGCTCCC | |
| | | AGGCTCCTCATCTATGGTGCATCCAGCAGGGCCACTGGCATCCCAGACAGGTTC | |
| | | AGTGGCAGTGGGTCTGGGACAGACTTCACTCTCACCATCAGCAGACTGGAGCCT | |
| | | GAAGATTTTGCAGTGTATTACTGTCAGCAGTATGGTAGCTCACCCCGGTACACT | |
| | | TTTGGCCAGGGGACCAAAGTGGATATCAAACGGACC | |
| | CAR | GAGGTGCAGCTGTTGGAGTCTGGGGGAGGCTTGGTACAGCCTGGGGGGTCCCTG | 27 |
| | | AGACTCTCCTGTGCAGCCTCTGGATTCACCTTTAGCAGCTATGCCATGAGCTGG | |
| | | GTCCGCCAGGCTCCAGGGAAGGGGCTGGAGTGGGTCTCAGCTATTAGTGGTAGT | |
| | | GGTGGTAGCACATACTACGCAGACTCCGTGAAGGGCCGGTTCACCATCTCCAGA | |
| | | GACAATTCCAAGAACACGCTGTATCTGCAAATGAACAGCCTGAGAGCCGAGGAC | |
| | | ACGGCCGTATATTACTGTGCGAAAACCCACAGCACCAGCTGGTACTACTTTGAC | |
| | | TACTGGGGCCGTGGCACCCTGGTCACTGTCTCGAGTGGTGGAGGCGGTTCAGGC | |
| | | GGAGGTGGCTCTGGCGGTGGCGCTAGCGAAATTGTGTTGACGCAGTCTCCAGGC | |
| | | ACCCTGTCTTTGTCTCCAGGGGAAAGAGCCACCCTCTCCTGCAGGGCCAGTCAG | |
| | | AGTGTTAGCAGCAGCTACTTAGCCTGGTACCAGCAGAAACCTGGCCAGGCTCCC | |
| | | AGGCTCCTCATCTATGGTGCATCCAGCAGGGCCACTGGCATCCCAGACAGGTTC | |
| | | AGTGGCAGTGGGTCTGGGACAGACTTCACTCTCACCATCAGCAGACTGGAGCCT | |
| | | GAAGATTTTGCAGTGTATTACTGTCAGCAGTATGGTAGCTCACCCCGGTACACT | |
| | | TTTGGCCAGGGGACCAAAGTGGATATCAAACGGACCGCGGCCGCAACCACAACC | |
| | | CCTGCTCCAAGGCCCCCCACACCCGCTCCAACTATAGCCAGCCAACCATTGAGC | |
| | | CTCAGACCTGAAGCTTGCAGGCCCGCAGCAGGAGGCGCCGTCCATACGCGAGGC | |
| | | CTGGACTTCGCGTGTGATATTTATATTTGGGCCCCTTTGGCCGGAACATGTGGG | |
| | | GTGTTGCTTCTCTCCCTTGTGATCACTCTGTATTGTAAGCGCGGGAGAAAGAAG | |
| | | CTCCTGTACATCTTCAAGCAGCCTTTTATGCGACCTGTGCAAACCACTCAGGAA | |
| | | GAAGATGGGTGTTCATGCCGCTTCCCCGAGGAGGAAGAAGGAGGGTGTGAACTG | |
| | | AGGGTGAAATTTTCTAGAAGCGCCGATGCTCCCGCATATCAGCAGGGTCAGAAT | |
| | | CAGCTCTACAATGAATTGAATCTCGGCAGGCGAGAAGAGTACGATGTTCTGGAC | |
| | | AAGAGACGGGGCAGGGATCCCGAGATGGGGGGAAAGCCCCGGAGAAAAAATCCT | |
| | | CAGGAGGGGTTGTACAATGAGCTGCAGAAGGACAAGATGGCTGAAGCCTATAGC | |
| | | GAGATCGGAATGAAAGGCGAAAGACGCAGAGGCAAGGGGCATGACGGTCTGTAC | |
| | | CAGGGTCTCTCTACAGCCACCAAGGACACTTATGATGCGTTGCATATGCAAGCC | |
| | | TTGCCACCCCGC | |
| | Lentiviral | CGTTCAGCCCGACCGCTGCGCCTTATCCGGTAACTATCGTCTTGAGTCCAACCC | 28 |
| | vector | GGTAAGACACGACTTATCGCCACTGGCAGCAGCCACTGGTAACAGGATTAGCAG | |
| | | AGCGAGGTATGTAGGCGGTGCTACAGAGTTCTTGAAGTGGTGGCCTAACTACGG | |
| | | CTACACTAGAAGAACAGTATTTGGTATCTGCGCTCTGCTGAAGCCAGTTACCTT | |
| | | CGGAAAAAGAGTTGGTAGCTCTTGATCCGGCAAACAAACCACCGCTGGTAGCGG | |
| | | TGGTTTTTTTGTTTGCAAGCAGCAGATTACGCGCAGAAAAAAAGGATCTCAAGA | |
| | | AGATCCTTTGATCTTTTCTACGGGGTCTGACGCTCAGTGGAACGAAAACTCACG | |
| | | TTAAGGGATTTTGGTCATGAGATTATCAAAAAGGATCTTCACCTAGATCCTTTT | |
| | | AAATTAAAAATGAAGTTTTAAATCAATCTAAAGTATATATGAGTAAACTTGGTC | |
| | | TGACAGTTAGAAAAACTCATCGAGCATCAAATGAAACTGCAATTTATTCATATC | |
| | | AGGATTATCAATACCATATTTTTGAAAAAGCCGTTTCTGTAATGAAGGAGAAAA | |
| | | CTCACCGAGGCAGTTCCATAGGATGGCAAGATCCTGGTATCGGTCTGCGATTCC | |
| | | GACTCGTCCAACATCAATACAACCTATTAATTTCCCCTCGTCAAAAATAAGGTT | |
| | | ATCAAGTGAGAAATCACCATGAGTGACGACTGAATCCGGTGAGAATGGCAAAAG | |
| | | TTTATGCATTTCTTTCCAGACTTGTTCAACAGGCCAGCCATTACGCTCGTCATC | |
| | | AAAATCACTCGCATCAACCAAACCGTTATTCATTCGTGATTGCGCCTGAGCGAG | |
| | | ACGAAATACGCGATCGCTGTTAAAAGGACAATTACAAACAGGAATCGAATGCAA | |
| | | CCGGCGCAGGAACACTGCCAGCGCATCAACAATATTTTCACCTGAATCAGGATA | |
| | | TTCTTCTAATACCTGGAATGCTGTTTTTCCGGGGATCGCAGTGGTGAGTAACCA | |
| | | TGCATCATCAGGAGTACGGATAAAATGCTTGATGGTCGGAAGAGGCATAAATTC | |
| | | CGTCAGCCAGTTTAGTCTGACCATCTCATCTGTAACATCATTGGCAACGCTACC | |
| | | TTTGCCATGTTTCAGAAACAACTCTGGCGCATCGGGCTTCCCATACAAGCGATA | |
| | | GATTGTCGCACCTGATTGCCCGACATTATCGCGAGCCCATTTATACCCATATAA | |
| | | ATCAGCATCCATGTTGGAATTTAATCGCGGCCTCGACGTTTCCCGTTGAATATG | |
| | | GCTCATACTCTTCCTTTTTCAATATTATTGAAGCATTTATCAGGGTTATTGTCT | |
| | | CATGAGCGGATACATATTTGAATGTATTTAGAAAAATAAACAAATAGGGGTTCC | |
| | | GCGCACATTTCCCCGAAAAGTGCCACCTGACGGATCGGGAGATCTCCCGATCCC | |
| | | CTATGGTGCACTCTCAGTACAATCTGCTCTGATGCCGCATAGTTAAGCCAGTAT | |
| | | CTGCTCCCTGCTTGTGTGTTGGAGGTCGCTGAGTAGTGCGCGAGCAAAATTTAA | |
| | | GCTACAACAAGGCAAGGCTTGACCGACAATTGCATGAAGAATCTGCTTAGGGTT | |
| | | AGGCGTTTTGCGCTGCTTCGCGATGTACGGGCCAGATATACCCGTTGACATTGA | |
| | | TTATTGACTAGTTATTAATAGTAATCAATTACGGGGTCATTAGTTCATAGCCCA | |
| | | TATATGGAGTTCCGCGTTACATAACTTACGGTAAATGGCCCGCCTGGCTGACCG | |
| | | CCCAACGACCCCCGCCCATTGACGTCAATAATGACGTATGTTCCCATAGTAACG | |
| | | CCAATAGGGACTTTCCATTGACGTCAATGGGTGGAGTATTTACGGTAAACTGCC | |
| | | CACTTGGCAGTACATCAAGTGTATCATATGCCAAGTACGCCCCCTATTGACGTC | |
| | | AATGACGGTAAATGGCCCGCCTGGCATTATGCCCAGTACATGACCTTATGGGAC | |
| | | TTTCCTACTTGGCAGTACATCTACGTATTAGTCATCGCTATTACCATGGTGATG | |
| | | CGGTTTTGGCAGTACATCAATGGGCGTGGATAGCGGTTTGACTCACGGGGATTT | |
| | | CCAAGTCTCCACCCCATTGACGTCAATGGGAGTTTGTTTTGGCACCAAAATCAA | |
| | | CGGGACTTTCCAAAATGTCGTAACAACTCCGCCCCATTGACGCAAATGGGCGGT | |
| | | AGGCGTGTACGGTGGGAGGTCTATATAAGCAGCGCGTTTTGCCTGTACTGGGTC | |
| | | TCTCTGGTTAGACCAGATCTGAGCCTGGGAGCTCTCTGGCTAACTAGGGAACCC | |
| | | ACTGCTTAAGCCTCAATAAAGCTTGCCTTGAGTGCTTCAAGTAGTGTGTGCCCG | |
| | | TCTGTTGTGTGACTCTGGTAACTAGAGATCCCTCAGACCCTTTTAGTCAGTGTG | |
| | | GAAAATCTCTAGCAGTGGCGCCCGAACAGGGACTTGAAAGCGAAAGGGAAACCA | |
| | | GAGGAGCTCTCTCGACGCAGGACTCGGCTTGCTGAAGCGCGCACGGCAAGAGGC | |
| | | GAGGGGCGGCGACTGGTGAGTACGCCAAAAATTTTGACTAGCGGAGGCTAGAAG | |
| | | GAGAGAGATGGGTGCGAGAGCGTCAGTATTAAGCGGGGGAGAATTAGATCGCGA | |
| | | TGGGAAAAAATTCGGTTAAGGCCAGGGGGAAAGAAAAAATATAAATTAAAACAT | |
| | | ATAGTATGGGCAAGCAGGGAGCTAGAACGATTCGCAGTTAATCCTGGCCTGTTA | |
| | | GAAACATCAGAAGGCTGTAGACAAATACTGGGACAGCTACAACCATCCCTTCAG | |
| | | ACAGGATCAGAAGAACTTAGATCATTATATAATACAGTAGCAACCCTCTATTGT | |
| | | GTGCATCAAAGGATAGAGATAAAAGACACCAAGGAAGCTTTAGACAAGATAGAG | |
| | | GAAGAGCAAAACAAAAGTAAGACCACCGCACAGCAAGCGGCCGCTGATCTTCAG | |
| | | ACCTGGAGGAGGAGATATGAGGGACAATTGGAGAAGTGAATTATATAAATATAA | |
| | | AGTAGTAAAAATTGAACCATTAGGAGTAGCACCCACCAAGGCAAAGAGAAGAGT | |
| | | GGTGCAGAGAGAAAAAAGAGCAGTGGGAATAGGAGCTTTGTTCCTTGGGTTCTT | |
| | | GGGAGCAGCAGGAAGCACTATGGGCGCAGCGTCAATGACGCTGACGGTACAGGC | |
| | | CAGACAATTATTGTCTGGTATAGTGCAGCAGCAGAACAATTTGCTGAGGGCTAT | |
| | | TGAGGCGCAACAGCATCTGTTGCAACTCACAGTCTGGGGCATCAAGCAGCTCCA | |
| | | GGCAAGAATCCTGGCTGTGGAAAGATACCTAAAGGATCAACAGCTCCTGGGGAT | |
| | | TTGGGGTTGCTCTGGAAAACTCATTTGCACCACTGCTGTGCCTTGGAATGCTAG | |
| | | TTGGAGTAATAAATCTCTGGAACAGATTTGGAATCACACGACCTGGATGGAGTG | |
| | | GGACAGAGAAATTAACAATTACACAAGCTTAATACACTCCTTAATTGAAGAATC | |
| | | GCAAAACCAGCAAGAAAAGAATGAACAAGAATTATTGGAATTAGATAAATGGGC | |
| | | AAGTTTGTGGAATTGGTTTAACATAACAAATTGGCTGTGGTATATAAAATTATT | |
| | | CATAATGATAGTAGGAGGCTTGGTAGGTTTAAGAATAGTTTTTGCTGTACTTTC | |
| | | TATAGTGAATAGAGTTAGGCAGGGATATTCACCATTATCGTTTCAGACCCACCT | |
| | | CCCAACCCCGAGGGGACCCGACAGGCCCGAAGGAATAGAAGAAGAAGGTGGAGA | |
| | | GAGAGACAGAGACAGATCCATTCGATTAGTGAACGGATCTCGACGGTATCGGTT | |
| | | AACTTTTAAAAGAAAAGGGGGGATTGGGGGGTACAGTGCAGGGGAAAGAATAGT | |
| | | AGACATAATAGCAACAGACATACAAACTAAAGAATTACAAAAACAAATTACAAA | |
| | | ATTCAAAATTTTATCGATACTAGTATTATGCCCAGTACATGACCTTATGGGACT | |
| | | TTCCTACTTGGCAGTACATCTACAAACCCCGGGTGCAAAGATGGATAAAGTTTT | |
| | | AAACAGAGAGGAATCTTTGCAGCTAATGGACCTTCTAGGTCTTGAAAGGAGTGG | |
| | | GAATTGGCTCCGGTGCCCGTCAGTGGGCAGAGCGCACATCGCCCACAGTCCCCG | |
| | | AGAAGTTGGGGGGAGGGGTCGGCAATTGATCCGGTGCCTAGAGAAGGTGGCGCG | |
| | | GGGTAAACTGGGAAAGTGATGTCGTGTACTGGCTCCGCCTTTTTCCCGAGGGTG | |
| | | GGGGAGAACCGTATATAAGTGCAGTAGTCGCCGTGAACGTTCTTTTTCGCAACG | |
| | | GGTTTGCCGCCAGAACACAGGTAAGTGCCGTGTGTGGTTCCCGCGGGCCTGGCC | |
| | | TCTTTACGGGTTATGGCCCTTGCGTGCCTTGAATTACTTCCACTGGCTGCAGTA | |
| | | CGTGATTCTTGATCCCGAGCTTCGGGTTGGAAGTGGGTGGGAGAGTTCGAGGCC | |
| | | TTGCGCTTAAGGAGCCCCTTCGCCTCGTGCTTGAGTTGAGGCCTGGCCTGGGCG | |
| | | CTGGGGCCGCCGCGTGCGAATCTGGTGGCACCTTCGCGCCTGTCTCGCTGCTTT | |
| | | CGATAAGTCTCTAGCCATTTAAAATTTTTGATGACCTGCTGCGACGCTTTTTTT | |
| | | CTGGCAAGATAGTCTTGTAAATGCGGGCCAAGATCTGCACACTGGTATTTCGGT | |
| | | TTTTGGGGCCGCGGGCGGCGACGGGGCCCGTGCGTCCCAGCGCACATGTTCGGC | |
| | | GAGGCGGGGCCTGCGAGCGCGGCCACCGAGAATCGGACGGGGGTAGTCTCAAGC | |
| | | TGGCCGGCCTGCTCTGGTGCCTGGCCTCGCGCCGCCGTGTATCGCCCCGCCCTG | |
| | | GGCGGCAAGGCTGGCCCGGTCGGCACCAGTTGCGTGAGCGGAAAGATGGCCGCT | |
| | | TCCCGGCCCTGCTGCAGGGAGCTCAAAATGGAGGACGCGGCGCTCGGGAGAGCG | |
| | | GGCGGGTGAGTCACCCACACAAAGGAAAAGGGCCTTTCCGTCCTCAGCCGTCGC | |
| | | TTCATGTGACTCCACGGAGTACCGGGCGCCGTCCAGGCACCTCGATTAGTTCTC | |
| | | GAGCTTTTGGAGTACGTCGTCTTTAGGTTGGGGGGAGGGGTTTTATGCGATGGA | |
| | | GTTTCCCCACACTGAGTGGGTGGAGACTGAAGTTAGGCCAGCTTGGCACTTGAT | |
| | | GTAATTCTCCTTGGAATTTGCCCTTTTTGAGTTTGGATCTTGGTTCATTCTCAA | |
| | | GCCTCAGACAGTGGTTCAAAGTTTTTTTCTTCCATTTCAGGTGTCGTGACGTAG | |
| | | CGGCCCTAATACGACTCACTATAGGGCCCGCGTACGTCTAGACCTCCAGGCATC | |
| | | GATTCCACCATGGCACTCCCCGTCACCGCCCTTCTCTTGCCCCTCGCCCTGCTG | |
| | | CTGCATGCTGCCAGGCCCGAGGTGCAGCTGTTGGAGTCTGGGGGAGGCTTGGTA | |
| | | CAGCCTGGGGGGTCCCTGAGACTCTCCTGTGCAGCCTCTGGATTCACCTTTAGC | |
| | | AGCTATGCCATGAGCTGGGTCCGCCAGGCTCCAGGGAAGGGGCTGGAGTGGGTC | |
| | | TCAGCTATTAGTGGTAGTGGTGGTAGCACATACTACGCAGACTCCGTGAAGGGC | |
| | | CGGTTCACCATCTCCAGAGACAATTCCAAGAACACGCTGTATCTGCAAATGAAC | |
| | | AGCCTGAGAGCCGAGGACACGGCCGTATATTACTGTGCGAAAACCCACAGCACC | |
| | | AGCTGGTACTACTTTGACTACTGGGGCCGTGGCACCCTGGTCACTGTCTCGAGT | |
| | | GGTGGAGGCGGTTCAGGCGGAGGTGGCTCTGGCGGTGGCGCTAGCGAAATTGTG | |
| | | TTGACGCAGTCTCCAGGCACCCTGTCTTTGTCTCCAGGGGAAAGAGCCACCCTC | |
| | | TCCTGCAGGGCCAGTCAGAGTGTTAGCAGCAGCTACTTAGCCTGGTACCAGCAG | |
| | | AAACCTGGCCAGGCTCCCAGGCTCCTCATCTATGGTGCATCCAGCAGGGCCACT | |
| | | GGCATCCCAGACAGGTTCAGTGGCAGTGGGTCTGGGACAGACTTCACTCTCACC | |
| | | ATCAGCAGACTGGAGCCTGAAGATTTTGCAGTGTATTACTGTCAGCAGTATGGT | |
| | | AGCTCACCCCGGTACACTTTTGGCCAGGGGACCAAAGTGGATATCAAACGGACC | |
| | | GCGGCCGCAACCACAACCCCTGCTCCAAGGCCCCCCACACCCGCTCCAACTATA | |
| | | GCCAGCCAACCATTGAGCCTCAGACCTGAAGCTTGCAGGCCCGCAGCAGGAGGC | |
| | | GCCGTCCATACGCGAGGCCTGGACTTCGCGTGTGATATTTATATTTGGGCCCCT | |
| | | TTGGCCGGAACATGTGGGGTGTTGCTTCTCTCCCTTGTGATCACTCTGTATTGT | |
| | | AAGCGCGGGAGAAAGAAGCTCCTGTACATCTTCAAGCAGCCTTTTATGCGACCT | |
| | | GTGCAAACCACTCAGGAAGAAGATGGGTGTTCATGCCGCTTCCCCGAGGAGGAA | |
| | | GAAGGAGGGTGTGAACTGAGGGTGAAATTTTCTAGAAGCGCCGATGCTCCCGCA | |
| | | TATCAGCAGGGTCAGAATCAGCTCTACAATGAATTGAATCTCGGCAGGCGAGAA | |
| | | GAGTACGATGTTCTGGACAAGAGACGGGGCAGGGATCCCGAGATGGGGGGAAAG | |
| | | CCCCGGAGAAAAAATCCTCAGGAGGGGTTGTACAATGAGCTGCAGAAGGACAAG | |
| | | ATGGCTGAAGCCTATAGCGAGATCGGAATGAAAGGCGAAAGACGCAGAGGCAAG | |
| | | GGGCATGACGGTCTGTACCAGGGTCTCTCTACAGCCACCAAGGACACTTATGAT | |
| | | GCGTTGCATATGCAAGCCTTGCCACCCCGCGGATCAGGAGCGACTAACTTCTCT | |
| | | TTGTTGAAGCAAGCGGGTGACGTTGAAGAGAATCCCGGTCCATCCGGGGGATCC | |
| | | ATGCGGATCTCCAAGCCCCACCTCCGAAGCATCAGTATCCAGTGTTATCTGTGC | |
| | | CTTCTTCTGAATTCCCATTTTTTGACAGAGGCAGGAATTCATGTATTCATATTG | |
| | | GGTTGTTTCTCCGCGGGGCTTCCTAAAACCGAAGCTAACTGGGTTAATGTCATT | |
| | | TCAGATCTGAAGAAAATTGAAGATCTTATTCAATCAATGCATATCGACGCGACC | |
| | | TTGTACACGGAGAGTGATGTCCACCCGAGCTGCAAGGTAACGGCCATGAAATGC | |
| | | TTCTTGCTGGAATTGCAGGTAATTAGTCTTGAGAGTGGAGATGCGTCTATACAC | |
| | | GACACCGTAGAGAATCTCATTATTTTGGCGAACAATTCTCTCAGCAGTAACGGC | |
| | | AATGTTACTGAATCAGGATGTAAGGAGTGTGAAGAGCTGGAAGAAAAGAACATT | |
| | | AAGGAATTTTTGCAGAGCTTCGTACATATCGTCCAAATGTTCATAAATACAAGC | |
| | | TAAGTCGACGGCCGGCCGCGAGCATCTTACCGCCATTTATACCCATATTTGTTC | |
| | | TGTTTTTCTTGATTTGGGTATACATTTAAATGTTAATAAAACAAAATGGTGGGG | |
| | | CAATCATTTACATTTTATAGGATATGTAATTACTAGTTCAGGTGTATTGCCACA | |
| | | AGACAAACATCTTAAGAAACTTTCCCGTTATTTACGCTCTGTTCCTGTTAATCA | |
| | | ACCTCTGGATTACAAAATTTGTGAAAGATTGACTGATATTCTTAACTATGTTGC | |
| | | TCCTTTTACGCTGTGTGGATATGCTGCTTTAATACCTCTGTATCATACTATTGC | |
| | | TTCCTGTACGGCTTTCGTTTTCTCCTCCTTGTATAAATCCTGGTTGCTGTCTCT | |
| | | TTATCAGGAGTTGTGGCCCGTTGTCCGTCAACGTGGCGTGGTGTGCTCTGTGTT | |
| | | TGCTGACGCAACCCCCACTGGCTGGGGCATTGCCACCACCTGTCAACTCCTTTC | |
| | | TGGGACTTTCGCTTTCCCCCTCCCGATCGCCACGGCAGAACTCATCGCCGCCTG | |
| | | CCTTGCCCGCTGCTGGACAGGGGCTAGGTTGCTGGGCACTGATAATTCCGTGGT | |
| | | GTTGTCCTGGTACCTTTAAGACCAATGACTTACAAGGCAGCTGTAGATCTTAGC | |
| | | CACTTTTTAAAAGAAAAGGGGGGACTGGAAGGGCTAATTCACTCCCAACGAAGA | |
| | | TAAGATCTGCTTTTTGCTTGTACTGGGTCTCTCTGGTTAGACCAGATCTGAGCC | |
| | | TGGGAGCTCTCTGGCTAACTAGGGAACCCACTGCTTAAGCCTCAATAAAGCTTG | |
| | | CCTTGAGTGCTTCAAGTAGTGTGTGCCCGTCTGTTGTGTGACTCTGGTAACTAG | |
| | | AGATCCCTCAGACCCTTTTAGTCAGTGTGGAAAATCTCTAGCAGTAGTAGTTCA | |
| | | TGTCATCTTATTATTCAGTATTTATAACTTGCAAAGAAATGAATATCAGAGAGT | |
| | | GAGAGGAACTTGTTTATTGCAGCTTATAATGGTTACAAATAAAGCAATAGCATC | |
| | | ACAAATTTCACAAATAAAGCATTTTTTTCACTGCATTCTAGTTGTGGTTTGTCC | |
| | | AAACTCATCAATGTATCTTATCATGTCTGGCTCTAGCTATCCCGCCCCTAACTC | |
| | | CGCCCATCCCGCCCCTAACTCCGCCCAGTTCCGCCCATTCTCCGCCCCATGGCT | |
| | | GACTAATTTTTTTTATTTATGCAGAGGCCGAGGCCGCCTCGGCCTCTGAGCTAT | |
| | | TCCAGAAGTAGTGAGGAGGCTTTTTTGGAGGCCTAGACTTTTGCAGAGACCAAA | |
| | | TTCGTAATCATGTCATAGCTGTTTCCTGTGTGAAATTGTTATCCGCTCACAATT | |
| | | CCACACAACATACGAGCCGGAAGCATAAAGTGTAAAGCCTGGGGTGCCTAATGA | |
| | | GTGAGCTAACTCACATTAATTGCGTTGCGCTCACTGCCCGCTTTCCAGTCGGGA | |
| | | AACCTGTCGTGCCAGCTGCATTAATGAATCGGCCAACGCGCGGGGAGAGGCGGT | |
| | | TTGCGTATTGGGCGCTCTTCCGCTTCCTCGCTCACTGACTCGCTGCGCTCGGTC | |
| | | GTTCGGCTGCGGCGAGCGGTATCAGCTCACTCAAAGGCGGTAATACGGTTATCC | |
| | | ACAGAATCAGGGGATAACGCAGGAAAGAACATGTGAGCAAAAGGCCAGCAAAAG | |
| | | GCCAGGAACCGTAAAAAGGCCGCGTTGCTGGCGTTTTTCCATAGGCTCCGCCCC | |
| | | CCTGACGAGCATCACAAAAATCGACGCTCAAGTCAGAGGTGGCGAAACCCGACA | |
| | | GGACTATAAAGATACCAGGCGTTTCCCCCTGGAAGCTCCCTCGTGCGCTCTCCT | |
| | | GTTCCGACCCTGCCGCTTACCGGATACCTGTCCGCCTTTCTCCCTTCGGGAAGC | |
| | | GTGGCGCTTTCTCATAGCTCACGCTGTAGGTATCTCAGTTCGGTGTAGGTCGTT | |
| | | CGCTCCAAGCTGGGCTGTGTGCACGAACCCCC | |
| E07 | VH | GAGGTGCAGCTGTTGGAGTCTGGGGGAGGCTTGGTACAGCCTGGGGGGTCCCTG | 63 |
| | | AGACTCTCCTGTGCAGCCTCTGGATTCACCTTTAAAAGCTATGTGATGAGCTGG | |
| | | GTCCGCCAGGCTCCAGGGAAGGGGCTGGAGTGGGTCTCAGCTATTGTGCCAAGT | |
| | | GGTGGTGATACATACTACGCAGACTCCGTGAAGGGCCGGTTCACCATCTCCAGA | |
| | | GACAATTCCAAGAACACGCTGTATCTGCAAATGAACAGCCTGAGAGCCGAGGAC | |
| | | ACGGCCGTATATTACTGTGCGAAAGATTACTACGGAGGGTGGTTCGACCCCTGG | |
| | | GGCCAGGGAACCCTGGTCACCGTCTCGAGT | |
| | VL | GAAATTGTGTTGACGCAGTCTCCAGGCACCCTGTCTTTGTCTCCAGGGGAAAGA | 64 |
| | | GCCACCCTCTCCTGCAGGGCCAGTCAGAGTGTTAGCAGCAGCTACTTAGCCTGG | |
| | | TACCAGCAGAAACCTGGCCAGGCTCCCAGGCTCCTCATCTATGGTGCATCCAGC | |
| | | AGGGCCACTGGCATCCCAGACAGGTTCAGTGGCAGTGGGTCTGGGACAGACTTC | |
| | | ACTCTCACCATCAGCAGACTGGAGCCTGAAGATTTTGCAGTGTATTACTGTCAG | |
| | | CAGTATGGTAGCTCACCTCGAACGTTCGGCCAAGGGACCAAGGTGGAGATCAAA | |
| | scFv | GAGGTGCAGCTGTTGGAGTCTGGGGGAGGCTTGGTACAGCCTGGGGGGTCCCTG | 65 |
| | | AGACTCTCCTGTGCAGCCTCTGGATTCACCTTTAAAAGCTATGTGATGAGCTGG | |
| | | GTCCGCCAGGCTCCAGGGAAGGGGCTGGAGTGGGTCTCAGCTATTGTGCCAAGT | |
| | | GGTGGTGATACATACTACGCAGACTCCGTGAAGGGCCGGTTCACCATCTCCAGA | |
| | | GACAATTCCAAGAACACGCTGTATCTGCAAATGAACAGCCTGAGAGCCGAGGAC | |
| | | ACGGCCGTATATTACTGTGCGAAAGATTACTACGGAGGGTGGTTCGACCCCTGG | |
| | | GGCCAGGGAACCCTGGTCACCGTCTCGAGTGGTGGAGGCGGTTCAGGCGGAGGT | |
| | | GGCTCTGGCGGTGGCGCTAGCGAAATTGTGTTGACGCAGTCTCCAGGCACCCTG | |
| | | TCTTTGTCTCCAGGGGAAAGAGCCACCCTCTCCTGCAGGGCCAGTCAGAGTGTT | |
| | | AGCAGCAGCTACTTAGCCTGGTACCAGCAGAAACCTGGCCAGGCTCCCAGGCTC | |
| | | CTCATCTATGGTGCATCCAGCAGGGCCACTGGCATCCCAGACAGGTTCAGTGGC | |
| | | AGTGGGTCTGGGACAGACTTCACTCTCACCATCAGCAGACTGGAGCCTGAAGAT | |
| | | TTTGCAGTGTATTACTGTCAGCAGTATGGTAGCTCACCTCGAACGTTCGGCCAA | |
| | | GGGACCAAGGTGGAGATCAAA | |
| | CAR | GAGGTGCAGCTGTTGGAGTCTGGGGGAGGCTTGGTACAGCCTGGGGGGTCCCTG | 66 |
| | | AGACTCTCCTGTGCAGCCTCTGGATTCACCTTTAAAAGCTATGTGATGAGCTGG | |
| | | GTCCGCCAGGCTCCAGGGAAGGGGCTGGAGTGGGTCTCAGCTATTGTGCCAAGT | |
| | | GGTGGTGATACATACTACGCAGACTCCGTGAAGGGCCGGTTCACCATCTCCAGA | |
| | | GACAATTCCAAGAACACGCTGTATCTGCAAATGAACAGCCTGAGAGCCGAGGAC | |
| | | ACGGCCGTATATTACTGTGCGAAAGATTACTACGGAGGGTGGTTCGACCCCTGG | |
| | | GGCCAGGGAACCCTGGTCACCGTCTCGAGTGGTGGAGGCGGTTCAGGCGGAGGT | |
| | | GGCTCTGGCGGTGGCGCTAGCGAAATTGTGTTGACGCAGTCTCCAGGCACCCTG | |
| | | TCTTTGTCTCCAGGGGAAAGAGCCACCCTCTCCTGCAGGGCCAGTCAGAGTGTT | |
| | | AGCAGCAGCTACTTAGCCTGGTACCAGCAGAAACCTGGCCAGGCTCCCAGGCTC | |
| | | CTCATCTATGGTGCATCCAGCAGGGCCACTGGCATCCCAGACAGGTTCAGTGGC | |
| | | AGTGGGTCTGGGACAGACTTCACTCTCACCATCAGCAGACTGGAGCCTGAAGAT | |
| | | TTTGCAGTGTATTACTGTCAGCAGTATGGTAGCTCACCTCGAACGTTCGGCCAA | |
| | | GGGACCAAGGTGGAGATCAAAGCGGCCGCAACCACAACCCCTGCTCCAAGGCCC | |
| | | CCCACACCCGCTCCAACTATAGCCAGCCAACCATTGAGCCTCAGACCTGAAGCT | |
| | | TGCAGGCCCGCAGCAGGAGGCGCCGTCCATACGCGAGGCCTGGACTTCGCGTGT | |
| | | GATATTTATATTTGGGCCCCTTTGGCCGGAACATGTGGGGTGTTGCTTCTCTCC | |
| | | CTTGTGATCACTCTGTATTGTAAGCGCGGGAGAAAGAAGCTCCTGTACATCTTC | |
| | | AAGCAGCCTTTTATGCGACCTGTGCAAACCACTCAGGAAGAAGATGGGTGTTCA | |
| | | TGCCGCTTCCCCGAGGAGGAAGAAGGAGGGTGTGAACTGAGGGTGAAATTTTCT | |
| | | AGAAGCGCCGATGCTCCCGCATATCAGCAGGGTCAGAATCAGCTCTACAATGAA | |
| | | TTGAATCTCGGCAGGCGAGAAGAGTACGATGTTCTGGACAAGAGACGGGGCAGG | |
| | | GATCCCGAGATGGGGGGAAAGCCCCGGAGAAAAAATCCTCAGGAGGGGTTGTAC | |
| | | AATGAGCTGCAGAAGGACAAGATGGCTGAAGCCTATAGCGAGATCGGAATGAAA | |
| | | GGCGAAAGACGCAGAGGCAAGGGGCATGACGGTCTGTACCAGGGTCTCTCTACA | |
| | | GCCACCAAGGACACTTATGATGCGTTGCATATGCAAGCCTTGCCACCCCGC | |
| | Lentiviral | CGTTCAGCCCGACCGCTGCGCCTTATCCGGTAACTATCGTCTTGAGTCCAACCC | 67 |
| | vector | GGTAAGACACGACTTATCGCCACTGGCAGCAGCCACTGGTAACAGGATTAGCAG | |
| | | AGCGAGGTATGTAGGCGGTGCTACAGAGTTCTTGAAGTGGTGGCCTAACTACGG | |
| | | CTACACTAGAAGAACAGTATTTGGTATCTGCGCTCTGCTGAAGCCAGTTACCTT | |
| | | CGGAAAAAGAGTTGGTAGCTCTTGATCCGGCAAACAAACCACCGCTGGTAGCGG | |
| | | TGGTTTTTTTGTTTGCAAGCAGCAGATTACGCGCAGAAAAAAAGGATCTCAAGA | |
| | | AGATCCTTTGATCTTTTCTACGGGGTCTGACGCTCAGTGGAACGAAAACTCACG | |
| | | TTAAGGGATTTTGGTCATGAGATTATCAAAAAGGATCTTCACCTAGATCCTTTT | |
| | | AAATTAAAAATGAAGTTTTAAATCAATCTAAAGTATATATGAGTAAACTTGGTC | |
| | | TGACAGTTAGAAAAACTCATCGAGCATCAAATGAAACTGCAATTTATTCATATC | |
| | | AGGATTATCAATACCATATTTTTGAAAAAGCCGTTTCTGTAATGAAGGAGAAAA | |
| | | CTCACCGAGGCAGTTCCATAGGATGGCAAGATCCTGGTATCGGTCTGCGATTCC | |
| | | GACTCGTCCAACATCAATACAACCTATTAATTTCCCCTCGTCAAAAATAAGGTT | |
| | | ATCAAGTGAGAAATCACCATGAGTGACGACTGAATCCGGTGAGAATGGCAAAAG | |
| | | TTTATGCATTTCTTTCCAGACTTGTTCAACAGGCCAGCCATTACGCTCGTCATC | |
| | | AAAATCACTCGCATCAACCAAACCGTTATTCATTCGTGATTGCGCCTGAGCGAG | |
| | | ACGAAATACGCGATCGCTGTTAAAAGGACAATTACAAACAGGAATCGAATGCAA | |
| | | CCGGCGCAGGAACACTGCCAGCGCATCAACAATATTTTCACCTGAATCAGGATA | |
| | | TTCTTCTAATACCTGGAATGCTGTTTTTCCGGGGATCGCAGTGGTGAGTAACCA | |
| | | TGCATCATCAGGAGTACGGATAAAATGCTTGATGGTCGGAAGAGGCATAAATTC | |
| | | CGTCAGCCAGTTTAGTCTGACCATCTCATCTGTAACATCATTGGCAACGCTACC | |
| | | TTTGCCATGTTTCAGAAACAACTCTGGCGCATCGGGCTTCCCATACAAGCGATA | |
| | | GATTGTCGCACCTGATTGCCCGACATTATCGCGAGCCCATTTATACCCATATAA | |
| | | ATCAGCATCCATGTTGGAATTTAATCGCGGCCTCGACGTTTCCCGTTGAATATG | |
| | | GCTCATACTCTTCCTTTTTCAATATTATTGAAGCATTTATCAGGGTTATTGTCT | |
| | | CATGAGCGGATACATATTTGAATGTATTTAGAAAAATAAACAAATAGGGGTTCC | |
| | | GCGCACATTTCCCCGAAAAGTGCCACCTGACGGATCGGGAGATCTCCCGATCCC | |
| | | CTATGGTGCACTCTCAGTACAATCTGCTCTGATGCCGCATAGTTAAGCCAGTAT | |
| | | CTGCTCCCTGCTTGTGTGTTGGAGGTCGCTGAGTAGTGCGCGAGCAAAATTTAA | |
| | | GCTACAACAAGGCAAGGCTTGACCGACAATTGCATGAAGAATCTGCTTAGGGTT | |
| | | AGGCGTTTTGCGCTGCTTCGCGATGTACGGGCCAGATATACCCGTTGACATTGA | |
| | | TTATTGACTAGTTATTAATAGTAATCAATTACGGGGTCATTAGTTCATAGCCCA | |
| | | TATATGGAGTTCCGCGTTACATAACTTACGGTAAATGGCCCGCCTGGCTGACCG | |
| | | CCCAACGACCCCCGCCCATTGACGTCAATAATGACGTATGTTCCCATAGTAACG | |
| | | CCAATAGGGACTTTCCATTGACGTCAATGGGTGGAGTATTTACGGTAAACTGCC | |
| | | CACTTGGCAGTACATCAAGTGTATCATATGCCAAGTACGCCCCCTATTGACGTC | |
| | | AATGACGGTAAATGGCCCGCCTGGCATTATGCCCAGTACATGACCTTATGGGAC | |
| | | TTTCCTACTTGGCAGTACATCTACGTATTAGTCATCGCTATTACCATGGTGATG | |
| | | CGGTTTTGGCAGTACATCAATGGGCGTGGATAGCGGTTTGACTCACGGGGATTT | |
| | | CCAAGTCTCCACCCCATTGACGTCAATGGGAGTTTGTTTTGGCACCAAAATCAA | |
| | | CGGGACTTTCCAAAATGTCGTAACAACTCCGCCCCATTGACGCAAATGGGCGGT | |
| | | AGGCGTGTACGGTGGGAGGTCTATATAAGCAGCGCGTTTTGCCTGTACTGGGTC | |
| | | TCTCTGGTTAGACCAGATCTGAGCCTGGGAGCTCTCTGGCTAACTAGGGAACCC | |
| | | ACTGCTTAAGCCTCAATAAAGCTTGCCTTGAGTGCTTCAAGTAGTGTGTGCCCG | |
| | | TCTGTTGTGTGACTCTGGTAACTAGAGATCCCTCAGACCCTTTTAGTCAGTGTG | |
| | | GAAAATCTCTAGCAGTGGCGCCCGAACAGGGACTTGAAAGCGAAAGGGAAACCA | |
| | | GAGGAGCTCTCTCGACGCAGGACTCGGCTTGCTGAAGCGCGCACGGCAAGAGGC | |
| | | GAGGGGCGGCGACTGGTGAGTACGCCAAAAATTTTGACTAGCGGAGGCTAGAAG | |
| | | GAGAGAGATGGGTGCGAGAGCGTCAGTATTAAGCGGGGGAGAATTAGATCGCGA | |
| | | TGGGAAAAAATTCGGTTAAGGCCAGGGGGAAAGAAAAAATATAAATTAAAACAT | |
| | | ATAGTATGGGCAAGCAGGGAGCTAGAACGATTCGCAGTTAATCCTGGCCTGTTA | |
| | | GAAACATCAGAAGGCTGTAGACAAATACTGGGACAGCTACAACCATCCCTTCAG | |
| | | ACAGGATCAGAAGAACTTAGATCATTATATAATACAGTAGCAACCCTCTATTGT | |
| | | GTGCATCAAAGGATAGAGATAAAAGACACCAAGGAAGCTTTAGACAAGATAGAG | |
| | | GAAGAGCAAAACAAAAGTAAGACCACCGCACAGCAAGCGGCCGCTGATCTTCAG | |
| | | ACCTGGAGGAGGAGATATGAGGGACAATTGGAGAAGTGAATTATATAAATATAA | |
| | | AGTAGTAAAAATTGAACCATTAGGAGTAGCACCCACCAAGGCAAAGAGAAGAGT | |
| | | GGTGCAGAGAGAAAAAAGAGCAGTGGGAATAGGAGCTTTGTTCCTTGGGTTCTT | |
| | | GGGAGCAGCAGGAAGCACTATGGGCGCAGCGTCAATGACGCTGACGGTACAGGC | |
| | | CAGACAATTATTGTCTGGTATAGTGCAGCAGCAGAACAATTTGCTGAGGGCTAT | |
| | | TGAGGCGCAACAGCATCTGTTGCAACTCACAGTCTGGGGCATCAAGCAGCTCCA | |
| | | GGCAAGAATCCTGGCTGTGGAAAGATACCTAAAGGATCAACAGCTCCTGGGGAT | |
| | | TTGGGGTTGCTCTGGAAAACTCATTTGCACCACTGCTGTGCCTTGGAATGCTAG | |
| | | TTGGAGTAATAAATCTCTGGAACAGATTTGGAATCACACGACCTGGATGGAGTG | |
| | | GGACAGAGAAATTAACAATTACACAAGCTTAATACACTCCTTAATTGAAGAATC | |
| | | GCAAAACCAGCAAGAAAAGAATGAACAAGAATTATTGGAATTAGATAAATGGGC | |
| | | AAGTTTGTGGAATTGGTTTAACATAACAAATTGGCTGTGGTATATAAAATTATT | |
| | | CATAATGATAGTAGGAGGCTTGGTAGGTTTAAGAATAGTTTTTGCTGTACTTTC | |
| | | TATAGTGAATAGAGTTAGGCAGGGATATTCACCATTATCGTTTCAGACCCACCT | |
| | | CCCAACCCCGAGGGGACCCGACAGGCCCGAAGGAATAGAAGAAGAAGGTGGAGA | |
| | | GAGAGACAGAGACAGATCCATTCGATTAGTGAACGGATCTCGACGGTATCGGTT | |
| | | AACTTTTAAAAGAAAAGGGGGGATTGGGGGGTACAGTGCAGGGGAAAGAATAGT | |
| | | AGACATAATAGCAACAGACATACAAACTAAAGAATTACAAAAACAAATTACAAA | |
| | | ATTCAAAATTTTATCGATACTAGTATTATGCCCAGTACATGACCTTATGGGACT | |
| | | TTCCTACTTGGCAGTACATCTACAAACCCCGGGTGCAAAGATGGATAAAGTTTT | |
| | | AAACAGAGAGGAATCTTTGCAGCTAATGGACCTTCTAGGTCTTGAAAGGAGTGG | |
| | | GAATTGGCTCCGGTGCCCGTCAGTGGGCAGAGCGCACATCGCCCACAGTCCCCG | |
| | | AGAAGTTGGGGGGAGGGGTCGGCAATTGATCCGGTGCCTAGAGAAGGTGGCGCG | |
| | | GGGTAAACTGGGAAAGTGATGTCGTGTACTGGCTCCGCCTTTTTCCCGAGGGTG | |
| | | GGGGAGAACCGTATATAAGTGCAGTAGTCGCCGTGAACGTTCTTTTTCGCAACG | |
| | | GGTTTGCCGCCAGAACACAGGTAAGTGCCGTGTGTGGTTCCCGCGGGCCTGGCC | |
| | | TCTTTACGGGTTATGGCCCTTGCGTGCCTTGAATTACTTCCACTGGCTGCAGTA | |
| | | CGTGATTCTTGATCCCGAGCTTCGGGTTGGAAGTGGGTGGGAGAGTTCGAGGCC | |
| | | TTGCGCTTAAGGAGCCCCTTCGCCTCGTGCTTGAGTTGAGGCCTGGCCTGGGCG | |
| | | CTGGGGCCGCCGCGTGCGAATCTGGTGGCACCTTCGCGCCTGTCTCGCTGCTTT | |
| | | CGATAAGTCTCTAGCCATTTAAAATTTTTGATGACCTGCTGCGACGCTTTTTTT | |
| | | CTGGCAAGATAGTCTTGTAAATGCGGGCCAAGATCTGCACACTGGTATTTCGGT | |
| | | TTTTGGGGCCGCGGGCGGCGACGGGGCCCGTGCGTCCCAGCGCACATGTTCGGC | |
| | | GAGGCGGGGCCTGCGAGCGCGGCCACCGAGAATCGGACGGGGGTAGTCTCAAGC | |
| | | TGGCCGGCCTGCTCTGGTGCCTGGCCTCGCGCCGCCGTGTATCGCCCCGCCCTG | |
| | | GGCGGCAAGGCTGGCCCGGTCGGCACCAGTTGCGTGAGCGGAAAGATGGCCGCT | |
| | | TCCCGGCCCTGCTGCAGGGAGCTCAAAATGGAGGACGCGGCGCTCGGGAGAGCG | |
| | | GGCGGGTGAGTCACCCACACAAAGGAAAAGGGCCTTTCCGTCCTCAGCCGTCGC | |
| | | TTCATGTGACTCCACGGAGTACCGGGCGCCGTCCAGGCACCTCGATTAGTTCTC | |
| | | GAGCTTTTGGAGTACGTCGTCTTTAGGTTGGGGGGAGGGGTTTTATGCGATGGA | |
| | | GTTTCCCCACACTGAGTGGGTGGAGACTGAAGTTAGGCCAGCTTGGCACTTGAT | |
| | | GTAATTCTCCTTGGAATTTGCCCTTTTTGAGTTTGGATCTTGGTTCATTCTCAA | |
| | | GCCTCAGACAGTGGTTCAAAGTTTTTTTCTTCCATTTCAGGTGTCGTGACGTAG | |
| | | CGGCCCTAATACGACTCACTATAGGGCCCGCGTACGTCTAGACCTCCAGGCATC | |
| | | GATTCCACCATGGCACTCCCCGTCACCGCCCTTCTCTTGCCCCTCGCCCTGCTG | |
| | | CTGCATGCTGCCAGGCCCGAGGTGCAGCTGTTGGAGTCTGGGGGAGGCTTGGTA | |
| | | CAGCCTGGGGGGTCCCTGAGACTCTCCTGTGCAGCCTCTGGATTCACCTTTAAA | |
| | | AGCTATGTGATGAGCTGGGTCCGCCAGGCTCCAGGGAAGGGGCTGGAGTGGGTC | |
| | | TCAGCTATTGTGCCAAGTGGTGGTGATACATACTACGCAGACTCCGTGAAGGGC | |
| | | CGGTTCACCATCTCCAGAGACAATTCCAAGAACACGCTGTATCTGCAAATGAAC | |
| | | AGCCTGAGAGCCGAGGACACGGCCGTATATTACTGTGCGAAAGATTACTACGGA | |
| | | GGGTGGTTCGACCCCTGGGGCCAGGGAACCCTGGTCACCGTCTCGAGTGGTGGA | |
| | | GGCGGTTCAGGCGGAGGTGGCTCTGGCGGTGGCGCTAGCGAAATTGTGTTGACG | |
| | | CAGTCTCCAGGCACCCTGTCTTTGTCTCCAGGGGAAAGAGCCACCCTCTCCTGC | |
| | | AGGGCCAGTCAGAGTGTTAGCAGCAGCTACTTAGCCTGGTACCAGCAGAAACCT | |
| | | GGCCAGGCTCCCAGGCTCCTCATCTATGGTGCATCCAGCAGGGCCACTGGCATC | |
| | | CCAGACAGGTTCAGTGGCAGTGGGTCTGGGACAGACTTCACTCTCACCATCAGC | |
| | | AGACTGGAGCCTGAAGATTTTGCAGTGTATTACTGTCAGCAGTATGGTAGCTCA | |
| | | CCTCGAACGTTCGGCCAAGGGACCAAGGTGGAGATCAAAGCGGCCGCAACCACA | |
| | | ACCCCTGCTCCAAGGCCCCCCACACCCGCTCCAACTATAGCCAGCCAACCATTG | |
| | | AGCCTCAGACCTGAAGCTTGCAGGCCCGCAGCAGGAGGCGCCGTCCATACGCGA | |
| | | GGCCTGGACTTCGCGTGTGATATTTATATTTGGGCCCCTTTGGCCGGAACATGT | |
| | | GGGGTGTTGCTTCTCTCCCTTGTGATCACTCTGTATTGTAAGCGCGGGAGAAAG | |
| | | AAGCTCCTGTACATCTTCAAGCAGCCTTTTATGCGACCTGTGCAAACCACTCAG | |
| | | GAAGAAGATGGGTGTTCATGCCGCTTCCCCGAGGAGGAAGAAGGAGGGTGTGAA | |
| | | CTGAGGGTGAAATTTTCTAGAAGCGCCGATGCTCCCGCATATCAGCAGGGTCAG | |
| | | AATCAGCTCTACAATGAATTGAATCTCGGCAGGCGAGAAGAGTACGATGTTCTG | |
| | | GACAAGAGACGGGGCAGGGATCCCGAGATGGGGGGAAAGCCCCGGAGAAAAAAT | |
| | | CCTCAGGAGGGGTTGTACAATGAGCTGCAGAAGGACAAGATGGCTGAAGCCTAT | |
| | | AGCGAGATCGGAATGAAAGGCGAAAGACGCAGAGGCAAGGGGCATGACGGTCTG | |
| | | TACCAGGGTCTCTCTACAGCCACCAAGGACACTTATGATGCGTTGCATATGCAA | |
| | | GCCTTGCCACCCCGCGGATCAGGAGCGACTAACTTCTCTTTGTTGAAGCAAGCG | |
| | | GGTGACGTTGAAGAGAATCCCGGTCCATCCGGGGGATCCATGCGGATCTCCAAG | |
| | | CCCCACCTCCGAAGCATCAGTATCCAGTGTTATCTGTGCCTTCTTCTGAATTCC | |
| | | CATTTTTTGACAGAGGCAGGAATTCATGTATTCATATTGGGTTGTTTCTCCGCG | |
| | | GGGCTTCCTAAAACCGAAGCTAACTGGGTTAATGTCATTTCAGATCTGAAGAAA | |
| | | ATTGAAGATCTTATTCAATCAATGCATATCGACGCGACCTTGTACACGGAGAGT | |
| | | GATGTCCACCCGAGCTGCAAGGTAACGGCCATGAAATGCTTCTTGCTGGAATTG | |
| | | CAGGTAATTAGTCTTGAGAGTGGAGATGCGTCTATACACGACACCGTAGAGAAT | |
| | | CTCATTATTTTGGCGAACAATTCTCTCAGCAGTAACGGCAATGTTACTGAATCA | |
| | | GGATGTAAGGAGTGTGAAGAGCTGGAAGAAAAGAACATTAAGGAATTTTTGCAG | |
| | | AGCTTCGTACATATCGTCCAAATGTTCATAAATACAAGCTAAGTCGACGGCCGG | |
| | | CCGCGAGCATCTTACCGCCATTTATACCCATATTTGTTCTGTTTTTCTTGATTT | |
| | | GGGTATACATTTAAATGTTAATAAAACAAAATGGTGGGGCAATCATTTACATTT | |
| | | TATAGGATATGTAATTACTAGTTCAGGTGTATTGCCACAAGACAAACATCTTAA | |
| | | GAAACTTTCCCGTTATTTACGCTCTGTTCCTGTTAATCAACCTCTGGATTACAA | |
| | | AATTTGTGAAAGATTGACTGATATTCTTAACTATGTTGCTCCTTTTACGCTGTG | |
| | | TGGATATGCTGCTTTAATACCTCTGTATCATACTATTGCTTCCTGTACGGCTTT | |
| | | CGTTTTCTCCTCCTTGTATAAATCCTGGTTGCTGTCTCTTTATCAGGAGTTGTG | |
| | | GCCCGTTGTCCGTCAACGTGGCGTGGTGTGCTCTGTGTTTGCTGACGCAACCCC | |
| | | CACTGGCTGGGGCATTGCCACCACCTGTCAACTCCTTTCTGGGACTTTCGCTTT | |
| | | CCCCCTCCCGATCGCCACGGCAGAACTCATCGCCGCCTGCCTTGCCCGCTGCTG | |
| | | GACAGGGGCTAGGTTGCTGGGCACTGATAATTCCGTGGTGTTGTCCTGGTACCT | |
| | | TTAAGACCAATGACTTACAAGGCAGCTGTAGATCTTAGCCACTTTTTAAAAGAA | |
| | | AAGGGGGGACTGGAAGGGCTAATTCACTCCCAACGAAGATAAGATCTGCTTTTT | |
| | | GCTTGTACTGGGTCTCTCTGGTTAGACCAGATCTGAGCCTGGGAGCTCTCTGGC | |
| | | TAACTAGGGAACCCACTGCTTAAGCCTCAATAAAGCTTGCCTTGAGTGCTTCAA | |
| | | GTAGTGTGTGCCCGTCTGTTGTGTGACTCTGGTAACTAGAGATCCCTCAGACCC | |
| | | TTTTAGTCAGTGTGGAAAATCTCTAGCAGTAGTAGTTCATGTCATCTTATTATT | |
| | | CAGTATTTATAACTTGCAAAGAAATGAATATCAGAGAGTGAGAGGAACTTGTTT | |
| | | ATTGCAGCTTATAATGGTTACAAATAAAGCAATAGCATCACAAATTTCACAAAT | |
| | | AAAGCATTTTTTTCACTGCATTCTAGTTGTGGTTTGTCCAAACTCATCAATGTA | |
| | | TCTTATCATGTCTGGCTCTAGCTATCCCGCCCCTAACTCCGCCCATCCCGCCCC | |
| | | TAACTCCGCCCAGTTCCGCCCATTCTCCGCCCCATGGCTGACTAATTTTTTTTA | |
| | | TTTATGCAGAGGCCGAGGCCGCCTCGGCCTCTGAGCTATTCCAGAAGTAGTGAG | |
| | | GAGGCTTTTTTGGAGGCCTAGACTTTTGCAGAGACCAAATTCGTAATCATGTCA | |
| | | TAGCTGTTTCCTGTGTGAAATTGTTATCCGCTCACAATTCCACACAACATACGA | |
| | | GCCGGAAGCATAAAGTGTAAAGCCTGGGGTGCCTAATGAGTGAGCTAACTCACA | |
| | | TTAATTGCGTTGCGCTCACTGCCCGCTTTCCAGTCGGGAAACCTGTCGTGCCAG | |
| | | CTGCATTAATGAATCGGCCAACGCGCGGGGAGAGGCGGTTTGCGTATTGGGCGC | |
| | | TCTTCCGCTTCCTCGCTCACTGACTCGCTGCGCTCGGTCGTTCGGCTGCGGCGA | |
| | | GCGGTATCAGCTCACTCAAAGGCGGTAATACGGTTATCCACAGAATCAGGGGAT | |
| | | AACGCAGGAAAGAACATGTGAGCAAAAGGCCAGCAAAAGGCCAGGAACCGTAAA | |
| | | AAGGCCGCGTTGCTGGCGTTTTTCCATAGGCTCCGCCCCCCTGACGAGCATCAC | |
| | | AAAAATCGACGCTCAAGTCAGAGGTGGCGAAACCCGACAGGACTATAAAGATAC | |
| | | CAGGCGTTTCCCCCTGGAAGCTCCCTCGTGCGCTCTCCTGTTCCGACCCTGCCG | |
| | | CTTACCGGATACCTGTCCGCCTTTCTCCCTTCGGGAAGCGTGGCGCTTTCTCAT | |
| | | AGCTCACGCTGTAGGTATCTCAGTTCGGTGTAGGTCGTTCGCTCCAAGCTGGGC | |
| | | TGTGTGCACGAACCCCC | |
| D01 | VH | GAGGTGCAGCTGTTGGAGTCTGGGGGAGGCTTGGTACAGCCTGGGGGGTCCCTG | 68 |
| | | AGACTCTCCTGTGCAGCCTCTGGATTCACCTTTAGCAGCTATGCCATGAGCTGG | |
| | | GTCCGCCAGGCTCCAGGGAAGGGGCTGGAGTGGGTCTCAGCTATTAGTGGTAGT | |
| | | GGTGGTAGCACATACTACGCAGACTCCGTGAAGGGCCGGTTCACCATCTCCAGA | |
| | | GACAATTCCAAGAACACGCTGTATCTGCAAATGAACAGCCTGAGAGCCGAGGAC | |
| | | ACGGCCGTATATTACTGTGCGAAAGATTACTACGGAGGGTGGTTCGACCCCTGG | |
| | | GGCCAGGGAACCCTGGTCACCGTCTCGAGT | |
| | VL | GAAATTGTGTTGACGCAGTCTCCAGGCACCCTGTCTTTGTCTCCAGGGGAAAGA | 69 |
| | | GCCACCCTCTCCTGCAGGGCCAGTCAGAGTGTTAGCAGCAGCTACTTAGCCTGG | |
| | | TACCAGCAGAAACCTGGCCAGGCTCCCAGGCTCCTCATCTATGGTGCATCCAGC | |
| | | AGGGCCACTGGCATCCCAGACAGGTTCAGTGGCAGTGGGTCTGGGACAGACTTC | |
| | | ACTCTCACCATCAGCAGACTGGAGCCTGAAGATTTTGCAGTGTATTACTGTCAG | |
| | | CAGTATGGTAGCTCACCTCGGACATTCGGCCACGGGACCAAGCTGGAGATCAAA | |
| | scFv | GAGGTGCAGCTGTTGGAGTCTGGGGGAGGCTTGGTACAGCCTGGGGGGTCCCTG | 70 |
| | | AGACTCTCCTGTGCAGCCTCTGGATTCACCTTTAGCAGCTATGCCATGAGCTGG | |
| | | GTCCGCCAGGCTCCAGGGAAGGGGCTGGAGTGGGTCTCAGCTATTAGTGGTAGT | |
| | | GGTGGTAGCACATACTACGCAGACTCCGTGAAGGGCCGGTTCACCATCTCCAGA | |
| | | GACAATTCCAAGAACACGCTGTATCTGCAAATGAACAGCCTGAGAGCCGAGGAC | |
| | | ACGGCCGTATATTACTGTGCGAAAGATTACTACGGAGGGTGGTTCGACCCCTGG | |
| | | GGCCAGGGAACCCTGGTCACCGTCTCGAGTGGTGGAGGCGGTTCAGGCGGAGGT | |
| | | GGCTCTGGCGGTGGCGCTAGCGAAATTGTGTTGACGCAGTCTCCAGGCACCCTG | |
| | | TCTTTGTCTCCAGGGGAAAGAGCCACCCTCTCCTGCAGGGCCAGTCAGAGTGTT | |
| | | AGCAGCAGCTACTTAGCCTGGTACCAGCAGAAACCTGGCCAGGCTCCCAGGCTC | |
| | | CTCATCTATGGTGCATCCAGCAGGGCCACTGGCATCCCAGACAGGTTCAGTGGC | |
| | | AGTGGGTCTGGGACAGACTTCACTCTCACCATCAGCAGACTGGAGCCTGAAGAT | |
| | | TTTGCAGTGTATTACTGTCAGCAGTATGGTAGCTCACCTCGGACATTCGGCCAC | |
| | | GGGACCAAGCTGGAGATCAAA | |
| | CAR | GAGGTGCAGCTGTTGGAGTCTGGGGGAGGCTTGGTACAGCCTGGGGGGTCCCTG | 71 |
| | | AGACTCTCCTGTGCAGCCTCTGGATTCACCTTTAGCAGCTATGCCATGAGCTGG | |
| | | GTCCGCCAGGCTCCAGGGAAGGGGCTGGAGTGGGTCTCAGCTATTAGTGGTAGT | |
| | | GGTGGTAGCACATACTACGCAGACTCCGTGAAGGGCCGGTTCACCATCTCCAGA | |
| | | GACAATTCCAAGAACACGCTGTATCTGCAAATGAACAGCCTGAGAGCCGAGGAC | |
| | | ACGGCCGTATATTACTGTGCGAAAGATTACTACGGAGGGTGGTTCGACCCCTGG | |
| | | GGCCAGGGAACCCTGGTCACCGTCTCGAGTGGTGGAGGCGGTTCAGGCGGAGGT | |
| | | GGCTCTGGCGGTGGCGCTAGCGAAATTGTGTTGACGCAGTCTCCAGGCACCCTG | |
| | | TCTTTGTCTCCAGGGGAAAGAGCCACCCTCTCCTGCAGGGCCAGTCAGAGTGTT | |
| | | AGCAGCAGCTACTTAGCCTGGTACCAGCAGAAACCTGGCCAGGCTCCCAGGCTC | |
| | | CTCATCTATGGTGCATCCAGCAGGGCCACTGGCATCCCAGACAGGTTCAGTGGC | |
| | | AGTGGGTCTGGGACAGACTTCACTCTCACCATCAGCAGACTGGAGCCTGAAGAT | |
| | | TTTGCAGTGTATTACTGTCAGCAGTATGGTAGCTCACCTCGGACATTCGGCCAC | |
| | | GGGACCAAGCTGGAGATCAAAGCGGCCGCAACCACAACCCCTGCTCCAAGGCCC | |
| | | CCCACACCCGCTCCAACTATAGCCAGCCAACCATTGAGCCTCAGACCTGAAGCT | |
| | | TGCAGGCCCGCAGCAGGAGGCGCCGTCCATACGCGAGGCCTGGACTTCGCGTGT | |
| | | GATATTTATATTTGGGCCCCTTTGGCCGGAACATGTGGGGTGTTGCTTCTCTCC | |
| | | CTTGTGATCACTCTGTATTGTAAGCGCGGGAGAAAGAAGCTCCTGTACATCTTC | |
| | | AAGCAGCCTTTTATGCGACCTGTGCAAACCACTCAGGAAGAAGATGGGTGTTCA | |
| | | TGCCGCTTCCCCGAGGAGGAAGAAGGAGGGTGTGAACTGAGGGTGAAATTTTCT | |
| | | AGAAGCGCCGATGCTCCCGCATATCAGCAGGGTCAGAATCAGCTCTACAATGAA | |
| | | TTGAATCTCGGCAGGCGAGAAGAGTACGATGTTCTGGACAAGAGACGGGGCAGG | |
| | | GATCCCGAGATGGGGGGAAAGCCCCGGAGAAAAAATCCTCAGGAGGGGTTGTAC | |
| | | AATGAGCTGCAGAAGGACAAGATGGCTGAAGCCTATAGCGAGATCGGAATGAAA | |
| | | GGCGAAAGACGCAGAGGCAAGGGGCATGACGGTCTGTACCAGGGTCTCTCTACA | |
| | | GCCACCAAGGACACTTATGATGCGTTGCATATGCAAGCCTTGCCACCCCGC | |
| | Lentiviral | CGTTCAGCCCGACCGCTGCGCCTTATCCGGTAACTATCGTCTTGAGTCCAACCC | 72 |
| | vector | GGTAAGACACGACTTATCGCCACTGGCAGCAGCCACTGGTAACAGGATTAGCAG | |
| | | AGCGAGGTATGTAGGCGGTGCTACAGAGTTCTTGAAGTGGTGGCCTAACTACGG | |
| | | CTACACTAGAAGAACAGTATTTGGTATCTGCGCTCTGCTGAAGCCAGTTACCTT | |
| | | CGGAAAAAGAGTTGGTAGCTCTTGATCCGGCAAACAAACCACCGCTGGTAGCGG | |
| | | TGGTTTTTTTGTTTGCAAGCAGCAGATTACGCGCAGAAAAAAAGGATCTCAAGA | |
| | | AGATCCTTTGATCTTTTCTACGGGGTCTGACGCTCAGTGGAACGAAAACTCACG | |
| | | TTAAGGGATTTTGGTCATGAGATTATCAAAAAGGATCTTCACCTAGATCCTTTT | |
| | | AAATTAAAAATGAAGTTTTAAATCAATCTAAAGTATATATGAGTAAACTTGGTC | |
| | | TGACAGTTAGAAAAACTCATCGAGCATCAAATGAAACTGCAATTTATTCATATC | |
| | | AGGATTATCAATACCATATTTTTGAAAAAGCCGTTTCTGTAATGAAGGAGAAAA | |
| | | CTCACCGAGGCAGTTCCATAGGATGGCAAGATCCTGGTATCGGTCTGCGATTCC | |
| | | GACTCGTCCAACATCAATACAACCTATTAATTTCCCCTCGTCAAAAATAAGGTT | |
| | | ATCAAGTGAGAAATCACCATGAGTGACGACTGAATCCGGTGAGAATGGCAAAAG | |
| | | TTTATGCATTTCTTTCCAGACTTGTTCAACAGGCCAGCCATTACGCTCGTCATC | |
| | | AAAATCACTCGCATCAACCAAACCGTTATTCATTCGTGATTGCGCCTGAGCGAG | |
| | | ACGAAATACGCGATCGCTGTTAAAAGGACAATTACAAACAGGAATCGAATGCAA | |
| | | CCGGCGCAGGAACACTGCCAGCGCATCAACAATATTTTCACCTGAATCAGGATA | |
| | | TTCTTCTAATACCTGGAATGCTGTTTTTCCGGGGATCGCAGTGGTGAGTAACCA | |
| | | TGCATCATCAGGAGTACGGATAAAATGCTTGATGGTCGGAAGAGGCATAAATTC | |
| | | CGTCAGCCAGTTTAGTCTGACCATCTCATCTGTAACATCATTGGCAACGCTACC | |
| | | TTTGCCATGTTTCAGAAACAACTCTGGCGCATCGGGCTTCCCATACAAGCGATA | |
| | | GATTGTCGCACCTGATTGCCCGACATTATCGCGAGCCCATTTATACCCATATAA | |
| | | ATCAGCATCCATGTTGGAATTTAATCGCGGCCTCGACGTTTCCCGTTGAATATG | |
| | | GCTCATACTCTTCCTTTTTCAATATTATTGAAGCATTTATCAGGGTTATTGTCT | |
| | | CATGAGCGGATACATATTTGAATGTATTTAGAAAAATAAACAAATAGGGGTTCC | |
| | | GCGCACATTTCCCCGAAAAGTGCCACCTGACGGATCGGGAGATCTCCCGATCCC | |
| | | CTATGGTGCACTCTCAGTACAATCTGCTCTGATGCCGCATAGTTAAGCCAGTAT | |
| | | CTGCTCCCTGCTTGTGTGTTGGAGGTCGCTGAGTAGTGCGCGAGCAAAATTTAA | |
| | | GCTACAACAAGGCAAGGCTTGACCGACAATTGCATGAAGAATCTGCTTAGGGTT | |
| | | AGGCGTTTTGCGCTGCTTCGCGATGTACGGGCCAGATATACCCGTTGACATTGA | |
| | | TTATTGACTAGTTATTAATAGTAATCAATTACGGGGTCATTAGTTCATAGCCCA | |
| | | TATATGGAGTTCCGCGTTACATAACTTACGGTAAATGGCCCGCCTGGCTGACCG | |
| | | CCCAACGACCCCCGCCCATTGACGTCAATAATGACGTATGTTCCCATAGTAACG | |
| | | CCAATAGGGACTTTCCATTGACGTCAATGGGTGGAGTATTTACGGTAAACTGCC | |
| | | CACTTGGCAGTACATCAAGTGTATCATATGCCAAGTACGCCCCCTATTGACGTC | |
| | | AATGACGGTAAATGGCCCGCCTGGCATTATGCCCAGTACATGACCTTATGGGAC | |
| | | TTTCCTACTTGGCAGTACATCTACGTATTAGTCATCGCTATTACCATGGTGATG | |
| | | CGGTTTTGGCAGTACATCAATGGGCGTGGATAGCGGTTTGACTCACGGGGATTT | |
| | | CCAAGTCTCCACCCCATTGACGTCAATGGGAGTTTGTTTTGGCACCAAAATCAA | |
| | | CGGGACTTTCCAAAATGTCGTAACAACTCCGCCCCATTGACGCAAATGGGCGGT | |
| | | AGGCGTGTACGGTGGGAGGTCTATATAAGCAGCGCGTTTTGCCTGTACTGGGTC | |
| | | TCTCTGGTTAGACCAGATCTGAGCCTGGGAGCTCTCTGGCTAACTAGGGAACCC | |
| | | ACTGCTTAAGCCTCAATAAAGCTTGCCTTGAGTGCTTCAAGTAGTGTGTGCCCG | |
| | | TCTGTTGTGTGACTCTGGTAACTAGAGATCCCTCAGACCCTTTTAGTCAGTGTG | |
| | | GAAAATCTCTAGCAGTGGCGCCCGAACAGGGACTTGAAAGCGAAAGGGAAACCA | |
| | | GAGGAGCTCTCTCGACGCAGGACTCGGCTTGCTGAAGCGCGCACGGCAAGAGGC | |
| | | GAGGGGCGGCGACTGGTGAGTACGCCAAAAATTTTGACTAGCGGAGGCTAGAAG | |
| | | GAGAGAGATGGGTGCGAGAGCGTCAGTATTAAGCGGGGGAGAATTAGATCGCGA | |
| | | TGGGAAAAAATTCGGTTAAGGCCAGGGGGAAAGAAAAAATATAAATTAAAACAT | |
| | | ATAGTATGGGCAAGCAGGGAGCTAGAACGATTCGCAGTTAATCCTGGCCTGTTA | |
| | | GAAACATCAGAAGGCTGTAGACAAATACTGGGACAGCTACAACCATCCCTTCAG | |
| | | ACAGGATCAGAAGAACTTAGATCATTATATAATACAGTAGCAACCCTCTATTGT | |
| | | GTGCATCAAAGGATAGAGATAAAAGACACCAAGGAAGCTTTAGACAAGATAGAG | |
| | | GAAGAGCAAAACAAAAGTAAGACCACCGCACAGCAAGCGGCCGCTGATCTTCAG | |
| | | ACCTGGAGGAGGAGATATGAGGGACAATTGGAGAAGTGAATTATATAAATATAA | |
| | | AGTAGTAAAAATTGAACCATTAGGAGTAGCACCCACCAAGGCAAAGAGAAGAGT | |
| | | GGTGCAGAGAGAAAAAAGAGCAGTGGGAATAGGAGCTTTGTTCCTTGGGTTCTT | |
| | | GGGAGCAGCAGGAAGCACTATGGGCGCAGCGTCAATGACGCTGACGGTACAGGC | |
| | | CAGACAATTATTGTCTGGTATAGTGCAGCAGCAGAACAATTTGCTGAGGGCTAT | |
| | | TGAGGCGCAACAGCATCTGTTGCAACTCACAGTCTGGGGCATCAAGCAGCTCCA | |
| | | GGCAAGAATCCTGGCTGTGGAAAGATACCTAAAGGATCAACAGCTCCTGGGGAT | |
| | | TTGGGGTTGCTCTGGAAAACTCATTTGCACCACTGCTGTGCCTTGGAATGCTAG | |
| | | TTGGAGTAATAAATCTCTGGAACAGATTTGGAATCACACGACCTGGATGGAGTG | |
| | | GGACAGAGAAATTAACAATTACACAAGCTTAATACACTCCTTAATTGAAGAATC | |
| | | GCAAAACCAGCAAGAAAAGAATGAACAAGAATTATTGGAATTAGATAAATGGGC | |
| | | AAGTTTGTGGAATTGGTTTAACATAACAAATTGGCTGTGGTATATAAAATTATT | |
| | | CATAATGATAGTAGGAGGCTTGGTAGGTTTAAGAATAGTTTTTGCTGTACTTTC | |
| | | TATAGTGAATAGAGTTAGGCAGGGATATTCACCATTATCGTTTCAGACCCACCT | |
| | | CCCAACCCCGAGGGGACCCGACAGGCCCGAAGGAATAGAAGAAGAAGGTGGAGA | |
| | | GAGAGACAGAGACAGATCCATTCGATTAGTGAACGGATCTCGACGGTATCGGTT | |
| | | AACTTTTAAAAGAAAAGGGGGGATTGGGGGGTACAGTGCAGGGGAAAGAATAGT | |
| | | AGACATAATAGCAACAGACATACAAACTAAAGAATTACAAAAACAAATTACAAA | |
| | | ATTCAAAATTTTATCGATACTAGTATTATGCCCAGTACATGACCTTATGGGACT | |
| | | TTCCTACTTGGCAGTACATCTACAAACCCCGGGTGCAAAGATGGATAAAGTTTT | |
| | | AAACAGAGAGGAATCTTTGCAGCTAATGGACCTTCTAGGTCTTGAAAGGAGTGG | |
| | | GAATTGGCTCCGGTGCCCGTCAGTGGGCAGAGCGCACATCGCCCACAGTCCCCG | |
| | | AGAAGTTGGGGGGAGGGGTCGGCAATTGATCCGGTGCCTAGAGAAGGTGGCGCG | |
| | | GGGTAAACTGGGAAAGTGATGTCGTGTACTGGCTCCGCCTTTTTCCCGAGGGTG | |
| | | GGGGAGAACCGTATATAAGTGCAGTAGTCGCCGTGAACGTTCTTTTTCGCAACG | |
| | | GGTTTGCCGCCAGAACACAGGTAAGTGCCGTGTGTGGTTCCCGCGGGCCTGGCC | |
| | | TCTTTACGGGTTATGGCCCTTGCGTGCCTTGAATTACTTCCACTGGCTGCAGTA | |
| | | CGTGATTCTTGATCCCGAGCTTCGGGTTGGAAGTGGGTGGGAGAGTTCGAGGCC | |
| | | TTGCGCTTAAGGAGCCCCTTCGCCTCGTGCTTGAGTTGAGGCCTGGCCTGGGCG | |
| | | CTGGGGCCGCCGCGTGCGAATCTGGTGGCACCTTCGCGCCTGTCTCGCTGCTTT | |
| | | CGATAAGTCTCTAGCCATTTAAAATTTTTGATGACCTGCTGCGACGCTTTTTTT | |
| | | CTGGCAAGATAGTCTTGTAAATGCGGGCCAAGATCTGCACACTGGTATTTCGGT | |
| | | TTTTGGGGCCGCGGGCGGCGACGGGGCCCGTGCGTCCCAGCGCACATGTTCGGC | |
| | | GAGGCGGGGCCTGCGAGCGCGGCCACCGAGAATCGGACGGGGGTAGTCTCAAGC | |
| | | TGGCCGGCCTGCTCTGGTGCCTGGCCTCGCGCCGCCGTGTATCGCCCCGCCCTG | |
| | | GGCGGCAAGGCTGGCCCGGTCGGCACCAGTTGCGTGAGCGGAAAGATGGCCGCT | |
| | | TCCCGGCCCTGCTGCAGGGAGCTCAAAATGGAGGACGCGGCGCTCGGGAGAGCG | |
| | | GGCGGGTGAGTCACCCACACAAAGGAAAAGGGCCTTTCCGTCCTCAGCCGTCGC | |
| | | TTCATGTGACTCCACGGAGTACCGGGCGCCGTCCAGGCACCTCGATTAGTTCTC | |
| | | GAGCTTTTGGAGTACGTCGTCTTTAGGTTGGGGGGAGGGGTTTTATGCGATGGA | |
| | | GTTTCCCCACACTGAGTGGGTGGAGACTGAAGTTAGGCCAGCTTGGCACTTGAT | |
| | | GTAATTCTCCTTGGAATTTGCCCTTTTTGAGTTTGGATCTTGGTTCATTCTCAA | |
| | | GCCTCAGACAGTGGTTCAAAGTTTTTTTCTTCCATTTCAGGTGTCGTGACGTAG | |
| | | CGGCCCTAATACGACTCACTATAGGGCCCGCGTACGTCTAGACCTCCAGGCATC | |
| | | GATTCCACCATGGCACTCCCCGTCACCGCCCTTCTCTTGCCCCTCGCCCTGCTG | |
| | | CTGCATGCTGCCAGGCCCGAGGTGCAGCTGTTGGAGTCTGGGGGAGGCTTGGTA | |
| | | CAGCCTGGGGGGTCCCTGAGACTCTCCTGTGCAGCCTCTGGATTCACCTTTAGC | |
| | | AGCTATGCCATGAGCTGGGTCCGCCAGGCTCCAGGGAAGGGGCTGGAGTGGGTC | |
| | | TCAGCTATTAGTGGTAGTGGTGGTAGCACATACTACGCAGACTCCGTGAAGGGC | |
| | | CGGTTCACCATCTCCAGAGACAATTCCAAGAACACGCTGTATCTGCAAATGAAC | |
| | | AGCCTGAGAGCCGAGGACACGGCCGTATATTACTGTGCGAAAGATTACTACGGA | |
| | | GGGTGGTTCGACCCCTGGGGCCAGGGAACCCTGGTCACCGTCTCGAGTGGTGGA | |
| | | GGCGGTTCAGGCGGAGGTGGCTCTGGCGGTGGCGCTAGCGAAATTGTGTTGACG | |
| | | CAGTCTCCAGGCACCCTGTCTTTGTCTCCAGGGGAAAGAGCCACCCTCTCCTGC | |
| | | AGGGCCAGTCAGAGTGTTAGCAGCAGCTACTTAGCCTGGTACCAGCAGAAACCT | |
| | | GGCCAGGCTCCCAGGCTCCTCATCTATGGTGCATCCAGCAGGGCCACTGGCATC | |
| | | CCAGACAGGTTCAGTGGCAGTGGGTCTGGGACAGACTTCACTCTCACCATCAGC | |
| | | AGACTGGAGCCTGAAGATTTTGCAGTGTATTACTGTCAGCAGTATGGTAGCTCA | |
| | | CCTCGGACATTCGGCCACGGGACCAAGCTGGAGATCAAAGCGGCCGCAACCACA | |
| | | ACCCCTGCTCCAAGGCCCCCCACACCCGCTCCAACTATAGCCAGCCAACCATTG | |
| | | AGCCTCAGACCTGAAGCTTGCAGGCCCGCAGCAGGAGGCGCCGTCCATACGCGA | |
| | | GGCCTGGACTTCGCGTGTGATATTTATATTTGGGCCCCTTTGGCCGGAACATGT | |
| | | GGGGTGTTGCTTCTCTCCCTTGTGATCACTCTGTATTGTAAGCGCGGGAGAAAG | |
| | | AAGCTCCTGTACATCTTCAAGCAGCCTTTTATGCGACCTGTGCAAACCACTCAG | |
| | | GAAGAAGATGGGTGTTCATGCCGCTTCCCCGAGGAGGAAGAAGGAGGGTGTGAA | |
| | | CTGAGGGTGAAATTTTCTAGAAGCGCCGATGCTCCCGCATATCAGCAGGGTCAG | |
| | | AATCAGCTCTACAATGAATTGAATCTCGGCAGGCGAGAAGAGTACGATGTTCTG | |
| | | GACAAGAGACGGGGCAGGGATCCCGAGATGGGGGGAAAGCCCCGGAGAAAAAAT | |
| | | CCTCAGGAGGGGTTGTACAATGAGCTGCAGAAGGACAAGATGGCTGAAGCCTAT | |
| | | AGCGAGATCGGAATGAAAGGCGAAAGACGCAGAGGCAAGGGGCATGACGGTCTG | |
| | | TACCAGGGTCTCTCTACAGCCACCAAGGACACTTATGATGCGTTGCATATGCAA | |
| | | GCCTTGCCACCCCGCGGATCAGGAGCGACTAACTTCTCTTTGTTGAAGCAAGCG | |
| | | GGTGACGTTGAAGAGAATCCCGGTCCATCCGGGGGATCCATGCGGATCTCCAAG | |
| | | CCCCACCTCCGAAGCATCAGTATCCAGTGTTATCTGTGCCTTCTTCTGAATTCC | |
| | | CATTTTTTGACAGAGGCAGGAATTCATGTATTCATATTGGGTTGTTTCTCCGCG | |
| | | GGGCTTCCTAAAACCGAAGCTAACTGGGTTAATGTCATTTCAGATCTGAAGAAA | |
| | | ATTGAAGATCTTATTCAATCAATGCATATCGACGCGACCTTGTACACGGAGAGT | |
| | | GATGTCCACCCGAGCTGCAAGGTAACGGCCATGAAATGCTTCTTGCTGGAATTG | |
| | | CAGGTAATTAGTCTTGAGAGTGGAGATGCGTCTATACACGACACCGTAGAGAAT | |
| | | CTCATTATTTTGGCGAACAATTCTCTCAGCAGTAACGGCAATGTTACTGAATCA | |
| | | GGATGTAAGGAGTGTGAAGAGCTGGAAGAAAAGAACATTAAGGAATTTTTGCAG | |
| | | AGCTTCGTACATATCGTCCAAATGTTCATAAATACAAGCTAAGTCGACGGCCGG | |
| | | CCGCGAGCATCTTACCGCCATTTATACCCATATTTGTTCTGTTTTTCTTGATTT | |
| | | GGGTATACATTTAAATGTTAATAAAACAAAATGGTGGGGCAATCATTTACATTT | |
| | | TATAGGATATGTAATTACTAGTTCAGGTGTATTGCCACAAGACAAACATCTTAA | |
| | | GAAACTTTCCCGTTATTTACGCTCTGTTCCTGTTAATCAACCTCTGGATTACAA | |
| | | AATTTGTGAAAGATTGACTGATATTCTTAACTATGTTGCTCCTTTTACGCTGTG | |
| | | TGGATATGCTGCTTTAATACCTCTGTATCATACTATTGCTTCCTGTACGGCTTT | |
| | | CGTTTTCTCCTCCTTGTATAAATCCTGGTTGCTGTCTCTTTATCAGGAGTTGTG | |
| | | GCCCGTTGTCCGTCAACGTGGCGTGGTGTGCTCTGTGTTTGCTGACGCAACCCC | |
| | | CACTGGCTGGGGCATTGCCACCACCTGTCAACTCCTTTCTGGGACTTTCGCTTT | |
| | | CCCCCTCCCGATCGCCACGGCAGAACTCATCGCCGCCTGCCTTGCCCGCTGCTG | |
| | | GACAGGGGCTAGGTTGCTGGGCACTGATAATTCCGTGGTGTTGTCCTGGTACCT | |
| | | TTAAGACCAATGACTTACAAGGCAGCTGTAGATCTTAGCCACTTTTTAAAAGAA | |
| | | AAGGGGGGACTGGAAGGGCTAATTCACTCCCAACGAAGATAAGATCTGCTTTTT | |
| | | GCTTGTACTGGGTCTCTCTGGTTAGACCAGATCTGAGCCTGGGAGCTCTCTGGC | |
| | | TAACTAGGGAACCCACTGCTTAAGCCTCAATAAAGCTTGCCTTGAGTGCTTCAA | |
| | | GTAGTGTGTGCCCGTCTGTTGTGTGACTCTGGTAACTAGAGATCCCTCAGACCC | |
| | | TTTTAGTCAGTGTGGAAAATCTCTAGCAGTAGTAGTTCATGTCATCTTATTATT | |
| | | CAGTATTTATAACTTGCAAAGAAATGAATATCAGAGAGTGAGAGGAACTTGTTT | |
| | | ATTGCAGCTTATAATGGTTACAAATAAAGCAATAGCATCACAAATTTCACAAAT | |
| | | AAAGCATTTTTTTCACTGCATTCTAGTTGTGGTTTGTCCAAACTCATCAATGTA | |
| | | TCTTATCATGTCTGGCTCTAGCTATCCCGCCCCTAACTCCGCCCATCCCGCCCC | |
| | | TAACTCCGCCCAGTTCCGCCCATTCTCCGCCCCATGGCTGACTAATTTTTTTTA | |
| | | TTTATGCAGAGGCCGAGGCCGCCTCGGCCTCTGAGCTATTCCAGAAGTAGTGAG | |
| | | GAGGCTTTTTTGGAGGCCTAGACTTTTGCAGAGACCAAATTCGTAATCATGTCA | |
| | | TAGCTGTTTCCTGTGTGAAATTGTTATCCGCTCACAATTCCACACAACATACGA | |
| | | GCCGGAAGCATAAAGTGTAAAGCCTGGGGTGCCTAATGAGTGAGCTAACTCACA | |
| | | TTAATTGCGTTGCGCTCACTGCCCGCTTTCCAGTCGGGAAACCTGTCGTGCCAG | |
| | | CTGCATTAATGAATCGGCCAACGCGCGGGGAGAGGCGGTTTGCGTATTGGGCGC | |
| | | TCTTCCGCTTCCTCGCTCACTGACTCGCTGCGCTCGGTCGTTCGGCTGCGGCGA | |
| | | GCGGTATCAGCTCACTCAAAGGCGGTAATACGGTTATCCACAGAATCAGGGGAT | |
| | | AACGCAGGAAAGAACATGTGAGCAAAAGGCCAGCAAAAGGCCAGGAACCGTAAA | |
| | | AAGGCCGCGTTGCTGGCGTTTTTCCATAGGCTCCGCCCCCCTGACGAGCATCAC | |
| | | AAAAATCGACGCTCAAGTCAGAGGTGGCGAAACCCGACAGGACTATAAAGATAC | |
| | | CAGGCGTTTCCCCCTGGAAGCTCCCTCGTGCGCTCTCCTGTTCCGACCCTGCCG | |
| | | CTTACCGGATACCTGTCCGCCTTTCTCCCTTCGGGAAGCGTGGCGCTTTCTCAT | |
| | | AGCTCACGCTGTAGGTATCTCAGTTCGGTGTAGGTCGTTCGCTCCAAGCTGGGC | |
| | | TGTGTGCACGAACCCCC | |
| B08 | VH | GAGGTGCAGCTGTTGGAGTCTGGGGGAGGCTTGGTACAGCCTGGGGGGTCCCTG | 73 |
| | | AGACTCTCCTGTGCAGCCTCTGGATTCACCTTTAGCAGCTATGCCATGAGCTGG | |
| | | GTCCGCCAGGCTCCAGGGAAGGGGCTGGAGTGGGTCTCAGGTATTAGTGAAGGT | |
| | | GGTGGTAGCACATACTACGCAGACTCCGTGAAGGGCCGGTTCACCATCTCCAGA | |
| | | GACAATTCCAAGAACACGCTGTATCTGCAAATGAACAGCCTGAGAGCCGAGGAC | |
| | | ACGGCCGTATATTACTGTGCGAAAGATTACTACGGAGGGTGGTTCGACCCCTGG | |
| | | GGCCGTGGCACCCTGGTCACTGTCTCGAGT | |
| | VL | GAAATTGTGTTGACGCAGTCTCCAGGCACCCTGTCTTTGTCTCCAGGGGAAAGA | 74 |
| | | GCCACCCTCTCCTGCAGGGCCAGTCAGAGTGTTAGCAGCAGCTACTTAGCCTGG | |
| | | TACCAGCAGAAACCTGGCCAGGCTCCCAGGCTCCTCATCTATGGTGCATCCAGC | |
| | | AGGGCCACTGGCATCCCAGACAGGTTCAGTGGCAGTGGGTCTGGGACAGACTTC | |
| | | ACTCTCACCATCAGCAGACTGGAGCCTGAAGATTTTGCAGTGTATTACTGTCAG | |
| | | CAGTATGGTAGCTCACCTCGAACTTTTGGCCAGGGGACACGACTGGAGATTAAA | |
| | | CGGACC | |
| | scFv | GAGGTGCAGCTGTTGGAGTCTGGGGGAGGCTTGGTACAGCCTGGGGGGTCCCTG | 75 |
| | | AGACTCTCCTGTGCAGCCTCTGGATTCACCTTTAGCAGCTATGCCATGAGCTGG | |
| | | GTCCGCCAGGCTCCAGGGAAGGGGCTGGAGTGGGTCTCAGGTATTAGTGAAGGT | |
| | | GGTGGTAGCACATACTACGCAGACTCCGTGAAGGGCCGGTTCACCATCTCCAGA | |
| | | GACAATTCCAAGAACACGCTGTATCTGCAAATGAACAGCCTGAGAGCCGAGGAC | |
| | | ACGGCCGTATATTACTGTGCGAAAGATTACTACGGAGGGTGGTTCGACCCCTGG | |
| | | GGCCGTGGCACCCTGGTCACTGTCTCGAGTGGTGGAGGCGGTTCAGGCGGAGGT | |
| | | GGCTCTGGCGGTGGCGCTAGCGAAATTGTGTTGACGCAGTCTCCAGGCACCCTG | |
| | | TCTTTGTCTCCAGGGGAAAGAGCCACCCTCTCCTGCAGGGCCAGTCAGAGTGTT | |
| | | AGCAGCAGCTACTTAGCCTGGTACCAGCAGAAACCTGGCCAGGCTCCCAGGCTC | |
| | | CTCATCTATGGTGCATCCAGCAGGGCCACTGGCATCCCAGACAGGTTCAGTGGC | |
| | | AGTGGGTCTGGGACAGACTTCACTCTCACCATCAGCAGACTGGAGCCTGAAGAT | |
| | | TTTGCAGTGTATTACTGTCAGCAGTATGGTAGCTCACCTCGAACTTTTGGCCAG | |
| | | GGGACACGACTGGAGATTAAACGGACC | |
| | CAR | GAGGTGCAGCTGTTGGAGTCTGGGGGAGGCTTGGTACAGCCTGGGGGGTCCCTG | 76 |
| | | AGACTCTCCTGTGCAGCCTCTGGATTCACCTTTAGCAGCTATGCCATGAGCTGG | |
| | | GTCCGCCAGGCTCCAGGGAAGGGGCTGGAGTGGGTCTCAGGTATTAGTGAAGGT | |
| | | GGTGGTAGCACATACTACGCAGACTCCGTGAAGGGCCGGTTCACCATCTCCAGA | |
| | | GACAATTCCAAGAACACGCTGTATCTGCAAATGAACAGCCTGAGAGCCGAGGAC | |
| | | ACGGCCGTATATTACTGTGCGAAAGATTACTACGGAGGGTGGTTCGACCCCTGG | |
| | | GGCCGTGGCACCCTGGTCACTGTCTCGAGTGGTGGAGGCGGTTCAGGCGGAGGT | |
| | | GGCTCTGGCGGTGGCGCTAGCGAAATTGTGTTGACGCAGTCTCCAGGCACCCTG | |
| | | TCTTTGTCTCCAGGGGAAAGAGCCACCCTCTCCTGCAGGGCCAGTCAGAGTGTT | |
| | | AGCAGCAGCTACTTAGCCTGGTACCAGCAGAAACCTGGCCAGGCTCCCAGGCTC | |
| | | CTCATCTATGGTGCATCCAGCAGGGCCACTGGCATCCCAGACAGGTTCAGTGGC | |
| | | AGTGGGTCTGGGACAGACTTCACTCTCACCATCAGCAGACTGGAGCCTGAAGAT | |
| | | TTTGCAGTGTATTACTGTCAGCAGTATGGTAGCTCACCTCGAACTTTTGGCCAG | |
| | | GGGACACGACTGGAGATTAAACGGACCGCGGCCGCAACCACAACCCCTGCTCCA | |
| | | AGGCCCCCCACACCCGCTCCAACTATAGCCAGCCAACCATTGAGCCTCAGACCT | |
| | | GAAGCTTGCAGGCCCGCAGCAGGAGGCGCCGTCCATACGCGAGGCCTGGACTTC | |
| | | GCGTGTGATATTTATATTTGGGCCCCTTTGGCCGGAACATGTGGGGTGTTGCTT | |
| | | CTCTCCCTTGTGATCACTCTGTATTGTAAGCGCGGGAGAAAGAAGCTCCTGTAC | |
| | | ATCTTCAAGCAGCCTTTTATGCGACCTGTGCAAACCACTCAGGAAGAAGATGGG | |
| | | TGTTCATGCCGCTTCCCCGAGGAGGAAGAAGGAGGGTGTGAACTGAGGGTGAAA | |
| | | TTTTCTAGAAGCGCCGATGCTCCCGCATATCAGCAGGGTCAGAATCAGCTCTAC | |
| | | AATGAATTGAATCTCGGCAGGCGAGAAGAGTACGATGTTCTGGACAAGAGACGG | |
| | | GGCAGGGATCCCGAGATGGGGGGAAAGCCCCGGAGAAAAAATCCTCAGGAGGGG | |
| | | TTGTACAATGAGCTGCAGAAGGACAAGATGGCTGAAGCCTATAGCGAGATCGGA | |
| | | ATGAAAGGCGAAAGACGCAGAGGCAAGGGGCATGACGGTCTGTACCAGGGTCTC | |
| | | TCTACAGCCACCAAGGACACTTATGATGCGTTGCATATGCAAGCCTTGCCACCC | |
| | | CGC | |
| | Lentiviral | CGTTCAGCCCGACCGCTGCGCCTTATCCGGTAACTATCGTCTTGAGTCCAACCC | 77 |
| | vector | GGTAAGACACGACTTATCGCCACTGGCAGCAGCCACTGGTAACAGGATTAGCAG | |
| | | AGCGAGGTATGTAGGCGGTGCTACAGAGTTCTTGAAGTGGTGGCCTAACTACGG | |
| | | CTACACTAGAAGAACAGTATTTGGTATCTGCGCTCTGCTGAAGCCAGTTACCTT | |
| | | CGGAAAAAGAGTTGGTAGCTCTTGATCCGGCAAACAAACCACCGCTGGTAGCGG | |
| | | TGGTTTTTTTGTTTGCAAGCAGCAGATTACGCGCAGAAAAAAAGGATCTCAAGA | |
| | | AGATCCTTTGATCTTTTCTACGGGGTCTGACGCTCAGTGGAACGAAAACTCACG | |
| | | TTAAGGGATTTTGGTCATGAGATTATCAAAAAGGATCTTCACCTAGATCCTTTT | |
| | | AAATTAAAAATGAAGTTTTAAATCAATCTAAAGTATATATGAGTAAACTTGGTC | |
| | | TGACAGTTAGAAAAACTCATCGAGCATCAAATGAAACTGCAATTTATTCATATC | |
| | | AGGATTATCAATACCATATTTTTGAAAAAGCCGTTTCTGTAATGAAGGAGAAAA | |
| | | CTCACCGAGGCAGTTCCATAGGATGGCAAGATCCTGGTATCGGTCTGCGATTCC | |
| | | GACTCGTCCAACATCAATACAACCTATTAATTTCCCCTCGTCAAAAATAAGGTT | |
| | | ATCAAGTGAGAAATCACCATGAGTGACGACTGAATCCGGTGAGAATGGCAAAAG | |
| | | TTTATGCATTTCTTTCCAGACTTGTTCAACAGGCCAGCCATTACGCTCGTCATC | |
| | | AAAATCACTCGCATCAACCAAACCGTTATTCATTCGTGATTGCGCCTGAGCGAG | |
| | | ACGAAATACGCGATCGCTGTTAAAAGGACAATTACAAACAGGAATCGAATGCAA | |
| | | CCGGCGCAGGAACACTGCCAGCGCATCAACAATATTTTCACCTGAATCAGGATA | |
| | | TTCTTCTAATACCTGGAATGCTGTTTTTCCGGGGATCGCAGTGGTGAGTAACCA | |
| | | TGCATCATCAGGAGTACGGATAAAATGCTTGATGGTCGGAAGAGGCATAAATTC | |
| | | CGTCAGCCAGTTTAGTCTGACCATCTCATCTGTAACATCATTGGCAACGCTACC | |
| | | TTTGCCATGTTTCAGAAACAACTCTGGCGCATCGGGCTTCCCATACAAGCGATA | |
| | | GATTGTCGCACCTGATTGCCCGACATTATCGCGAGCCCATTTATACCCATATAA | |
| | | ATCAGCATCCATGTTGGAATTTAATCGCGGCCTCGACGTTTCCCGTTGAATATG | |
| | | GCTCATACTCTTCCTTTTTCAATATTATTGAAGCATTTATCAGGGTTATTGTCT | |
| | | CATGAGCGGATACATATTTGAATGTATTTAGAAAAATAAACAAATAGGGGTTCC | |
| | | GCGCACATTTCCCCGAAAAGTGCCACCTGACGGATCGGGAGATCTCCCGATCCC | |
| | | CTATGGTGCACTCTCAGTACAATCTGCTCTGATGCCGCATAGTTAAGCCAGTAT | |
| | | CTGCTCCCTGCTTGTGTGTTGGAGGTCGCTGAGTAGTGCGCGAGCAAAATTTAA | |
| | | GCTACAACAAGGCAAGGCTTGACCGACAATTGCATGAAGAATCTGCTTAGGGTT | |
| | | AGGCGTTTTGCGCTGCTTCGCGATGTACGGGCCAGATATACCCGTTGACATTGA | |
| | | TTATTGACTAGTTATTAATAGTAATCAATTACGGGGTCATTAGTTCATAGCCCA | |
| | | TATATGGAGTTCCGCGTTACATAACTTACGGTAAATGGCCCGCCTGGCTGACCG | |
| | | CCCAACGACCCCCGCCCATTGACGTCAATAATGACGTATGTTCCCATAGTAACG | |
| | | CCAATAGGGACTTTCCATTGACGTCAATGGGTGGAGTATTTACGGTAAACTGCC | |
| | | CACTTGGCAGTACATCAAGTGTATCATATGCCAAGTACGCCCCCTATTGACGTC | |
| | | AATGACGGTAAATGGCCCGCCTGGCATTATGCCCAGTACATGACCTTATGGGAC | |
| | | TTTCCTACTTGGCAGTACATCTACGTATTAGTCATCGCTATTACCATGGTGATG | |
| | | CGGTTTTGGCAGTACATCAATGGGCGTGGATAGCGGTTTGACTCACGGGGATTT | |
| | | CCAAGTCTCCACCCCATTGACGTCAATGGGAGTTTGTTTTGGCACCAAAATCAA | |
| | | CGGGACTTTCCAAAATGTCGTAACAACTCCGCCCCATTGACGCAAATGGGCGGT | |
| | | AGGCGTGTACGGTGGGAGGTCTATATAAGCAGCGCGTTTTGCCTGTACTGGGTC | |
| | | TCTCTGGTTAGACCAGATCTGAGCCTGGGAGCTCTCTGGCTAACTAGGGAACCC | |
| | | ACTGCTTAAGCCTCAATAAAGCTTGCCTTGAGTGCTTCAAGTAGTGTGTGCCCG | |
| | | TCTGTTGTGTGACTCTGGTAACTAGAGATCCCTCAGACCCTTTTAGTCAGTGTG | |
| | | GAAAATCTCTAGCAGTGGCGCCCGAACAGGGACTTGAAAGCGAAAGGGAAACCA | |
| | | GAGGAGCTCTCTCGACGCAGGACTCGGCTTGCTGAAGCGCGCACGGCAAGAGGC | |
| | | GAGGGGCGGCGACTGGTGAGTACGCCAAAAATTTTGACTAGCGGAGGCTAGAAG | |
| | | GAGAGAGATGGGTGCGAGAGCGTCAGTATTAAGCGGGGGAGAATTAGATCGCGA | |
| | | TGGGAAAAAATTCGGTTAAGGCCAGGGGGAAAGAAAAAATATAAATTAAAACAT | |
| | | ATAGTATGGGCAAGCAGGGAGCTAGAACGATTCGCAGTTAATCCTGGCCTGTTA | |
| | | GAAACATCAGAAGGCTGTAGACAAATACTGGGACAGCTACAACCATCCCTTCAG | |
| | | ACAGGATCAGAAGAACTTAGATCATTATATAATACAGTAGCAACCCTCTATTGT | |
| | | GTGCATCAAAGGATAGAGATAAAAGACACCAAGGAAGCTTTAGACAAGATAGAG | |
| | | GAAGAGCAAAACAAAAGTAAGACCACCGCACAGCAAGCGGCCGCTGATCTTCAG | |
| | | ACCTGGAGGAGGAGATATGAGGGACAATTGGAGAAGTGAATTATATAAATATAA | |
| | | AGTAGTAAAAATTGAACCATTAGGAGTAGCACCCACCAAGGCAAAGAGAAGAGT | |
| | | GGTGCAGAGAGAAAAAAGAGCAGTGGGAATAGGAGCTTTGTTCCTTGGGTTCTT | |
| | | GGGAGCAGCAGGAAGCACTATGGGCGCAGCGTCAATGACGCTGACGGTACAGGC | |
| | | CAGACAATTATTGTCTGGTATAGTGCAGCAGCAGAACAATTTGCTGAGGGCTAT | |
| | | TGAGGCGCAACAGCATCTGTTGCAACTCACAGTCTGGGGCATCAAGCAGCTCCA | |
| | | GGCAAGAATCCTGGCTGTGGAAAGATACCTAAAGGATCAACAGCTCCTGGGGAT | |
| | | TTGGGGTTGCTCTGGAAAACTCATTTGCACCACTGCTGTGCCTTGGAATGCTAG | |
| | | TTGGAGTAATAAATCTCTGGAACAGATTTGGAATCACACGACCTGGATGGAGTG | |
| | | GGACAGAGAAATTAACAATTACACAAGCTTAATACACTCCTTAATTGAAGAATC | |
| | | GCAAAACCAGCAAGAAAAGAATGAACAAGAATTATTGGAATTAGATAAATGGGC | |
| | | AAGTTTGTGGAATTGGTTTAACATAACAAATTGGCTGTGGTATATAAAATTATT | |
| | | CATAATGATAGTAGGAGGCTTGGTAGGTTTAAGAATAGTTTTTGCTGTACTTTC | |
| | | TATAGTGAATAGAGTTAGGCAGGGATATTCACCATTATCGTTTCAGACCCACCT | |
| | | CCCAACCCCGAGGGGACCCGACAGGCCCGAAGGAATAGAAGAAGAAGGTGGAGA | |
| | | GAGAGACAGAGACAGATCCATTCGATTAGTGAACGGATCTCGACGGTATCGGTT | |
| | | AACTTTTAAAAGAAAAGGGGGGATTGGGGGGTACAGTGCAGGGGAAAGAATAGT | |
| | | AGACATAATAGCAACAGACATACAAACTAAAGAATTACAAAAACAAATTACAAA | |
| | | ATTCAAAATTTTATCGATACTAGTATTATGCCCAGTACATGACCTTATGGGACT | |
| | | TTCCTACTTGGCAGTACATCTACAAACCCCGGGTGCAAAGATGGATAAAGTTTT | |
| | | AAACAGAGAGGAATCTTTGCAGCTAATGGACCTTCTAGGTCTTGAAAGGAGTGG | |
| | | GAATTGGCTCCGGTGCCCGTCAGTGGGCAGAGCGCACATCGCCCACAGTCCCCG | |
| | | AGAAGTTGGGGGGAGGGGTCGGCAATTGATCCGGTGCCTAGAGAAGGTGGCGCG | |
| | | GGGTAAACTGGGAAAGTGATGTCGTGTACTGGCTCCGCCTTTTTCCCGAGGGTG | |
| | | GGGGAGAACCGTATATAAGTGCAGTAGTCGCCGTGAACGTTCTTTTTCGCAACG | |
| | | GGTTTGCCGCCAGAACACAGGTAAGTGCCGTGTGTGGTTCCCGCGGGCCTGGCC | |
| | | TCTTTACGGGTTATGGCCCTTGCGTGCCTTGAATTACTTCCACTGGCTGCAGTA | |
| | | CGTGATTCTTGATCCCGAGCTTCGGGTTGGAAGTGGGTGGGAGAGTTCGAGGCC | |
| | | TTGCGCTTAAGGAGCCCCTTCGCCTCGTGCTTGAGTTGAGGCCTGGCCTGGGCG | |
| | | CTGGGGCCGCCGCGTGCGAATCTGGTGGCACCTTCGCGCCTGTCTCGCTGCTTT | |
| | | CGATAAGTCTCTAGCCATTTAAAATTTTTGATGACCTGCTGCGACGCTTTTTTT | |
| | | CTGGCAAGATAGTCTTGTAAATGCGGGCCAAGATCTGCACACTGGTATTTCGGT | |
| | | TTTTGGGGCCGCGGGCGGCGACGGGGCCCGTGCGTCCCAGCGCACATGTTCGGC | |
| | | GAGGCGGGGCCTGCGAGCGCGGCCACCGAGAATCGGACGGGGGTAGTCTCAAGC | |
| | | TGGCCGGCCTGCTCTGGTGCCTGGCCTCGCGCCGCCGTGTATCGCCCCGCCCTG | |
| | | GGCGGCAAGGCTGGCCCGGTCGGCACCAGTTGCGTGAGCGGAAAGATGGCCGCT | |
| | | TCCCGGCCCTGCTGCAGGGAGCTCAAAATGGAGGACGCGGCGCTCGGGAGAGCG | |
| | | GGCGGGTGAGTCACCCACACAAAGGAAAAGGGCCTTTCCGTCCTCAGCCGTCGC | |
| | | TTCATGTGACTCCACGGAGTACCGGGCGCCGTCCAGGCACCTCGATTAGTTCTC | |
| | | GAGCTTTTGGAGTACGTCGTCTTTAGGTTGGGGGGAGGGGTTTTATGCGATGGA | |
| | | GTTTCCCCACACTGAGTGGGTGGAGACTGAAGTTAGGCCAGCTTGGCACTTGAT | |
| | | GTAATTCTCCTTGGAATTTGCCCTTTTTGAGTTTGGATCTTGGTTCATTCTCAA | |
| | | GCCTCAGACAGTGGTTCAAAGTTTTTTTCTTCCATTTCAGGTGTCGTGACGTAG | |
| | | CGGCCCTAATACGACTCACTATAGGGCCCGCGTACGTCTAGACCTCCAGGCATC | |
| | | GATTCCACCATGGCACTCCCCGTCACCGCCCTTCTCTTGCCCCTCGCCCTGCTG | |
| | | CTGCATGCTGCCAGGCCCGAGGTGCAGCTGTTGGAGTCTGGGGGAGGCTTGGTA | |
| | | CAGCCTGGGGGGTCCCTGAGACTCTCCTGTGCAGCCTCTGGATTCACCTTTAGC | |
| | | AGCTATGCCATGAGCTGGGTCCGCCAGGCTCCAGGGAAGGGGCTGGAGTGGGTC | |
| | | TCAGGTATTAGTGAAGGTGGTGGTAGCACATACTACGCAGACTCCGTGAAGGGC | |
| | | CGGTTCACCATCTCCAGAGACAATTCCAAGAACACGCTGTATCTGCAAATGAAC | |
| | | AGCCTGAGAGCCGAGGACACGGCCGTATATTACTGTGCGAAAGATTACTACGGA | |
| | | GGGTGGTTCGACCCCTGGGGCCGTGGCACCCTGGTCACTGTCTCGAGTGGTGGA | |
| | | GGCGGTTCAGGCGGAGGTGGCTCTGGCGGTGGCGCTAGCGAAATTGTGTTGACG | |
| | | CAGTCTCCAGGCACCCTGTCTTTGTCTCCAGGGGAAAGAGCCACCCTCTCCTGC | |
| | | AGGGCCAGTCAGAGTGTTAGCAGCAGCTACTTAGCCTGGTACCAGCAGAAACCT | |
| | | GGCCAGGCTCCCAGGCTCCTCATCTATGGTGCATCCAGCAGGGCCACTGGCATC | |
| | | CCAGACAGGTTCAGTGGCAGTGGGTCTGGGACAGACTTCACTCTCACCATCAGC | |
| | | AGACTGGAGCCTGAAGATTTTGCAGTGTATTACTGTCAGCAGTATGGTAGCTCA | |
| | | CCTCGAACTTTTGGCCAGGGGACACGACTGGAGATTAAACGGACCGCGGCCGCA | |
| | | ACCACAACCCCTGCTCCAAGGCCCCCCACACCCGCTCCAACTATAGCCAGCCAA | |
| | | CCATTGAGCCTCAGACCTGAAGCTTGCAGGCCCGCAGCAGGAGGCGCCGTCCAT | |
| | | ACGCGAGGCCTGGACTTCGCGTGTGATATTTATATTTGGGCCCCTTTGGCCGGA | |
| | | ACATGTGGGGTGTTGCTTCTCTCCCTTGTGATCACTCTGTATTGTAAGCGCGGG | |
| | | AGAAAGAAGCTCCTGTACATCTTCAAGCAGCCTTTTATGCGACCTGTGCAAACC | |
| | | ACTCAGGAAGAAGATGGGTGTTCATGCCGCTTCCCCGAGGAGGAAGAAGGAGGG | |
| | | TGTGAACTGAGGGTGAAATTTTCTAGAAGCGCCGATGCTCCCGCATATCAGCAG | |
| | | GGTCAGAATCAGCTCTACAATGAATTGAATCTCGGCAGGCGAGAAGAGTACGAT | |
| | | GTTCTGGACAAGAGACGGGGCAGGGATCCCGAGATGGGGGGAAAGCCCCGGAGA | |
| | | AAAAATCCTCAGGAGGGGTTGTACAATGAGCTGCAGAAGGACAAGATGGCTGAA | |
| | | GCCTATAGCGAGATCGGAATGAAAGGCGAAAGACGCAGAGGCAAGGGGCATGAC | |
| | | GGTCTGTACCAGGGTCTCTCTACAGCCACCAAGGACACTTATGATGCGTTGCAT | |
| | | ATGCAAGCCTTGCCACCCCGCGGATCAGGAGCGACTAACTTCTCTTTGTTGAAG | |
| | | CAAGCGGGTGACGTTGAAGAGAATCCCGGTCCATCCGGGGGATCCATGCGGATC | |
| | | TCCAAGCCCCACCTCCGAAGCATCAGTATCCAGTGTTATCTGTGCCTTCTTCTG | |
| | | AATTCCCATTTTTTGACAGAGGCAGGAATTCATGTATTCATATTGGGTTGTTTC | |
| | | TCCGCGGGGCTTCCTAAAACCGAAGCTAACTGGGTTAATGTCATTTCAGATCTG | |
| | | AAGAAAATTGAAGATCTTATTCAATCAATGCATATCGACGCGACCTTGTACACG | |
| | | GAGAGTGATGTCCACCCGAGCTGCAAGGTAACGGCCATGAAATGCTTCTTGCTG | |
| | | GAATTGCAGGTAATTAGTCTTGAGAGTGGAGATGCGTCTATACACGACACCGTA | |
| | | GAGAATCTCATTATTTTGGCGAACAATTCTCTCAGCAGTAACGGCAATGTTACT | |
| | | GAATCAGGATGTAAGGAGTGTGAAGAGCTGGAAGAAAAGAACATTAAGGAATTT | |
| | | TTGCAGAGCTTCGTACATATCGTCCAAATGTTCATAAATACAAGCTAAGTCGAC | |
| | | GGCCGGCCGCGAGCATCTTACCGCCATTTATACCCATATTTGTTCTGTTTTTCT | |
| | | TGATTTGGGTATACATTTAAATGTTAATAAAACAAAATGGTGGGGCAATCATTT | |
| | | ACATTTTATAGGATATGTAATTACTAGTTCAGGTGTATTGCCACAAGACAAACA | |
| | | TCTTAAGAAACTTTCCCGTTATTTACGCTCTGTTCCTGTTAATCAACCTCTGGA | |
| | | TTACAAAATTTGTGAAAGATTGACTGATATTCTTAACTATGTTGCTCCTTTTAC | |
| | | GCTGTGTGGATATGCTGCTTTAATACCTCTGTATCATACTATTGCTTCCTGTAC | |
| | | GGCTTTCGTTTTCTCCTCCTTGTATAAATCCTGGTTGCTGTCTCTTTATCAGGA | |
| | | GTTGTGGCCCGTTGTCCGTCAACGTGGCGTGGTGTGCTCTGTGTTTGCTGACGC | |
| | | AACCCCCACTGGCTGGGGCATTGCCACCACCTGTCAACTCCTTTCTGGGACTTT | |
| | | CGCTTTCCCCCTCCCGATCGCCACGGCAGAACTCATCGCCGCCTGCCTTGCCCG | |
| | | CTGCTGGACAGGGGCTAGGTTGCTGGGCACTGATAATTCCGTGGTGTTGTCCTG | |
| | | GTACCTTTAAGACCAATGACTTACAAGGCAGCTGTAGATCTTAGCCACTTTTTA | |
| | | AAAGAAAAGGGGGGACTGGAAGGGCTAATTCACTCCCAACGAAGATAAGATCTG | |
| | | CTTTTTGCTTGTACTGGGTCTCTCTGGTTAGACCAGATCTGAGCCTGGGAGCTC | |
| | | TCTGGCTAACTAGGGAACCCACTGCTTAAGCCTCAATAAAGCTTGCCTTGAGTG | |
| | | CTTCAAGTAGTGTGTGCCCGTCTGTTGTGTGACTCTGGTAACTAGAGATCCCTC | |
| | | AGACCCTTTTAGTCAGTGTGGAAAATCTCTAGCAGTAGTAGTTCATGTCATCTT | |
| | | ATTATTCAGTATTTATAACTTGCAAAGAAATGAATATCAGAGAGTGAGAGGAAC | |
| | | TTGTTTATTGCAGCTTATAATGGTTACAAATAAAGCAATAGCATCACAAATTTC | |
| | | ACAAATAAAGCATTTTTTTCACTGCATTCTAGTTGTGGTTTGTCCAAACTCATC | |
| | | AATGTATCTTATCATGTCTGGCTCTAGCTATCCCGCCCCTAACTCCGCCCATCC | |
| | | CGCCCCTAACTCCGCCCAGTTCCGCCCATTCTCCGCCCCATGGCTGACTAATTT | |
| | | TTTTTATTTATGCAGAGGCCGAGGCCGCCTCGGCCTCTGAGCTATTCCAGAAGT | |
| | | AGTGAGGAGGCTTTTTTGGAGGCCTAGACTTTTGCAGAGACCAAATTCGTAATC | |
| | | ATGTCATAGCTGTTTCCTGTGTGAAATTGTTATCCGCTCACAATTCCACACAAC | |
| | | ATACGAGCCGGAAGCATAAAGTGTAAAGCCTGGGGTGCCTAATGAGTGAGCTAA | |
| | | CTCACATTAATTGCGTTGCGCTCACTGCCCGCTTTCCAGTCGGGAAACCTGTCG | |
| | | TGCCAGCTGCATTAATGAATCGGCCAACGCGCGGGGAGAGGCGGTTTGCGTATT | |
| | | GGGCGCTCTTCCGCTTCCTCGCTCACTGACTCGCTGCGCTCGGTCGTTCGGCTG | |
| | | CGGCGAGCGGTATCAGCTCACTCAAAGGCGGTAATACGGTTATCCACAGAATCA | |
| | | GGGGATAACGCAGGAAAGAACATGTGAGCAAAAGGCCAGCAAAAGGCCAGGAAC | |
| | | CGTAAAAAGGCCGCGTTGCTGGCGTTTTTCCATAGGCTCCGCCCCCCTGACGAG | |
| | | CATCACAAAAATCGACGCTCAAGTCAGAGGTGGCGAAACCCGACAGGACTATAA | |
| | | AGATACCAGGCGTTTCCCCCTGGAAGCTCCCTCGTGCGCTCTCCTGTTCCGACC | |
| | | CTGCCGCTTACCGGATACCTGTCCGCCTTTCTCCCTTCGGGAAGCGTGGCGCTT | |
| | | TCTCATAGCTCACGCTGTAGGTATCTCAGTTCGGTGTAGGTCGTTCGCTCCAAG | |
| | | CTGGGCTGTGTGCACGAACCCCC | |
| H02 | VH | GAGGTGCAGCTGTTGGAGTCTGGGGGAGGCTTGGTACAGCCTGGGGGGTCCCTG | 78 |
| | | AGACTCTCCTGTGCAGCCTCTGGATTCACCTTTAGCAGCTATGCCATGAGCTGG | |
| | | GTCCGCCAGGCTCCAGGGAAGGGGCTGGAGTGGGTCTCAGCTATTAGTGGTAGT | |
| | | GGTGGTAGCACATACTACGCAGACTCCGTGAAGGGCCGGTTCACCATCTCCAGA | |
| | | GACAATTCCAAGAACACGCTGTATCTGCAAATGAACAGCCTGAGAGCCGAGGAC | |
| | | ACGGCCGTATATTACTGTGCGAAAGATCTCAGCTGGTACGGAGCCAACTTTGAC | |
| | | TACTGGGGCCAGGGCACCCTGGTCACCGTCTCGAGT | |
| | VL | GAAATTGTGTTGACGCAGTCTCCAGGCACCCTGTCTTTGTCTCCAGGGGAAAGA | 79 |
| | | GCCACCCTCTCCTGCAGGGCCAGTCAGAGTGTTAGCAGCAGCTACTTAGCCTGG | |
| | | TACCAGCAGAAACCTGGCCAGGCTCCCAGGCTCCTCATCTATGGTGCATCCAGC | |
| | | AGGGCCACTGGCATCCCAGACAGGTTCAGTGGCAGTGGGTCTGGGACAGACTTC | |
| | | ACTCTCACCATCAGCAGACTGGAGCCTGAAGATTTTGCAGTGTATTACTGTCAG | |
| | | CAGTATGGTAGCTCACCGGTCATGTACACTTTTGGCCAGGGGACCAAGGTGGAG | |
| | | ATCAAA | |
| | scFv | GAGGTGCAGCTGTTGGAGTCTGGGGGAGGCTTGGTACAGCCTGGGGGGTCCCTG | 80 |
| | | AGACTCTCCTGTGCAGCCTCTGGATTCACCTTTAGCAGCTATGCCATGAGCTGG | |
| | | GTCCGCCAGGCTCCAGGGAAGGGGCTGGAGTGGGTCTCAGCTATTAGTGGTAGT | |
| | | GGTGGTAGCACATACTACGCAGACTCCGTGAAGGGCCGGTTCACCATCTCCAGA | |
| | | GACAATTCCAAGAACACGCTGTATCTGCAAATGAACAGCCTGAGAGCCGAGGAC | |
| | | ACGGCCGTATATTACTGTGCGAAAGATCTCAGCTGGTACGGAGCCAACTTTGAC | |
| | | TACTGGGGCCAGGGCACCCTGGTCACCGTCTCGAGTGGTGGAGGCGGTTCAGGC | |
| | | GGAGGTGGCTCTGGCGGTGGCGCTAGCGAAATTGTGTTGACGCAGTCTCCAGGC | |
| | | ACCCTGTCTTTGTCTCCAGGGGAAAGAGCCACCCTCTCCTGCAGGGCCAGTCAG | |
| | | AGTGTTAGCAGCAGCTACTTAGCCTGGTACCAGCAGAAACCTGGCCAGGCTCCC | |
| | | AGGCTCCTCATCTATGGTGCATCCAGCAGGGCCACTGGCATCCCAGACAGGTTC | |
| | | AGTGGCAGTGGGTCTGGGACAGACTTCACTCTCACCATCAGCAGACTGGAGCCT | |
| | | GAAGATTTTGCAGTGTATTACTGTCAGCAGTATGGTAGCTCACCGGTCATGTAC | |
| | | ACTTTTGGCCAGGGGACCAAGGTGGAGATCAAA | |
| | CAR | GAGGTGCAGCTGTTGGAGTCTGGGGGAGGCTTGGTACAGCCTGGGGGGTCCCTG | 81 |
| | | AGACTCTCCTGTGCAGCCTCTGGATTCACCTTTAGCAGCTATGCCATGAGCTGG | |
| | | GTCCGCCAGGCTCCAGGGAAGGGGCTGGAGTGGGTCTCAGCTATTAGTGGTAGT | |
| | | GGTGGTAGCACATACTACGCAGACTCCGTGAAGGGCCGGTTCACCATCTCCAGA | |
| | | GACAATTCCAAGAACACGCTGTATCTGCAAATGAACAGCCTGAGAGCCGAGGAC | |
| | | ACGGCCGTATATTACTGTGCGAAAGATCTCAGCTGGTACGGAGCCAACTTTGAC | |
| | | TACTGGGGCCAGGGCACCCTGGTCACCGTCTCGAGTGGTGGAGGCGGTTCAGGC | |
| | | GGAGGTGGCTCTGGCGGTGGCGCTAGCGAAATTGTGTTGACGCAGTCTCCAGGC | |
| | | ACCCTGTCTTTGTCTCCAGGGGAAAGAGCCACCCTCTCCTGCAGGGCCAGTCAG | |
| | | AGTGTTAGCAGCAGCTACTTAGCCTGGTACCAGCAGAAACCTGGCCAGGCTCCC | |
| | | AGGCTCCTCATCTATGGTGCATCCAGCAGGGCCACTGGCATCCCAGACAGGTTC | |
| | | AGTGGCAGTGGGTCTGGGACAGACTTCACTCTCACCATCAGCAGACTGGAGCCT | |
| | | GAAGATTTTGCAGTGTATTACTGTCAGCAGTATGGTAGCTCACCGGTCATGTAC | |
| | | ACTTTTGGCCAGGGGACCAAGGTGGAGATCAAAGCGGCCGCAACCACAACCCCT | |
| | | GCTCCAAGGCCCCCCACACCCGCTCCAACTATAGCCAGCCAACCATTGAGCCTC | |
| | | AGACCTGAAGCTTGCAGGCCCGCAGCAGGAGGCGCCGTCCATACGCGAGGCCTG | |
| | | GACTTCGCGTGTGATATTTATATTTGGGCCCCTTTGGCCGGAACATGTGGGGTG | |
| | | TTGCTTCTCTCCCTTGTGATCACTCTGTATTGTAAGCGCGGGAGAAAGAAGCTC | |
| | | CTGTACATCTTCAAGCAGCCTTTTATGCGACCTGTGCAAACCACTCAGGAAGAA | |
| | | GATGGGTGTTCATGCCGCTTCCCCGAGGAGGAAGAAGGAGGGTGTGAACTGAGG | |
| | | GTGAAATTTTCTAGAAGCGCCGATGCTCCCGCATATCAGCAGGGTCAGAATCAG | |
| | | CTCTACAATGAATTGAATCTCGGCAGGCGAGAAGAGTACGATGTTCTGGACAAG | |
| | | AGACGGGGCAGGGATCCCGAGATGGGGGGAAAGCCCCGGAGAAAAAATCCTCAG | |
| | | GAGGGGTTGTACAATGAGCTGCAGAAGGACAAGATGGCTGAAGCCTATAGCGAG | |
| | | ATCGGAATGAAAGGCGAAAGACGCAGAGGCAAGGGGCATGACGGTCTGTACCAG | |
| | | GGTCTCTCTACAGCCACCAAGGACACTTATGATGCGTTGCATATGCAAGCCTTG | |
| | | CCACCCCGC | 82 |
| | Lentiviral | CGTTCAGCCCGACCGCTGCGCCTTATCCGGTAACTATCGTCTTGAGTCCAACCC | |
| | vector | GGTAAGACACGACTTATCGCCACTGGCAGCAGCCACTGGTAACAGGATTAGCAG | |
| | | AGCGAGGTATGTAGGCGGTGCTACAGAGTTCTTGAAGTGGTGGCCTAACTACGG | |
| | | CTACACTAGAAGAACAGTATTTGGTATCTGCGCTCTGCTGAAGCCAGTTACCTT | |
| | | CGGAAAAAGAGTTGGTAGCTCTTGATCCGGCAAACAAACCACCGCTGGTAGCGG | |
| | | TGGTTTTTTTGTTTGCAAGCAGCAGATTACGCGCAGAAAAAAAGGATCTCAAGA | |
| | | AGATCCTTTGATCTTTTCTACGGGGTCTGACGCTCAGTGGAACGAAAACTCACG | |
| | | TTAAGGGATTTTGGTCATGAGATTATCAAAAAGGATCTTCACCTAGATCCTTTT | |
| | | AAATTAAAAATGAAGTTTTAAATCAATCTAAAGTATATATGAGTAAACTTGGTC | |
| | | TGACAGTTAGAAAAACTCATCGAGCATCAAATGAAACTGCAATTTATTCATATC | |
| | | AGGATTATCAATACCATATTTTTGAAAAAGCCGTTTCTGTAATGAAGGAGAAAA | |
| | | CTCACCGAGGCAGTTCCATAGGATGGCAAGATCCTGGTATCGGTCTGCGATTCC | |
| | | GACTCGTCCAACATCAATACAACCTATTAATTTCCCCTCGTCAAAAATAAGGTT | |
| | | ATCAAGTGAGAAATCACCATGAGTGACGACTGAATCCGGTGAGAATGGCAAAAG | |
| | | TTTATGCATTTCTTTCCAGACTTGTTCAACAGGCCAGCCATTACGCTCGTCATC | |
| | | AAAATCACTCGCATCAACCAAACCGTTATTCATTCGTGATTGCGCCTGAGCGAG | |
| | | ACGAAATACGCGATCGCTGTTAAAAGGACAATTACAAACAGGAATCGAATGCAA | |
| | | CCGGCGCAGGAACACTGCCAGCGCATCAACAATATTTTCACCTGAATCAGGATA | |
| | | TTCTTCTAATACCTGGAATGCTGTTTTTCCGGGGATCGCAGTGGTGAGTAACCA | |
| | | TGCATCATCAGGAGTACGGATAAAATGCTTGATGGTCGGAAGAGGCATAAATTC | |
| | | CGTCAGCCAGTTTAGTCTGACCATCTCATCTGTAACATCATTGGCAACGCTACC | |
| | | TTTGCCATGTTTCAGAAACAACTCTGGCGCATCGGGCTTCCCATACAAGCGATA | |
| | | GATTGTCGCACCTGATTGCCCGACATTATCGCGAGCCCATTTATACCCATATAA | |
| | | ATCAGCATCCATGTTGGAATTTAATCGCGGCCTCGACGTTTCCCGTTGAATATG | |
| | | GCTCATACTCTTCCTTTTTCAATATTATTGAAGCATTTATCAGGGTTATTGTCT | |
| | | CATGAGCGGATACATATTTGAATGTATTTAGAAAAATAAACAAATAGGGGTTCC | |
| | | GCGCACATTTCCCCGAAAAGTGCCACCTGACGGATCGGGAGATCTCCCGATCCC | |
| | | CTATGGTGCACTCTCAGTACAATCTGCTCTGATGCCGCATAGTTAAGCCAGTAT | |
| | | CTGCTCCCTGCTTGTGTGTTGGAGGTCGCTGAGTAGTGCGCGAGCAAAATTTAA | |
| | | GCTACAACAAGGCAAGGCTTGACCGACAATTGCATGAAGAATCTGCTTAGGGTT | |
| | | AGGCGTTTTGCGCTGCTTCGCGATGTACGGGCCAGATATACCCGTTGACATTGA | |
| | | TTATTGACTAGTTATTAATAGTAATCAATTACGGGGTCATTAGTTCATAGCCCA | |
| | | TATATGGAGTTCCGCGTTACATAACTTACGGTAAATGGCCCGCCTGGCTGACCG | |
| | | CCCAACGACCCCCGCCCATTGACGTCAATAATGACGTATGTTCCCATAGTAACG | |
| | | CCAATAGGGACTTTCCATTGACGTCAATGGGTGGAGTATTTACGGTAAACTGCC | |
| | | CACTTGGCAGTACATCAAGTGTATCATATGCCAAGTACGCCCCCTATTGACGTC | |
| | | AATGACGGTAAATGGCCCGCCTGGCATTATGCCCAGTACATGACCTTATGGGAC | |
| | | TTTCCTACTTGGCAGTACATCTACGTATTAGTCATCGCTATTACCATGGTGATG | |
| | | CGGTTTTGGCAGTACATCAATGGGCGTGGATAGCGGTTTGACTCACGGGGATTT | |
| | | CCAAGTCTCCACCCCATTGACGTCAATGGGAGTTTGTTTTGGCACCAAAATCAA | |
| | | CGGGACTTTCCAAAATGTCGTAACAACTCCGCCCCATTGACGCAAATGGGCGGT | |
| | | AGGCGTGTACGGTGGGAGGTCTATATAAGCAGCGCGTTTTGCCTGTACTGGGTC | |
| | | TCTCTGGTTAGACCAGATCTGAGCCTGGGAGCTCTCTGGCTAACTAGGGAACCC | |
| | | ACTGCTTAAGCCTCAATAAAGCTTGCCTTGAGTGCTTCAAGTAGTGTGTGCCCG | |
| | | TCTGTTGTGTGACTCTGGTAACTAGAGATCCCTCAGACCCTTTTAGTCAGTGTG | |
| | | GAAAATCTCTAGCAGTGGCGCCCGAACAGGGACTTGAAAGCGAAAGGGAAACCA | |
| | | GAGGAGCTCTCTCGACGCAGGACTCGGCTTGCTGAAGCGCGCACGGCAAGAGGC | |
| | | GAGGGGCGGCGACTGGTGAGTACGCCAAAAATTTTGACTAGCGGAGGCTAGAAG | |
| | | GAGAGAGATGGGTGCGAGAGCGTCAGTATTAAGCGGGGGAGAATTAGATCGCGA | |
| | | TGGGAAAAAATTCGGTTAAGGCCAGGGGGAAAGAAAAAATATAAATTAAAACAT | |
| | | ATAGTATGGGCAAGCAGGGAGCTAGAACGATTCGCAGTTAATCCTGGCCTGTTA | |
| | | GAAACATCAGAAGGCTGTAGACAAATACTGGGACAGCTACAACCATCCCTTCAG | |
| | | ACAGGATCAGAAGAACTTAGATCATTATATAATACAGTAGCAACCCTCTATTGT | |
| | | GTGCATCAAAGGATAGAGATAAAAGACACCAAGGAAGCTTTAGACAAGATAGAG | |
| | | GAAGAGCAAAACAAAAGTAAGACCACCGCACAGCAAGCGGCCGCTGATCTTCAG | |
| | | ACCTGGAGGAGGAGATATGAGGGACAATTGGAGAAGTGAATTATATAAATATAA | |
| | | AGTAGTAAAAATTGAACCATTAGGAGTAGCACCCACCAAGGCAAAGAGAAGAGT | |
| | | GGTGCAGAGAGAAAAAAGAGCAGTGGGAATAGGAGCTTTGTTCCTTGGGTTCTT | |
| | | GGGAGCAGCAGGAAGCACTATGGGCGCAGCGTCAATGACGCTGACGGTACAGGC | |
| | | CAGACAATTATTGTCTGGTATAGTGCAGCAGCAGAACAATTTGCTGAGGGCTAT | |
| | | TGAGGCGCAACAGCATCTGTTGCAACTCACAGTCTGGGGCATCAAGCAGCTCCA | |
| | | GGCAAGAATCCTGGCTGTGGAAAGATACCTAAAGGATCAACAGCTCCTGGGGAT | |
| | | TTGGGGTTGCTCTGGAAAACTCATTTGCACCACTGCTGTGCCTTGGAATGCTAG | |
| | | TTGGAGTAATAAATCTCTGGAACAGATTTGGAATCACACGACCTGGATGGAGTG | |
| | | GGACAGAGAAATTAACAATTACACAAGCTTAATACACTCCTTAATTGAAGAATC | |
| | | GCAAAACCAGCAAGAAAAGAATGAACAAGAATTATTGGAATTAGATAAATGGGC | |
| | | AAGTTTGTGGAATTGGTTTAACATAACAAATTGGCTGTGGTATATAAAATTATT | |
| | | CATAATGATAGTAGGAGGCTTGGTAGGTTTAAGAATAGTTTTTGCTGTACTTTC | |
| | | TATAGTGAATAGAGTTAGGCAGGGATATTCACCATTATCGTTTCAGACCCACCT | |
| | | CCCAACCCCGAGGGGACCCGACAGGCCCGAAGGAATAGAAGAAGAAGGTGGAGA | |
| | | GAGAGACAGAGACAGATCCATTCGATTAGTGAACGGATCTCGACGGTATCGGTT | |
| | | AACTTTTAAAAGAAAAGGGGGGATTGGGGGGTACAGTGCAGGGGAAAGAATAGT | |
| | | AGACATAATAGCAACAGACATACAAACTAAAGAATTACAAAAACAAATTACAAA | |
| | | ATTCAAAATTTTATCGATACTAGTATTATGCCCAGTACATGACCTTATGGGACT | |
| | | TTCCTACTTGGCAGTACATCTACAAACCCCGGGTGCAAAGATGGATAAAGTTTT | |
| | | AAACAGAGAGGAATCTTTGCAGCTAATGGACCTTCTAGGTCTTGAAAGGAGTGG | |
| | | GAATTGGCTCCGGTGCCCGTCAGTGGGCAGAGCGCACATCGCCCACAGTCCCCG | |
| | | AGAAGTTGGGGGGAGGGGTCGGCAATTGATCCGGTGCCTAGAGAAGGTGGCGCG | |
| | | GGGTAAACTGGGAAAGTGATGTCGTGTACTGGCTCCGCCTTTTTCCCGAGGGTG | |
| | | GGGGAGAACCGTATATAAGTGCAGTAGTCGCCGTGAACGTTCTTTTTCGCAACG | |
| | | GGTTTGCCGCCAGAACACAGGTAAGTGCCGTGTGTGGTTCCCGCGGGCCTGGCC | |
| | | TCTTTACGGGTTATGGCCCTTGCGTGCCTTGAATTACTTCCACTGGCTGCAGTA | |
| | | CGTGATTCTTGATCCCGAGCTTCGGGTTGGAAGTGGGTGGGAGAGTTCGAGGCC | |
| | | TTGCGCTTAAGGAGCCCCTTCGCCTCGTGCTTGAGTTGAGGCCTGGCCTGGGCG | |
| | | CTGGGGCCGCCGCGTGCGAATCTGGTGGCACCTTCGCGCCTGTCTCGCTGCTTT | |
| | | CGATAAGTCTCTAGCCATTTAAAATTTTTGATGACCTGCTGCGACGCTTTTTTT | |
| | | CTGGCAAGATAGTCTTGTAAATGCGGGCCAAGATCTGCACACTGGTATTTCGGT | |
| | | TTTTGGGGCCGCGGGCGGCGACGGGGCCCGTGCGTCCCAGCGCACATGTTCGGC | |
| | | GAGGCGGGGCCTGCGAGCGCGGCCACCGAGAATCGGACGGGGGTAGTCTCAAGC | |
| | | TGGCCGGCCTGCTCTGGTGCCTGGCCTCGCGCCGCCGTGTATCGCCCCGCCCTG | |
| | | GGCGGCAAGGCTGGCCCGGTCGGCACCAGTTGCGTGAGCGGAAAGATGGCCGCT | |
| | | TCCCGGCCCTGCTGCAGGGAGCTCAAAATGGAGGACGCGGCGCTCGGGAGAGCG | |
| | | GGCGGGTGAGTCACCCACACAAAGGAAAAGGGCCTTTCCGTCCTCAGCCGTCGC | |
| | | TTCATGTGACTCCACGGAGTACCGGGCGCCGTCCAGGCACCTCGATTAGTTCTC | |
| | | GAGCTTTTGGAGTACGTCGTCTTTAGGTTGGGGGGAGGGGTTTTATGCGATGGA | |
| | | GTTTCCCCACACTGAGTGGGTGGAGACTGAAGTTAGGCCAGCTTGGCACTTGAT | |
| | | GTAATTCTCCTTGGAATTTGCCCTTTTTGAGTTTGGATCTTGGTTCATTCTCAA | |
| | | GCCTCAGACAGTGGTTCAAAGTTTTTTTCTTCCATTTCAGGTGTCGTGACGTAG | |
| | | CGGCCCTAATACGACTCACTATAGGGCCCGCGTACGTCTAGACCTCCAGGCATC | |
| | | GATTCCACCATGGCACTCCCCGTCACCGCCCTTCTCTTGCCCCTCGCCCTGCTG | |
| | | CTGCATGCTGCCAGGCCCGAGGTGCAGCTGTTGGAGTCTGGGGGAGGCTTGGTA | |
| | | CAGCCTGGGGGGTCCCTGAGACTCTCCTGTGCAGCCTCTGGATTCACCTTTAGC | |
| | | AGCTATGCCATGAGCTGGGTCCGCCAGGCTCCAGGGAAGGGGCTGGAGTGGGTC | |
| | | TCAGCTATTAGTGGTAGTGGTGGTAGCACATACTACGCAGACTCCGTGAAGGGC | |
| | | CGGTTCACCATCTCCAGAGACAATTCCAAGAACACGCTGTATCTGCAAATGAAC | |
| | | AGCCTGAGAGCCGAGGACACGGCCGTATATTACTGTGCGAAAGATCTCAGCTGG | |
| | | TACGGAGCCAACTTTGACTACTGGGGCCAGGGCACCCTGGTCACCGTCTCGAGT | |
| | | GGTGGAGGCGGTTCAGGCGGAGGTGGCTCTGGCGGTGGCGCTAGCGAAATTGTG | |
| | | TTGACGCAGTCTCCAGGCACCCTGTCTTTGTCTCCAGGGGAAAGAGCCACCCTC | |
| | | TCCTGCAGGGCCAGTCAGAGTGTTAGCAGCAGCTACTTAGCCTGGTACCAGCAG | |
| | | AAACCTGGCCAGGCTCCCAGGCTCCTCATCTATGGTGCATCCAGCAGGGCCACT | |
| | | GGCATCCCAGACAGGTTCAGTGGCAGTGGGTCTGGGACAGACTTCACTCTCACC | |
| | | ATCAGCAGACTGGAGCCTGAAGATTTTGCAGTGTATTACTGTCAGCAGTATGGT | |
| | | AGCTCACCGGTCATGTACACTTTTGGCCAGGGGACCAAGGTGGAGATCAAAGCG | |
| | | GCCGCAACCACAACCCCTGCTCCAAGGCCCCCCACACCCGCTCCAACTATAGCC | |
| | | AGCCAACCATTGAGCCTCAGACCTGAAGCTTGCAGGCCCGCAGCAGGAGGCGCC | |
| | | GTCCATACGCGAGGCCTGGACTTCGCGTGTGATATTTATATTTGGGCCCCTTTG | |
| | | GCCGGAACATGTGGGGTGTTGCTTCTCTCCCTTGTGATCACTCTGTATTGTAAG | |
| | | CGCGGGAGAAAGAAGCTCCTGTACATCTTCAAGCAGCCTTTTATGCGACCTGTG | |
| | | CAAACCACTCAGGAAGAAGATGGGTGTTCATGCCGCTTCCCCGAGGAGGAAGAA | |
| | | GGAGGGTGTGAACTGAGGGTGAAATTTTCTAGAAGCGCCGATGCTCCCGCATAT | |
| | | CAGCAGGGTCAGAATCAGCTCTACAATGAATTGAATCTCGGCAGGCGAGAAGAG | |
| | | TACGATGTTCTGGACAAGAGACGGGGCAGGGATCCCGAGATGGGGGGAAAGCCC | |
| | | CGGAGAAAAAATCCTCAGGAGGGGTTGTACAATGAGCTGCAGAAGGACAAGATG | |
| | | GCTGAAGCCTATAGCGAGATCGGAATGAAAGGCGAAAGACGCAGAGGCAAGGGG | |
| | | CATGACGGTCTGTACCAGGGTCTCTCTACAGCCACCAAGGACACTTATGATGCG | |
| | | TTGCATATGCAAGCCTTGCCACCCCGCGGATCAGGAGCGACTAACTTCTCTTTG | |
| | | TTGAAGCAAGCGGGTGACGTTGAAGAGAATCCCGGTCCATCCGGGGGATCCATG | |
| | | CGGATCTCCAAGCCCCACCTCCGAAGCATCAGTATCCAGTGTTATCTGTGCCTT | |
| | | CTTCTGAATTCCCATTTTTTGACAGAGGCAGGAATTCATGTATTCATATTGGGT | |
| | | TGTTTCTCCGCGGGGCTTCCTAAAACCGAAGCTAACTGGGTTAATGTCATTTCA | |
| | | GATCTGAAGAAAATTGAAGATCTTATTCAATCAATGCATATCGACGCGACCTTG | |
| | | TACACGGAGAGTGATGTCCACCCGAGCTGCAAGGTAACGGCCATGAAATGCTTC | |
| | | TTGCTGGAATTGCAGGTAATTAGTCTTGAGAGTGGAGATGCGTCTATACACGAC | |
| | | ACCGTAGAGAATCTCATTATTTTGGCGAACAATTCTCTCAGCAGTAACGGCAAT | |
| | | GTTACTGAATCAGGATGTAAGGAGTGTGAAGAGCTGGAAGAAAAGAACATTAAG | |
| | | GAATTTTTGCAGAGCTTCGTACATATCGTCCAAATGTTCATAAATACAAGCTAA | |
| | | GTCGACGGCCGGCCGCGAGCATCTTACCGCCATTTATACCCATATTTGTTCTGT | |
| | | TTTTCTTGATTTGGGTATACATTTAAATGTTAATAAAACAAAATGGTGGGGCAA | |
| | | TCATTTACATTTTATAGGATATGTAATTACTAGTTCAGGTGTATTGCCACAAGA | |
| | | CAAACATCTTAAGAAACTTTCCCGTTATTTACGCTCTGTTCCTGTTAATCAACC | |
| | | TCTGGATTACAAAATTTGTGAAAGATTGACTGATATTCTTAACTATGTTGCTCC | |
| | | TTTTACGCTGTGTGGATATGCTGCTTTAATACCTCTGTATCATACTATTGCTTC | |
| | | CTGTACGGCTTTCGTTTTCTCCTCCTTGTATAAATCCTGGTTGCTGTCTCTTTA | |
| | | TCAGGAGTTGTGGCCCGTTGTCCGTCAACGTGGCGTGGTGTGCTCTGTGTTTGC | |
| | | TGACGCAACCCCCACTGGCTGGGGCATTGCCACCACCTGTCAACTCCTTTCTGG | |
| | | GACTTTCGCTTTCCCCCTCCCGATCGCCACGGCAGAACTCATCGCCGCCTGCCT | |
| | | TGCCCGCTGCTGGACAGGGGCTAGGTTGCTGGGCACTGATAATTCCGTGGTGTT | |
| | | GTCCTGGTACCTTTAAGACCAATGACTTACAAGGCAGCTGTAGATCTTAGCCAC | |
| | | TTTTTAAAAGAAAAGGGGGGACTGGAAGGGCTAATTCACTCCCAACGAAGATAA | |
| | | GATCTGCTTTTTGCTTGTACTGGGTCTCTCTGGTTAGACCAGATCTGAGCCTGG | |
| | | GAGCTCTCTGGCTAACTAGGGAACCCACTGCTTAAGCCTCAATAAAGCTTGCCT | |
| | | TGAGTGCTTCAAGTAGTGTGTGCCCGTCTGTTGTGTGACTCTGGTAACTAGAGA | |
| | | TCCCTCAGACCCTTTTAGTCAGTGTGGAAAATCTCTAGCAGTAGTAGTTCATGT | |
| | | CATCTTATTATTCAGTATTTATAACTTGCAAAGAAATGAATATCAGAGAGTGAG | |
| | | AGGAACTTGTTTATTGCAGCTTATAATGGTTACAAATAAAGCAATAGCATCACA | |
| | | AATTTCACAAATAAAGCATTTTTTTCACTGCATTCTAGTTGTGGTTTGTCCAAA | |
| | | CTCATCAATGTATCTTATCATGTCTGGCTCTAGCTATCCCGCCCCTAACTCCGC | |
| | | CCATCCCGCCCCTAACTCCGCCCAGTTCCGCCCATTCTCCGCCCCATGGCTGAC | |
| | | TAATTTTTTTTATTTATGCAGAGGCCGAGGCCGCCTCGGCCTCTGAGCTATTCC | |
| | | AGAAGTAGTGAGGAGGCTTTTTTGGAGGCCTAGACTTTTGCAGAGACCAAATTC | |
| | | GTAATCATGTCATAGCTGTTTCCTGTGTGAAATTGTTATCCGCTCACAATTCCA | |
| | | CACAACATACGAGCCGGAAGCATAAAGTGTAAAGCCTGGGGTGCCTAATGAGTG | |
| | | AGCTAACTCACATTAATTGCGTTGCGCTCACTGCCCGCTTTCCAGTCGGGAAAC | |
| | | CTGTCGTGCCAGCTGCATTAATGAATCGGCCAACGCGCGGGGAGAGGCGGTTTG | |
| | | CGTATTGGGCGCTCTTCCGCTTCCTCGCTCACTGACTCGCTGCGCTCGGTCGTT | |
| | | CGGCTGCGGCGAGCGGTATCAGCTCACTCAAAGGCGGTAATACGGTTATCCACA | |
| | | GAATCAGGGGATAACGCAGGAAAGAACATGTGAGCAAAAGGCCAGCAAAAGGCC | |
| | | AGGAACCGTAAAAAGGCCGCGTTGCTGGCGTTTTTCCATAGGCTCCGCCCCCCT | |
| | | GACGAGCATCACAAAAATCGACGCTCAAGTCAGAGGTGGCGAAACCCGACAGGA | |
| | | CTATAAAGATACCAGGCGTTTCCCCCTGGAAGCTCCCTCGTGCGCTCTCCTGTT | |
| | | CCGACCCTGCCGCTTACCGGATACCTGTCCGCCTTTCTCCCTTCGGGAAGCGTG | |
| | | GCGCTTTCTCATAGCTCACGCTGTAGGTATCTCAGTTCGGTGTAGGTCGTTCGC | |
| | | TCCAAGCTGGGCTGTGTGCACGAACCCCC | |

In some embodiments, a single-chain antibody (e.g., an anti FAP scFv) can be prepared via recombinant technology by linking a nucleotide sequence coding for a heavy chain variable region and a nucleotide sequence coding for a light chain variable region. Preferably, a flexible linker is incorporated between the two variable regions.

In some embodiments, an anti-FAP antibody is produced by expressing a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 19, and/or a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 20. In some embodiments, an anti-FAP antibody is produced by expressing a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 24, and/or a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 25. In some embodiments, an anti-FAP antibody is produced by expressing a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 63, and/or a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 64. In some embodiments, an anti-FAP antibody is produced by expressing a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 68, and/or a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 69. In some embodiments, an anti-FAP antibody is produced by expressing a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 73, and/or a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 74. In some embodiments, an anti-FAP antibody is produced by expressing a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 78, and/or a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 79.

In some embodiments, an anti-FAP scFv is produced by expressing a nucleic acid at least 60% (e.g., 60%, 65%, 70%, 75%, 80%, 85%, 90%, 91%, 92%, 93%, 94%, 95%, 96%, 97%, 98%, 99%, or 100%) identical to any one of SEQ ID NOs: 21,26, 65, 70, 75, or 80.

In some embodiments, an anti-FAP CAR is expressed by a cell (e.g., an iNKT cell) by delivering to the cell a nucleic acid at least 60% (e.g., 60%, 65%, 70%, 75%, 80%, 85%, 90%, 91%, 92%, 93%, 94%, 95%, 96%, 97%, 98%, 99%, or 100%) identical to any one of SEQ ID NOs: 22,27, 66, 71, 76, or 81.

In some embodiments, the present disclosure also provides vectors (e.g., lentiviral vectors) for expressing the anti-FAP CAR in a cell (e.g., an iNKT cell). In some embodiments, the lentiviral vector comprises a nucleic acid sequence at least 60% (e.g., 60%, 65%, 70%, 75%, 80%, 85%, 90%, 91%, 92%, 93%, 94%, 95%, 96%, 97%, 98%, 99%, or 100%) identical to any one of SEQ ID NOs: 23, 28, 67, 72, 77, or 82.

In some embodiments, an anti-FAP CAR is expressed by a cell (e.g., iNKT cell) by delivering to the cell a nucleic acid at least 60% (e.g., 60%, 65%, 70%, 75%, 80%, 85%, 90%, 91%, 92%, 93%, 94%, 95%, 96%, 97%, 98%, 99%, or 100%) identical to any one of the coding sequence encoding any of the FAP binding moieties known in the art, for example, the FAP binding moiety as provided in Table 3. It is within the skill of a skilled artisan to design nucleic acid coding sequence based on the known amino acid sequences of any of the FAP binding moieties.

In some aspects, the present disclosure also provides methods for preparing a population of immune cells (e.g., iNKT cells) expressing the CAR (e.g., anti-FAP CAR). An initial population of immune cells can be obtained from any source, such as peripheral blood mononuclear cells (PBMCs), bone marrow, tissues such as spleen, lymph node, thymus, or tumor tissue. A source suitable for obtaining the type of cell desired would be evident to one of skill in the art. In some embodiments, the population of immune cells (e.g., iNKT cells) is derived from PBMCs. In some embodiments, a blood sample or an apheresis is taken from a generally healthy subject. In some embodiments, a blood sample or an apheresis is taken from a generally healthy subject who is at risk of developing a disease, but who has not yet developed a disease, and the cells of interest are isolated and frozen for later use. In some embodiments, the iNKT cells may be expanded, frozen, and used at a later time. In some embodiments, samples are collected from a patient shortly after diagnosis of a particular disease as described herein but prior to any treatments. In some embodiments, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH®, anti-CD3 antibodies, Cytoxan, fludarabine, cyclosporin, FK506, rapamycin, mycophenolic acid, steroids, FR901228, and irradiation. These drugs inhibit either the calcium dependent phosphatase calcineurin (cyclosporine and FK506) or inhibit the p70S6 kinase that is important for growth factor induced signaling (rapamycin) (Liu et al., Cell 66:807-815, 1991; Henderson et al., Immun. 73:316-321, 1991; Bierer et al., Curr. Opin. Immun. 5:763-773, 1993). In some embodiments, the cells are isolated for a patient and frozen for later use in conjunction with (e.g., before, simultaneously or following) bone marrow or stem cell transplantation. T cell ablative therapy using either chemotherapy agents such as, fludarabine, external-beam radiation therapy (XRT), cyclophosphamide, or antibodies such as OKT3 or CAMPATH®. In some embodiments, a blood sample or an apheresis are taken from a healthy donor, and the iNKT cells isolated from a third-party healthy donor can be used “off-the-shelf”, e.g., as described by Li et al., Off-the-shelf third-party HSC-engineered iNKT cells for ameliorating GvHD while preserving GvL effect in the treatment of blood cancers, iScience. 2022 Sep. 16; 25 (9): 104859.

In some embodiments, an initial population of the iNKT cells are purified from PBMCs using any suitable methods known in the art (e.g., FACS or MACS). In some embodiments, the initial population of the iNKT cells are stimulated by α-galactosylceramide (α-GalCer) or any modified glycolipid thereof, e.g., as described by Zhang et al., α-GalCer and iNKT Cell-Based Cancer Immunotherapy: Realizing the Therapeutic Potentials, Front Immunol. 2019 Jun. 6; 10:1126; Schafer et al., iNKT cell stimulation by glycolipid ligands modified from α-galactosylceramide results in differential interleukin-2 secretion profiles, J Immunol May 1, 2019, 202 (1 Supplement) 177.1) for activation and expansion. In some embodiments, the iNKT cells are transduced with any one of the lentiviral vector described herein to express an anti-FAP CAR (e.g., any one of the anti-FAP CAR as described herein). In some embodiments, after transduction, there are more than 1%, more than 2%, more than 5%, more than 8%, more than 10%, more than 12%, or more than 15%, more than 18%, more than 20%, more than 22%, more than 25%, more than 28%, or more than 30% iNKT cells expressing the anti-FAP CAR. In some embodiments, after transduction, the iNKT cells are further stimulated and expanded. In some embodiments, further stimulation and expansion comprises stimulating the iNKT cells expressing anti-FAP CAR with a feeder carrying FAP. Feeders can be any suitable feeder cell known in the art, e.g., feeder cells, or cell-derived membrane vesicles (Ukrainskaya et al., Antigen-Specific Stimulation and Expansion of CAR-T Cells Using Membrane Vesicles as Target Cell Surrogates, Small. 2021 November; 17 (45): e2102643). In some embodiments, the feeder is a feeder cell (e.g., K562 feeder cell, major histocompatibility complex class I chain-related protein A (MICA) feeder cells, or membrane-bound IL-21 human B-lymphoblastoid cell-line 721.221 (hereinafter, 221)-based artificial feeder (221-mIL-21) cells. In some embodiments, the feeder cell is a K562 feeder cell. Expansion of NK cells using genetically engineered K562 feeder cells has been previously described, see, e.g., Phan et al., Expansion of NK Cells Using Genetically Engineered K562 Feeder Cells, Natural Killer Cells pp 167-174. In some embodiments, the K562 cells are irradiated prior co-culture with the iNKT cell expressing the anti-FAP CAR. In some embodiments, the K562 cells are engineered to express FAP on the cell surface. In some embodiments, after co-culturing with feeder cells (e.g., irradiated K562 cells expressing FAP), at least 60%, at least 65%, at least 70%, at least 75%, at least 80%, at least 85%, at least 90%, at least 92%, at least 95%, at least 98%, at least 99%, or more iNKT cells express the anti-FAP CAR.

Without further elaboration, it is believed that one skilled in the art can, based on the above description, utilize the present disclosure to its fullest extent. Particular embodiments are, therefore, to be construed as merely illustrative, and not limitative of the remainder of the disclosure in any way whatsoever.

### Examples

The following examples are provided for illustrative purposes and are not intended to limit the scope of the disclosure.

Example 1: Selection of Anti-FAP CAR Candidates iNKT Cells

Expression of FAP is an important characteristic of CAFs (Garin-Chesa et al., Cell surface glycoprotein of reactive stromal fibroblasts as a potential antibody target in human epithelial cancers. Proc Natl Acad Sci USA. 1990; 87 (18): 7235-7239). FAP activity impacts the secreted CAF proteome, leading to reduced levels of anti-angiogenic factors (e.g., PEDF, angiopoietin-1, VEGFC), and regulates matrix processing enzymes (e.g., Koczorowska et al., Fibroblast activation protein-alpha, a stromal cell surface protease, shapes key features of cancer associated fibroblasts through proteome and degradome alterations. Mol Oncol. 2016; 10 (1): 40-58). Further, FAP remodels the ECM through the endopeptidase activity by cleaving the collagen and modifying bioactive signaling peptides in cancer (Kelly et al., Fibroblast activation protein-alpha and dipeptidyl peptidase IV (CD26): cell-surface proteases that activate cell signaling and are potential targets for cancer therapy. Drug Resist Updat. 2005; 8 (1-2): 51-58). FAP expression in cancer is thought to be associated with poor prognosis. In some embodiments, high level expression of FAP in the TME plays an immunosuppressive role (Kraman et al., Suppression of antitumor immunity by stromal cells expressing fibroblast activation protein-alpha. Science. 2010; 330 (6005): 827-830). Not wishing to be bound to any particular theory, FAP drives the CAFs towards mediating tumor immunosuppression by recruiting myeloid-derived suppressor cells (MDSCs) in the TME (e.g., Yang et al., FAP promotes immunosuppression by cancer-associated fibroblasts in the tumor microenvironment via STAT3-CCL2 signaling. Cancer Res. 2016; 76 (14): 4124-4135).

FAP-targeting CAR-T cells have been engineered to target CAFs in various solid cancers, such as mesothelioma, lung and pancreatic cancers (e.g., Bughda et al., Fibroblast Activation Protein (FAP)-Targeted CAR-T Cells: Launching an Attack on Tumor Stroma, ImmunoTargets and Therapy 2021:10 313-323; Wang et al., Targeting Fibroblast Activation Protein in Tumor Stroma with Chimeric Antigen Receptor T Cells Can Inhibit Tumor Growth and Augment Host Immunity without Severe Toxicity, Cancer Immunol Res; 2 (2) February 2014; Rodriguez-Garcia A, Palazon A, Noguera-Ortega E, Powell D J, Guedan S. CAR-T cells hit the tumor microenvironment: strategies to overcome tumor escape. Front Immunol. 2020; 11). However, on-target off-tumor toxicity was observed in these studies because while FAP is highly expressed in these cancers, it is also found to be expressed at low level in normal cells (e.g., skeletal muscle, adipose tissue, and pancreas, etc).

Various cancers express high FAP in CAFs in the TME, e.g., non-small cell lung cancer (NSCLC), breast cancer, head and neck cancer, colorectal cancer, prostate cancer, stomach cancer, pancreatic cancer, thyroid cancer, cervical cancer, renal cancer, urothelial cancers, or sarcoma.

Tumors with CAF could promote drug resistance (chemotherapy) through the secretion of IL-6 and other cytokines, as well as Snails from zinc-finger transcription factor superfamily. In some instances, fibrotic tumors with CAF can also promote EMT. It was not determined if CAF mediated resistance to checkpoint modulators, although CAF could be mediated by the ECM that play the role in a barrier limiting access of cancer cells to T cells. A high level of immunosuppressive cytokines suppressing T cells and a high infiltration of immune and stromal cell populations suggests that certain cancers (e.g., non-small cell lung cancer) may be targeted when FAP targeting agent (e.g., anti-FAP CAR iNKT cells) is administered in combination with sequential immune checkpoint inhibitor therapy (e.g., PD-1/CTLA-4 inhibitors). In addition, intrinsic activity of iNKT could remodel suppressive myeloid cells and kill tumor cells.

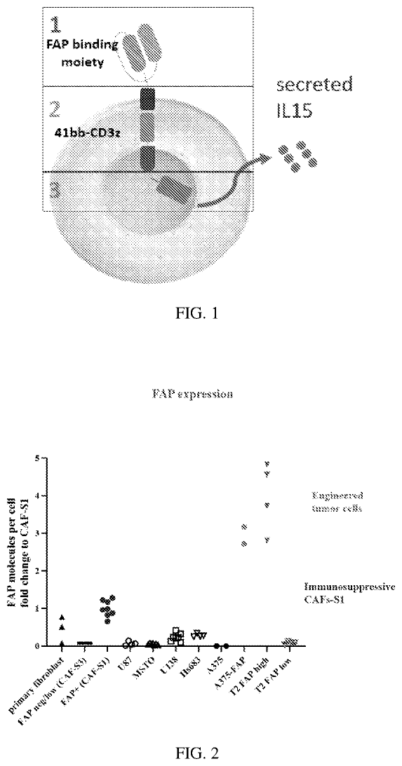

The present disclosure, at least in part, is based on the engineering of anti-FAP CAR iNKT cells using FAP binding moieties (e.g., anti-FAP scFv). iNKT cells can be engineered to express an anti-FAP CAR using known FAP binding moieties, e.g., exemplary known FAP binding moieties as described in Table 3, or any FAP-binding moieties described in the present disclosure. FIG. 1 shows an exemplary anti-FAP CAR iNKT cell of the present disclosure.

To figure out the therapeutic window for targeting FAP in treating cancer expressing FAP, cryopreserved dissociated tumor cells were used to evaluate FAP expression in tumor stroma. A number of antigen surface molecules expressed by primary cells and cell lines were quantified using a QUANTIBRITE™ kit. The known four CAF subsets-CAF-S1 to CAF-S4 (e.g., Kieffer et al., Single-Cell Analysis Reveals Fibroblast Clusters Linked to Immunotherapy Resistance in Cancer, Cancer Discov (2020) 10 (9): 1330-1351) were analyzed for FAP expression. CAF-S1, which show immunosuppressive function in the TME, expressed high level of FAP. CAF-S1 can be exemplary target cells of anti-FAP CAR iNKT cells (FIG. 2).

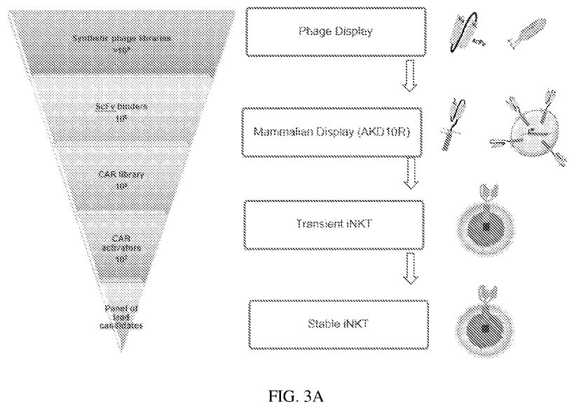

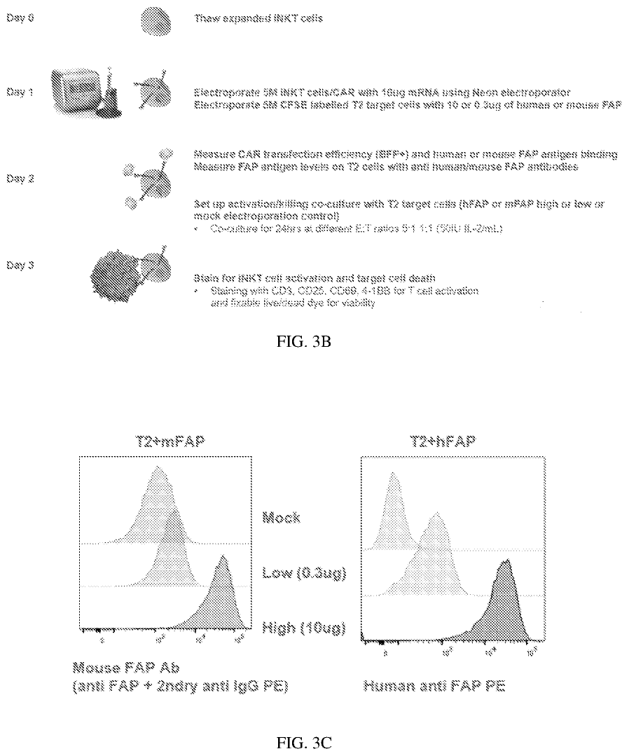

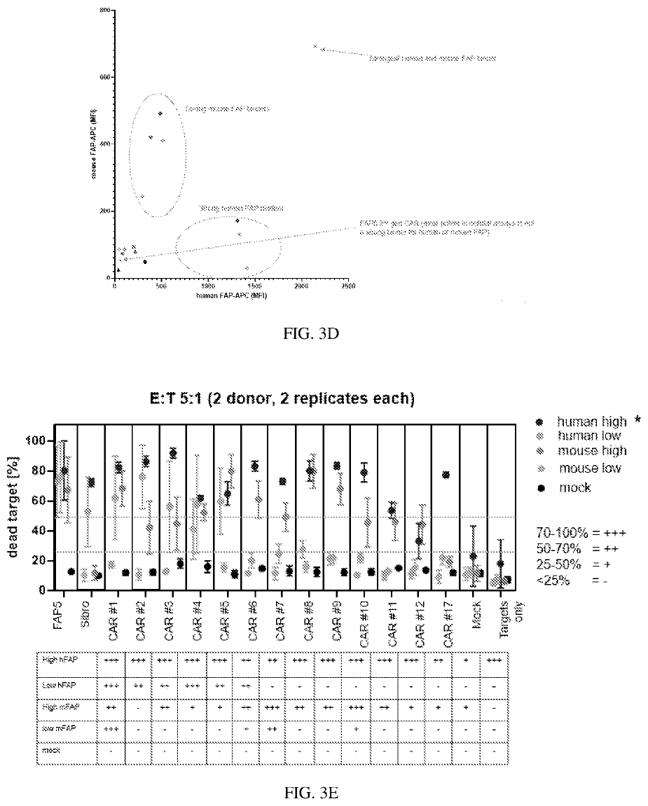

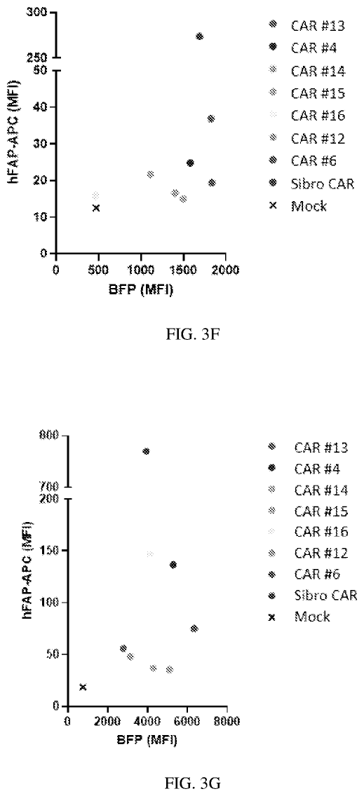

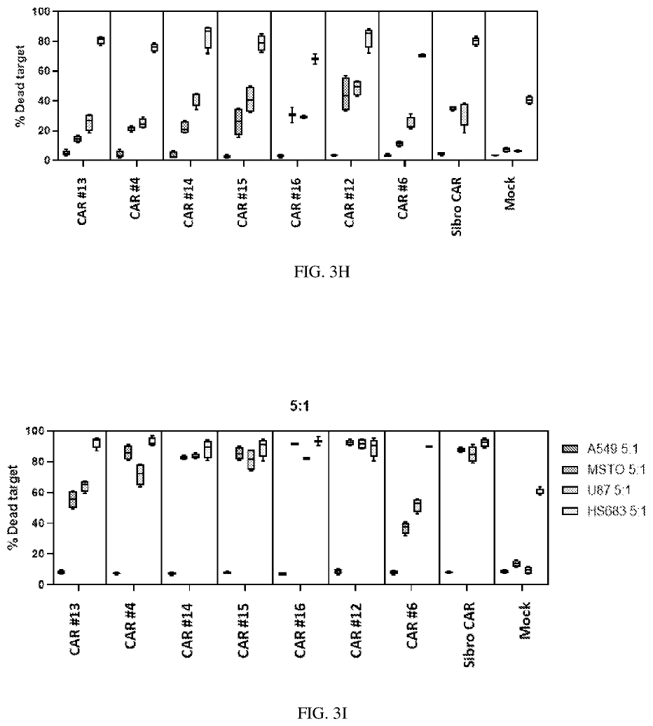

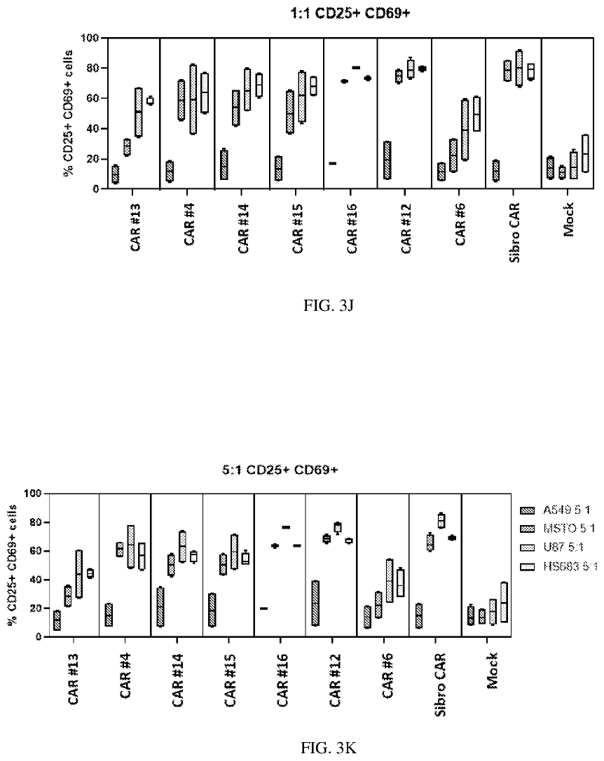

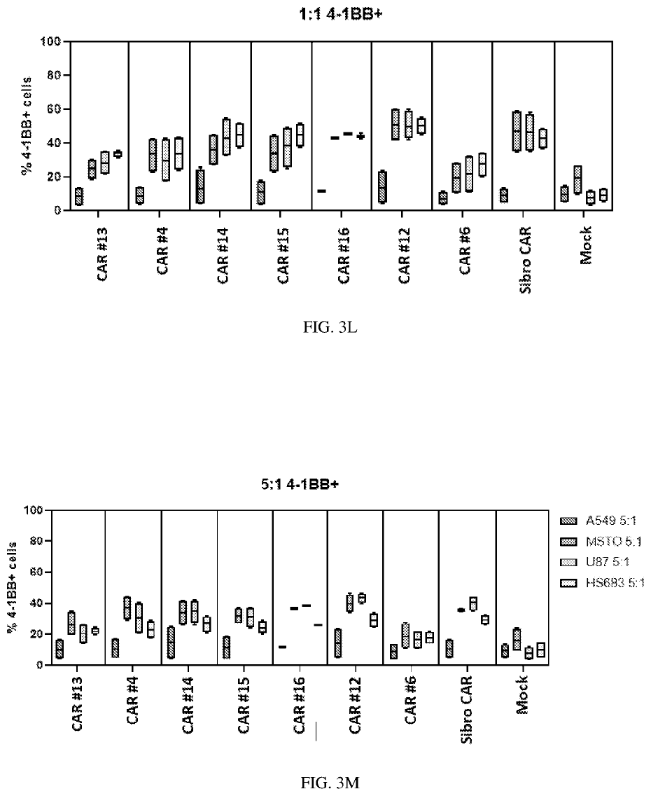

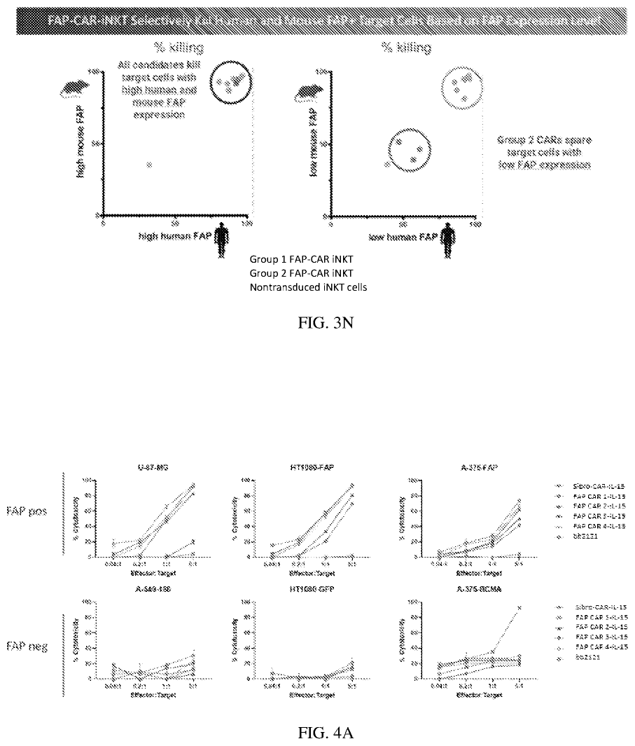

In some aspects, the present disclosure identified anti-FAP antibodies that specifically bind to human FAP, and has equivalent binding activity to mouse FAP. The exemplary anti-FAP scFv were engineered to be expressed by iNKT cells in a chimeric antigen receptor targeting FAP. The process of identifying a panel of anti-FAP candidate scFv is described in FIG. 3A. Briefly, a synthetic phage library displaying more than 10⁹ scFvs were screened for their binding ability to human and mouse FAP. The screening identified approximately 10⁶ FAP binders. The FAP-binders derived by Phage Display were converted into a mammalian Display library essentially as described in Breous-Nystrom et al., (Retrocyte Display technology: generation and screening of a high diversity cellular antibody library, Methods (2014) 65 (1): 57-67). The method described by Breous-Nystrom was adapted to mammalian Display in CAR format in NFAT-GFP reporter cells. The resulting CAR mammalian display library was further screened for binding to human and mouse FAP, resulting in a population of a ˜10⁵ reporter cells expressing ScFv-CAR molecules recognizing both human and mouse FAP. This population of human-mouse cross-reactive CAR-expressing NFAT-GFP reporter cells were further tested to evaluate whether they can be activated by co-culture with mouse and human FAP expressing target cells. The reporter cells expressing scFvs-CAR molecules that showed good activation by FAP expressing target cells (˜10²) were recovered by single cell cloning and the FAP-specific ScFvs were isolated from these single cell clones. The recovered ScFvs were subsequently inserted in a CAR-expression vector and transiently expressed by iNKT cells. The iNKT cells transiently expressing the anti-FAP CAR leads were tested for their ability of being activated by the target cells and killing of the target cells. Through these experiments, a lead panel of scFv was selected for stable expression by iNKT cells. The iNKT cells stably expressing the lead ScFv-CAR molecules were tested in a panel of in vitro and in vivo assays (e.g., activation by and killing of target cells, cell exhaustion, cell expansion, cytokine profiling, and next generation sequencing (NGS)).

After the initial phage display screening, and the mammalian cell display experiments, a group of exemplary scFvs, including the scFvs selected from the process described in FIG. 3A were engineered to be transiently expressed by iNKT cells in the format of a CAR. FAP binding moieties derived from previous known anti-FAP antibodies FAP5 and sibrotuzumab were also engineered to be expressed by iNKT cells in the format of a CAR. The iNKT cells expressing the anti-FAP CARs were evaluated for FAP binding on target cells and killing of target cells. The workflow of the transient anti-FAP iNKT assays is described in FIG. 3B. On Day 0, previously expanded and frozen iNKT cells from two donors were thawed, and cultured. On Day 1, 5 million iNKT cells were electroporated with 10 μg mRNA encoding an anti-FAP CAR, and 5 million CFSE labelled target cells were electroporated with 0.3 μg or 10 μg either human or mouse FAP. The iNKT cells expressing the anti-FAP CAR were cocultured with the T2 target cells expressing human FAP or mouse FAP at high or low level at different Effector: Target (E:T) ratio, for example, E:T ratio of 5:1 or 1:1 for 24 hours. IL-2 was added to the culture at 50 IU per ml of culture medium. The cells were subsequently stained for iNKT cell activation (e.g., with CD3, CD25, CD69, 4-1BB), and target cell death (e.g., by fixable live/dead dye).

After target cells were electroporated with human or mouse FAP, the expression levels of FAP on the target cells were evaluated. The results showed that the target cells that received 0.3 μg of mouse or human FAP expressed low level of FAP whereas the target cells that received 10 μg of mouse or human FAP expressed high level of FAP (FIG. 3C).

Binding of exemplary anti-FAP CAR iNKT cells to target cells expressing FAP was tested. As shown in FIG. 3D, exemplary anti-FAP CAR were separated as groups that show strong mouse FAP binding, strong human FAP binding, and strong human and mouse FAP binding. Sibrotuzumab CAR was identified as a strong human FAP binder. FAP5 is not a strong binder for human or mouse FAP, but later showed potent cytotoxicity. The same pool of anti-FAP CAR candidates were also tested for their potency in killing target cells expressing different levels of human or mouse FAP. The results showed that FAP5-CAR iNKT cells were capable of killing target cells expressing both high and low level of mouse and human FAP. Sibro-CAR iNKT cells were able to kill target cells expressing high and low level of human FAP but did not kill target cells expressing mouse FAP. Other candidates showed variable potency in killing target cells expressing high or low levels of human or mouse FAP, as shown in FIG. 3E.

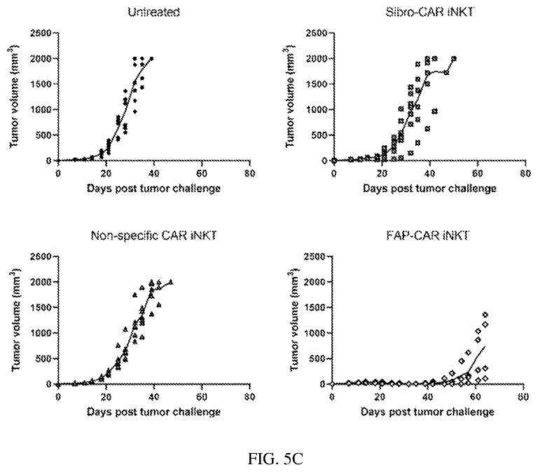

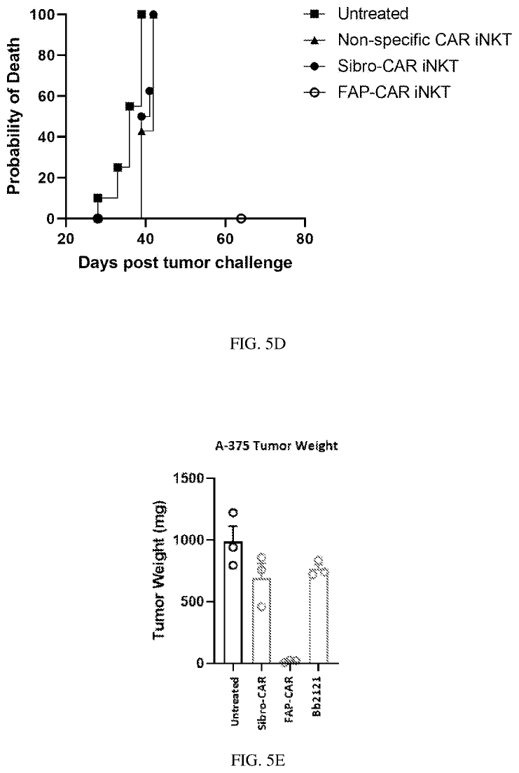

The exemplary CARs were further tested for human FAP binding and in cell activation assays. FIGS. 3F-3G show binding of human FAP by exemplary anti-FAP CAR iNKT cells derived from two different donors. The same pool of candidates was tested for target cell induced activation and target cell killing. Target cells include: A549 cells (no FAP expression), MSTO cells (low FAP expression), U87 cells (high FAP expression), and HS683 cells (high FAP expression). FIGS. 3H-3I show target cell killing at E:T ratio of 1:1 (FIG. 3H), or 5:1 (FIG. 3I). All anti-FAP candidate CAR iNKT were able to kill more U87 and HS683 cells compared to mock control. No obvious killing of A549 cells were observed. Certain candidates (e.g., CAR #16, CAR #12. sibro-CAR) are more efficient in killing MSTO cells than others. FIGS. 3J-3K show target cell induced iNKT cell activation by measuring the surface markers CD25 and CD69 at E:T ratio of 1:1 (FIG. 3J) or 5:1 (FIG. 3K). All candidates showed target cell induced iNKT cell activation but express CD25 and CD69 at various levels. FIGS. 3L-3M show target cell induced iNKT cell activation by measuring 4-1BB at E:T ratio of 1:1 (FIG. 3L) or 5:1 (FIG. 3M). All candidates showed target cell induced iNKT cell activation but express 4-1BB at various levels. In FIGS. 3E-3M, CAR #1 corresponds to G03, CAR #2 corresponds to F02, CAR #3 corresponds to G09, CAR #4 corresponds to G07, CAR #5 corresponds to D07, CAR #6 corresponds to D05, CAR #7 corresponds to F01, CAR #8 corresponds to D09, CAR #9 corresponds to D08, CAR #10 corresponds to G02, CAR #11 corresponds to E05, CAR #12 corresponds to F08, CAR #13 corresponds to F09, CAR #14 corresponds to H06, CAR #15 corresponds to A07, CAR #16 corresponds to F02, and CAR #17 corresponds to G11.

The candidate selection process identified two distinct groups of anti-FAP-CARs, as shown in FIG. 3N. Group 1 candidates showed high potency killing on FAP expressing cells, both high and low. Group 2 candidates offered selective killing of FAP-high-expressing cancer associated cells while sparing FAP-low cells (e.g, at E:T ratio 1:1).

Example 2: Construction and Evaluation of iNKT Cells Expressing Anti-FAP CAR In Vitro

iNKT cells stably expressing anti-FAP CAR were produced using the following method. Blood was taken from donors by apheresis, and iNKT cells were isolated from peripheral blood mononuclear cells (PBMC). The initial population of the iNKT cells were stimulated by 100 ng/ml α-galactosylceramide (α-GalCer) for activation and expansion. The cells were cultured for 10-18 days. At this point, the cells were stimulated by anti-CD3/anti-CD28 stimulation and transduced using a lentiviral vector encoding the anti-FAP CAR and soluble IL-15. The transduced iNKT cells were further enriched using irradiated feeder cells engineered to express human FAP (IF-FAP) and cells were co-cultured with iF-FAP cells for 10-18 days. On Day 7, the cells were fed with media containing IL-2 and other cytokines every 48 hours. Between Day 10-18, the iNKT cells were harvested and CAR-expression was evaluated. The cells were then evaluated in vitro cytotoxicity, activation, proliferation assay, and in vivo assays.

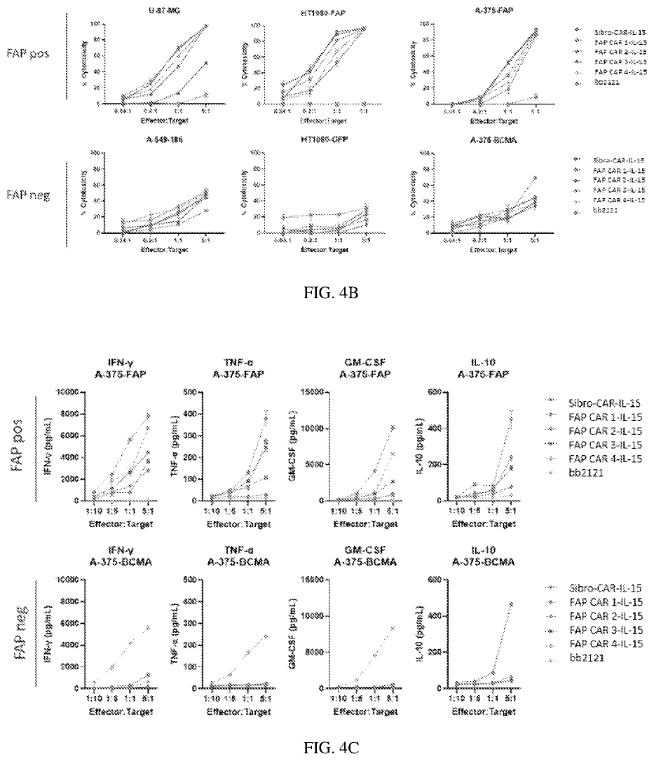

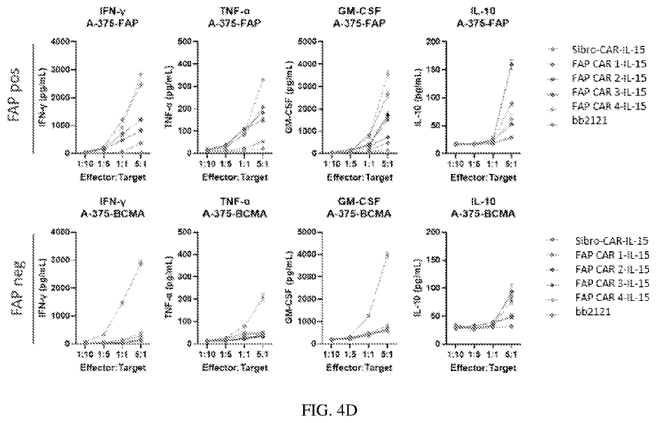

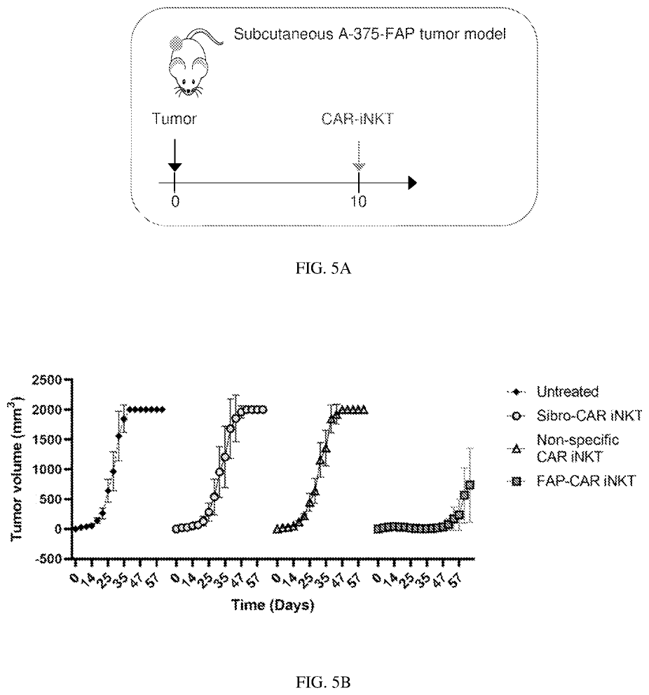

iNKT cells from two donors were transduced to express anti-FAP CAR and IL-15, and these cells were validated in cytotoxicity assays. The results show that FAP-CAR iNKT candidates respond to FAP+ tumors in vitro (FIG. 4A (donor 1) and FIG. 4B (donor 2)). FIGS. 4C and 4D show that anti-FAP CAR iNKT cells were polyfunctional in response to A-375-FAP tumor cells in vitro (FIG. 4C (donor 1) and FIG. 4D (donor 2)). Taken together, these results demonstrate that FAP-CAR iNKT candidates can be activated by FAP-expressing cell lines (A-375-FAP, HT1080-FAP, U87-MG) in vitro across two different donors. In FIGS. 4A-4D, FAP CAR 1 corresponds to D01, FAP CAR 2 corresponds to D05, FAP CAR 3 corresponds to A07, and FAP CAR 4 corresponds to H02.

Example 3: Evaluation of iNKT Cells Expressing Anti-FAP CAR In Vivo

The effect of anti-FAP CAR iNKT cells expressing D01 as FAP binding moiety of the CAR were tested in vivo to evaluate whether treatment influenced tumor growth. An exemplary experimental design using A-375-FAP tumor model is illustrated in FIG. 5A. One million A-375-FAP tumor cells were injected subcutaneously in HBSS containing 50% Matrigel in NOG OD.Cg-Prkdcscid Il2rgtm1Sug Tg(CMV-IL2/IL15) 1-1 Jic/JicTac mice. FAP-CAR iNKT cells and non-specific CAR iNKT cells targeting human BCMA were injected intravenously 11 days post tumor challenge. Tumor growth was monitored twice a week from day 7 onwards using external caliper measurements for a period of 65 days. At day 11, 10 million CAR+ FAP-CAR-iNKT cells, Sibrotuzumab (Sibro)-CAR iNKT cells or non-specific-CAR iNKT cells were injected intravenously. Sibrotuzumab-CAR iNKT or non-specific-CAR iNKT-treated mice showed no tumor control and demonstrated control levels similar to untreated animals. However, mice treated with FAP-CAR iNKT cells showed a significant tumor control up to 65 days post-tumor implantation (FIG. 5B). Mice treated with Sibrotuzumab-CAR iNKT or non-specific-CAR iNKT cells showed equivalent, homogeneous tumor growth as compared to untreated mice, while MiNK FAP-CAR iNKT-treated mice showed complete tumor control up to 40 days. Post 40 days, tumor growth was heterogeneous between mice with some showing complete tumor control and some showing various level of relapse. FIG. 5D shows the survival analysis of A-375-FAP tumor-bearing mice either left untreated or, treated with FAP-CAR iNKT cells, sibrotuzumab-CAR NKT cells, or control non-specific-CAR iNKT cells. At 65 days post-tumor challenge, mice treated with FAP-CAR showed 100% survival while non-specific-CAR iNKT and Sibrotuzumab-CAR iNKT cells treated mice rapidly succumbed to tumor outgrowth 35 days post-tumor implantation, similar to untreated mice (FIG. 5D-5E). Further, iNKT cell proliferation was observed at tumor site as evidenced by increased iNKT cell percentage of total cells and iNKT numbers. CAR expression was downregulated in anti-FAP-CAR iNKT cells homed to the tumor site but not in peripheral tissues, suggesting antigen-mediated activation of CAR-iNKTs. Further, anti-FAP CAR iNKT cells showed increased Ki67 in tumors suggesting that increased proliferation was associated with anti-tumor response. A strong INF-r response was observed from serum of mise received the anti-FAP CAR iNKT cells. Taken together, the data suggests that anti-tumor responses by FAP-CAR iNKT cells is associated with increased iNKT infiltration/homing to the tumor, increased proliferation, and heightened IFN-γ in the serum. These effects led to delayed tumor outgrowth and improved overall survival (of 100%) out to day 49 post tumor injection.

Example 4: Combination Therapy Using Anti-FAP CAR iNKT Cells and Anti-Tumor CAR T Cells

Aspects of the present disclosure also contemplate combination therapy of anti-FAP CAR iNKT cells, in combination with (e.g., followed by) an anti-tumor T cell therapy.

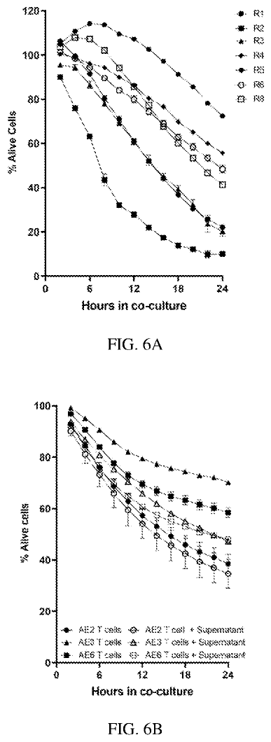

iNKT cells have been observed to have the capacity to enhance T cell responses through their ability to secrete large amounts of cytokine. Therefore, a 2D co-culture assay was designed to evaluate the impact of iNKT enhancement of T cell cytotoxicity against tumor cells. T cells expressing transgenic TCR targeting NYESO-1 were co-cultured with A549 non-small cell lung cancer (NSCLC) tumor cells which were engineered to express expressing HLA-A*02:01-SLLMWITQC (NYESO-1 antigen-MHC complex) and GFP/nano-luciferase. T cells were antigen exposed (AE) to A549-NYESO-1 expressing tumor cells over multiple rounds of stimulation and evaluated for cytotoxic capacity. NYESO-1 T cells gradually lost cytotoxic capacity over increasing antigenic exposure rounds in vitro (FIG. 6A). To determine whether FAP-CAR-IL-15 iNKT cells expressing a anti-FAP CAR comprising D01 could enhance the cytotoxic potential of T cells, NYESO-1 T cells which had undergone two, three, or six rounds of antigenic exposure (AE2, AE3, and AE6, respectively) were co-cultured against A549-NYESO-1 expressing tumor cells either in media alone (solid symbols) or with supernatant (open symbols) from previously activated FAP-CAR-IL-15 iNKT cells. In the presence of FAP-CAR-IL-15 iNKT cell supernatant, NYESO-1-specific T cells were capable of increased killing of tumor cell targets (FIG. 6B).

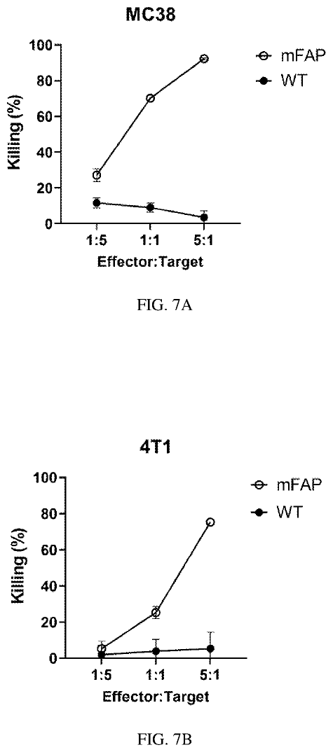

Subsequently, a non-small cell lung cancer (NSCLC) xenograft model was designed to illustrate the effectiveness of a combination therapy of anti-FAP CAR iNKT cells and anti-tumor TCR T cells. FAP-CAR iNKT cells expressing an anti-FAP CAR comprising E07 and sIL-15 were evaluated for ability to kill mouse-FAP expressing cells by 2D in vitro co-culture. Two murine tumor cell lines, MC38 and 4T1 were engineered to express GFP/nano-luciferase and/or mouse FAP. FAP-CAR-IL-15 iNKT cells were co-cultured with FAP-expressing (open circles) or control (closed circles) tumor cells and cytotoxicity was evaluated. FAP-CAR-IL-15 iNKT cells were able to kill mouse FAP-expressing cells at various effector-to-target (E:T) ratios in a FAP-specific manner over non-FAP expressing controls (FIGS. 7A and 7B).

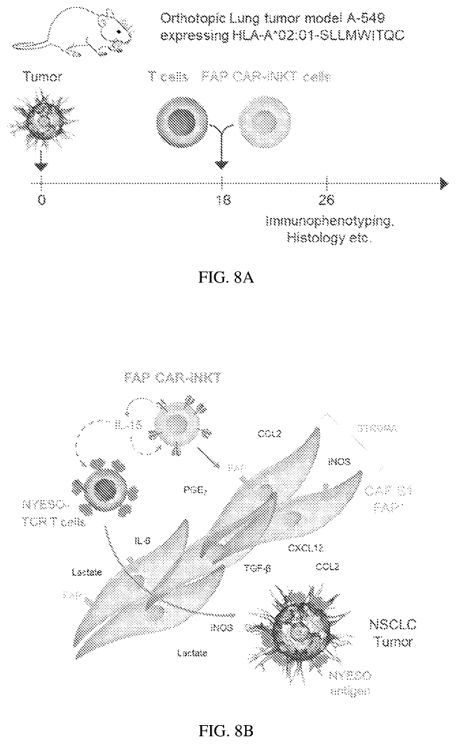

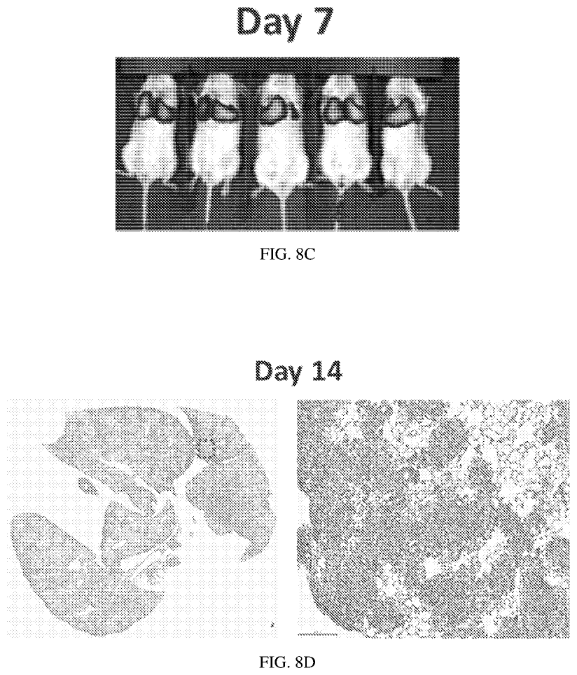

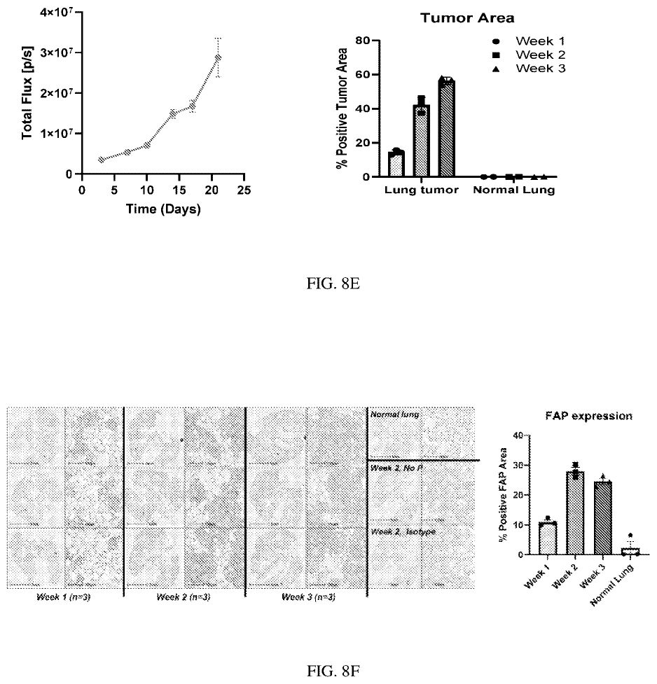

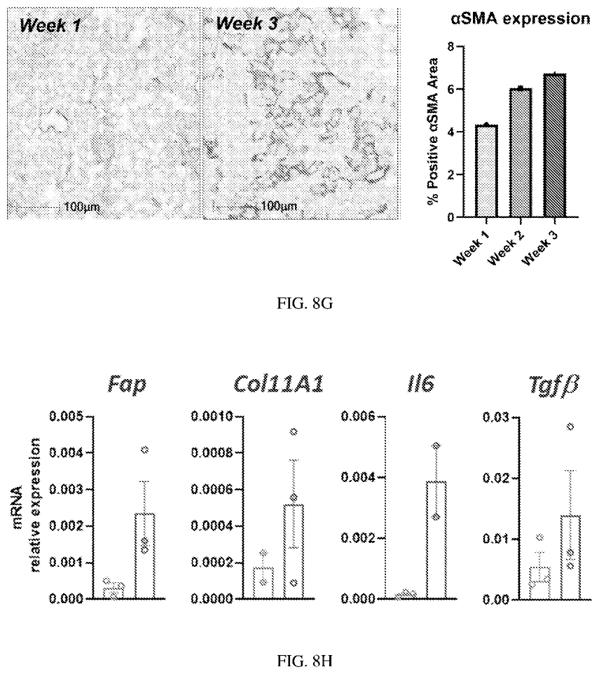

FIG. 8A shows an experimental design of NSCLC xenograft tumor model treated with FAP-CAR-IL-15 iNKT cells. Two million A549 expressing HLA-A*02:01-SLLMWITQC (NYESO antigen-MHC complex) were injected intravenously at day 0. At day 18 post tumor challenge, T cells expressing NYESO-TCR targeting HLA-A*02:01-SLLMWITQC and FAP-CAR-IL-15-iNKT cells were injected intravenously. At day 26, lung tissues were analyzed for histology and immunophenotyping. Survival is monitored over 100 days.

Not wishing to be bound to a particular theory, and as shown in FIG. 8B, CAF S1 (large spindle shape cells in the center) build the immunosuppressive stroma around the tumor cells (e.g., NSCLC cells) resulting in a dense extracellular matrix and an immunosuppressive environment with presence of inhibitory molecules such as CXCL-12, iNOS, PGE2, lactate etc. FAP-CAR-IL-15 iNKT cells target FAP+ CAF S1 reducing the T cell repressive environment. T cells expressing an NYESO TCR can then infiltrate efficiently into the tumor tissue to kill tumor cells. FAP-CAR-IL-15 iNKT cells express constitutively IL-15 which increase iNKT persistence within the tissue and potentiate cytotoxic activity of T cells.

NOG mice were injected with 2 million A549 tumor cells expressing HLA-A*02:01-SLLMWITQC and nano luciferase intravenously. 7 days post tumor challenge, luminescence was recorded via IVIS® Spectrum in vivo imaging. Substrate was injected subcutaneously and images were recorded 4 to 10 minutes later. Images showed increased luminescence in the thoracic area corresponding to the lungs indicating lung tumor engraftment (FIG. 8C). Lungs of mice administered with A-549 tumor cells were harvested and analyzed via H&E staining 14 days post tumor implantation. Images showed large and dense tumor areas in the lungs (FIG. 8D). FIG. 8E shows increase in luminescence of mice implanted with tumor cells recorded over 21 days correlating with the increase of tumor mass, which is supported by histological quantification of excised lungs at such timepoints. Histological examination of excised lungs from tumor-bearing animals demonstrated immunosuppressive CAF recruitment as FAP expression increased in correlation with tumor progression (FIG. 8F). Accordingly, non-immunosuppressive CAFs appeared to be largely unaltered, with modest increases in α-SMA expression observed from weeks 1 to 3 (FIG. 8G). Lungs were further analyzed by QPCR for murine fap, col11A1, 116 and Tgfb expression. Mice implanted with tumor showed increased expression of these molecules suggesting establishment of a suppressive stroma (FIG. 8H).

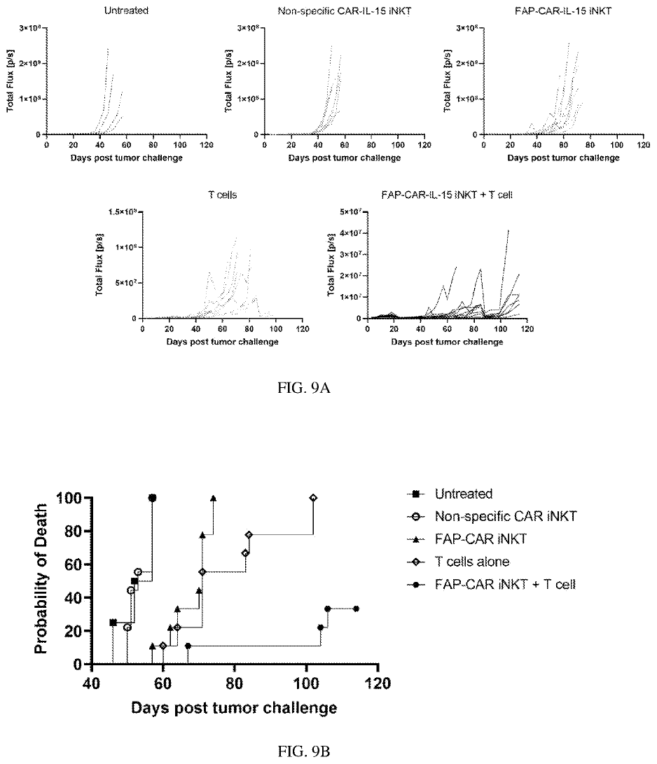

NOG mice challenged with A549 tumor cells expressing HLA-A*02:01-SLLMWITQC were either left untreated or treated with non-specific-CAR-IL-15 iNKT cells, FAP-CAR-IL-15 iNKT cells expressing B08 it its anti-FAP CAR, T cells expressing NYESO-1 TCR, or FAP-CAR-IL-15 iNKT cells plus T cells expressing NYESO-1 TCR and were monitored for tumor growth by IVIS imaging. While mice treated with FAP-CAR-IL-15 alone and NYESO-1-specific T cells alone did result in significant tumor growth delay, mice harboring NYESO-1-specific T cells and treated with FAP-CAR-IL-15 iNKT cells demonstrated robust and sustained tumor control (FIG. 9A). FAP-CAR-IL-15 iNKT cell plus T cells treated mice showed up to 88.8% survival compared to mice untreated (0%—Day 57). Mice treated with FAP-CAR-IL-15 did not survive (0%—Day 74), and mice treated with T cell only showed 22.2% survival rate on Day 84 (FIG. 9B).

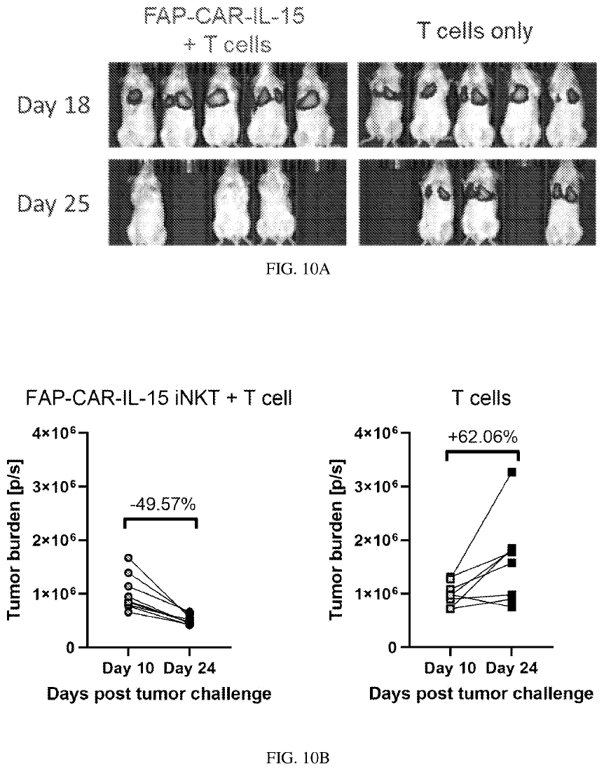

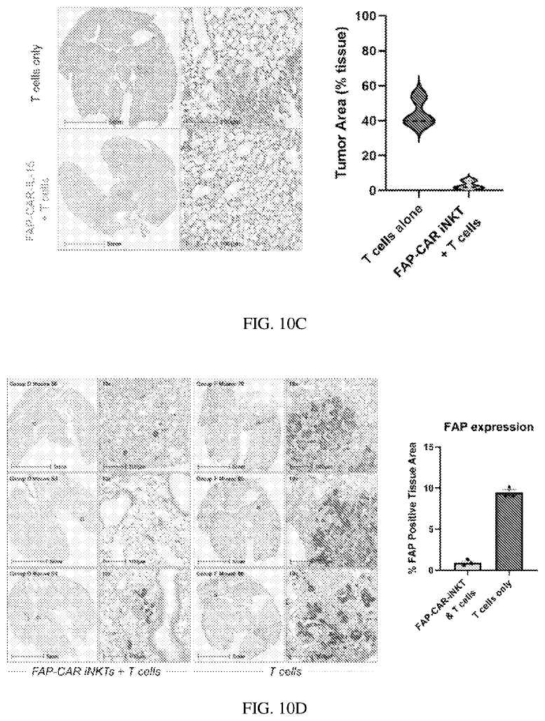

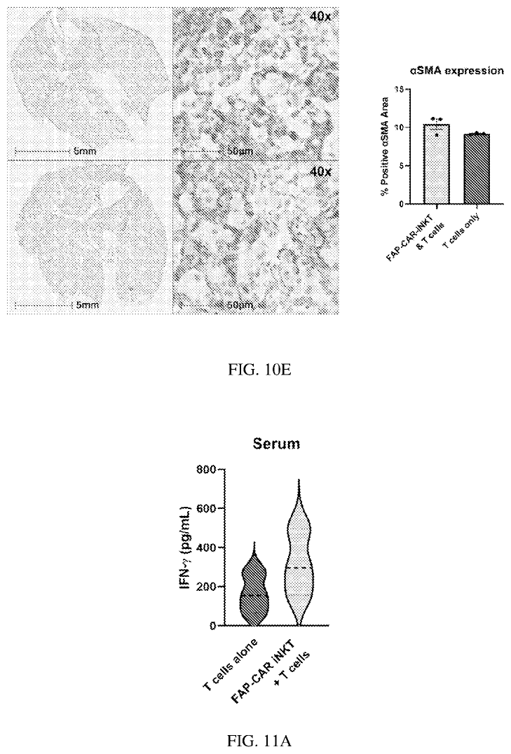

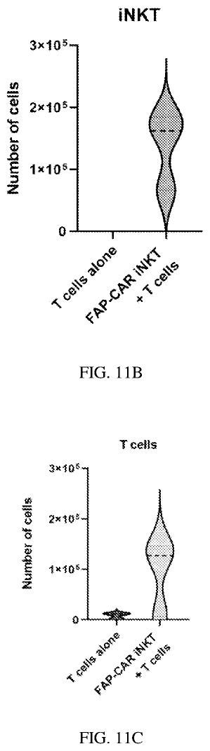

Bioluminescence imaging of mice treated with FAP-CAR-IL-15 iNKT+ T cells (left panel) and T cells only (right panel) at day 18, time of iNKT and T cells infusion, and day 25 post tumor injection, seven days post iNKT and T cells injection. While tumors are detectable in both group at day 18, tumors are only detectable in the group T cell only at day 25 (FIG. 10A). Tumor burden progression was evaluated from day 10 (pre-treatment) to day 25 (7 days post-treatment) post-tumor injection in both group T cell only and FAP-CAR-IL-15 iNKT+T cells. The FAP-CAR-IL-15 iNKT cells+T cells group at Day 25 showed decreased luminescence signal compared to the T cell group that showed an increase in bioluminescent signal (FIG. 10B). Lungs of mice treated with FAP-CAR-IL-15 iNKT cells+T cells or T cells only were harvested at day 25 and processed for H&E staining. Tumor patches (dark areas) remained in the T cells only group (FIG. 10C), while minimal tumor was identified in mice treated with FAP-CAR-IL-15 iNKT cells+T cells (FIG. 10C, left panel), which was supported by quantification (FIG. 10C, right panel).

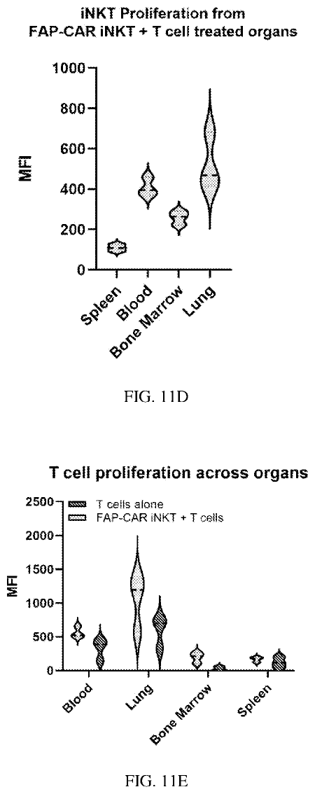

Animals treated with FAP-CAR-IL-15 iNKT cells+T cells showed higher levels of immune cell activation and tumor tissue infiltration. Measurement of IFN-γ in the serum of mice treated with FAP-CAR-IL-15 iNKT cells+T cells or T cells only showed increased level of IFN-γ in FAP-CAR-IL-15 iNKT cell+T cell group (FIG. 11A). Quantification of the number of iNKT detected by flow cytometry in lung tissues from groups FAP-CAR-IL-15 iNKT cell+T cells or T cells only demonstrated, as expected, that iNKT are detected only in lung tissues in FAP-CAR-IL-15 iNKT+T cells group (FIG. 11B). Quantification of the number of T cells detected by flow cytometry in lung tissues from harvested mice showed increased T cells number in animals treated with both T cells alone or FAP-CAR-IL-15 iNKT cells+T cells, however, T cell numbers were higher in mice that had been treated with FAP-CAR-IL-15 iNKT cells indicating that the treatment of FAP-CAR-IL-15 iNKT facilitates T cell infiltration to the tumor tissue (FIG. 11C). Activation and proliferation of iNKT cells were measured in blood, lung, spleen, and bone marrow tissues of animal treated with FAP-CAR-IL-15 iNKT+T cells. iNKT show high Ki67 expression indicating higher level of activation/proliferation in the lung tissue (FIG. 11D). Evaluation of the activation and proliferation of T cells in both groups were measured in the blood, lung, spleen, and bone marrow tissues of animal treated with FAP-CAR-IL-15 iNKT+T cells compared to T cells only. Here, it was found that Ki67 expression was higher in T cells isolated from the lung when co-administered with FAP-CAR-Il-15 iNKT cells (FIG. 11E).

Example 5. Development of an Allogenic FAP CAR iNKT Product to Target Tumor Stroma and Modulate the Tumor Micro Environment

### BACKGROUND

A novel allogeneic CAR-iNKT cell (invariant natural killer T cell) product targeting Fibroblast Activating Protein (FAP) and secreting IL-15 was developed for the treatment of solid tumors. The emergence of antibodies targeting checkpoint modulators such as PD-1 and CTLA-4 has revolutionized solid tumor treatment and highlighted the importance of effectively engaging the immune system to drive durable responses. However, to date well over 50% of solid tumor patients do not respond adequately to checkpoint modulators and this is generally ascribed to active immune suppression in the tumor microenvironment (TME). To overcome suppression and increase tumor control, a highly selective and precisely tuned Chimeric Antigen Receptor (CAR) targeting FAP expressed on tumor cells and on immune-suppressive Cancer Associated Fibroblasts (CAFs) was developed. A key element of the approach is the use of iNKT cells. Their natural resistance to exhaustion, tissue homing properties, selective cytotoxicity towards M2 macrophages and stimulation of dendritic cell maturation make them highly effective vehicles for solid tumor cell therapy. They also show intrinsic CD1d- and NK receptor ligand targeted cytotoxicity and do not cause Graft versus Host Disease due to their invariant T cell receptor.

### Methods

A proprietary CARDIS™ platform combines screening of highly diverse (>10¹⁰) fully human scFv libraries with library-based direct functional selection in CAR format using mammalian display. Candidates can be further optimized using affinity tuning to ensure optimal and highly selective on-target/on-tumor activity and full mouse cross-reactivity. A scalable manufacturing approach was developed to engineer and specifically expand CAR and soluble IL-15-expressing allogeneic iNKT cells.

### Results

Using our CARDIS™ platform, a panel of fully human, potent, and highly selective anti-human FAP CARs with equivalent cross-reactivity towards mouse FAP was generated. In xenograft mouse models, the FAP-CAR iNKT cells effectively control FAP-expressing tumors, as well as FAP negative tumors with immune-suppressive CAFs, while soluble IL-15 prolongs persistence in immunocompromised mice. In addition, FAP-CAR iNKT cells was able to potentiate the recruitment and enhance anti-tumor activity of tumor-specific T-cells.

### CONCLUSIONS

The CARDIS™ and iNKT platform was used to develop a CAR-iNKT therapy effectively and selectively targeting FAP positive tumor cells and suppressive CAF subsets in the TME. Combining the activity of our FAP-CAR with the potent natural activity of iNKT cells will enable a level of tumor control and immune engagement to solid tumor patients beyond currently available treatments.

Example 6. Construction of Modular FAP-CAR iNKT Library

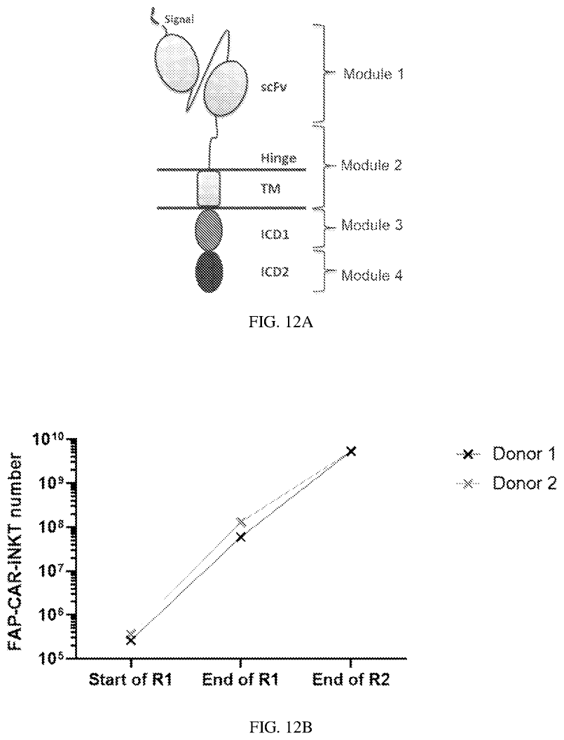

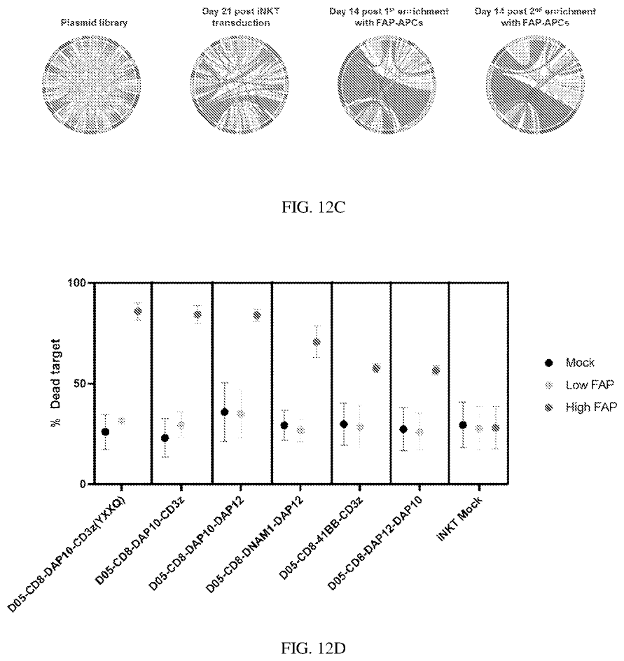

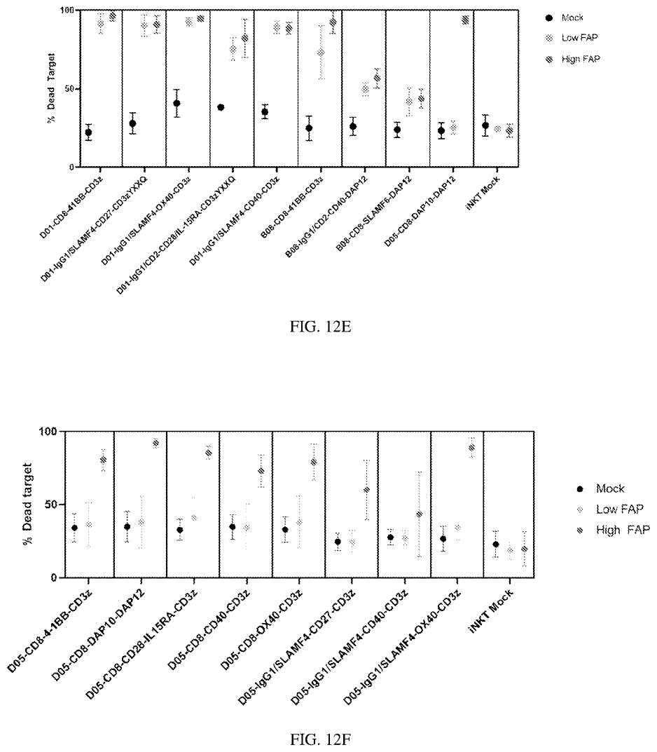

FAP-CAR iNKT library was packaged as lentivirus and transduced into iNKT cells and FAP-CAR+ iNKTs were enriched by sorting on day 17 post transduction. A modular FAP-CAR iNKT library comprised of 4 different modules was generated according to the structure of a 2nd generation CAR (scFv, TM/Hinge region, 1st ICD region and 2nd ICD region). Each region had the following number of possible domains: scFv (3), TM/hinge (3), 1st ICD region (24), 2nd ICD region (24) to give a library with a total possible diversity of 5,184 combinations (FIG. 12A).

On day 21 post transduction, iNKT cells were co-cultured with FAP expressing antigen presenting cells from 2 donors to specifically activate FAP-CAR iNKTs and enrich for ICDs having good activation/proliferation. On day 35 post transduction, a further round of co-culture with FAP expressing antigen presenting cells was performed for a further 14 days, giving over 15000 enrichment over the 4 weeks of co-culture (FIG. 12B).

DNA from the initial plasmid library and DNA extracted from aliquots taken on day 21 post iNKT transduction and day 14 post 1^{st} and 2^{nd} enrichment round were recovered by nanopore sequencing. 5,178 of 5,184 expected sequences were detected on the plasmid library and clear enrichment in certain domain combinations was observed. In particular, the ICD position 2 was highly enriched for CD3z, CD3z(YXXQ) and DAP12 (FIG. 12C).

iNKT cells were electroporated with mRNA encoding FAP-CAR constructs (or water for mock) with the different domains of the tested CARs listed on the x-axis. Target cells (T2) were electroporated with mRNA encoding FAP at high levels (2 μg mRNA) or low levels (100 ng mRNA) or mock (water). At 24 hrs post electroporation, target cells were labelled with CFSE and were then mixed with iNKT cells at a 5:1 effector: target ratio and co-cultured for 24 hrs. Killing was then assessed by flow cytometry by analyzing the % of CFSE+ cells staining positive with live/dead dye (FIGS. 12D-12F).

### Example 7. Kinetic Analysis and Epitope Mapping

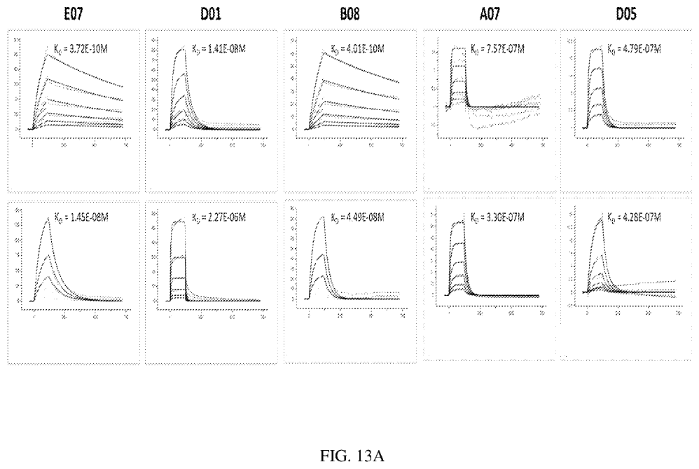

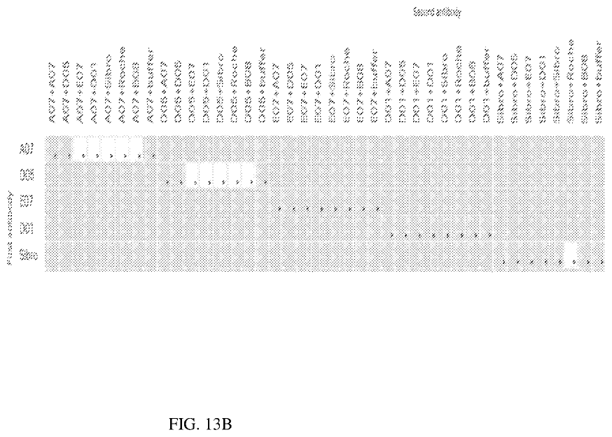

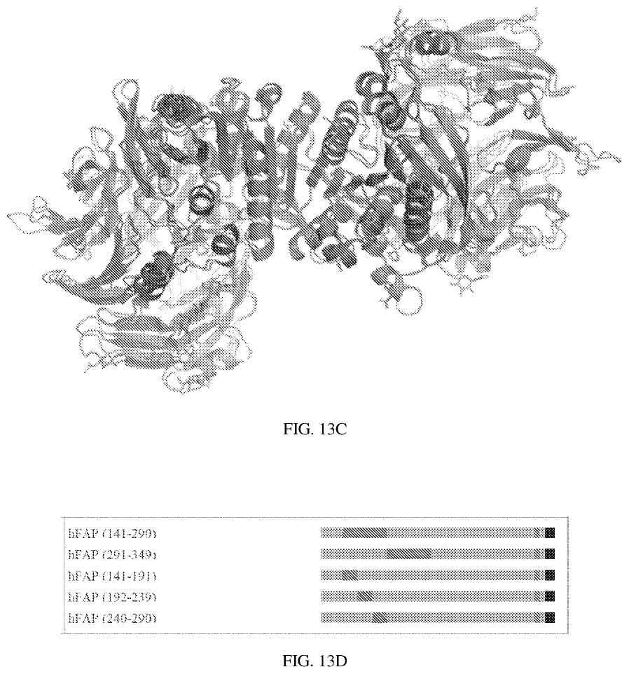

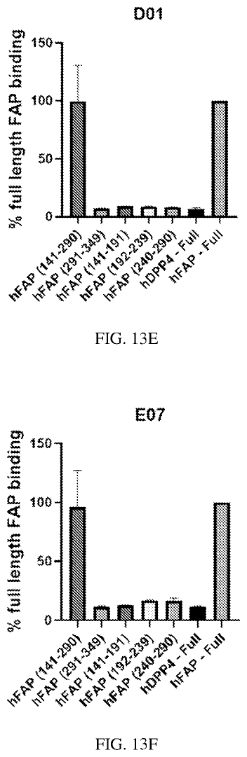

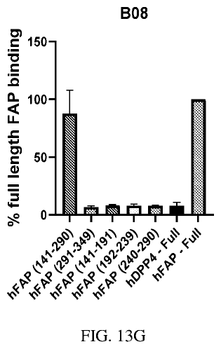

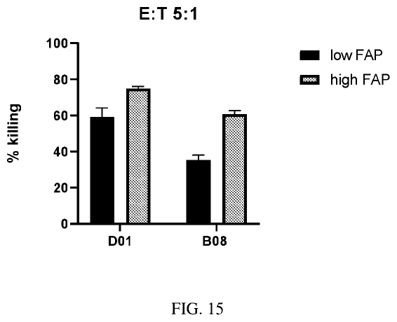

Surface plasmon resonance (SPR) analysis was used to study the binding affinities of E07, D01, B08, A07 and D05 to human and mouse FAP. 2.5 μg/mL human FAP was immobilized to flow cells 1-4 on CM5 chip by amine coupling. Fabs were flowed over at different starting concentrations; E07 (5 nM), B08 (5 nM), D01 (20 nM), A07 (500 nM) and D05 (500 nM), followed by 2-fold dilution series. 5 μg/mL mouse FAP was immobilized to flow cells 5-8 on CM5 chip by amine coupling. Immobilization reached 4000 RU. Fabs were flowed over at different starting concentrations; E07 (10 nM), B08 (10 nM), D01 (500 nM), A07 (500 nM) and D05 (20 nM), followed by 2-fold dilution series. Regeneration was done with 2.5 mM NaOH, 1M NaCl. Plots generated by SPR kinetic analysis demonstrate the association and dissociation characteristics between immobilized human (top panel) or mouse FAP (bottom panel) and analytes. K_{D} values are indicated on the graphs (FIG. 13A). It was observed that D01 binds to human FAP with lower affinity as compared to B08. Surprisingly, iNKT cells expressing CAR comprising FAP binding moiety derived from D01 performed better in in vitro cancer cell killing assays as compared to B08 (FIG. 15). E07, which binds to mouse FAP with a similar KD as D01 to human FAP, was selected as murine surrogate for certain experiments. Results show that iNKT cells expressing a CAR comprising E07 was capable of killing MC38 and 4T1 cells modified to express mouse FAP but do not kill wild type MC38 or 4T1 cells.

Epitope binning was used to characterize the binding of a set of FAP binders; E07, D01, Sibrotuzumab (Sibro), A07 and D05 in the form of Fab or IgG antibodies to a human FAP antigen. FAP antibodies were tested in a pairwise, competition binding assay. All antibodies were competitively assessed versus all other antibodies in the set to determine which antibodies block the same epitopes on a human FAP antigen or not. The antibodies that block binding to the same epitope are “binned” together.

In the first epitope binning experiment, huFAP was immobilized to a CM5 chip and IgGs were flowed over the chip in two steps; First, Fab1 was injected to saturation levels (3 uM) followed by injection of a mixture of Fab1 and Fab2 at a concentration of 3 uM each. Additional binding response in step two indicates that the clones do not share epitope and can bind simultaneously to FAP antigen.

The results are described in FIG. 13B. Fabs of all samples reached saturation, evident by no or very little additional binding when flowing the same clone at the second step. The data shows that E07, B08 and D01 all compete with each other and with Sibro, a commercial anti-FAP antibody. Therefore, they were binned together. A07 and D05 do not compete with any of the tested antibodies, therefore, were binned separately.

In the second epitope binning experiment we used the premix method; IgG1 was captured to a Protein A chip. Then, A premix of huFAP at a concentration of 0.1 uM and saturated levels of Fabs at 10 uM were flowed over the chip. In this case, any binding response indicates that the clones do not share an epitope and can bind simultaneously to FAP antigen.

As observed in the previous experiment, E07, B08 and D01 all compete with each other and with Sibro. Therefore, they were binned together. A07 do not compete with any of the tested antibodies, therefore, was binned separately and the D05 results were inconclusive.

Even though FAP and its related homolog DPP4 share high structural homology (FIG. 13C), D01, B08 and E07 bind specifically to FAP. This observation was exploited to map the epitope of the above antibodies by using human FAP (hFAP) and DPP4 (hDPP4) chimeric constructs. The constructs were designed on the DPP4 backbone and different FAP fragments were introduced to replace the corresponding DPP4 fragment as shown in in FIG. 13D. BFP was used as a marker of transfection and His-tag to confirm expression.

The constructs were transiently expressed in HEK cells and the different IgGs were used to stain the cells. As expected, D01, E07 and B08 do not bind to the human DPP4. However, there is clear gain of binding in the presence of the human FAP domain from amino acid 141 to 290 (FIGS. 13E-13G).

### Example 8. FAP-CAR-IL15-iNKT Cell Transduction

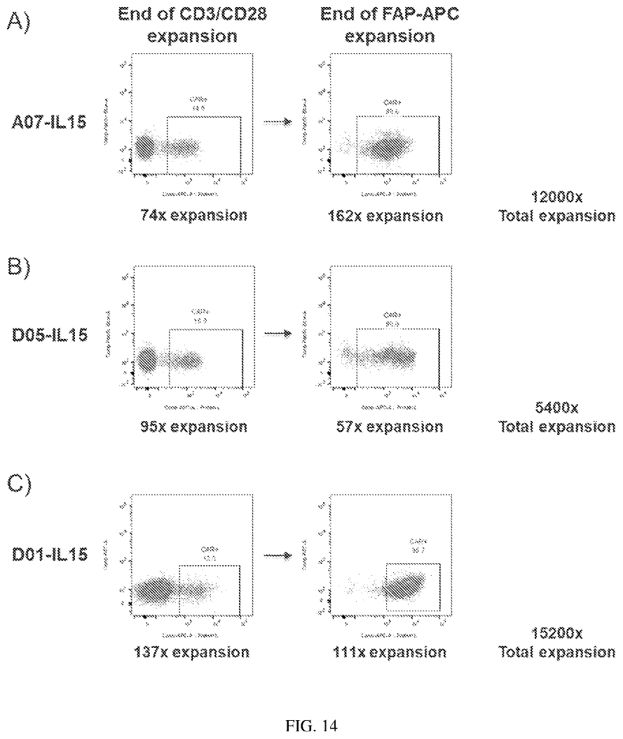

FAP-CAR-IL15-iNKT cells can be successfully transduced using lentivirus and expanded to high purity. iNKT cells were transduced with FAP-CAR-IL15 lentivirus and activated and expanded using CD3/CD28 stimulation for 14 days. On day 14, transduction efficiency is typically 10-20%. iNKT cells were then co-cultured with irradiated monoclonal FAP expressing antigen presenting cells (FAP-APC) to specifically activate and enrich FAP-CAR-IL15 expressing iNKTs over (A-B) 13 days and (C) 12 days. Throughout the manufacturing process, flow cytometric analysis of CAR expression was performed by a two-step staining process. In step 1, cells were washed and then stained with protein-L-biotin (5 μg/ml) in PBS+2% FCS for 30 mins. In step 2, cells were washed and then stained with streptavidin-APC (0.4 μg/ml) and live/dead dye in PBS+2% FCS for 30 mins. FIG. 14 flow cytometry plots show live, single cells and are gated for streptavidin-APC+ cells on the X-axis, while Y-axis shows an unstained flow channel. All CARs tested could be enriched to over 80% purity at the end of FAP-APC expansion. Fold change in total iNKTs is shown for the CD3/CD28 expansion phase and the FAP-APC expansion phase and well as the combined total fold expansion.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms. Definition of terms are disclosed throughout the specification, including but not limited to the “Definition” section.

The section heads are not meant to be interpreted to limit the scope of the present disclosure.

All references, patents and patent applications disclosed herein are incorporated by reference with respect to the subject matter for which each is cited, which in some cases may encompass the entirety of the document.

The indefinite articles “a” and “an.” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either.” “one of,” “only one of,” or “exactly one of.” “Consisting essentially of.” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

What is claimed is:

1. An invariant natural killer T (iNKT) cell comprising a chimeric antigen receptor (CAR) that specifically binds fibroblast activation protein (FAP), wherein the chimeric antigen receptor comprises:
(a) a CDRH1 having the amino acid sequence of SEQ ID NO: 1, a CDRH2 having the amino acid sequence of SEQ ID NO: 2, a CDRH3 having the amino acid sequence of SEQ ID NO: 3, a CDRL1 having the amino acid sequence of SEQ ID NO: 4, a CDRL2 having the amino acid sequence of SEQ ID NO: 5, and a CDRL3 having the amino acid sequence of SEQ ID NO: 6;
(b) a CDRH1 having the amino acid sequence of SEQ ID NO: 10, a CDRH2 having the amino acid sequence of SEQ ID NO: 11, a CDRH3 having the amino acid sequence of SEQ ID NO: 12, a CDRL1 having the amino acid sequence of SEQ ID NO: 4, a CDRL2 having the amino acid sequence of SEQ ID NO: 5, and a CDRL3 having the amino acid sequence of SEQ ID NO: 13;
(c) a CDRH1 having the amino acid sequence of SEQ ID NO: 40, a CDRH2 having the amino acid sequence of SEQ ID NO: 41, a CDRH3 having the amino acid sequence of SEQ ID NO: 42, a CDRL1 having the amino acid sequence of SEQ ID NO: 4, a CDRL2 having the amino acid sequence of SEQ ID NO: 5, and a CDRL3 having the amino acid sequence of SEQ ID NO: 43;
(d) a CDRH1 having the amino acid sequence of SEQ ID NO: 10, a CDRH2 having the amino acid sequence of SEQ ID NO: 11, a CDRH3 having the amino acid sequence of SEQ ID NO: 42, a CDRL1 having the amino acid sequence of SEQ ID NO: 4, a CDRL2 having the amino acid sequence of SEQ ID NO: 5, and a CDRL3 having the amino acid sequence of SEQ ID NO: 43; or
(e) a CDRH1 having the amino acid sequence of SEQ ID NO: 10, a CDRH2 having the amino acid sequence of SEQ ID NO: 11, a CDRH3 having the amino acid sequence of SEQ ID NO: 54, a CDRL1 having the amino acid sequence of SEQ ID NO: 4, a CDRL2 having the amino acid sequence of SEQ ID NO: 5, and a CDRL3 having the amino acid sequence of SEQ ID NO: 55.

2. The iNKT cell of claim 1, wherein the CAR comprises a CDRH1 having the amino acid sequence of SEQ ID NO: 10, a CDRH2 having the amino acid sequence of SEQ ID NO: 11, a CDRH3 having the amino acid sequence of SEQ ID NO: 42, a CDRL1 having the amino acid sequence of SEQ ID NO: 4, a CDRL2 having the amino acid sequence of SEQ ID NO: 5, and a CDRL3 having the amino acid sequence of SEQ ID NO: 43.

3. The iNKT cell of claim 1 or 2, wherein the CAR comprises:
(a) a heavy chain variable region (VH) comprising the amino acid sequence of SEQ ID NO: 7 and a light chain variable region (VL) comprising the amino acid sequence of SEQ ID NO: 8;
(b) a VH comprising the amino acid sequence of SEQ ID NO: 14 and a VL comprising the amino acid sequence of SEQ ID NO: 15;
(c) a VH comprising the amino acid sequence of SEQ ID NO: 44 and a VL comprising the amino acid sequence of SEQ ID NO: 45;
(d) a VH comprising the amino acid sequence of SEQ ID NO: 47 and a VL comprising the amino acid sequence of SEQ ID NO: 48; or
(e) a VH comprising the amino acid sequence of SEQ ID NO: 56 and a VL comprising the amino acid sequence of SEQ ID NO: 57.

4. The iNK cell of any one of claims 1-3, wherein the CAR comprises a VH comprising the amino acid sequence of SEQ ID NO: 47 and a VL comprising the amino acid sequence of SEQ ID NO: 48.

5. The iNKT cell of any one of claims 1-4, wherein the CAR comprises:
(a) a scFv comprising the amino acid sequence of SEQ ID NO: 9;
(b) a scFv comprising the amino acid sequence of SEQ ID NO: 16;
(c) a scFv comprising the amino acid sequence of SEQ ID NO: 46;
(d) a scFv comprising the amino acid sequence of SEQ ID NO: 49; or
(e) a scFv comprising the amino acid sequence of SEQ ID NO: 58.

6. The iNKT cell of any one of claims 1-6, wherein the CAR comprises a scFv comprising the amino acid sequence of SEQ ID NO: 49.

7. The iNKT cell of any one of claims 1-6, wherein the CAR further comprises a hinge region.

8. The iNKT cell of claim 7, wherein the hinge region comprises an amino acid sequence at least 80% identical to SEQ ID NO: 30.

9. The iNKT cell of any one of claims 1-8, wherein the CAR further comprises a transmembrane domain.

10. The iNKT cell of claim 9, wherein the transmembrane domain comprises an amino acid sequence at least 80% identical to SEQ ID NO: 31.

11. The iNKT cell of any one of claims 1-7, wherein the CAR comprises a hinge/transmembrane domain comprising the amino acid sequence of any one of SEQ ID NOs: 83, 85, or 87.

12. The iNKT cell of any of claims 1-11, wherein the CAR further comprises one or more cytoplasmic domain.

13. The iNKT cell of claim 12 wherein the one or more cytoplasmic domain comprises the amino acid sequence of any one of SEQ ID NO: 32, 33, 39, 90, 92, 94, 96, 98, 100, 102, or 105.

14. The iNKT cell of claim 12 or 13, wherein the cytoplasmic domain comprises an amino acid sequence at least 80% identical to SEQ ID NO: 32.

15. The iNKT cell of any one of claims 1-14, wherein the CAR further comprises a intracellular co-stimulatory domain.

16. The iNKT cell of claim 15, wherein the co-stimulatory domain comprises an amino acid sequence at least 80% identical to SEQ ID NO: 33.

17. The iNKT cell of any one of claims 1-16, wherein the CAR comprises the amino acid sequence of any one of SEQ ID NOs: 17, 18, 59, 60, or 62.

18. The iNKT cell of any one of claims 1-17, wherein the CAR comprises the amino acid sequence of SEQ ID NO: 60.

19. The iNKT cell of any one of claims 1-18, wherein the iNKT cell is engineered to express one or more immunoregulatory gene products.

20. The iNKT cell of claim 19, wherein the one or more immunoregulatory gene products comprises IL-15, IL-12, CD40L, or 4-1BB, IL-18, or IL-21.

21. The iNKT cell of claim 19 or 20, wherein the immunoregulatory gene product is a soluble IL-15 (sIL-15).

22. The iNKT cell of any one of claims 1-21, wherein the CAR binds to FAP with a KD in the range of 1E-10 M to 10E-7 M.

23. The iNKT cell of any one of claims 1-22, wherein the CAR binds to FAP with a KD in the range of 1E-8 M to 3E-8 M, or 3E-8 M to 4E-7 M.

24. An invariant natural killer T (iNKT) cell comprising a chimeric antigen receptor (CAR) that specifically binds fibroblast activation protein (FAP), wherein the iNKT cell is engineered to express IL-15.

25. A composition comprising the iNKT cell of any one of claims 1-24.

26. The composition of claim 26, further comprising a pharmaceutically acceptable carrier.

27. A method for killing a cell, the method comprising contacting the cell with the iNKT cell of any one of claims 1-24, or the composition of claim 25 or 26.

28. The method of claim 26, wherein the cell is a cancer cell.

29. The method of claim 28, wherein the cancer cell is a lung cancer cell, a breast cancer cell, a colorectal cancer cell, a prostate cancer cell, a stomach cancer cell, a pancreatic cancer cell, a prostate cancer cell, a thyroid cancer cell, a cervical cancer cell, a urothelial cancer cell, or a sarcoma cell.

30. The method of any one of claims 26-29, wherein the lung cancer is non-small cell lung cancer.

31. The method of any one of claims 26-30, wherein the cell in the tumor microenvironment express FAP.

32. A method for treating tumor in a subject, the method comprising administering to a subject having or suspected of having cancer, the iNKT of any one of claims 1-24, or the composition of claim 25 or 26.

33. A method for reducing tumor growth, the method comprising contacting the tumor in a subject with the iNKT of any one of claims 1-24, or the composition of claim 25 or 26.

34. The method of claim 32 or 33, wherein the subject is a human.

35. The method of any one of claims 32-34, wherein the tumor is a solid tumor.

36. The method of claim 35, wherein the solid tumor is lung cancer tumor, breast cancer tumor, colorectal cancer tumor, prostate cancer tumor, stomach cancer tumor, pancreatic cancer tumor, prostate cancer tumor, thyroid cancer tumor, cervical cancer tumor, urothelial cancer tumor, or sarcoma.

37. The method of any one of claim 36, wherein the lung cancer is non-small cell lung cancer.

38. The method of any one of claims 32-37, wherein the administration is via injection,

39. The method of claim 38, wherein the injection is intraperitoneal injection, intravenous injection, or intratumoral injection.

40. The method of any one of claims 32-39, wherein the iNKT cell is administered in combination with a therapeutic agent.

41. The method of claim 40, wherein the therapeutic agent is a CAR T cell specific for a tumor antigen.

42. The method of claim 41, wherein the therapeutic agent is an immune checkpoint inhibitor or agonist.

43. A method for treating a tumor in a subject, the method comprising administering the subject a composition comprising an invariant natural killer T (iNKT) cell comprising a chimeric antigen receptor (CAR) having an anti-FAP binding moiety.

44. A method for killing tumor cells, the method comprising contacting the tumor cells with a composition comprising an invariant natural killer T (iNKT) cell comprising a chimeric antigen receptor (CAR) having an anti-FAP binding moiety.

45. A method for reducing immunosuppression in the tumor microenvironment (TME) of a subject relative to immunosuppression in the subject prior to the administration, the method comprising administering the subject a composition comprising an invariant natural killer T (iNKT) cell comprising a chimeric antigen receptor (CAR) having an anti-FAP binding moiety.

46. The method of any one of claims 43-45, wherein the anti-FAP CAR iNKT cells are engineered to an armoring molecule.

47. The method of claim 46, wherein the armoring molecule is soluble IL-15 (sIL-15).

48. The method of any one of claims 43-47, wherein the tumor is a solid tumor.

49. The method of any one of claims 43-48, wherein the tumor is of epithelial cell origin.

50. The method of 49, wherein the solid tumor is lung cancer tumor, breast cancer tumor, colorectal cancer tumor, prostate cancer tumor, stomach cancer tumor, pancreatic cancer tumor, prostate cancer tumor, thyroid cancer tumor, cervical cancer tumor, urothelial cancer tumor, or sarcoma tumor.

51. The method of any one of claims 43-50, wherein the anti-FAP CAR iNKT cells kill FAP expressing cells in the TME.

52. The method of claim 51, wherein the FAP expressing cells in the TME comprise tumor cells expressing FAP, and/or cancer-associated fibroblasts.

53. The method of claim 51 or 52, wherein the anti-FAP CAR iNKT cells kill FAP expressing cells directly.

54. The method of claim 51 or 52, wherein the anti-FAP CAR iNKT cells kill FAP expressing cells indirectly.

55. The method of any one of claims 51-54, wherein the killing of cancer-associated fibroblasts results in reduced immunosuppression in the TME relative to immunosuppression in the subject prior to the administration.

56. The method of any one of claims 52-55, wherein the killing of cancer-associated fibroblasts results in increased immune cell infiltration in the TME relative to immune cell infiltration in the subject prior to the administration.

57. The method of any one of claims 43-46, wherein the method further comprises administering the subject a CAR T cell targeting a tumor antigen comprising NY-ESO-1, or BCMA.

58. The method of any one of claims 43-57, wherein the administration of the anti-FAP CAR iNKT cells leads to reduced tumor burden relative to tumor burden in the subject prior to the administration.

59. The method of any one of claims 43-58, wherein the administration of the anti-FAP CAR iNKT cells leads to resistance to T cell exhaustion, enhancement of tissue homing of anti-FAP iNKT cells, selective cytotoxicity towards M2 macrophages, and/or stimulation of dendritic cell maturation.

60. The method of any one of claims 43-59, wherein the anti-FAP CAR iNKT cells retain response to CD1d and/or NK receptor ligand.

61. The method of any one of claims 43-60, wherein the anti-FAP CAR iNKT cells comprise a CAR comprising any one of the FAP binding moieties derived from the anti-FAP antibodies set forth in Table 3.

62. The method of any one of claims 43-61, wherein the subject is a human.

63. The method of any one of claims 43-63, wherein the subject is diagnosed as having a solid tumor comprising lung cancer, breast cancer, colorectal cancer, prostate cancer, stomach cancer, pancreatic cancer, prostate cancer, thyroid cancer, cervical cancer, urothelial cancer, or sarcoma.


SEQUENCE LISTING (107 sequences)

SEQ ID NO: 1 - protein, 5 aa, Synthetic construct
SYAIS

SEQ ID NO: 2 - protein, 17 aa, Synthetic construct
GIIPIFGTANYAQKFQG

SEQ ID NO: 3 - protein, 18 aa, Synthetic construct
PRDCSSTSCYAGIDAFDI

SEQ ID NO: 4 - protein, 12 aa, Synthetic construct
RASQSVSSSYLA

SEQ ID NO: 5 - protein, 7 aa, Synthetic construct
GASSRAT

SEQ ID NO: 6 - protein, 9 aa, Synthetic construct
QQYGNSPTT

SEQ ID NO: 7 - protein, 127 aa, Synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCASPRDCSSTSCYAGIDAFDIWGQGTMVTVSS

SEQ ID NO: 8 - protein, 110 aa, Synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGNSPTTFGGGTKLEIKRT

SEQ ID NO: 9 - protein, 252 aa, Synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCASPRDCSSTSCYAGIDAFDIWGQGTMVTVSSGGGGSGGGGSGGGASEIVLTQSPGTLSLSPGERATLSCRASQSVSSSYLAWYQQKPGQAPRLLIYGASSRATGIPDRFSGSGSGTDFTLTISRLEPEDFAVYYCQQYGNSPTTFGGGTKLEIKRT

SEQ ID NO: 10 - protein, 5 aa, Synthetic construct
SYAMS

SEQ ID NO: 11 - protein, 17 aa, Synthetic construct
AISGSGGSTYYADSVKG

SEQ ID NO: 12 - protein, 11 aa, Synthetic construct
THSTSWYYFDY

SEQ ID NO: 13 - protein, 10 aa, Synthetic construct
QQYGSSPRYT

SEQ ID NO: 14 - protein, 120 aa, Synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKTHSTSWYYFDYWGRGTLVTVSS

SEQ ID NO: 15 - protein, 111 aa, Synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPRYTFGQGTKVDIKRT

SEQ ID NO: 16 - protein, 246 aa, Synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKTHSTSWYYFDYWGRGTLVTVSSGGGGSGGGGSGGGASEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPRYTFGQGTKVDIKRT

SEQ ID NO: 17 - protein, 499 aa, Synthetic construct
MALPVTALLLPLALLLHAARPQVQLVQSGAEVKKPGSSVKVSCKASGGTFSSYAISWVRQAPGQGLEWMGGIIPIFGTANYAQKFQGRVTITADESTSTAYMELSSLRSEDTAVYYCASPRDCSSTSCYAGIDAFDIWGQGTMVTVSSGGGGSGGGGSGGGASEIVLTQSPGTLSLSPGERATLSCRASQSVSSSYLAWYQQKPGQAPRLLIYGASSRATGIPDRFSGSGSGTDFTLTISRLEPEDFAVYYCQQYGNSPTTFGGGTKLEIKRTAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - protein, 493 aa, Synthetic construct
MALPVTALLLPLALLLHAARPEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKTHSTSWYYFDYWGRGTLVTVSSGGGGSGGGGSGGGASEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPRYTFGQGTKVDIKRTAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - DNA, 381 bp, Synthetic construct
  source 1..381: mol_type = other DNA
caggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgagcccccgcgattgtagtagtaccagctgctatgcggggattgatgcttttgatatctggggccaagggacaatggtcaccgtctcgagt

SEQ ID NO: 20 - DNA, 330 bp, Synthetic construct
  source 1..330: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattattgtcagcagtatggtaactcacctaccactttcggcggagggaccaagctggagatcaaacggacc

SEQ ID NO: 21 - DNA, 756 bp, Synthetic construct
  source 1..756: mol_type = other DNA
caggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgagcccccgcgattgtagtagtaccagctgctatgcggggattgatgcttttgatatctggggccaagggacaatggtcaccgtctcgagtggtggaggcggttcaggcggaggtggctctggcggtggcgctagcgaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattattgtcagcagtatggtaactcacctaccactttcggcggagggaccaagctggagatcaaacggacc

SEQ ID NO: 22 - DNA, 1434 bp, Synthetic construct
  source 1..1434: mol_type = other DNA
caggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgagcccccgcgattgtagtagtaccagctgctatgcggggattgatgcttttgatatctggggccaagggacaatggtcaccgtctcgagtggtggaggcggttcaggcggaggtggctctggcggtggcgctagcgaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattattgtcagcagtatggtaactcacctaccactttcggcggagggaccaagctggagatcaaacggaccgcggccgcaaccacaacccctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgc

SEQ ID NO: 23 - DNA, 9392 bp, Synthetic construct
  source 1..9392: mol_type = other DNA
cgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacccgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatactagtattatgcccagtacatgaccttatgggactttcctacttggcagtacatctacaaaccccgggtgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtagcggccctaatacgactcactatagggcccgcgtacgtctagacctccaggcatcgattccaccatggcactccccgtcaccgcccttctcttgcccctcgccctgctgctgcatgctgccaggccccaggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgagcccccgcgattgtagtagtaccagctgctatgcggggattgatgcttttgatatctggggccaagggacaatggtcaccgtctcgagtggtggaggcggttcaggcggaggtggctctggcggtggcgctagcgaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattattgtcagcagtatggtaactcacctaccactttcggcggagggaccaagctggagatcaaacggaccgcggccgcaaccacaacccctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgcggatcaggagcgactaacttctctttgttgaagcaagcgggtgacgttgaagagaatcccggtccatccgggggatccatgcggatctccaagccccacctccgaagcatcagtatccagtgttatctgtgccttcttctgaattcccattttttgacagaggcaggaattcatgtattcatattgggttgtttctccgcggggcttcctaaaaccgaagctaactgggttaatgtcatttcagatctgaagaaaattgaagatcttattcaatcaatgcatatcgacgcgaccttgtacacggagagtgatgtccacccgagctgcaaggtaacggccatgaaatgcttcttgctggaattgcaggtaattagtcttgagagtggagatgcgtctatacacgacaccgtagagaatctcattattttggcgaacaattctctcagcagtaacggcaatgttactgaatcaggatgtaaggagtgtgaagagctggaagaaaagaacattaaggaatttttgcagagcttcgtacatatcgtccaaatgttcataaatacaagctaagtcgacggccggccgcgagcatcttaccgccatttatacccatatttgttctgtttttcttgatttgggtatacatttaaatgttaataaaacaaaatggtggggcaatcatttacattttataggatatgtaattactagttcaggtgtattgccacaagacaaacatcttaagaaactttcccgttatttacgctctgttcctgttaatcaacctctggattacaaaatttgtgaaagattgactgatattcttaactatgttgctccttttacgctgtgtggatatgctgctttaatacctctgtatcatactattgcttcctgtacggctttcgttttctcctccttgtataaatcctggttgctgtctctttatcaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcctggtacctttaagaccaatgacttacaaggcagctgtagatcttagccactttttaaaagaaaaggggggactggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagacttttgcagagaccaaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccc

SEQ ID NO: 24 - DNA, 360 bp, Synthetic construct
  source 1..360: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaaacccacagcaccagctggtactactttgactactggggccgtggcaccctggtcactgtctcgagt

SEQ ID NO: 25 - DNA, 333 bp, Synthetic construct
  source 1..333: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccccggtacacttttggccaggggaccaaagtggatatcaaacggacc

SEQ ID NO: 26 - DNA, 738 bp, Synthetic construct
  source 1..738: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaaacccacagcaccagctggtactactttgactactggggccgtggcaccctggtcactgtctcgagtggtggaggcggttcaggcggaggtggctctggcggtggcgctagcgaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccccggtacacttttggccaggggaccaaagtggatatcaaacggacc

SEQ ID NO: 27 - DNA, 1416 bp, Synthetic construct
  source 1..1416: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaaacccacagcaccagctggtactactttgactactggggccgtggcaccctggtcactgtctcgagtggtggaggcggttcaggcggaggtggctctggcggtggcgctagcgaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccccggtacacttttggccaggggaccaaagtggatatcaaacggaccgcggccgcaaccacaacccctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgc

SEQ ID NO: 28 - DNA, 9374 bp, Synthetic construct
  source 1..9374: mol_type = other DNA
cgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacccgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatactagtattatgcccagtacatgaccttatgggactttcctacttggcagtacatctacaaaccccgggtgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtagcggccctaatacgactcactatagggcccgcgtacgtctagacctccaggcatcgattccaccatggcactccccgtcaccgcccttctcttgcccctcgccctgctgctgcatgctgccaggcccgaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaaacccacagcaccagctggtactactttgactactggggccgtggcaccctggtcactgtctcgagtggtggaggcggttcaggcggaggtggctctggcggtggcgctagcgaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccccggtacacttttggccaggggaccaaagtggatatcaaacggaccgcggccgcaaccacaacccctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgcggatcaggagcgactaacttctctttgttgaagcaagcgggtgacgttgaagagaatcccggtccatccgggggatccatgcggatctccaagccccacctccgaagcatcagtatccagtgttatctgtgccttcttctgaattcccattttttgacagaggcaggaattcatgtattcatattgggttgtttctccgcggggcttcctaaaaccgaagctaactgggttaatgtcatttcagatctgaagaaaattgaagatcttattcaatcaatgcatatcgacgcgaccttgtacacggagagtgatgtccacccgagctgcaaggtaacggccatgaaatgcttcttgctggaattgcaggtaattagtcttgagagtggagatgcgtctatacacgacaccgtagagaatctcattattttggcgaacaattctctcagcagtaacggcaatgttactgaatcaggatgtaaggagtgtgaagagctggaagaaaagaacattaaggaatttttgcagagcttcgtacatatcgtccaaatgttcataaatacaagctaagtcgacggccggccgcgagcatcttaccgccatttatacccatatttgttctgtttttcttgatttgggtatacatttaaatgttaataaaacaaaatggtggggcaatcatttacattttataggatatgtaattactagttcaggtgtattgccacaagacaaacatcttaagaaactttcccgttatttacgctctgttcctgttaatcaacctctggattacaaaatttgtgaaagattgactgatattcttaactatgttgctccttttacgctgtgtggatatgctgctttaatacctctgtatcatactattgcttcctgtacggctttcgttttctcctccttgtataaatcctggttgctgtctctttatcaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcctggtacctttaagaccaatgacttacaaggcagctgtagatcttagccactttttaaaagaaaaggggggactggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagacttttgcagagaccaaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccc

SEQ ID NO: 29 - protein, 15 aa, Synthetic construct
GGGGSGGGGSGGGAS

SEQ ID NO: 30 - protein, 45 aa, Synthetic construct
TTTPAPRPPTPAPTIASQPLSLRPEACRPAAGGAVHTRGLDFACD

SEQ ID NO: 31 - protein, 24 aa, Synthetic construct
IYIWAPLAGTCGVLLLSLVITLYC

SEQ ID NO: 32 - protein, 112 aa, Synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - protein, 42 aa, Synthetic construct
KRGRKKLLYIFKQPFMRPVQTTQEEDGCSCRFPEEEEGGCEL

SEQ ID NO: 34 - protein, 486 aa, Synthetic construct
ATGCGGATCTCCAAGCCCCACCTCCGAAGCATCAGTATCCAGTGTTATCTGTGCCTTCTTCTGAATTCCCATTTTTTGACAGAGGCAGGAATTCATGTATTCATATTGGGTTGTTTCTCCGCGGGGCTTCCTAAAACCGAAGCTAACTGGGTTAATGTCATTTCAGATCTGAAGAAAATTGAAGATCTTATTCAATCAATGCATATCGACGCGACCTTGTACACGGAGAGTGATGTCCACCCGAGCTGCAAGGTAACGGCCATGAAATGCTTCTTGCTGGAATTGCAGGTAATTAGTCTTGAGAGTGGAGATGCGTCTATACACGACACCGTAGAGAATCTCATTATTTTGGCGAACAATTCTCTCAGCAGTAACGGCAATGTTACTGAATCAGGATGTAAGGAGTGTGAAGAGCTGGAAGAAAAGAACATTAAGGAATTTTTGCAGAGCTTCGTACATATCGTCCAAATGTTCATAAATACAAGC

SEQ ID NO: 35 - protein, 760 aa, Synthetic construct [sequence omitted]

SEQ ID NO: 36 - protein, 761 aa, Mus musculus
MKTWLKTVFGVTTLAALALVVICIVLRPSRVYKPEGNTKRALTLKDILNGTFSYKTYFPNWISEQEYLHQSEDDNIVFYNIETRESYIILSNSTMKSVNATDYGLSPDRQFVYLESDYSKLWRYSYTATYYIYDLQNGEFVRGYELPRPIQYLCWSPVGSKLAYVYQNNIYLKQRPGDPPFQITYTGRENRIFNGIPDWVYEEEMLATKYALWWSPDGKFLAYVEFNDSDIPIIAYSYYGDGQYPRTINIPYPKAGAKNPVVRVFIVDTTYPHHVGPMEVPVPEMIASSDYYFSWLTWVSSERVCLQWLKRVQNVSVLSICDFREDWHAWECPKNQEHVEESRTGWAGGFFVSTPAFSQDATSYYKIFSDKDGYKHIHYIKDTVENAIQITSGKWEAIYIFRVTQDSLFYSSNEFEGYPGRRNIYRISIGNSPPSKKCVTCHLRKERCQYYTASFSYKAKYYALVCYGPGLPISTLHDGRTDQEIQVLEENKELENSLRNIQLPKVEIKKLKDGGLTFWYKMILPPQFDRSKKYPLLIQVYGGPCSQSVKSVFAVNWITYLASKEGIVIALVDGRGTAFQGDKFLHAVYRKLGVYEVEDQLTAVRKFIEMGFIDEERIAIWGWSYGGYVSSLALASGTGLFKCGIAVAPVSSWEYYASIYSERFMGLPTKDDNLEHYKNSTVMARAEYFRNVDYLLIHGTADDNVHFQNSAQIAKALVNAQVDFQAMWYSDQNHGISSGRSQNHLYTHMTHFLKQCFSLSD

SEQ ID NO: 37 - protein, 736 aa, Mus musculus
LRPSRVYKPEGNTKRALTLKDILNGTFSYKTYFPNWISEQEYLHQSEDDNIVFYNIETRESYIILSNSTMKSVNATDYGLSPDRQFVYLESDYSKLWRYSYTATYYIYDLQNGEFVRGYELPRPIQYLCWSPVGSKLAYVYQNNIYLKQRPGDPPFQITYTGRENRIFNGIPDWVYEEEMLATKYALWWSPDGKFLAYVEFNDSDIPIIAYSYYGDGQYPRTINIPYPKAGAKNPVVRVFIVDTTYPHHVGPMEVPVPEMIASSDYYFSWLTWVSSERVCLQWLKRVQNVSVLSICDFREDWHAWECPKNQEHVEESRTGWAGGFFVSTPAFSQDATSYYKIFSDKDGYKHIHYIKDTVENAIQITSGKWEAIYIFRVTQDSLFYSSNEFEGYPGRRNIYRISIGNSPPSKKCVTCHLRKERCQYYTASFSYKAKYYALVCYGPGLPISTLHDGRTDQEIQVLEENKELENSLRNIQLPKVEIKKLKDGGLTFWYKMILPPQFDRSKKYPLLIQVYGGPCSQSVKSVFAVNWITYLASKEGIVIALVDGRGTAFQGDKFLHAVYRKLGVYEVEDQLTAVRKFIEMGFIDEERIAIWGWSYGGYVSSLALASGTGLFKCGIAVAPVSSWEYYASIYSERFMGLPTKDDNLEHYKNSTVMARAEYFRNVDYLLIHGTADDNVHFQNSAQIAKALVNAQVDFQAMWYSDQNHGISSGRSQNHLYTHMTHFLKQCFSLSD

SEQ ID NO: 38 - protein, 761 aa, Mus musculus
MKTWLKTVFGVTTLAALALVVICIVLRPSRVYKPEGNTKRALTLKDILNGTFSYKTYFPNWISEQEYLHQSEDDNIVFYNIETRESYIILSNSTMKSVNATDYGLSPDRQFVYLESDYSKLWRYSYTATYYIYDLQNGEFVRGYELPRPIQYLCWSPVGSKLAYVYQNNIYLKQRPGDPPFQITYTGRENRIFNGIPDWVYEEEMLATKYALWWSPDGKFLAYVEFNDSDIPIIAYSYYGDGQYPRTINIPYPKAGAKNPVVRVFIVDTTYPHHVGPMEVPVPEMIASSDYYFSWLTWVSSERVCLQWLKRVQNVSVLSICDFREDWHAWECPKNQEHVEESRTGWAGGFFVSTPAFSQDATSYYKIFSDKDGYKHIHYIKDTVENAIQITSGKWEAIYIFRVTQDSLFYSSNEFEGYPGRRNIYRISIGNSPPSKKCVTCHLRKERCQYYTASFSYKAKYYALVCYGPGLPISTLHDGRTDQEIQVLEENKELENSLRNIQLPKVEIKKLKDGGLTFWYKMILPPQFDRSKKYPLLIQVYGGPCSQSVKSVFAVNWITYLASKEGIVIALVDGRGTAFQGDKFLHAVYRKLGVYEVEDQLTAVRKFIEMGFIDEERIAIWGWSYGGYVSSLALASGTGLFKCGIAVAPVSSWEYYASIYSERFMGLPTKDDNLEHYKNSTVMARAEYFRNVDYLLIHGTADDNVHFQNSAQIAKALVNAQVDFQAMWYSDQNHGILSGRSQNHLYTHMTHFLKQCFSLSD

SEQ ID NO: 39 - protein, 85 aa, Synthetic construct
LRKRRDSLSLSTQRTQGPAESARNLEYVSVSPTNNTVYASVTHSNRETEIWTPRENDTITIYSTINHSKESKPTFSRATALDNVV

SEQ ID NO: 40 - protein, 5 aa, Synthetic construct
SYVMS

SEQ ID NO: 41 - protein, 17 aa, Synthetic construct
AIVPSGGDTYYADSVKG

SEQ ID NO: 42 - protein, 9 aa, Synthetic construct
DYYGGWFDP

SEQ ID NO: 43 - protein, 9 aa, Synthetic construct
QQYGSSPRT

SEQ ID NO: 44 - protein, 118 aa, Synthetic construct
EVQLLESGGGLVQPGGSLRLSCAASGFTFKSYVMSWVRQAPGKGLEWVSAIVPSGGDTYYADSVKGRFTISRDNSKNTLYLQMNSLRAEDTAVYYCAKDYYGGWFDPWGQGTLVTVSS

SEQ ID NO: 45 - protein, 108 aa, Synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPRTFGQGTKVEIK

SEQ ID NO: 46 - protein, 241 aa, Synthetic construct
EVQLLESGGGLVQPGGSLRLSCAASGFTFKSYVMSWVRQAPGKGLEWVSAIVPSGGDTYYADSVKGRFTISRDNSKNTLYLQMNSLRAEDTAVYYCAKDYYGGWFDPWGQGTLVTVSSGGGGSGGGGSGGGASEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPRTFGQGTKVEIK

SEQ ID NO: 47 - protein, 118 aa, Synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDYYGGWFDPWGQGTLVTVSS

SEQ ID NO: 48 - protein, 108 aa, Synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPRTFGHGTKLEIK

SEQ ID NO: 49 - protein, 241 aa, Synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDYYGGWFDPWGQGTLVTVSSGGGGSGGGGSGGGASEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPRTFGHGTKLEIK

SEQ ID NO: 50 - protein, 17 aa, Synthetic construct
GISEGGGSTYYADSVKG

SEQ ID NO: 51 - protein, 118 aa, Synthetic construct
EVQLLESGGGLVQPGGSLRLSCAASGFTFSSYAMSWVRQAPGKGLEWVSGISEGGGSTYYADSVKGRFTISRDNSKNTLYLQMNSLRAEDTAVYYCAKDYYGGWFDPWGRGTLVTVSS

SEQ ID NO: 52 - protein, 108 aa, Synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPRTFGQGTRLEIK

SEQ ID NO: 53 - protein, 241 aa, Synthetic construct
EVQLLESGGGLVQPGGSLRLSCAASGFTFSSYAMSWVRQAPGKGLEWVSGISEGGGSTYYADSVKGRFTISRDNSKNTLYLQMNSLRAEDTAVYYCAKDYYGGWFDPWGRGTLVTVSSGGGGSGGGGSGGGASEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPRTFGQGTRLEIK

SEQ ID NO: 54 - protein, 11 aa, Synthetic construct
DLSWYGANFDY

SEQ ID NO: 55 - protein, 11 aa, Synthetic construct
QQYGSSPVMYT

SEQ ID NO: 56 - protein, 120 aa, Synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDLSWYGANFDYWGQGTLVTVSS

SEQ ID NO: 57 - protein, 110 aa, Synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPVMYTFGQGTKVEIK

SEQ ID NO: 58 - protein, 245 aa, Synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDLSWYGANFDYWGQGTLVTVSSGGGGSGGGGSGGGASEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPVMYTFGQGTKVEIK

SEQ ID NO: 59 - protein, 488 aa, Synthetic construct
MALPVTALLLPLALLLHAARPEVQLLESGGGLVQPGGSLRLSCAASGFTFKSYVMSWVRQAPGKGLEWVSAIVPSGGDTYYADSVKGRFTISRDNSKNTLYLQMNSLRAEDTAVYYCAKDYYGGWFDPWGQGTLVTVSSGGGGSGGGGSGGGASEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPRTFGQGTKVEIKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 60 - protein, 488 aa, Synthetic construct
MALPVTALLLPLALLLHAARPEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDYYGGWFDPWGQGTLVTVSSGGGGSGGGGSGGGASEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPRTFGHGTKLEIKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 61 - protein, 488 aa, Synthetic construct
MALPVTALLLPLALLLHAARPEVQLLESGGGLVQPGGSLRLSCAASGFTFSSYAMSWVRQAPGKGLEWVSGISEGGGSTYYADSVKGRFTISRDNSKNTLYLQMNSLRAEDTAVYYCAKDYYGGWFDPWGRGTLVTVSSGGGGSGGGGSGGGASEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPRTFGQGTRLEIKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 62 - protein, 492 aa, Synthetic construct
MALPVTALLLPLALLLHAARPEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDLSWYGANFDYWGQGTLVTVSSGGGGSGGGGSGGGASEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPVMYTFGQGTKVEIKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 63 - DNA, 354 bp, Synthetic construct
  source 1..354: mol_type = other DNA
gaggtgcagctgttggagtctgggggaggcttggtacagcctggggggtccctgagactctcctgtgcagcctctggattcacctttaaaagctatgtgatgagctgggtccgccaggctccagggaaggggctggagtgggtctcagctattgtgccaagtggtggtgatacatactacgcagactccgtgaagggccggttcaccatctccagagacaattccaagaacacgctgtatctgcaaatgaacagcctgagagccgaggacacggccgtatattactgtgcgaaagattactacggagggtggttcgacccctggggccagggaaccctggtcaccgtctcgagt

SEQ ID NO: 64 - DNA, 324 bp, Synthetic construct
  source 1..324: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctcgaacgttcggccaagggaccaaggtggagatcaaa

SEQ ID NO: 65 - DNA, 723 bp, Synthetic construct
  source 1..723: mol_type = other DNA
gaggtgcagctgttggagtctgggggaggcttggtacagcctggggggtccctgagactctcctgtgcagcctctggattcacctttaaaagctatgtgatgagctgggtccgccaggctccagggaaggggctggagtgggtctcagctattgtgccaagtggtggtgatacatactacgcagactccgtgaagggccggttcaccatctccagagacaattccaagaacacgctgtatctgcaaatgaacagcctgagagccgaggacacggccgtatattactgtgcgaaagattactacggagggtggttcgacccctggggccagggaaccctggtcaccgtctcgagtggtggaggcggttcaggcggaggtggctctggcggtggcgctagcgaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctcgaacgttcggccaagggaccaaggtggagatcaaa

SEQ ID NO: 66 - DNA, 1401 bp, Synthetic construct
  source 1..1401: mol_type = other DNA
gaggtgcagctgttggagtctgggggaggcttggtacagcctggggggtccctgagactctcctgtgcagcctctggattcacctttaaaagctatgtgatgagctgggtccgccaggctccagggaaggggctggagtgggtctcagctattgtgccaagtggtggtgatacatactacgcagactccgtgaagggccggttcaccatctccagagacaattccaagaacacgctgtatctgcaaatgaacagcctgagagccgaggacacggccgtatattactgtgcgaaagattactacggagggtggttcgacccctggggccagggaaccctggtcaccgtctcgagtggtggaggcggttcaggcggaggtggctctggcggtggcgctagcgaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctcgaacgttcggccaagggaccaaggtggagatcaaagcggccgcaaccacaacccctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgc

SEQ ID NO: 67 - DNA, 9359 bp, Synthetic construct
  source 1..9359: mol_type = other DNA
cgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacccgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatactagtattatgcccagtacatgaccttatgggactttcctacttggcagtacatctacaaaccccgggtgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtagcggccctaatacgactcactatagggcccgcgtacgtctagacctccaggcatcgattccaccatggcactccccgtcaccgcccttctcttgcccctcgccctgctgctgcatgctgccaggcccgaggtgcagctgttggagtctgggggaggcttggtacagcctggggggtccctgagactctcctgtgcagcctctggattcacctttaaaagctatgtgatgagctgggtccgccaggctccagggaaggggctggagtgggtctcagctattgtgccaagtggtggtgatacatactacgcagactccgtgaagggccggttcaccatctccagagacaattccaagaacacgctgtatctgcaaatgaacagcctgagagccgaggacacggccgtatattactgtgcgaaagattactacggagggtggttcgacccctggggccagggaaccctggtcaccgtctcgagtggtggaggcggttcaggcggaggtggctctggcggtggcgctagcgaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctcgaacgttcggccaagggaccaaggtggagatcaaagcggccgcaaccacaacccctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgcggatcaggagcgactaacttctctttgttgaagcaagcgggtgacgttgaagagaatcccggtccatccgggggatccatgcggatctccaagccccacctccgaagcatcagtatccagtgttatctgtgccttcttctgaattcccattttttgacagaggcaggaattcatgtattcatattgggttgtttctccgcggggcttcctaaaaccgaagctaactgggttaatgtcatttcagatctgaagaaaattgaagatcttattcaatcaatgcatatcgacgcgaccttgtacacggagagtgatgtccacccgagctgcaaggtaacggccatgaaatgcttcttgctggaattgcaggtaattagtcttgagagtggagatgcgtctatacacgacaccgtagagaatctcattattttggcgaacaattctctcagcagtaacggcaatgttactgaatcaggatgtaaggagtgtgaagagctggaagaaaagaacattaaggaatttttgcagagcttcgtacatatcgtccaaatgttcataaatacaagctaagtcgacggccggccgcgagcatcttaccgccatttatacccatatttgttctgtttttcttgatttgggtatacatttaaatgttaataaaacaaaatggtggggcaatcatttacattttataggatatgtaattactagttcaggtgtattgccacaagacaaacatcttaagaaactttcccgttatttacgctctgttcctgttaatcaacctctggattacaaaatttgtgaaagattgactgatattcttaactatgttgctccttttacgctgtgtggatatgctgctttaatacctctgtatcatactattgcttcctgtacggctttcgttttctcctccttgtataaatcctggttgctgtctctttatcaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcctggtacctttaagaccaatgacttacaaggcagctgtagatcttagccactttttaaaagaaaaggggggactggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagacttttgcagagaccaaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccc

SEQ ID NO: 68 - DNA, 354 bp, Synthetic construct
  source 1..354: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagattactacggagggtggttcgacccctggggccagggaaccctggtcaccgtctcgagt

SEQ ID NO: 69 - DNA, 324 bp, Synthetic construct
  source 1..324: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctcggacattcggccacgggaccaagctggagatcaaa

SEQ ID NO: 70 - DNA, 723 bp, Synthetic construct
  source 1..723: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagattactacggagggtggttcgacccctggggccagggaaccctggtcaccgtctcgagtggtggaggcggttcaggcggaggtggctctggcggtggcgctagcgaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctcggacattcggccacgggaccaagctggagatcaaa

SEQ ID NO: 71 - DNA, 1401 bp, Synthetic construct
  source 1..1401: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagattactacggagggtggttcgacccctggggccagggaaccctggtcaccgtctcgagtggtggaggcggttcaggcggaggtggctctggcggtggcgctagcgaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctcggacattcggccacgggaccaagctggagatcaaagcggccgcaaccacaacccctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgc

SEQ ID NO: 72 - DNA, 9359 bp, Synthetic construct
  source 1..9359: mol_type = other DNA
cgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacccgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatactagtattatgcccagtacatgaccttatgggactttcctacttggcagtacatctacaaaccccgggtgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtagcggccctaatacgactcactatagggcccgcgtacgtctagacctccaggcatcgattccaccatggcactccccgtcaccgcccttctcttgcccctcgccctgctgctgcatgctgccaggcccgaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagattactacggagggtggttcgacccctggggccagggaaccctggtcaccgtctcgagtggtggaggcggttcaggcggaggtggctctggcggtggcgctagcgaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctcggacattcggccacgggaccaagctggagatcaaagcggccgcaaccacaacccctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgcggatcaggagcgactaacttctctttgttgaagcaagcgggtgacgttgaagagaatcccggtccatccgggggatccatgcggatctccaagccccacctccgaagcatcagtatccagtgttatctgtgccttcttctgaattcccattttttgacagaggcaggaattcatgtattcatattgggttgtttctccgcggggcttcctaaaaccgaagctaactgggttaatgtcatttcagatctgaagaaaattgaagatcttattcaatcaatgcatatcgacgcgaccttgtacacggagagtgatgtccacccgagctgcaaggtaacggccatgaaatgcttcttgctggaattgcaggtaattagtcttgagagtggagatgcgtctatacacgacaccgtagagaatctcattattttggcgaacaattctctcagcagtaacggcaatgttactgaatcaggatgtaaggagtgtgaagagctggaagaaaagaacattaaggaatttttgcagagcttcgtacatatcgtccaaatgttcataaatacaagctaagtcgacggccggccgcgagcatcttaccgccatttatacccatatttgttctgtttttcttgatttgggtatacatttaaatgttaataaaacaaaatggtggggcaatcatttacattttataggatatgtaattactagttcaggtgtattgccacaagacaaacatcttaagaaactttcccgttatttacgctctgttcctgttaatcaacctctggattacaaaatttgtgaaagattgactgatattcttaactatgttgctccttttacgctgtgtggatatgctgctttaatacctctgtatcatactattgcttcctgtacggctttcgttttctcctccttgtataaatcctggttgctgtctctttatcaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcctggtacctttaagaccaatgacttacaaggcagctgtagatcttagccactttttaaaagaaaaggggggactggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagacttttgcagagaccaaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccc

SEQ ID NO: 73 - DNA, 354 bp, Synthetic construct
  source 1..354: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcaggtattagtgaaggtggtggtagcacatactacgcagactccgtgaagggccggttcaccatctccagagacaattccaagaacacgctgtatctgcaaatgaacagcctgagagccgaggacacggccgtatattactgtgcgaaagattactacggagggtggttcgacccctggggccgtggcaccctggtcactgtctcgagt

SEQ ID NO: 74 - DNA, 330 bp, Synthetic construct
  source 1..330: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctcgaacttttggccaggggacacgactggagattaaacggacc

SEQ ID NO: 75 - DNA, 729 bp, Synthetic construct
  source 1..729: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcaggtattagtgaaggtggtggtagcacatactacgcagactccgtgaagggccggttcaccatctccagagacaattccaagaacacgctgtatctgcaaatgaacagcctgagagccgaggacacggccgtatattactgtgcgaaagattactacggagggtggttcgacccctggggccgtggcaccctggtcactgtctcgagtggtggaggcggttcaggcggaggtggctctggcggtggcgctagcgaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctcgaacttttggccaggggacacgactggagattaaacggacc

SEQ ID NO: 76 - DNA, 1407 bp, Synthetic construct
  source 1..1407: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcaggtattagtgaaggtggtggtagcacatactacgcagactccgtgaagggccggttcaccatctccagagacaattccaagaacacgctgtatctgcaaatgaacagcctgagagccgaggacacggccgtatattactgtgcgaaagattactacggagggtggttcgacccctggggccgtggcaccctggtcactgtctcgagtggtggaggcggttcaggcggaggtggctctggcggtggcgctagcgaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctcgaacttttggccaggggacacgactggagattaaacggaccgcggccgcaaccacaacccctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgc

SEQ ID NO: 77 - DNA, 9365 bp, Synthetic construct
  source 1..9365: mol_type = other DNA
cgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacccgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatactagtattatgcccagtacatgaccttatgggactttcctacttggcagtacatctacaaaccccgggtgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtagcggccctaatacgactcactatagggcccgcgtacgtctagacctccaggcatcgattccaccatggcactccccgtcaccgcccttctcttgcccctcgccctgctgctgcatgctgccaggcccgaggtgcagctgttggagtctgggggaggcttggtacagcctggggggtccctgagactctcctgtgcagcctctggattcacctttagcagctatgccatgagctgggtccgccaggctccagggaaggggctggagtgggtctcaggtattagtgaaggtggtggtagcacatactacgcagactccgtgaagggccggttcaccatctccagagacaattccaagaacacgctgtatctgcaaatgaacagcctgagagccgaggacacggccgtatattactgtgcgaaagattactacggagggtggttcgacccctggggccgtggcaccctggtcactgtctcgagtggtggaggcggttcaggcggaggtggctctggcggtggcgctagcgaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctcgaacttttggccaggggacacgactggagattaaacggaccgcggccgcaaccacaacccctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgcggatcaggagcgactaacttctctttgttgaagcaagcgggtgacgttgaagagaatcccggtccatccgggggatccatgcggatctccaagccccacctccgaagcatcagtatccagtgttatctgtgccttcttctgaattcccattttttgacagaggcaggaattcatgtattcatattgggttgtttctccgcggggcttcctaaaaccgaagctaactgggttaatgtcatttcagatctgaagaaaattgaagatcttattcaatcaatgcatatcgacgcgaccttgtacacggagagtgatgtccacccgagctgcaaggtaacggccatgaaatgcttcttgctggaattgcaggtaattagtcttgagagtggagatgcgtctatacacgacaccgtagagaatctcattattttggcgaacaattctctcagcagtaacggcaatgttactgaatcaggatgtaaggagtgtgaagagctggaagaaaagaacattaaggaatttttgcagagcttcgtacatatcgtccaaatgttcataaatacaagctaagtcgacggccggccgcgagcatcttaccgccatttatacccatatttgttctgtttttcttgatttgggtatacatttaaatgttaataaaacaaaatggtggggcaatcatttacattttataggatatgtaattactagttcaggtgtattgccacaagacaaacatcttaagaaactttcccgttatttacgctctgttcctgttaatcaacctctggattacaaaatttgtgaaagattgactgatattcttaactatgttgctccttttacgctgtgtggatatgctgctttaatacctctgtatcatactattgcttcctgtacggctttcgttttctcctccttgtataaatcctggttgctgtctctttatcaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcctggtacctttaagaccaatgacttacaaggcagctgtagatcttagccactttttaaaagaaaaggggggactggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagacttttgcagagaccaaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccc

SEQ ID NO: 78 - DNA, 360 bp, Synthetic construct
  source 1..360: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagatctcagctggtacggagccaactttgactactggggccagggcaccctggtcaccgtctcgagt

SEQ ID NO: 79 - DNA, 330 bp, Synthetic construct
  source 1..330: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccggtcatgtacacttttggccaggggaccaaggtggagatcaaa

SEQ ID NO: 80 - DNA, 735 bp, Synthetic construct
  source 1..735: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagatctcagctggtacggagccaactttgactactggggccagggcaccctggtcaccgtctcgagtggtggaggcggttcaggcggaggtggctctggcggtggcgctagcgaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccggtcatgtacacttttggccaggggaccaaggtggagatcaaa

SEQ ID NO: 81 - DNA, 1413 bp, Synthetic construct
  source 1..1413: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagatctcagctggtacggagccaactttgactactggggccagggcaccctggtcaccgtctcgagtggtggaggcggttcaggcggaggtggctctggcggtggcgctagcgaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccggtcatgtacacttttggccaggggaccaaggtggagatcaaagcggccgcaaccacaacccctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgc

SEQ ID NO: 82 - DNA, 9371 bp, Synthetic construct
  source 1..9371: mol_type = other DNA
cgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacccgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatactagtattatgcccagtacatgaccttatgggactttcctacttggcagtacatctacaaaccccgggtgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtagcggccctaatacgactcactatagggcccgcgtacgtctagacctccaggcatcgattccaccatggcactccccgtcaccgcccttctcttgcccctcgccctgctgctgcatgctgccaggcccgaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagatctcagctggtacggagccaactttgactactggggccagggcaccctggtcaccgtctcgagtggtggaggcggttcaggcggaggtggctctggcggtggcgctagcgaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccggtcatgtacacttttggccaggggaccaaggtggagatcaaagcggccgcaaccacaacccctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgcggatcaggagcgactaacttctctttgttgaagcaagcgggtgacgttgaagagaatcccggtccatccgggggatccatgcggatctccaagccccacctccgaagcatcagtatccagtgttatctgtgccttcttctgaattcccattttttgacagaggcaggaattcatgtattcatattgggttgtttctccgcggggcttcctaaaaccgaagctaactgggttaatgtcatttcagatctgaagaaaattgaagatcttattcaatcaatgcatatcgacgcgaccttgtacacggagagtgatgtccacccgagctgcaaggtaacggccatgaaatgcttcttgctggaattgcaggtaattagtcttgagagtggagatgcgtctatacacgacaccgtagagaatctcattattttggcgaacaattctctcagcagtaacggcaatgttactgaatcaggatgtaaggagtgtgaagagctggaagaaaagaacattaaggaatttttgcagagcttcgtacatatcgtccaaatgttcataaatacaagctaagtcgacggccggccgcgagcatcttaccgccatttatacccatatttgttctgtttttcttgatttgggtatacatttaaatgttaataaaacaaaatggtggggcaatcatttacattttataggatatgtaattactagttcaggtgtattgccacaagacaaacatcttaagaaactttcccgttatttacgctctgttcctgttaatcaacctctggattacaaaatttgtgaaagattgactgatattcttaactatgttgctccttttacgctgtgtggatatgctgctttaatacctctgtatcatactattgcttcctgtacggctttcgttttctcctccttgtataaatcctggttgctgtctctttatcaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcctggtacctttaagaccaatgacttacaaggcagctgtagatcttagccactttttaaaagaaaaggggggactggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagacttttgcagagaccaaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccc

SEQ ID NO: 83 - protein, 69 aa, Synthetic construct
TTTPAPRPPTPAPTIASQPLSLRPEACRPAAGGAVHTRGLDFACDIYIWAPLAGTCGVLLLSLVITLYC

SEQ ID NO: 84 - protein, 207 aa, Synthetic construct
ACCACAACCCCTGCTCCAAGGCCCCCCACACCCGCTCCAACTATAGCCAGCCAACCATTGAGCCTCAGACCTGAAGCTTGCAGGCCCGCAGCAGGAGGCGCCGTCCATACGCGAGGCCTGGACTTCGCGTGTGATATTTATATTTGGGCCCCTTTGGCCGGAACATGTGGGGTGTTGCTTCTCTCCCTTGTGATCACTCTGTATTGT

SEQ ID NO: 85 - protein, 41 aa, Synthetic construct
EPKSPDKTHTCPPCPIYLIIGICGGGSLLMVFVALLVFYIT

SEQ ID NO: 86 - protein, 123 aa, Synthetic construct
GAGCCGAAAAGTCCGGACAAAACTCATACATGTCCACCATGCCCAATTTACCTCATAATCGGCATTTGTGGAGGAGGCTCCCTTCTTATGGTGTTTGTTGCTCTGCTGGTATTTTATATTACC

SEQ ID NO: 87 - protein, 36 aa, Synthetic construct
EPKSPDKTHTCPPCPFLVIIVILSALFLGTLACFCV

SEQ ID NO: 88 - protein, 108 aa, Synthetic construct
GAGCCGAAAAGTCCGGACAAAACTCATACATGTCCACCATGCCCATTCCTTGTTATCATAGTAATTTTGTCCGCGCTTTTTTTGGGGACCTTGGCATGTTTCTGTGTT

SEQ ID NO: 89 - DNA, 336 bp, Synthetic construct
  source 1..336: mol_type = other DNA
agggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgc

SEQ ID NO: 90 - protein, 112 aa, Synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPR

SEQ ID NO: 91 - DNA, 336 bp, Synthetic construct
  source 1..336: mol_type = other DNA
agagttaaattttctagaagcgcggatgcgcccgcatatcaacaaggacaaaatcagctctacaacgaactgaacctcggacgcagggaagaatatgacgtattggacaaaaggaggggaagagatcctgagatgggaggcaagcctagacggaaaaacccgcaagaaggcctctataacgaactccagaaagacaagatggcggaagcatattcagaaataggcatgaaaggagagcgcagaaggggaaagggacatgacggcctgtatcagggcctttccacggcaacaaaggatacttacgacgcttataggcatcaggctctacctccgcga

SEQ ID NO: 92 - protein, 69 aa, Synthetic construct
ILVIFSGMFLVFTLAGALFLHQRRKYRSNKGESPVEPAEPCHYSCPREEEGSTIPIQEDYRKPEPACSP

SEQ ID NO: 93 - DNA, 207 bp, Synthetic construct
  source 1..207: mol_type = other DNA
atcctcgttatattctcaggaatgtttctggtgttcaccctcgcgggggcgctttttctccatcagcggaggaaatataggtccaacaagggggagtcccctgtggaaccggctgagccgtgccactactcctgtccgcgcgaagaggaagggtctacgattccgatccaagaggattataggaagcccgagcctgcgtgcagtcct

SEQ ID NO: 94 - protein, 80 aa, Synthetic construct
RSKRSRLLHSDYMNMTPRRPGPTRKHYQPYAPPRDFAAYRSKSRQTPPLASVEMEAMEALPVTWGTSSRDEDLENCSHHL

SEQ ID NO: 95 - DNA, 240 bp, Synthetic construct
  source 1..240: mol_type = other DNA
aggtccaaaagaagtcggctgcttcatagtgactacatgaatatgactccacgccgccctgggccgaccagaaagcactaccaaccttacgctcccccgcgagacttcgccgcttaccgctctaagtcaaggcaaactcccccgctggccagcgttgaaatggaagcgatggaggctctgccggtgacttgggggaccagcagcagagatgaggacttggaaaactgctctcaccaccta

SEQ ID NO: 96 - protein, 62 aa, Synthetic construct
KKVAKKPTNKAPHPKQEPQEINFPDDLPGSNTAAPVQETLHGCQPVTQEDGKESRISVQERQ

SEQ ID NO: 97 - DNA, 186 bp, Synthetic construct
  source 1..186: mol_type = other DNA
aaaaaggtggccaagaagccaaccaataaggccccccaccccaagcaggaaccccaggagatcaattttcccgacgatcttcctggctccaacactgctgctccagtgcaggagactttacatggatgccaaccggtcacccaggaggatggcaaagagagtcgcatctcagtgcaggagagacag

SEQ ID NO: 98 - protein, 24 aa, Synthetic construct
LCARPRRSPAQEDGKVYINMPGRG

SEQ ID NO: 99 - DNA, 72 bp, Synthetic construct
  source 1..72: mol_type = other DNA
ttgtgtgctcgcccaaggcgcagtccggcccaagaagatggaaaagtctatattaacatgcctgggagaggt

SEQ ID NO: 100 - protein, 52 aa, Synthetic construct
YFLGRLVPRGRGAAEAATRKQRITETESPYQELQGQRSDVYSDLNTQRPYYK

SEQ ID NO: 101 - DNA, 156 bp, Synthetic construct
  source 1..156: mol_type = other DNA
tacttcttgggccgactggtacccaggggacggggggccgcagaagcggctacacgaaaacagagaatcacggaaacagagtcaccttatcaggaattgcagggccagaggagcgatgtttatagtgaccttaatacacaacgaccgtactacaaa

SEQ ID NO: 102 - protein, 61 aa, Synthetic construct
NRRRRRERRDLFTESWDTQKAPNNYRSPISTSQPTNQSMDDTREDIYVNYPTFSRRPKTRV

SEQ ID NO: 103 - DNA, 183 bp, Synthetic construct
  source 1..183: mol_type = other DNA
aataggagaaggcgacgcgaaaggcgagatttgttcacggagtcatgggatactcagaaagcgccgaacaattaccgaagccctatttcaacgagtcagccaacaaatcagtctatggatgatacaagggaagatatatatgtaaactacccaacattcagccggcgccccaaaacgcgagtt

SEQ ID NO: 104 - DNA, 126 bp, Synthetic construct
  source 1..126: mol_type = other DNA
aagcgcgggagaaagaagctcctgtacatcttcaagcagccttttatgcgacctgtgcaaaccactcaggaagaagatgggtgttcatgccgcttccccgaggaggaagaaggagggtgtgaactg

SEQ ID NO: 105 - protein, 42 aa, Synthetic construct
ALYLLRRDQRLPPDAHKPPGGGSFRTPIQEEQADAHSTLAKI

SEQ ID NO: 106 - DNA, 126 bp, Synthetic construct
  source 1..126: mol_type = other DNA
gccctgtacctgctccggagggaccagaggctgccccccgatgcccacaagccccctgggggaggcagtttccggacccccatccaagaggagcaggccgacgcccactccaccctggccaagatc

SEQ ID NO: 107 - DNA, 255 bp, Synthetic construct
  source 1..255: mol_type = other DNA
ttgaggaaaagaagagattccctatctttgtctactcagcgaacacagggccccgcagagtccgcaaggaacctagagtatgtttcagtgtctccaacgaacaacactgtgtatgcttcagtcactcattcaaacagggaaacagaaatctggacacctagagaaaatgatactatcacaatttactccacaattaatcattccaaagagagtaaacccactttttccagggcaactgcccttgacaatgtcgtg